SEQUENCE LISTING (44 sequences)

SEQ ID NO: 1 - DNA, 1435 bp, Homo sapiens
ggcggggcctgcttctcctcagcttcaggcggctgcgacgagccctcaggcgaacctctcggctttcccgcgcggcgccgcctcttgctgcgcctccgcctcctcctctgctccgccaccggcttcctcctcctgagcagtcagcccgcgcgccggccggctccgttatggcgacccgcagccctggcgtcgtgattagtgatgatgaaccaggttatgaccttgatttattttgcatacctaatcattatgctgaggatttggaaagggtgtttattcctcatggactaattatggacaggactgaacgtcttgctcgagatgtgatgaaggagatgggaggccatcacattgtagccctctgtgtgctcaaggggggctataaattctttgctgacctgctggattacatcaaagcactgaatagaaatagtgatagatccattcctatgactgtagattttatcagactgaagagctattgtaatgaccagtcaacaggggacataaaagtaattggtggagatgatctctcaactttaactggaaagaatgtcttgattgtggaagatataattgacactggcaaaacaatgcagactttgctttccttggtcaggcagtataatccaaagatggtcaaggtcgcaagcttgctggtgaaaaggaccccacgaagtgttggatataagccagactttgttggatttgaaattccagacaagtttgttgtaggatatgcccttgactataatgaatacttcagggatttgaatcatgtttgtgtcattagtgaaactggaaaagcaaaatacaaagcctaagatgagagttcaagttgagtttggaaacatctggagtcctattgacatcgccagtaaaattatcaatgttctagttctgtggccatctgcttagtagagctttttgcatgtatcttctaagaattttatctgttttgtactttagaaatgtcagttgctgcattcctaaactgtttatttgcactatgagcctatagactatcagttccctttgggcggattgttgtttaacttgtaaatgaaaaaattctcttaaaccacagcactattgagtgaaacattgaactcatatctgtaagaaataaagagaagatatattagttttttaattggtattttaatttttatatatgcaggaaagaatagaagtgattgaatattgttaattataccaccgtgtgttagaaaagtaagaagcagtcaattttcacatcaaagacagcatctaagaagttttgttctgtcctggaattattttagtagtgtttcagtaatgttgactgtattttccaacttgttcaaattattaccagtgaatctttgtcagcagttcccttttaaatgcaaatcaataaattcccaaaaatttaaaaaaaaaaaaaaaaaaaaaa

SEQ ID NO: 2 - DNA, 1316 bp, Homo sapiens
gggacagggggagccctataattggacaagtctgggatccttgagtcctactcagccccagcggaggtgaaggacgtccttccccaggagccggtgagaagcgcagtcgggggcacggggatgagctcaggggcctctagaaagagctgggaccctgggaacccctggcctccagactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaaaaaaaaaaaaaaaa

SEQ ID NO: 3 - RNA, 23 nt, Artificial sequence
  Synthetic oligonucleotide
auaaaaucuacagucauaggaau

SEQ ID NO: 4 - RNA, 21 nt, Artificial sequence
  Synthetic oligonucleotide
uccuaugacuguagauuuuau

SEQ ID NO: 5 - RNA, 23 nt, Artificial sequence
  Synthetic oligonucleotide
ggcucgaaccagcucuugaggcg

SEQ ID NO: 6 - DNA, 23 bp, Artificial sequence
  Synthetic oligonucleotide
  misc_feature (2)..(23): bases at these positions are RNA
tgcucgaaccagcucuugaggcg

SEQ ID NO: 7 - RNA, 21 nt, Artificial sequence
  Synthetic oligonucleotide
ccucaagagcugguucgagcc

SEQ ID NO: 8 - RNA, 21 nt, Artificial sequence
  Synthetic oligonucleotide
ccucaagagcugguucgagca

SEQ ID NO: 9 - DNA, 23 bp, Artificial sequence
  Synthetic oligonucleotide
  misc_feature (2)..(23): bases at these positions are RNA
tuaaaaucuacagucauaggaau

SEQ ID NO: 10 - RNA, 21 nt, Artificial sequence
  Synthetic oligonucleotide
uccuaugacuguagauuuuaa

SEQ ID NO: 11 - DNA, 30 bp, Artificial sequence
  Synthetic oligonucleotide
  misc_feature (1)..(21): bases at these positions are RNA
uccuaugacuguagauuuuaatactactac

SEQ ID NO: 12 - DNA, 23 bp, Artificial sequence
  Synthetic oligonucleotide
  misc_feature (2)..(23): bases at these positions are RNA
tuaaaaucuacagtcauaggaau

SEQ ID NO: 13 - DNA, 21 bp, Artificial seqeunce
  Synthetic oligonculeotide
  misc_feature (1)..(9): bases at these positions are RNA
  misc_feature (10)..(21): bases at these positions are RNA
uccuaugactguagauuuuaa

SEQ ID NO: 14 - DNA, 33523 bp, Mus musculus
ttacctcactgctttccggagcggtagcacctcctccgccggcttcctcctcagaccgctttttgccgcgagccgaccggtcccgtcatgccgacccgcagtcccagcgtcgtggtgagccaaggggactccagcagagccccacagccgggccccatgcgcccggtggcacagtgacgcaggccggagccctgaggctcccgaggcccgctctgggcgctggggcggccgggactctttgatggcgggggcggtcagggattcctggagtctggctagatgccccatgagggcggcgggcaggacgggatggcggggttcgggggtggggatgcccctccagtgagccccgacagagagagagggcaggctgcgtgtggaccaggaaggtggcaatggcgttggacttgacatgtgcggccggcggacgcgcgcggtggcaagcggcggagggcggcttcaggaaggatcaggcgcggggagggcgggggagctctcctgggagaggcccagctctacaagtttggggtttggcggccagggaacacctctagaaggagcttcggttcccaagacgaccgcatgagagggcatgttcaggcctggactttgcccctaattctgcagatgccataattcatatttgtgggaatgcgcaatcactccgttgaatcagcgctgcggcccaggcagtgaaaatcacgcctattttccacctgaagagtggaaaggcggctcgttttttgttttgttttgttttgtttttttacccccccatggcccagaataatgggctttggcttcagatacacttgacttgggagcccatgcccttggatgctgtgggaatttaaaaaaaaaaaaaagtatggatttggttgttacgttggatgacttttttccacactcatcttcagatatcctgccttttctcccattggctcaattttttctaacctgccaagctctgtctcccctatcaatcccaacgccataaccttgctgatcatgcttgctagaacatcccatagctcctgcttctgattaacgactttagcctgctctattcttattcttgcctctggtactccttaaatgtcatattatgctttatatactacagatttagcaggtatctggttacttgttggttgactttcccactcacacgtactttatcttgatgttttcaggcagggtcttactatgtagctctgtctggtctgtaattggtaatgtagaccatcaagctggccatgaacttgtgacagtcatcctgactcttgcctcccaaatgttgcatcactgagtcttgctggttttttaaaaattcattttagggaatgaacccttgttctactgggtaagcaagctctctactctactgggccaaaagccaggcccctaaacaagcctcagctttaggtatagcacaatgtctggtttgtggcaagttttcactaatagtttgttgggtgaattaataaccatttgagcccttaaaaaggtgttctctcatagtgtattagatattgtggcttaaggcaaaaccactgtgttcttaatgacccagaatcctgcttgcttagaatatgttctatgtggccttcagtatcttcacctagactgaagcttccttttcagacatttcttcctgtgcaaccattgccctaagtagaccaggccttgaactttcagccatcttgcttctacctcagtgtgggaattacaagcaagcatatgctatcaccaggttgtacatttttcttacacctgagatgctggctgctggtattgttaaaaggactgtaagataataaacaacacttatatagtccctgagtgggaatagcataatagccatcattttgtttacatatatctatccttattagttttcaaaataatcctgtaacaaagggcatgttcacattatcagacacaagttattaagagggtagagagctcccctcaattatgtactaaaccctagttttgatcaccagcaccagaagcaaggatacattttagaaaatggagttctgtctcaattcttggtctgtgtctgaggtctatgcaaataaatactatggtaggattcagtagataatgtagtaggtgctacttttattcggatatggctgcttttaagcttggagatatggtacttgttcagtgtagagttaattagttctttgtgctagaagtggctattcttagttttgtttgaacattaagaaattcacagtatttttagtttgtaaattcctgttcctttttttttttttttttgcttccttcccctaaattgtctgcatgctaggaataacaaacacttaaaaagtgcttactacatgctgcttgctcttctaagcccgttagcagatttgacttcttttatttctattttttttcctgtatatggctgctttgcctatatgtatgcatgtatgccatgtgcttgctttggagtccaaaagagctgccataggggttatgctcatggattggcaggatcaatatagtaaaaatggctatcttgccaaaagcaatctacagattcaatgcaatccccatcaaaattccaactcaattcttcaacgaattagaaagggcaatcggcagattcatctggaataacaaaaaaactaggatagcaaaaacctaggtcccctgaagaacaatacgtactctgagccatccttcaacccctacttgactccatcttttaaaaaaattacacagacaaggtaaataacttgcccatatcagtcagtgctaccacatttgaacctgttaatctggccccagattctacccttaaccagtagacttgttaggagctacagtgaatgtttatgaaatacttgttctgtcttcagcaatgacaaatgttttgtggactttacctaataccaataagttacataataagtgccccttgtgtagatgaacatgactggggtgatacaaggtagaggaagtaaatataagctacattactttcatgaaatgggcccctagatatgcttgagagctacaaaaacgtgctgcttgtaactatcagtctagaaaaggcttctggcttcttacaaactaatgtgaaacatcagatatgttttatatggtatacacttgaacttcaaaatagcccagatgaaagtataatatactaactgaacttaaaccttgttaatgaatttttcaaaatttatagtgtcttgagacaaagacacactctgtaaattaatgtggcctgctggccttaaactaacagtccttctgccttagtcttccatgtgctagagttacaatcatgagccaccacccttggctcagatgatttttttgagatacatactctgtagttttaggctggtctcctaactcactttgtgagaataagagacacattctaccatgcttggtatgctaatgaaagtgaatctgtgttttggtgatctaacttgaggtcttgtgctaataagcatgtttcaccattaagctatacctttatctatctgctaatttttttcagtttttaaagatttcttgttatgtgtatgagtgttttgcctgcattatatgtgtgtgtgtaccattaggatgtctgtggatgtcagaagaaggcatttcatctcctggaactggagttacagactattgtgagcctccacgtgggtttggggaactaaatctagatcccctgcaagagcaagtacttttataaaattaattctttttaattcactttatgtcctgatcaccctccctcctctctccccagtcctaccctaccatccttacaaatccctccccctccctcagagaaggggagcccccactttgctcataccccactctgggcatctagtcccagcaggtctcccacggaggcctaaccaggcagtctaagtactcctaaccattgagccgtctttctagcccatggttttttttttctctcagaattggttaaatattgtagtttttgcctaacactattttattattatcaatggtgtctaaaataacatccatttataaataagaccaaaatgaattattttgatcaccaaaaaatgggaatagtccctcttagaggtttctttatgtaaattttatactcttgttacctctgttatatgtctatcttatttggataaaaataatctggattattgtagcaggtgggaaaaagacttaattatatcagaaaaaagatgccttacagttttattattgttttgggtggggttctttttgtgagggattagtttactttatttagtctgggatatattttctttatataggccaggcttgtctcagactgggaatccgttttagtctcttaagtgttgggattatgagcatagactgctatgcctagtcttgtagtttttttttttttttactttgtaaattttagatgaaagaaaaaacagctaacttattttattaatgactatcatgaattgaattttcacttttttatttttgtgcaattggtagttcagagacctttacagtttttttttaaatttcttttccttttttttctttttggtttttcaagacagggtttctctgtatagccctggcagtccttgaactcactctgtagaccaggctggcctcgaactcagaaatctgcctgcctctgccttccaagtgctaggattcaaggtatgctatgagtcacaacacttgacaaattaaatggaattatatcattttccccttcccttttctccctccagttcttcccatttgcccaccctagaatctttttctttgattatcattgttacctacacatgtgtatgtatgtatgtatgtatgtatgtatgtatgtgcacatatacttgtttgtgtgtaaatgggttcaaggttgctcactccgcaatggatagccaaaaagggagctcttcctgggcgtgactaattcttctcacagttgtcattggttgcctgtagttctttgtctaggggtgggaccaagagaaatttctcaaacctggaatcctcctgccttagtctcctgactaacttgccttagttgattctgtaattctctgatgtagatccttaggattaaattcagattaagtttggcagcaagttcttttatccaccctaccgtctcagcagcctgagatgggatctggctgtgttgcccaggctagccctgccttaattttccaggtacttaggactacaggtgtatcaccctattcccagtggaatgtgttttattccataattaattagattaaacaatggtccatgtcatttttctcccctatagtaaaggggtattgaaattgtgtttgttctgtctttctgtaattttatgttaggtggatttactaacattgatagatcacatagaagtgttaattactaccagtaatagtaatattgagatacacttgttattttcctgttgaggactaccaaaataactaccatttaaataatttgttactagtaggaattcagacacttcaaattgctttgaaaactactttcagttttttcttgctgtttagtgtcaaaatatctatgattctactatgcatattagtaaagggaaagattttttttttttttttttttttggtttttcaagaccgggtttctctttatagccctggctgtcctggaactcactctgtagaccaggctggcctcgaactcagaaatccacctgcctctacttcccgagtcctgggattaaaggcgtgtgctaccaccgcccggcaagatttttttttttaattcatgtccttacagatatggtagttacaggcctacttgcctagagtggttataacttattcctgggctgagtagtctactaccttattttaatttttcttatagctttaacccttggaaaggaaacaaatactctatgtatgttatttgtaggactgataaaaaaaaagaaaatgaacatagtaccattttaagttgttttcatatttaggagtatctatcaataaattataatttttgtttaatcatatccagacataatataaattatctcaaggatagcttatcatctgctttttttgagattttttttaaaaagttttatttgtttttgaaaaattttaatatatttgaattaagctagtgtctcactgtgtagccaagaactagtagttttctgtctcagacttctaaatgctgagtttataggtatgtgccaccatgcccagcacaaatccctattataaaatgtgcacatttgtaataatggcaatcacatgaaagacaatccttgtgatttacacataaaatttcctgttacacaggtacattatcagaaataatatgaaaaaggaaatgaacctaagctaatacgacctttggaatgtcaggttattgtctacaatttggattgtttaagtctctttatagtaaaatataacattgttcatgagcccaaacaagactccattaaatggaagtgagtcctaaaatgactacttgggagacagggggagaaggttcagctacatagtgagttcaaagccagcctgagctcatgagactgtctcagagaagaaaactattgtgaattaagagttgtatagtaccaggcagtggtggtgcatgcctttgaacccagcacttgggaggcagaggcaggcagatttctgagttcgaggccagcccggtctacagagtgagttctaggacagccagggctatacagagaaaccctgtctcgaaaaaccctccaaaacaaaacaaaaaatccccaacaaggacaggaagagttccaagccagcccgggtcacatagtgttgagacaggaaagggtgtgtgtgtgagagagaataatagatagtagaaaaattgttttgaattttatgactagtaatcagcatgttcaaagttttagaatggacagaaatgtgaccgtcacatctgtgcacctgtgttcacacatgcatgaccacataaatacacacacacacacacacacacacacacacacaatctcaagtgaagagttgtaagactttttgcattttggagaagaattatgttgatatggctttaataaactgggtatgatggttcatacccataaacccatcatatgagaagccgaggcagaaagattgccccgggtttgaggatagcttagactatgtcgtgagattaagtttatggtgtactgcagagccaaggcaatctgaaatcagaaacaaattttttaatgttaaaataactataagcatttcagaggaacaaaacaaaaattctcagatatgaatttgttttaattagaaatcaagacttttgcattggtgtgttgatggaaatagaattcctatttttgagtaattcctgatgtttgtattcttagactacttgggtgcacatctgtaatcccagcactcagaggcagagggaggaagatcatagcaagtttgaggccaacctaatctatatagctagttccagtacagctagagttacatagtaagaccctgtctcaaacacacataaaaagactgggatgaacttggcagagttcacattggatttgtctttaattgaaacagtctttgaagttcttaggtaggttacctgtagaacctgtaaccaggcattgagctattttagtaggtgcagtagtctgatgaaacctgggtgtgataggcttaaggcagaaacagagtgagtgggcagagggtatgagaatgcctcaggaaagtcattgtggtattctaattttgattctaaggtactttgaaataatccagctttatttttgtcaggtagtacataagttctcatgagcctggcctgaatatttattgttgttatcttataaaattgagcagtctgtcatctttccccaaaacatgaactaaaatttgctttatagaacttacaaaaatgctttttttgtaaaatactctttgaatattcctttgtatgatacaaatgcccaatttctatttttgttactaaatgtcctaatataaatattaagtgaatgttaagcaggtttttttagctctctaattttttaaagaaattgccataatgtttcacaaatttttctgattatgaaaaaaaatacatgtgtatttaagaataaatcagtattctgcatgctgcaggttcttgtgaacagacctaaaaataaataaaattctatcaaacattgagctcaaaatagaccttttgtgactaaacagagagacagacagactatacaacttttaatttttttctctgggagaaacctcaaaatatatctgaattctgagctgggtgttgacagtcccagtacttggaaagtagaagaagggttgtgagtttgaaaccagcatgagctatgcaatgcaattccatctcaaaaacaatggcaagtgaattctgcaaagaacgtaaacttatgtaattttaatttttttcaaagtcaaaccacttgtgacgaaagcacctaataaaacgttaactgaaaggaagaactctgtatgaattcacagatatgatgaaatttagttatgatttggccaatattccttgaaaactgccagagcgtagacaaccaggtcttgtagtctattcggtttagaggattttttgggatgggggcaggagctctctatgtaggtcaccttggcctttaactcgtgattatccctgcctttgtacccgagtgctggcattagagatgtgtgacaccatgtgcagcttgatgctttagttcttacaacaccgtgccagggactgctgcttcttagatggggcttaaaagttgggaatgtcatgtaagattgagtagtctaattcttcctggcttatttctctcaataagtttttttaaaaagatttttatttattattatagataagtacactgttggctgtcttcagacacaccagaagggggcatcagatctcattccagatgattgtgagccaccgtgaggttgctgggatttgaactcaggaccttcggaagagcagtcagtgttcttacccgctgagccatctctccagcccctctcattaagttttgattaatagattaacttatgatcttttaagcttttcttgtagtttagtttttgttttgcttttagtgtaaagagggctggagatgagcttggttgctggagtgcttgcccggtatgcatgaaaccctgggtttgacacctagcatcgcataaactgggtgtgatatactcatgctcataatctaaaagaaggagaagcttcagaagttgaaggtcaactgcatagtgagttttaggccagtttaggatccatgacacccccccgccccccatatctcactataattcactgtcaaatggcttttatgatttggagtttggggatacttttaaagacagggtcttggtagctcctagatggcttgggattcacttagtaacccttgctggcctaaaattcaggaacttctacctcatcttcccaagtactgggttagtagggatgtccagcatatttagcttgctttaatgattttgctgtacttttttctgtgtgtacaggtgcatgtatatttataaaactaacataatatattttacccccaaagtaccataaccctaattaatgctattaaatgttttgtatgctgggctctgggcatactaatataaccaatattatacaatgcaaaacaattttggaatatcttctgtaaaattatcttattagtaagttgtaaccatctcagacttctaagtttgagtttgtaaaggatcagttttttttggtgacctacacacacacacacacacacacacacacacacacacacacacacacacacaggggggtggattctgagaatttagagaatttagatggttaataccagtttttatttttattctgtgtgatatttatttattaagatttatttatttattacatgtaggtacactgtatctgtcttcagacacaccagaagagggcatcagatctcgttacagatggttgtgagtcaccatgtggttgctgggatttgaactcaggacctctggaagaacagtcagtgctcttaaccgttgagccatctcaccagccccgtgatattttatttaacttactagaatatactgtgttaaaacatattttaggggattggggagatggcccattctgtaaagtgtctgccatgtcatcttcaggacctgcgtttgtaaccccagcatggagaagacagagacagggagacccctgggcttcctggcaagctagactagcaaatttggttaactaggttcaatgagagactcagtctcaaaacataagaagtgaattttttagacccaatttcaatctctgaactccatacgtatgcacatgtacctgcatataaatatatatacacacccataaacacatgcacacacaaaaaatggttcaaaagttcttaaaatgtataagagtagaaagtgtagagccgggagtggtggcgcacgcctttaatcccagcacttgggagcagaggcaggtggatttctgagttcgaggccagcctggtctacagagtgagttccaggacagccagagctatacagagaaatcctgcctcaaaaaaccaaaaataaagtgtatatactaatctttcatgcctaaaatatttatttttgccacatttttcatacctctttaatatttttctttttctctttgtgcttcaaaggaaattttgtgtcatgttttacttttcattcttgactctattcagcagtaagacgcagcatggctttcttagagaagtataatctattacaaaacggtaacatttaactgctctacataccctcttgtagctaaattcctctgttaaactaagacttattatctaaacgtaattaaatgttgatatcttataatgctccactttgaaacagctgtctttcttttgcagattagcgatgatgaaccaggttatgacctagatttgttttgtatacctaatcattatgccgaggatttggaaaaagtgtttattcctcatggactgattatggacaggttagtaagacctcgattgaagttatttttagagggtgtgtgtgtggtgtgtgtgtgtgtgtgtgtgtgtgtgtgtgtgtgtgtgtgtgtgtgtgtgtaagagagagaggagaggcatgtgtgatatatatgtgaagtgtattgcatgtacatggaatccagagaaggacattaggtgtcctactctgttgctctcttattcccttgaatcagtgtctcttgctgaacctggggcatacatactgatttggctaggctgcctggctagcaagacccagggatctgcttgtctctgccccccagtgctgacgttacaagcaggtgcaaccacatctggtttttatatgggtactgggggatctgaacttagggcctgatgcgtgcccaagtgctctcacccgccaagccacctctccagccaaagggttttttttcttgtattaattctaacttagcaactgttgagtagttgatgtttgaatataagctaagccaacttattagaataaatgttttctattttttacttgtatgtgtgagtgcctgcatgtatgtatgtgaagcatatgtattccaggatgtcagaattgtgatggattccctggaactggagttacagaccattgtgaaccactgtgtatgggtgctgggaactgaactttaggtcctctgcaaaaacagtaaggatcttaactgataaactatctttctagccctaaggctttctacttttaaagttcgtttctctttctctctctctctctctctctctctctctctctctctccctctccctccctcctcctcctcctcctctttcccccctctcgcctcctctttctctctcccctccctctctctctctcacacacacacacagacagagagaggagagagaaaggaagccttactaattaattacctagttttagacataactagtgggaatccatagtaagtaaatcaacattcttgaagagaggttagaagaattggctctcgttgtccatcgaggctgtataagattattcttctgttttctcagcttaccatacatattttctacgtagacaataccaattcaaatacactgtacgtaaagtatatgcgcacacacacacacacacacacacacacacacacacacacaagttacatattgtcatatagtactaaaatttatggactttttcatattaatacatctagagctatcatgttttttatcttttaaaaattactttacggggctacagagatgtctcagcagttaggagcactgactgctcttccagaggtcctgagttcaattcccagcaaccacatggtggctcacaaccatctgtaatgggatctgatccactcttatggtgtgtctgaagacagtgacagtgtactcatatacatgaaataaatctttaaaaaattactttaccatcagggtatatatatatatatatatatgtgtgtgtgtgtgtgtgtgtgtatgaagtatttgctatttgagagggtctcactgtttagctcagtctggcttggaacgttctgtatagcccagactggcctcaactcagcagtccccctgaaaggctctcatatgtggagattacaggtatgtgctactgtgcccagccctacatgttactttgtttttgaaatagggtatcaaatagcttagactggcttctaagtcagtgtgtagctaaggatggtctgagcttctgaacctgctacttcaactcctggtgtgccatcatggccagtttgcacagttgtgagagtagaactcaggacctcctatgtgttactagacatgttctctaccagctgagctacatcccaattcgcctacatttacttttattttttaaaagatttatttatgtatacagcactctgcctgcatgtacatctgcatgccagaagagggcaccagatctcatcatacatagttgtgagccaccatgtggttgctggggattgaactcagggcctctggaagagcagtcagtgctcttaacctctgagccatctctgtagccccctacatttattttaaagattataggctattctattcatagatgtgccataactgatttaaccagatcttcattattttgtacttagtagagtatattagagggaatgaatcttaaatacacattttgacacttgctcagctttagacaagtaaaaatcactggtcaaggaaatggtgcttagtggatgcttctgaactgtcaaattgtcctccaacaagttacatcattttatacttccaacgttatgtgagagttcatttttctttaggctcttctagtattttatatttgagaaataaaatttaaccaaaatttgccaatctagtaagcttgtttttatttgtatctttaaattttatgatagatgttatagtgtactctcctctcccctcccctcccctctcctcctcagcaggatcttgttatgtagccctaagggatctaatacttactctgtagtccaggctgtccttcagctcatgagacacaccatcatgcttggcatgtttcttgttctgaaaggtatctggttttaattttggttgagctggtgtgttggtgtatatctgtaatctcagcacaggcaagtagagacaaggattatgattacagtccagcctgggctatttagagggattctctttaatctttcctcatgccccaaaatcttacctttggtatatgaaaaatagtctccacttctgcaaaatattgctttatgaagtaagaattcccttcatagagacaaggaatgtgtcctgtaaaagtttaatgtgtaagaagtatttgttataaaagataaatattcagaatcttctttttaattcctgattttatttctataggactgaaagacttgctcgagatgtcatgaaggagatgggaggccatcacattgtggccctctgtgtgctcaaggggggctataagttctttgctgacctgctggattacattaaagcactgaatagaaatagtgatagatccattcctatgactgtagattttatcagactgaagagctactgtgtaagtataattaacttataattaaaaaaatagggccattctagttttatctatatttttttaaacttgtgcaaactatgctacataatcaacttacttgattatgtaatatatatagtaatagttggaattactattattacatatttatttttctgtgctggggtagaacccagggaactaagtactgtaataaccgagcttcattcatcctagtcatttttaatttttttttttttttttgttttttcgagacaaggtttctctgtgtagccctggctgtcctggaactcactctgtagaccaggctggcctcgaactcagaaatctgcctgcctctgcctcccagtgctgagattaatgacgtgcaccaccactgcctggcattttttttaaagttttaatttgagaccctttctcactaacttgctcacactatcctcgaactcattacgtgggctggtctagaaatttcagtcaacttgcctcagcttctcaagtagctgggattacaggcaggtgctaccacttctggccatgatcattgttgttgttgctattattattattattatttcactgatacttcatatcacaacacagaatagtaactgtagttcatgaccaacaaaaaagttgtacagtttctctgttgtcttattttgatctggattttgatttgtttcatggcactgggcaggctaatctcaaactgcatggctcaaatagtccttctgtcttaatctcccatgttctggaagtataggtatcactacacctagctgtattttcatatttaaaaaaaaacgcctaagaattgctattgaatgaggcggagagatggctcagcagtcacaagtacttgtagaggactggggtttggttactagcacctcaaagatggcttacaattatctgtagctccaattccagaggatctgtgtggtaaagctgcatgtatatgggcatgcaggtaaaactgctaaacatataaaaatagctcagtcttaagagaattctaggcctggtgaggtggctcactagtcaaaagcactggctgctcttcagaggactcaggttcaattcctagcacttacatagtggctcacagagtctgtccctttagtcccagggatctgccactctcttctgtcctgcaagggcacctggcaatcacataggaaacaaacttgcagcagaaacccccatacacataaaattaagaaaaaaagaattgctattgagacttgatgttttctcaactgtgtatgctggtggcaaatactctcactggagataattgctctgagaggaatcctaagttatttgaagcaaagccttaattattaaggttaatgattatttcttggaaatcagaaaatacttttggcctgggaccactttacctttgccagagtttgaaatgatttagagtattgacagtatgatgaaacaaaataaatgtcatctttatatgtaaaaacttcaccattcacaattatgatggtatagtttgtttcttcctttcctttccttcccttcccttcccttcccttcccttcccttcccttcccttcccttcccttcccttcccttcccttcccttccccctttttccccctttcccctttttccctttcccttcttccccccttcccctttcccctcttccccccttccccctcttccccttcccccttcccatcctttgactctctgtgcagtcccagctgtcctggaactcactatgtagaccaggctagccttgaacttagtgattcccccccttcaatcccagcactaggagtgcaccatcactgcctggttaaaatatgtatttattttaaaattaaaactgcagaaagttatagaaaaaaaagattgtctttccaaagtctactttttaaaaaatagatataattttattttatgtttctgcggtttttttgtgcacatgagtatatggatcatcttgagtgcctcatgtctgcagaggccagaagaatacatgggatcccctggaaatggagtttcaggtgtttgtgagtcaccatgtgggtgctgggaactgagcctgggtactctatgagaatagcacatacttttaactgttggaccatctctctaactcctgtcatcactttttattttgaatattaagagttttctctatatatacatgtccccacatacaactatagcttacataatcagtattagcccaggaggtatttaacaatccaagtgcaaaacagcctaaggagtccaaatatctattttattaaatgaaaaagtccactaattaaaaccactgtatttgtgtgcttttaagaaagaaactacctccagttatagttgtatggtgccatgaattatgatgtttacagaggtagtaaagtatgaatgttcatcttcaaacagtgaaatatagtgttatatatttggtgacattttttctgtccctagtgctctgtaactttcagaaaaatgaaacatttgataagtacacaaatgtgtcctattttgacttttatttcaattttataaaattctaaaatcgtcgaagaacaaaactagaccttgagagaaagggttgttagtaaacaagtaatggtttttaaaagatttattttattttatgtatatgagtacactgtagctgtacagatgattgtgaaccatcctgtggttgctgggaattgaactcaggacctctgtttgttccggcccagcttgctccaacccaaagatttatttattattatatgtaagtacactgtagctgtcttcagactcaccagaagagggcgtcagatatcattacatatgtttgtgagccaccatgtggttgctcaagacctgtggaagagcagtcagtgctcttaaccgctgagccatctctccacccacaagtaatgttttgagttttatttttaagtttgtaattatttgagtgttggatgaatctaaggtcgtgaatatgctaagcaaatatgctctaaatcctgagctcttgtttctatttagaggttgccagaaatcctagtgatggaaaaataggaatacaacttctttcttgtcactcccacttttccatctagactctaagaggtagacaaagtagaaagaaacattacggctgtatttatagaaacctgagcttctggggaaaatatatataaagtgagtgttttgcctgtatacatgtctgtgtaccatatgtgtgcttgaagtccacagagggcagaatagggtgtctggacccctagaatatagttatagactgttgaaatgctggatgctggaaattgaacctgggtcttctggaagccgttgctctgaaccaccccttcagcctcctctggggatttttggtagttgacagtagtgacacctgaatgaaggatgtgagtatactgtagctgtacagatggttgtgagccttcatgtggttgttgagaattgaatttttaagacctctgcttgttccaggcaactctgctcactcaggccctgcttgttctggcccaaagatttatttattgttatacataagtacactcactgtaggtgacttcagacacaccagaagagggcatcagatctcattgtgggtggttgtgagccaccatgtggttgctgggatttgaactaaggaccttgggaagagcagtcgggtgctcttaaccactgatccatttcaccagcccatataacaactattaaatgaaactttttttctctttatagtgtccctttaaaaggaagctgtttccttaacaaaaacttcagggaagcataagctgaaacactgaatgggactgtggaaacgtgaacatcttagcgtgcaagtgttctgtgtgctttggagttctgttgcctcggaccctatagcaatagcctttggatcacacttgcattcttagatatttattccttatgtctgtcctttgaaagaggaagtacttggcattgggctgtaataccatttttctacagcaggtgtcagtatttatcagggtttgttattactatgtagcactcagtgtaatgccatcttaactaggttcaggttttgtacagttctttttctttgtgtcattaatcagttgtgtgtgtttgaaattctgagttttaggctaaactagtttaaattaaaaagccaacatttagtttacagcttggactagtagtattttttgtttattttgttcttctccactccccttaaacatgctaggtaaacattctaccaactgagctatcattttaaaaaaaaattttgagatagggttttgaataccgaataaaaatagttttataggtagtatatttgtctgctacttggtgttactaattagtattgatccacataagaatatattagattgtgtcattctttgtaatagtttcatagtattatttatgtgtattaatttctttaagtaattcccttttaaagacatgctgggtttttttttgttttgtttttgatgactacctgatatagtgaacatttttgaatgtttcccttgcatgtttgtggatatttttgtaacactgattcttggaaatgggattgttagatcaaaataatttgcatgtttataattttaataggcattactatattttttcaaaagaggtcatattgctttctgtaatctaaccagcagcttgagtatttgtttctcctgaatgtattatcaggcttaattgctgtaaatgtggatgaatgaacagtagaatcacatgtagaatctgtcaggtggccatcagttgtctatttgtattctaatagttcttttttttaaagagatttgtttatcttgcttcatgtgtatgtgtgaatgccagcatatatgtatttacaccatatggatgcctgatacatttgagctcagaagaggggatcagatcacctgaaactggaggtataggcatatgaactgccatgtgggtgctgggaactgaacccaggtccttggcaagatcagcaagtgctattaattctgatctctctcctgtcccacctggtaggttcttattttgagcagaatttacattaggaatagtggttacagatctttttagttggagttcatgactagttttttatcctttctaagaatacaggggaaaattatatatattcctgggggctgagagatggctcagcagttaagaacactgtctcttcttccagaggtcctgcgtctgggaattgaacgaattcccagaaaccacacggtggctcacaaccatctgttatgggatctgatgccctcttctggtgtgtctgaagacagctacagtgtattcatatacataaaataaataaatctttaaaataaaatatattcttacattttcttctaatccacttatagttctattttttacatttagattttagtccatctggaatttatttttgtgcatggtgtgaggtaggaaccatgctttattttttcccaaatggataagtaattgaccaaataccatttgttgaataattcatctcttaattactgactcaaaatatcatctttgtcatatactaaattctcatgtttttgggtctgtttctggattttattgggttccttgaactttttgtcattcactgtgctggttctataccaagctaattgctgcagtttttggtatgctttgctgtcccagtagagtaagtccacactcttttaatcatcagttttctgatttaataccagcacatccaaattatagctattcccaccaactctagtgagtgttaatcactctcctttgaaacctctgtaaggttttctgctaatttctctacaagcagaaagtgggaacctaagaaggatgttctaatagactagtaatcagacctggtttctcagacagttctgaagatgacctggatatgggcatatccaggatatggacatattttatgggcatatcttcagggttgaatgtgatatagaccagctcctacacttgtgtgactcacaggaatggttcagggccagaagcagacacctgtcatgctaataaattgtcctttgtagttgagaagtacctgattgatgagtagctataatatgcccactcagaaatagatgtactctagttttagaactcttcagagcctcaaatactagtaatggtcaattaagatttttaatttttactaatctctatgtaaaatttacctagtcattcatttctagaaacctaattcttatactttataatttagaatagcataattttgtggccattagatagctaactttttaaatcttatagcttatatctaataatttgagaagcttatatatatatacacaagaatttgcatttaagtgatgttcttttttcaactagaatgatcagtcaacgggggacataaaagttattggtggagatgatctctcaactttaactggaaaggtatgtaacttgaagaggaaagaaagaacatttttataccaagaaacattagtaactgctttcaaccacttacttagaggtactttattagtaaaagtaagctggtattgtttaactcttagaagtacttgatatagattaatattatctcctggactttatagccaaatgacagcagaatataccattaaccctcacagtccatattgctaattactgctttattttggttttttcgagacagggtttctctgtgtagcccaggctgtcctggaactcactctgtagaccaggttcgcctcaaactcagaaatccacttgcctctgcccccaagtgctgggatcaaaggcatgcgccaccactcccggctttaattactgctttctataatactactttgaaactctaatttgggatccttcagaaaatatagctggagagggacagtcttttttttttaatagattcaaatgtagtttcatttgaaggtgtgtgtgcgcgcacacatgcttctctatgtgtatttgtttttctaagacacagtcttacttagagtctacaccatctttgaacatctcttatgtaagtcaggttggccttgacatggtggtcctgcttcagcctcccaaatgctgggattgcaagcataagcccccatactagactgtgtatgttttctaagcggctggttattttttacttgtaggcactgtgccatatataaatgtattaacctggttactttcacagtaatctagtgaaggcagttatatttccattttgcagatcaggaaatgaaacaatacacttacagtttcaaaaactccttgggattatgagatgtaaacaatctctcgaagtcacgcaaccaggaagtagaaaagcttgcatgctgacaccaaattctttctttcctctagcttttaggtttagtgctcactgctttcttgcactacttactcactactcactacttccttcactgaaactctgtagggaactttaacataaaatgctggtattctggaggcctgtatcagggtcacgccaaactaatgtcgctgtcagcagtgtttagaacctccaggggcgaatttgtaatctgttcgttctcgctctctgcctctctgctgtccatttggtgtacaactgccagacctctttcccttttagcatggtttgtccatatttagaagctattcgaaacctgcccttctataatccgttttcggggctggcaggggcctagccttcgaagcctgctgggatttagtttgaccttcttcacctaatggctctgtctctacacagcgctgtgatcttaaacagagctctgcttctaaaataacttctcagagcagccacaatagattctttttattgtggaaaacatcaaggaatacaaatttagtaaagttaaatcttgatgatgagattatgggtttatttaaaacattctaattttttgtggaaatttatcttatgtgtatgcaagtacagacataatgcacacattttcccattaatacttgaaacggtagtctttggatccaaaataacctttattcaaacccctttctcttgcccttctcaatgtttactactttaggtagttcatttgactactcagcctcattgtttccattattaagtagacatgtagctatttgacaaaataaactctcagtggtagcagttctctctctctctctgtattagaacaatagtctttaaaatgtcccaaatttttatatctttttaaaattttctatgcatatcctgacctttattttttgaaagatttacctacttatttaatttatttatttctatgtatgtgagtacactgtacagatggttatgagccttcatgtggttgttgggaattgcgttttaggacctctgctcactccagttggtcctgtttactctggtcaactccatttgctcggtccctgctcactcaggcccagagatttatttgttactataagtacactgttgctgtcttcaggcacatcagaagagggcaatggatcccattacagatggttgtgagtcaccatgtggttgctgggaactgaactcaggacctcgggaagagcagccagtgctcttacccactgagccatctcaccagcccctgacctttactttaaagtggctttttcctgcctatagaatgggatgcagttgtagcttgtttgccagaagttctgggcttacattcttagacctgtatagcactgtaagatactgccttctatggtaaactactttgaaacatggacatattagatgtttttcttgattataatttccttttaacgcacagctttgattctccaaatgatttacccacagcatatgtatttttaaattacacttgagttttggtagtaatttatcttgtaaagtacttaaactataggctaaaggagatgaagtatgtctttagttactttgaatacacacaataccaaggcaacttataaaagagaacatgtaattgggagcttgcttacagtttcagagggtaagtctgtaatcatcctggagggtagcctggtggcaggcaggcagcatgacactggagcagtatctagagcatacatattacatgcacattggatgcagagagtactagagactagacccggtgtggcttttgaaatgtcacttttgaagcccattcccagtatatgcctcccctaacaaggccacacctattaacccttctcaaaaacagttccaccaacaagggatcaaacattccaatgtaagagcctatggggggaatttttcattcaaactacaaggtgagaaagaattttgaattttcatggactccaatggaggctatgtactctaccttgataatcactaacttaaagtaataaagaagacttggcaattctgatgttatggttgtttggcagagtgatttgagaactatagactaacagtataaaataaatctgtccagtgagtattgtgggcatgtgaaatgtctgtcaccttatctctctaactagcaactcagaccaggcatgataaatcacaattgcaatcttactattcagtaggttgaggcagaaatggtgctgccagtttgaggacatttttggctacattatgagttctaggtcaacctgggctacagagtaaaattaactagaagattgagttggggttggggtgggtttaagggctctggtggatgtattgatatcttttcccagtttcccaaatacgcacacaaccatatactagttttagcttgatggagaaataaaaaatttgcattttatattatgttagatgcattattttaaaagacatttatacagattacagttcttggaatattagagtcaaaatacataaaaggagagctggttcaagtttttcagtttacatatgagtcactgagatcctgagaaatttcccaagtatgaatttgggtaatttcttagaatttgtttaatgatttcactgataattgggaatactgtctcacttcatatactaattttcatttctctgttttcttctagaatgtcttgattgttgaagtaagttctacgtttacttttaatatactgtctacaactttctaacttagtgtgtatggtaataaaggtaagtcgggggcgcttgtgttttctaaccctaagagaagccaggtacagtagggcatgtctggaatcccagatcctcaggaggctcatgaattcctgggttagcctgagttgcatagtgagtttaaagaccgagaaaatccttattttaaaaacaaaacaaaaagacacaaacaaaaaatatattttgtaagtgatgtgttttgctgctcatgaaaaaagttatatacttcctttttcagttttgatttttttcttttaaaactagttataactatatagagaagggtgtgtgtgtgtgtgtgtgtgtgtgtgtgtgtgtgtgtgtgtgtgagtgagtgtgtgatttcatgtgttaagttttattttatggaatgttacaagatttttaacatttcatctaaataatataaaagtatcaaatatttctggcaagtgagtggagtaaagactcaaaccgctactgtctggagcccattgcttttcaatgtgtaaaaaaccaaaagcattagccaggagcttatggattcagaatctttgtgataaaaaccatcaatttttgtttgagcaaaccttgagctgattccgatacatgctgagctttaagacccattgatttactcagaagaaaatgagaagacactgtctcattaaggccttctttggctgtcctacataaaaccgtacatttccatgtcttcatcttcctattcctttccatacttttttttttatcactgcctacatgcagactttattttttgcctgtggcttccctgtttcacatctgtctgacttgctgaactctatacatgtacgtctataccacagattgtggtcatcctcctaccttgactctattccccagttcttcaggatgcatcttaatagattctcattgtggcttcagaatatatatcagcttcattccccatcatttttcattctttatactttttgcatttcctagagcatcatttttctatcttcattttctgtccctagtagttgcttttcatttttgtaatctcttgtttcctttccactttttgtagtatttatgtttgtgcattgttttgtgtgtctgactgggtatgtatgtgtgtgcaagtatgcatgctagtgcataagaacagaggccagtagaggatgtgtgtgccatcctcttgcacttcctattctatgaggcagggtctctccctgaacctgaggctcacattttcttacataggccggaagctagcaaaccccagcaatcctcctgcctcctgtcttttcagacctgagttgtgatggaagcaggatgcctggattttgtgtgagtcctgcaatgcaaactccagctactatagttaaccactgagctacctctccagccccaatgcccctattttttttttaaacaaaagaatttttattctttcttttatgtgtggtgctagggatcaaacccagagcatgctaaataagtgctctaccaccaggcgtcacccttgactctttagggttttttgtttctttgtttgtaagaggtaggtaaatatctcaggctagactcttactccctccatcccacctgcctgtacctctcaagtcaagtgctgggattataggtatctaccatcatttgcgtctcttcttccctccctctttctcttattccatccctgctgtcttcctcccttccctccctccctctctcctttcttcttttcttcttttctcccttcagcatgatcttgtcatacaactcaagatgagtgtttttccttctatgagacctggtcttaatgtagcctcctcatctgtcctgggtctcactatgtggaccaggagcctaagccttctgaatgctatccattacaattgtgtgccaccatacctgccctccaagctggctttgaacctactgtgtaacctgtgctgcctgtgaactcataacctctctgcctcagcttttcacatgatggaattataggtgtgtgccaccattggttggtttgtatttctttttaagatctcttctcttttttcaattagttttagtctactgcattttgattatgtttgcgccctctctctactcctcccagatctctcttcccttccctacctacccaactttgtgtttctttatttaaaacccatcaacacaatttgtgctgctcctgtactcttggatgtgtgaccatccatcactagaatggtcaacccaccaggagacacatccttaaagaaagctgagtcctctagccgcagctatcaatgatagcttcctcactatgggtgggacatgatgtccacctcccccactccatgctgggaatttgtctgtctttgaccctgcacaggttgtgtgcatgcattctcaactgatgtcagttgataagtgcaaatgttgtactgtgacagaaaacattgtttctttatagttgtccaccacctctggttctttctctttaattaaaaacaaaacacctttgtgcttgcatgagtaagcatgacacagaatgcacatggaggtcagaagagcgctttgtataactgcttctctcctttaccacttacatggaatctagggattgaacttagctcactaggcttgcatggtaaaatgtacccacaaagccagctatctgggcgccgttcttttaagtaacacatctcatctccctccctacagagcaatcagtatccttcgccatcatttcttatttctttctcccccttctccactttctaaggtttattttttgaggacataggtgagtgggattacaccgtaattgccctttgtgtttgaagaggatctcactatttctctctggctgtcctggaacttgctatgtaaaccaggctgtccttgaaccatggcaggtttataaacacacacacactccacaaataaaataaacgtagctttttaaaaaaagttcatgtaggatgatagcactgtacagtttctggtgtgattggccagatgagggacaatttctccctgactatccaggggtagctctcaactacaaagatcatccctcaagtcctacgacacagccctgtcacagcatcacagctttttaaaccagcaggagagttaggtttcgagccctgatattcgttgtggtctatctgttttatggatttttatctgttttctgattttttttatggtactggagaatctagggcataatgggtgccatgtgggtgttttatcactgataaagtcattttatatagtaaaacttattacttagatgcttttaaagataatacaaagtgggctggagaaatggctcagaagttaagagcattgtctgcccttctagaggttctgagttcaattcccagcaaccacatgttgactcacacccatctatagttagatttggtgccctcttctggcctgccagcataatgcaggcagaacattatatacataataaatctttctgtaaaaagataatacaaagcaatgtttcttagagtatcaaagtttgggatgttaagagtccctatctttaatttacaaaaaaacctactatatcttttacagaattaaacctggtaatttatgcctgcaaccctacctcttaggagtctaaagtagggggatcttgagtttaaggccaccaacctgaactacatagcaagaccctgtgtgaaatacaacagaaagatttgtgtatgtgtgcagtgcttacatgtagacgttccatttactgtttttgaccttgccacatctatgcaatctagttctggggactgacattacctctgcttacttagagccagataggttacaagttccagactatctctgtgtgaattgtggtgagattttgtgtatactaaaaatgaatggcattgtgtcgtggaatactagaatattgcatttggaattgtatgtttgtaataaggatgattttttttaaattgtggtggttttatttaccattaaatgtctcttttctttttaaaaggatataattgacactggtaaaacaatgcaaactttgctttccctggttaagcagtacagccccaaaatggttaaggttgcaaggtatgtatgccactttaaagtagaatgctttccttatttagaatattaagtgatagcttctttgtaaggagacatgctttttcagctttcgttttatcattacagagtaatgtaatcttaaaactcaagacagtagcctttgtaattttattgtgtattaataagccattatagtcaattccttgtctaatacagtatatataaggccttggatctagaactacctcagatgctaaactccctagatgttcaggtccttcataaagaatgtcagatgagctgctgagatggctcagtgggtaaagtcatctgctgccaagcctgacaacctgagttcaatcctgggacgcatgtggtagacagagaggactgactccctctgacctccatgtgtgccacactcaagtaaaataagtgcaattgttaatggcaggagtgtttgcacagaatccatgcatactctcccatacactgtaatacctaccacagtgttaatacatataattacgctaaattatttagggaaatacatatctttatagtatgggttcttagaatattgtttatttatgtgtgtagtatatgtattatgtgcaggtgagtacacactcatcatgcagatatgtgaagggcagggcaggactgaatatcctgtactgccctttactctgttgcctgagacatggtctctaactgagctgtaagctcaaagttttagccatgcaggctcattggtgaggtttttgaatctatctctagccccagcgctggggtcacgaacacatacagccatgggcaactttttaatgtgaatattaggagtcaaactcaagtgcttgtgcttgcagaccaaatagtcttatccatggagccacctccccagtccctagaatgtctagtcttgatacacaagtttttagaaatggaactctcacatatatacagagctatgtaacaattgctgtttcagcatgttacttatatcaaatttatcctgagtgacatggcatatgatctgaggtccccttgtgaaattgaaataaatcataccaaggtagtaacaagtggtggacactgcacatctgaatcttctggattagagtggtgagtgaggcattctattttgtgttgtcactgaaattgtaccatgcttgcttcctttttctgaagtctcaaatcttactatatctgggtatttggcttgtgctttcatagttttctgtttttatccttggttatagtttgattctacaacagttgttatctccaccttatagataaggatactgaagcctagggaattgcccacagacacagagctaataagctatagagagaagatttgaacacagggaacctgtcacagttgatgcttctgctattttaagttgggttttgttgttaaaagtgacatcttttttgttgtgttgttgctattgctttttgctttatttttactttatagtttcatttttactttaatatctttgactgctctgtgctttttgtgtagaggagaggtcgggtcttttatttttgtcttttcctgaatgattaattggctcttaattttttttaaagactttacttatttatttattatatgagtacactgttgctgtcttcagacacaccagaagagggcgtcagatctcattacggatggttgtgagccaccatatggttgctgggatttgaacccaggaccttcggaagagcagtcagtgctcttacctgctgagccatctctccagctcccggttcttaatttttgaaaatttacacttatttattgtggcaggaatattggcacgtgccacttgcacttgtgttggtcagaggacagcctaagattctttctaccttgttggtcctgggtcggggtggaacatagtttggcagtcttgatagcaatagtctcatatacactgtgctatgtctgctgaccaattaaactagctttataaaatcgttttaaaagatcaaagtatctataaagtctttgaatctctataattttaataaaacttatttcagcaatttgtcttcatggactttttggatagatggcacaaatatacctcagtgagtttttgccatgctgaaggtgagatgtgcccagagaatacttgggtaatttttgatgatgtagttgtcctacgcaagtgtgttttggtctttccccaaactaaaaattaagtcttatttggtagtattgtaagatagcgtggtcactctagaaagcagttttttttaaaaactgcacagggaactaccagaagaccccagacgttgtgctgagtacttatccctgagaagtgaccatgttgctataaaaatctgtattggtttatagtcaggtatggtggttcacccctgtaatctcagattttggaaactgaaggcaggaggatcacactgaatttgagacaagcagcctgagctatatagtgaagacctgtcttctatctccctactcaaatacgactgttcacgtcatttgtgataatgcagaattaagaagaacaaaatttgaacaaatttgaagcacatttataccacgaaatcagcaataaataggaacagccatcaatatgcagcagttgagatgagtgttactggagctgtacagtgcttcaggaggacataaagcaggtgttcgctctttctgtttaaatgcttacctgtctcccagttttgatatgcccaagatttttaatttttttcaaggctattataatagtaggacaccaaaggcaaagcaataaaactgtatgattctacttatataaccccgtttgaaatgacagatgagaatggattaggggttaccaggggttgagagagaatacaggagggagccttgcatgagtcagagtatcttgattgtggtagtgattagactagtctctatgtgattaaaattgatgaaatctgtacaaggccgcttagttcaccaaggcccatgttctggttctgataatctgctgaaatacgacactagagacctttgtgtgtgatttttttttttcttttgtgacttcagtgaaaacaaaaataaaaattttgaaagccattaactttttttgggcgatattagggtttcaactctggttgtatgcatatatgacaagcattagataactagcatttaactttgtagttttatataattatgtaatcaaactacattcataaattcatgatttctataaataataaaggtataaatttaaaaataaaactgcttaaaacttacatttttagcaagagtcaagtagttgaaaatgtaattaactaaaaaattgagtgagtaactcagtggtcgagtacattcttaatgtgatcaaggctctgagttcagtatccactgcaaaaaattcccattggtgagccagttattacaagtcctgatgaaagagcatgtcttaccttctttagttaggattctgctctcagaagcatcttaccaggttccaggtggtgatcagtcgggggccttgaccttcatgtgacctctctgtgatttagttcttctccctttcagccttcctatgggtctgcgaagctgtccttattctgaatgcagatgatactacttaagaacactgatgttttctttgtaatgctctgtagtctttccatatgccacatgtaattttttgtaattaacagcttgctggtgaaaaggacctctcgaagtgttggatacaggccagactgtaagtagatgccctttggccaatcatttatccatcttataactgaaaaagttaactatgaaatggcttagggttactttagagaacatttgtgaggaaggtacatttgcaagaattagaattgaaagtattattttctctaaagaataaactatgattatttatttacagttgttggatttgaaattccagacaagtttgttgttggatatgcccttgactataatgagtacttcagggatttgaatgtaagtaatgcttttttttttctcgctctcatttttcaaaaacccacataaaaattgaggaaagggaagaattgttttctccttccagcacctcgtaatttgacccgactgatggttcccattagtcacataaagctgtagtcaagtacagacgtccttagaactggaacttggccaggctacggtgacacttcttgctggctgaagtagttgaacagctttaatatacaacaactgtcttattgttatttcacatgatatagtcataagtaagaaataaacgattcagtttcaattttgtaattgactctgcttcagatccctgcctcccattctggaggcaggaaggagtcttctgggtgagtttataagcatggtccaggagcagttagaaagtctccatgggcaatattacctaaagcgtattttttatttcacttacctgttttgatttcagctttatgtttctcaaagttacgtactaagacaaagaaaaagcaaactaatggactatagttctgctttgttttcaaattatgtcctcttcaagttgctggtccaaaataccttgtttggtagaaacccagacaacgtaggaggaccctttaatgctgttattacctctttagaatccgcttttctaacgagaatgtcttttattttatagcacgtttgtgtcattagtgaaactggaaaagccaaatacaaagcctaagatgagcgcaagttgaatctgcaaatacgaggagtcctgttgatgttgccagtaaaattagcaggtgttctagtcctgtggccatctgcctagtaaagctttttgcatgaaccttctatgaatgttactgttttatttttagaaatgtcagttgctgcgtccccagacttttgatttgcactatgagcctataggccagcctaccctctggtagattgtcgcttatcttgtaagaaaaacaaatctcttaaattaccacttttaaataataatactgagattgtatctgtaagaaggatttaaagagaagctatattagttttttaattggtattttaatttttatatattcaggagagaaagatgtgattgatattgttaatttagacgagtctgaagctctcgatttcctatcagtaacagcatctaagaggttttgctcagtggaataaacatgtttcagcagtgttggctgtattttcccactttcagtaaatcgttgtcaacagttccttttaaatgcaaataaataaattctaaaaatt

SEQ ID NO: 15 - DNA, 20 bp, Artificial sequence
  Primer
ctcctcagaccgctttttgc

SEQ ID NO: 16 - DNA, 24 bp, Artificial seqeunce
  Primer
taacctggttcatcatcgctaatc

SEQ ID NO: 17 - DNA, 20 bp, Artificial sequence
  Probe
ccgtcatgccgacccgcagt

SEQ ID NO: 18 - DNA, 15 bp, Artificial seqeunce
  Primer
tcgccgcttgctgca

SEQ ID NO: 19 - DNA, 17 bp, Artificial seqeucne
  Primer
atcggccgtgatgtcga

SEQ ID NO: 20 - DNA, 23 bp, Artificial sequence
  Probe
ccatggtcaaccccaccgtgttc

SEQ ID NO: 21 - DNA, 22 bp, Artificial sequence
  Primer
ttgttgtaggatatgcccttga

SEQ ID NO: 22 - DNA, 21 bp, Artificial sequence
  Primer
gcgatgtcaataggactccag

SEQ ID NO: 23 - DNA, 31 bp, Artificial sequence
  Probe
agcctaagatgagagttcaagttgagtttgg

SEQ ID NO: 24 - RNA, 23 nt, Artificial sequence
  Synthetic oligonucleotide
uaaagcacuuuauugaguuucug

SEQ ID NO: 25 - DNA, 15000 bp, Mus musculus
tctcaggggaaaggtatcctggattcagtaaccaaggaacatcacaatgtcacattgcacaacaaacctcacacaagagatttattgggagagatacaagaaggtggctgcctttccttggtggagaaacagcaggaaacggagtgggaggcaagctttcttggtggtggagttttccagggtggaaatttctagggaggggattggtaaggtttggtgagttttcaaaccctgaggtgtgtgcttggaccttacaccctacacatgcacctcatgatacaacgcccacttcccatacggagccctgtgctcaccacagtccttcttggactggctgaatttaggctcctgcataaccttgtagcccttccctcctttcctactgcaaaaccctcttagcttgctgtggtaaatacacttctgtaattcccgtaattaggaggtggaggcagggaaatagcaaagttaggtcactctagatcaagtccctcttgcaaaaacaaagaaaaaaaccacgccctctatggtacaaaccagttcctcttccctagagttctggcccctccccgggaggatgtggtcatagggtcaggtcctggggccaaggtctgtcctggtacaaccccggactgaccggtttctttcactttggtcggctctgctatgtggctcggccttgcttttgtgatggctaacgttatgatggcagttaattttatgtttaaaagtttaaaatgtctgtgcttaagaaaactataaagcgttaagagtggttaaggcttagaatttgtttttacaactattttggtttctataaatgagcttgttttgctccagaggaccctctgcttggcccagaacaaattgtaactttgtattccttgcctttataaacccttgactgaggtagctgggtgctgcattagggaatcccaacttgcaaatacggacctggatggagccagtatctgaataaaagcctcttctttgttagaatgtattaatggtcagactggcgagtctctgaatgaccccagacccataacactgtcttcacgtcctagtggcaatcaccaaaagtcccttttgaaaccaaaagttgaataagtgtcccgttgttcctccggtcttgcaaggccttgcaaagctgatcacagccttcctctcttcctggagcaccaggaacctgttatagtctgcctttattgtctataaaatgaaatcacctatgaagattccccaccctctcttccctggttgcttggctttttcttctcagactgtcccagaccagtgatagatgcccaccaacccggatcagtggtagatgcccactaacaatcccagaccctagtaagggagggtaagggggtgggggtgggggttggggctggtggcacttgcctttaatccctgcattcaggaggcagacgaaagcttatttctcagttgtagtctagagatgccatgctctggctcttgaactaagaaggtaccactggcaactctggaagcccaggagggagggagggagggagggaggaagggagggagggagggaggaagggagggagggagggagggagggagggagggagggagggagggagggttggttcctactgagcaaaataaatgctgggtagaaaaagccacagtgtcctgctccccaccttctgcccagcacttcagctgtaagacaaggcaatgccctttgatttagcacttcactctggaaagagtacaattctgagatgacagcgagatcatcatccctgtgtatttctgcctgctcccagtgacccaagttgaccgtgattgaggttattttgtaaaatatttccttgaggcaccgggaatcaaggccatgcatcctaggcgagcattctgccactgagtctcatgcttccaccgaggagccacatccccagtccttttgtaagagaatttgccgatgtaggtggccatggtggcacaggaaattagggaggctgaagcaggcaggagaatcacaattaagtcaacctgagaaacatagtgtctccagaggaaaagaatttgcaactgtgattaagggcccatattgagtctggctacacagccctgactaaccagaactcactgtgtagacagctggctggcctcgaactcatacagacctacctgcctttgcatcatggcagtaaaatcatgtaccgccatggactctcactctgtgggagtatccttgatgggcctgacccaatcggatgagccctgaaaagggaccagaagtttcctggcaaagcagacacagtgtgaagcaattcaatgcagggacattctctgctccaccctctgttttattgcttttaattcttaaaattaatttatttattctatgtacaatgttttccctcatgcatgcccatgcaccatgtgcatgcctgatacctgcagagtccagaagaggcaatcagaacccctggaattggagttatagacagctctgagccctcctgtaggtagatgggaatagagcctggatcctttggatctaaagtccatacctgctgagcgacacctccagccctgacactggctttaaagttagaggaagtaggtggccagaaatgtagaaatcacacgtagccgtggtcgacagctgagaagaagctgggacatctgtcctgcagccacatggaagtgagttctgaccaggacctggggggggatctcactttagaggctccagagacttcagcttgaacctcagcgttctgatgatctgggcagaaactctaactatgcagcaccaagtgtttacccatgggtctgtgcatccgtaaaagtagttttggggggcattgttttagggtgctcagagtgtggtaattggtcacacagcagaaggaagctgcatgaaggaaggtcacataagcattctgtaagattctgaaagcaggttttgtcccatcattcttaccttaccctcaacaggacctggaacaccatcctagtaaagtcaggaaaatggagggcaggaacctaactcttcagtccgctactcccaccagtgactccctgctcaaaacacttctacagcgctgttctgctgggtctcccatgaagcccttgctcctcagacccaggagcaagcttgaccaatctctacctctgaagcttctgagacctttgcccgcatctattgatcctcactcctgggcaggcagcggggccatcggcagacgccatgacggctctgttgttcctggggtctctgctgatgagtctggatctgacactttcggtgaggacggtggacactgggattgagccagagtggcagtgaggggtccctgttccggatttagcctctgcctgtcacctgctagttcaggtgactgagagacttgtggtgggggtgggggttgggggaaggcacagctagcccagaagatccaaacaaaggaggtagcagcccatgctggtggatgtgaggacacaggctacttttgtgggggagtcaatgagaagagaaaggaaggctacactttgtgaggaaatcctgtactttggcgaggtggcaggaggttgaggcctataggatcaagggaagtctaacattgtgggcaatgttcctgccttctctctcctataggctccaccatggaaagactccaagaaatttaaggacgcacctgatgggcccacagtgggtaagtgatctggcctcttttcctaatctactctgtggggatggctgtgctttagcctgcgtggtctcaggcctctggagcaaggagggatcctgagccaagctgttccgttgacatttaaacctgggtgcctacagattcagatgtgtgcccggagtgccaggcttgggctgtcctggatgcctgtgttagagcctggcaggtactctgtagtgttggagatgatgtcatctgatgatgtcatcacacctgcacaccaacaagtgctcgccagtttccccatcttcctccctttcctttcaacactcctggagaatgtcagacataaccggtagagttcagagcttcagtgggcgatgggagccagaacagagctttagactcacccaagatcacagacaccccaagttgtctccaaaccctaactttctcacttgtgagtttactctctgcttcatttttccctgtctgaaaagttagacgggttgctgaggagaaagggttcctcattgttgtagatgctgctgccaggaaaatgccctaacatgttagctcttaggctagggcatagctcactggcagagcacttgcccaacatgcacgcacacacacctgggtttggtctgcagcactgtgaggggaaaaaattccttttttttttaattttaaagaaaaaattctttcttaagctggagagataactcagaggttaagagcactggctgctcttccagaggtcctgagttcaattcccaacaaccacatggtggctcacaaccatctatctgtactgggatctgatgcctttttctggtctgtctgaagacagcaacacatatagtaaataattaattaattaaagaaagaacaattcttttttaaagatttatttatttattttatgtatgagtgttctatctgcatgtacacctgtatgccagaagagggtatcagatctcattacagatagctgtgagccaccatgtggttgctgggagttgaactcaggacctctacaagaacagacagtagatctcaaatgctgagccatttttccagcccaagaaaaaaaatcttaacttttagtaaatttgacttaggtgaagggttccacccccatcctgccccaaaccctgctataggacttgtgagtacagaactctaccacagtgagccttgtctgtggggtcaagctgaggcttgtagtgtgatgtctctcactgaagcccaactcagtgtgaccctccaggcttctgtcctaaccactctaagccaatcagctgctgtccctttcaacctgacccaccatggccccctgtctccaagttctaggacccttccctctctctatcaccctgttgctaaattgccactaaaaggagaaaagaagaagaagaagaagaagaagaagaagaagaagaagaagaagaagaagaagaagaggaagaaacaaaacacatttgttgagcacctactgagtcctggactcaatgtcccaagcattgccactaagcctcacggtgttcccctgaagtagacagtgatttctttttcattttctctttgagattataatataattacatcactgggcagtggtggcacatgcctttaatcctagcacttgggaggcagaggcaggcagatttctgagttcgaggccagcctgctctacacagtgagttccaggacagccagggctacacagagaaaccctgtctcgaaaacaaaaacaatatatatatatatatatatatatatatatatatacacatatatatgtgtgtgtgtgtgtgtatacgtatatatatatatacacatatatatatacacatatatatatacatatatataatcatatcatccccctccctttcttccctccaaaccctcccatagccccaataattatttctttttttttttaagatttatttatttattatatgtaagtacactgtagctgtcctcagatactccagaagagggcatcagattttgttacggatggttgtgagccaccatgtggttgctgggatttgaactcgggaccttcggaagagcagtcggcgctcttaaccactgagccatctcgccagccccaataattatttcttatgattaactatacttttctcagctaccatgaacatcaagttcacaaattcagtgattccgttcacaaattaggttccgtgagcctctggctatgacattttcaccaatcattagcatgtaatcttgttacatctggattctgtttaaagacattattcagtgtgcatgttgttgatcccttaaacactaatctaatggccgatgccactattactgcctgcctgacaaggtgtccctgacacgttttcttcataaggcgcatacaacttcttgtgttcttgcaagtaggagcctcaggtaacacttgggcatacacatagaaccactctgtctccctgaagtactgaggatcgaacctagagccttgtgtatgccaggcaaatgcttggccactgagttaaaactctggccctctttaaaaagaagtgtgtgtgtatgtgtgtgtgcaagtgcatgtgtgtatgtgtatgtacatatgtgtgtgtgtttgtgtgtgtgtatgtgtgtatatgtgtgcgtgtgtgtgtgcaagtacatgtgtgtgtatgtgtgtgtgttcgagcatctttcactgacccattcaattccataaagatcatgaaggcactgtgaacgttggcagtttctttggctttctttggccttgtgctgacttgtgagcaagcactacacgactgacctatatcctcagcctgagtcacaagtattgggttggagggggcttacaaataaatgtttacaaagcaggcaggggcagtggtgggggggtgcctttaatcccagcacttggcaggcagaggcaggtgaatttctgagtttgaggccagcctggtctacacagtgagttccaggacagccagggctacacaaagaaaccctgtcttgggggtgggggtgggggaacaaaacaaaaaacaaaacaaaacaaagcaggcaggttcacaaatataaagtcagtgaggattggcagttgcattactgttgccagtttactaagccaaaaatggccaagggacctaagtgtttgtttgtttgtttgtttgtttgtttgtttgtttgtttgttttgtagacaggctatctctgtgtaatagacctggctgtcctggaactcattctgtagaccaggctggccttgaactcacagagatccgcctgcctcagcctcccaaattctgggatgaaaggcgtgtgacctaaatctttaatgagctcaactttgtgacatgcacctgtgtttcagacttgctcagggtcagatatctattccagctgtctgactctagaagcctactttctaagcaggatgcagtcttgaatgcatagctgacctttttccaaatcaggccctgtgaggcagagggtgggcagctgggtctcagggcagggctctgaggtgtggtgttcctacagttctcactgtggatgggaggctctgccattttccctttcagtaccaccgtcagctacaccacaaatgcatccacaaaaggcggccaggctcccgcccctggtaagacattttctaataggggttgggggagggggctggatgaaatggactctgtctagctatctaagcatcgtgtttggtcaaaaggtgtgtgtgtgtgtgtgtgtgtgtgtgtgtgtgtgtttgtgtgtgtgtgtgaagcaaggctagcctagccagcctcaggcactgcagggaaacctcatctatcccccaggtgtgctaccacccccaactttgatgaagatcagcaatggggatactgcttggagcccaagaaagtgaaaggtatgtatggcatgcaggccccgggtggctcaaggctgtgtgtgtcttgctgctcactccgttcccacacacctactcacctactaagcatcatcagatgccaaacacttgggattctgggccccgcccttttccttctcttacagcattctttcaaaagtcagaggaagaattctggggaaagaggccaccattgtccagatgagcttgagggtctagaagcttggggttctatggtttgatgacaagcaggtgcaggcagggtattggtccctacatatgtccttccgactccggaagtgcttcgctcttgttatctacctctcagaccattgcagcaaacacaacccgtgccacaaaggagggacatgtatcaacacccccaatgggccacactgtctctgccctgaacacctcactgggaaacattgccagaaaggtaagaggaactgcctcccagcaagatgtccctggagacccggtgctttgccatggtcccattgacttccttgtgtccccagagaaatgctttgagcctcagcttctcaagttcttccacgagaatgagctatggtttagaacggggccaggaggtgtggccaggtgcgagtgcaaaggttctgaggctcactgcaagccggtggccagccagggtaagtgggtgtgcagggactgtggggaggagggcagagagtcaggaacccctggtagaaggctgggtgcaatgatgtacacaggtaaggctcagtttgcacctctccccaccccacccccagcctgcagcatcaatccgtgccttaatgggggcagctgcctcctcgtggaggaccacccactgtgccgttgccctacaggctacactggatatttttgcgacttgggtgagtaagaccccgtgtggaaaggcttgcggaggtggatagagagaatggaagtgaaccagagggctccaacagactcatccgccgactgcagggagccatctctctttctctagacctttgggcgacctgctatgaaggcagggggctcagctaccggggccaggctggaactacgcaatcgggtgcgccatgtcagcggtggaccgtggaggccacctaccggaacatgactgagaagcaagcgctaagctggggcctgggccaccacgcattttgccggttcgcgagaagggaccgggcaggggaacttgctttctcttagggtcctcgagggcctccccacgttctaacagtgctccctcttgagattgcaggaacccagataatgacacacgtccatggtgcttcgtctggagtggcgacaggctgagctgggactattgcggcctggagcagtgccagacgccaacgtttgcacctctagttgtccctgagagtcaggaggagtccccgtcccaggcaccatctctgtcccatgcaccaaatggttaggcagaggagggggtcccggcgcagaggacatgggtctctcttattcctggcagcccgtgccaggtatccatggcctcagccagtctctccttccacagactcgaccgatcatcagacttctctgtccaagaccaacacgatgggctgcggacagaggttccgcaagggactgtcctcgttcatgcgcgtggtgggcggactagtggctctgcctgggtcgcacccctacatcgctgcactgtactggggtaacaacttctgcgcgggcagtctcatcgccccctgttgggtgctgaccgcggctcactgcctgcagaatcggcaagtgccaccctcggtgaccccctagaccgctcctaccgtacccgcaccctactctttccctgcccgccattcttgagctccctcgaggggttggaaactaaggcacccccagagcatttgtagccggtctgagcctgctgcctgtcccccacccgactgcaggccagcgcccgaggaactgacagtggtacttggtcaagatcgccacaaccagagctgcgagtggtgccagactctggctgtgcgctcctaccgccttcacgagggcttctcctccatcacctaccagcacgacttgggtggggtggccctacagggatagggagaaaggatggcggagggctggggccctatgtcgccatctaacctttgcctctcggggtagctctgctgcgcctgcaggaaagcaaaaccaacagttgcgcgatcctgtcacctcacgttcagcctgtgtgtctacccagcggcgcggccccaccctctgagacagtgctctgcgaggtggccggctggggtcaccagttcgagggtaggcacaactgttgggcgctggttggagacttttggttatctaggagcgcagttggtacgccccgatgaatctgggggacaagtttcactgacatgacagttgtaaaaaacgcacagagcccttgtctctgtagcgtgactttcccagattctagaattctctgtcgagattccagagccccttgaggtttgttctagttttttgccttctattgtggcgttaaacaccatgaccaaatccagcttaggcaggaaaggttttatttggcttccgaggttgcagggtttcttgctgtccaccattgaggaagccacaacagggagtggaggcaggaactgaggcaggaaacggaaagggacactccttgctgcctttctcttcatggcttgttcagctttctttttaaaaaattcacttatttttattttatgtgcatgtgagtctgcctacaagcatatgtgtgcaccatatgtacatctggtacaggggaagccagatctgtaatacaggttaaaaaccacggtgtgggggctgggaacggaaccaaggccctctgcaagaacagcaagagctcttaacctctgagctagcccccaacaccttgcggcttgcttactttgctttgcttttctcttttcctcttcctcttcctcttcctcttcctcctcctcctctttctccccctcttcctcttgttttgtttttgttttttgttttgaaggcagggtttctctgtgtagtcccagctgtccgggagctgtccaggagctgtccagctctgtagaccaggctgccttcaaccttaagcactccatctgcctttgcctcccaagtgctgggattaaaggcttgcacctttctggctcagtttctttcttacacaatccaggcccacctgttcagggcggcatcactcacagtgggcggggccctttcacatcaaccattaatcaagaaaatgcccggggctggtgagatggctcagtgggtaagagcacccgactgctcttccgaaggtccagagttcaaatcccagcaaccacatggtggctcacaaccatctgtaacgagatctggcgccttcttctggagtgtctgaagacagctacagtgtacttacatataattaataaataaatctttaaaaaaaaaaaaaaagaaaatgccctacagatattgccctaggccaatcttctggaggcatctcttcgggtgaggttccttccccccctagatgactcttgagtgcgtcaaattgacagacagtaacccagcacaaggattacagggagacttgaactgtcttgtgcttcttttgggctccaaattataaaggcttaaccaggactttgtccccatgctggagcaatggagacaatggagagaactattctgggagccaggttctgagccactgtgtggaagaggataggaagtgcttcctgtgttttaagccctgctcttctctgggcttcagtgtccttgccatgaaatacttattggcaggtccctatcactcaggcttgctgtgagggagcaaagcggagtaggtggggaattgtctggtagcctggcccacgcagcaagctcaggtcctcccctctgatttgcaggggctgaagaatactccaccttcctgcaggaggcacaggttccctttatcgccctggatcgctgctccaactctaacgtgcacggagacgccattctccctgggatgctttgcgctggcttcttggagggaggcaccgatgcctgccaggtcagccctggggtcctggtaggtaccttggtccctgcctgtcaagcataaggcaagaaccacgtgctgcctgttccccacccagggtgactccgggggccctctggtgtgtgaggaaggaactgcagaacatcagctcaccctgcgcggagtcatcagctggggctccggctgtggtgaccgcaacaagcccggagtctacacagacgtggccaactacctggcttggatccagaagcatattgcttcataactaaccaggctttatccttccctccttgtgtgctccttgggatgggacgatgaatgtggcatgctgggtcacagtgaagctagtgccccgacactgggggcacagaaactcaataaagtgctttgaaaacgttcctcagaattctgtcttgaaacgtcaagtgggagcacaggtaagccaactccctcgttgcctggacaaggcaactagccagatgtcagcataagaggcgtagactcttgtccggaccaccatattttctcatccttactttgggtgagcttttgccgcccatgttcaagtccacctgaggtcaattaactggactctagggagaaggcagtcttggcatatttttaaaacgctacgggtgattccttatgcaatagaggttgggaactgtagttaagagtgttccagacgggaatcgtggtgcggatagtagctgctgagcatgtgtgagaggccctgggttagatcccagcaccaccattaaaagaagggggaggggttgtgttgggttgtgtgggttttgtttgtttgttttgaactttaagcctggcatggtggtgcaagttggtgtcaaggaaaatctgaggctgcatctgatgcctctaagtacaagctgggttaggagccagccagccagagccttcggggatgcggtgactccaaagcgaggtttgtagttcccttgcaggagaggagcctagttgtccagatgagtacatttcttgcttttaaaaatgtccttctgtttattgatttattgagatatctgtcgtcctaatgccgaaattataggcctatacaatcacccagtttatgtggtgcagggggatcaatcccaaaactttgttcttggtctagccaggccctctgtcaactgcactgtactgactgcgctacactccccgtccttacacccatctttgcacagcatcttactgcatagcttttgtctggcctgtgacttgctaggtagccttgaacttgtacagatccccttgcttcatgagtgacaggtttaagtacaagccaccatgtctggcttttttttttttttttaattaagaaaaaaaaatggggctggtgagatggctcagtgggtaagagcacccaactgctcttctgaaggtctggagttcaaatcccagcaaccacatggtggctcacaaccatccgtaatgagatctgatgccctcttctggagcgtctgaagacagctacagtgtacttacatgtaataaataaataaataaataaataaatcttaaaaaaaaaaagaaaaaaatgtggtttgtgttatgagtgtgtcacagtgtggtgtgaaggtcagagcacaactctgtggagccagttctctccttctgcctttgtgggggctctggtgataaaactcaggccaccaggcttataagacaatgcacccttactgtctgagccttttttttttttttttatggtttttcgagacaggctttctctgtgtagccctggctgtcctggaactcactttgtagaccaggctggcctcgaactcagaaatccgcccgcctctgcctcctgaatgctgggattaaaggcgtgcaccaccatgcctggcacttttttaataattaaaaacctgagaatttgggctggagagagaatgactcagtggttaagagcacggactgctcttccagaggtcctgagttcaattcccagcaatcacatgatggctcacaaccatctgtaatgggatccaatgccctcttctggtatgtctgaagacagcgacattgtactcatatacataaaataaatacatttcttttattatatctaagtacactgtagctgtcttcagatgcaccggaagagagcgtcagagctcattaaggatggttgtgagccaccatgtggttgctgggatttgaactcaggaccttcagaagagcagtcagtgctcttaaccactgagccatctctccagtcctaaatgtgtctttaaaaaaaaaaaagttaaaatttcaagacttttgagctggtgagatggctcagtgggtaagagcacccgactgctcttccgaaggtccgaagttcaaatcccagcaaccacatggtggctcacaaccacccgtaatgagatctgatgccctcttctggtgtgtctgaagacagctacagtgtacttacatataataaataaatcttaaaaaaattttttcaagacttttaaattgctgggcagtggtggcgcatgcctttaatcccagcactcggaggaagaggcaggcggatttctgagttcgaggccagcctggtctacaaagtgagttccaggacagccagggctatacagagaaaaaacaaaaaacaaaacaaaaacaaaaaaaatcaagacttttatgtgtttctgtggacatgagtgcaggtgcccaagcaagccagatgcactgctttacctgggtgctgggaattgaactctggtcctctgaaaatggcactcagttgcgggacctttttttatttatttttttattttattttattttaaatcatgtgtgtgtttgtgtgtgagcttgcctcaggagttgtcagatctctcgagtgggagttacaggctttgtaggccaaaagaggtgatgctgggaacggaactcaggctctgtgtaatgaatgctctttcttaacgctgagccacctttccagcctctcatccatcttcttccatttcctcctacatgctacttttagaaccgcccccgaacccccaccaactctaccgccctctccttctcctcctagttagatgttctgtgacgtttgccttacgtcacagccccgccctgcgaacttcgggcatgctgccagtgttcttagctctgaatgcagccggccaccccccctaatgcaagcacaagttgctgggactgactgagcggagatgagtgactggaagggctacatcagtgcagtgctgcgggatcagcggatcgatgacgtggctatcgtgggccactcggacaatcgctgtgtgtgggcatcacggcctgggggtctgctggctgccatctccccgcaggaggtgggtgtgctcaccgggccagacc

SEQ ID NO: 26 - RNA, 21 nt, Artificial sequeance
  Synthetic oligonucleotide
gaaacucaauaaagugcuuua

SEQ ID NO: 27 - DNA, 24 bp, Artificial sequence
  Primer
caaaggagggacatgtatcaacac

SEQ ID NO: 28 - DNA, 20 bp, Artificial sequence
  Primer
ctggcaatgtttcccagtga

SEQ ID NO: 29 - DNA, 24 bp, Artificial sequence
  Probe
cccaatgggccacactgtctctgc

SEQ ID NO: 30 - RNA, 23 nt, Artificial sequence
  Synthetic oligonucleotide
uuaaaaucuacagucauaggaau

SEQ ID NO: 31 - DNA, 23 bp, Artificial seqeunce
  Synthetic oligonucleotide
  misc_feature (2)..(23): bases at these positions are RNA
taaagcacuuuauugaguuucug

SEQ ID NO: 32 - DNA, 23 bp, Artificial sequence
  Synthetic oligonucleotide
  misc_feature (1)..(13): bases at these positions are RNA
  misc_feature (15)..(23): bases at these positions are RNA
uaaagcacuuuautgaguuucug

SEQ ID NO: 33 - DNA, 21 bp, Artificial sequence
  Synthetic oligonucleotide
  misc_feature (1)..(9): bases at these positions are RNA
  misc_feature (11)..(21): bases at these positions are RNA
gaaacucaataaagugcuuua

SEQ ID NO: 34 - DNA, 23 bp, Artificial seqeunce
  Synthetic oligonucleotide
  misc_feature (1)..(21): bases at these positions are RNA
auaaaaucuacagucauaggatt

SEQ ID NO: 35 - DNA, 23 bp, Artificial seqeunce
  Synthetic oligonucleotide
  misc_feature (2)..(21): bases at these positions are RNA
tuaaaaucuacagucauaggatt

SEQ ID NO: 36 - DNA, 1289 bp, Mus musculus
ttacctcactgctttccggagcggtagcacctcctccgccggcttcctcctcagaccgctttttgccgcgagccgaccggtcccgtcatgccgacccgcagtcccagcgtcgtgattagcgatgatgaaccaggttatgacctagatttgttttgtatacctaatcattatgccgaggatttggaaaaagtgtttattcctcatggactgattatggacaggactgaaagacttgctcgagatgtcatgaaggagatgggaggccatcacattgtggccctctgtgtgctcaaggggggctataagttctttgctgacctgctggattacattaaagcactgaatagaaatagtgatagatccattcctatgactgtagattttatcagactgaagagctactgtaatgatcagtcaacgggggacataaaagttattggtggagatgatctctcaactttaactggaaagaatgtcttgattgttgaagatataattgacactggtaaaacaatgcaaactttgctttccctggttaagcagtacagccccaaaatggttaaggttgcaagcttgctggtgaaaaggacctctcgaagtgttggatacaggccagactttgttggatttgaaattccagacaagtttgttgttggatatgcccttgactataatgagtacttcagggatttgaatcacgtttgtgtcattagtgaaactggaaaagccaaatacaaagcctaagatgagcgcaagttgaatctgcaaatacgaggagtcctgttgatgttgccagtaaaattagcaggtgttctagtcctgtggccatctgcctagtaaagctttttgcatgaaccttctatgaatgttactgttttatttttagaaatgtcagttgctgcgtccccagacttttgatttgcactatgagcctataggccagcctaccctctggtagattgtcgcttatcttgtaagaaaaacaaatctcttaaattaccacttttaaataataatactgagattgtatctgtaagaaggatttaaagagaagctatattagttttttaattggtattttaatttttatatattcaggagagaaagatgtgattgatattgttaatttagacgagtctgaagctctcgatttcctatcagtaacagcatctaagaggttttgctcagtggaataaacatgtttcagcagtgttggctgtattttcccactttcagtaaatcgttgtcaacagttccttttaaatgcaaataaataaattctaaaaatt

SEQ ID NO: 37 - DNA, 23 bp, Artificial sequence
  Synthetic oligonuclotide
attcctatgactgtagattttat

SEQ ID NO: 38 - DNA, 23 bp, Artificial sequence
  Synthetic oligonucleotide
cgcctcaagagctggttcgagcc

SEQ ID NO: 39 - DNA, 23 bp, Artificial sequence
  Synthetic oligonucleotide
cagaaactcaataaagtgctttg

SEQ ID NO: 40 - RNA, 23 nt, Artificial sequence
  Synthetic oligonucleotide
uuaaaaucuacagucauaggauu

SEQ ID NO: 41 - RNA, 23 nt, Artificial sequence
  Synthetic oligonucleotide
uuaaaaucuacagucauaggaaa

SEQ ID NO: 42 - DNA, 23 bp, Artificial sequence
  Synthetic oligonucleotide
  misc_feature (1)..(21): bases at these positions are RNA
uuaaaaucuacagucauaggatt

SEQ ID NO: 43 - DNA, 23 bp, Artificial sequence
  Synthetic oligonucleotide
  misc_feature (2)..(21): bases at these positions are RNA
taaagcacuuuauugaguuuctt

SEQ ID NO: 44 - DNA, 23 bp, Artificial sequence
  Synthetic oligonucleotide
  misc_feature (2)..(13): bases at these positions are RNA
  misc_feature (15)..(23): bases at these positions are RNA
taaagcacuuuautgaguuucug

(19) United States
(12) Patent Application Publication
(10) Pub. No.: US 2025/0154506 A1
(43) Pub. Date: May 15, 2025

(54) LINKAGE MODIFIED OLIGOMERIC COMPOUNDS AND USES THEREOF

(71) Applicant: IONIS PHARMACEUTICALS, INC., Carlsbad, CA (US)

(72) Inventors: Thazha P. Prakash, Carlsbad, CA (US); Michael Oestergaard, Carlsbad, CA (US); Mehran Nikan, Oceanside, CA (US); Graeme C. Freestone, La Jolla, CA (US); Michael T. Migawa, Carlsbad, CA (US); Brooke A. Anderson, San Diego, CA (US); Punit P. Seth, Carlsbad, CA (US); Eric E. Swayze, Encinitas, CA (US)

(73) Assignee: Ionis Pharmaceuticals, Inc., Carlsbad, CA (US)

(21) Appl. No.: 18/264,705

(22) PCT Filed: Feb. 11, 2022
(86) PCT No.: PCT/US2022/016143

§ 371 Date: Aug. 8, 2023

Related U.S. Application Data
(60) Provisional application No. 63/287,874, filed on Dec. 9, 2021.
(60) Provisional application No. 63/282,489, filed on Nov. 23, 2021.
(60) Provisional application No. 63/234,466, filed on Aug. 18, 2021.
(60) Provisional application No. 63/220,081, filed on Jul. 9, 2021.
(60) Provisional application No. 63/148,513, filed on Feb. 11, 2021.

(51) Int. Cl. C12N 15/113; A61K 31/712

(52) U.S. Cl. CPC C12N 15/113; A61K 31/712; C12N 2310/14; C12N 2310/314; C12N 2310/315; C12N 2310/3231; C12N 2310/343

(57) ABSTRACT

The present disclosure provides oligomeric compounds (including oligomeric compounds that are antisense agents or portions thereof) comprising a modified oligonucleotide having at least one chemical modification.

### SEQUENCE LISTING

The present application is being filed along with a Sequence Listing in electronic format. The Sequence Listing is provided as a file entitled CHEM0102WOSEQ_ST25.txt created Feb. 11, 2022, which is 77 kb in size. The information in the electronic format of the sequence listing is incorporated herein by reference in its entirety.

### FIELD

The present disclosure provides RNAi agents comprising at least one modified oligonucleotide having at least one chemical modification.

### BACKGROUND

The principle behind antisense technology is that an antisense compound hybridizes to a target nucleic acid and modulates the amount, activity, and/or function of the target nucleic acid. For example, in certain instances, antisense compounds result in altered transcription or translation of a target. Such modulation of expression can be achieved by, for example, target RNA degradation or occupancy-based inhibition. An example of modulation of RNA target function by degradation is RNase H-based degradation of the target RNA upon hybridization with a DNA-like antisense compound.

Another example of modulation of gene expression by target degradation is RNA interference (RNAi). RNAi refers to antisense-mediated gene silencing through a mechanism that utilizes the RNA-induced silencing complex (RISC). An additional example of modulation of RNA target function is by an occupancy-based mechanism such as is employed naturally by microRNA. MicroRNAs are small non-coding RNAs that regulate the expression of protein-coding RNAs. The binding of an antisense compound to a microRNA prevents that microRNA from binding to its messenger RNA targets, and thus interferes with the function of the microRNA. MicroRNA mimics can enhance native microRNA function. Certain antisense compounds alter splicing of pre-mRNA. Another example of modulation of gene expression is the use of antisense compounds in a CRISPR system. Regardless of the specific mechanism, sequence-specificity makes antisense compounds attractive as tools for target validation and gene functionalization, as well as therapeutics to selectively modulate the expression of genes involved in the pathogenesis of disease.

Antisense technology is an effective means for modulating the expression of one or more specific gene products and can therefore prove to be uniquely useful in a number of therapeutic, diagnostic, and research applications. Chemically modified nucleosides may be incorporated into antisense compounds to enhance one or more properties, such as nuclease resistance, tolerability, pharmacokinetics, or affinity for a target nucleic acid.

### SUMMARY

The present disclosure provides oligomeric compounds (including oligomeric compounds that are antisense agents or portions thereof) comprising modified oligonucleotides consisting of linked nucleosides linked through internucleoside linking groups, wherein at least one of the internucleoside linking groups has Formula I:

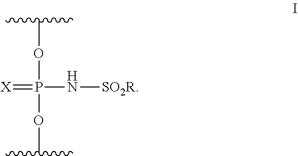

### DETAILED DESCRIPTION

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting.

The section headings used herein are for organizational purposes only and are not to be construed as limiting the subject matter described. All documents, or portions of documents, cited in this application, including, but not limited to, patents, patent applications, articles, books, treatises, and GenBank and NCBI reference sequence records are hereby expressly incorporated by reference for the portions of the document discussed herein, as well as in their entirety.

It is understood that the sequence set forth in each SEQ ID NO contained herein is independent of any modification to a sugar moiety, an internucleoside linkage, or a nucleobase. As such, compounds defined by a SEQ ID NO may comprise, independently, one or more modifications to a sugar moiety, an internucleoside linkage, or a nucleobase. Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH(H) sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, a modified oligonucleotide having the nucleobase sequence “ATCGATCG” encompasses any modified oligonucleotides having such nucleobase sequence, whether modified or unmodified, including, but not limited to, such compounds comprising RNA bases, such as those having sequence “AUCGAUCG” and those having some DNA bases and some RNA bases such as “AUCGATCG” and modified oligonucleotides having other modified nucleobases, such as “AT^{m}CGAUCG,” wherein ^{m}C indicates a cytosine base comprising a methyl group at the 5-position.

As used herein, “2′-substituted” in reference to a furanosyl sugar moiety or nucleoside comprising a furanosyl sugar moiety means the furanosyl sugar moiety or nucleoside comprising the furanosyl sugar moiety comprises a substituent other than H or OH at the 2′-position and is a non-bicyclic furanosyl sugar moiety. 2′-substituted furanosyl sugar moieties do not comprise additional substituents at other positions of the furanosyl sugar moiety other than a nucleobase and/or internucleoside linkage(s) when in the context of an oligonucleotide.

As used herein, “4′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 4′-position and is a non-bicyclic furanosyl sugar moiety. 4′-substituted furanosyl sugar moieties do not comprise additional substituents at other positions of the furanosyl sugar moiety other than a nucleobase and/or internucleoside linkage(s) when in the context of an oligonucleotide.

As used herein, “5′-substituted” in reference to a furanosyl sugar moiety or nucleoside comprising a furanosyl sugar moiety means the furanosyl sugar moiety or nucleoside comprising the furanosyl sugar moiety comprises a substituent other than H at the 5′-position and is a non-bicyclic furanosyl sugar moiety. 5′-substituted furanosyl sugar moieties do not comprise additional substituents at other positions of the furanosyl sugar moiety other than a nucleobase and/or internucleoside linkage(s) when in the context of an oligonucleotide.

As used herein, “administration” or “administering” refers to routes of introducing a compound or composition provided herein to a subject to perform its intended function. Examples of routes of administration that can be used include, but are not limited to, administration by inhalation, subcutaneous injection, intrathecal injection, and oral administration.

As used herein, “antisense activity” means any detectable and/or measurable change attributable to the hybridization of an antisense oligonucleotide to its target nucleic acid. In certain embodiments, antisense activity is a decrease in the amount or expression of a target nucleic acid or protein encoded by such target nucleic acid compared to target nucleic acid levels or target protein levels in the absence of the antisense oligonucleotide.

As used herein, “antisense agent” means an antisense oligonucleotide or an oligonucleotide duplex comprising an antisense oligonucleotide.

As used herein, “antisense compound” means an antisense oligonucleotide or an oligonucleotide duplex comprising an antisense oligonucleotide.

As used herein, “antisense oligonucleotide” means an oligonucleotide that is complementary to a target nucleic acid and is capable of achieving at least one antisense activity. Antisense oligonucleotides include but are not limited to RNAi antisense modified oligonucleotides and RNase H antisense modified oligonucleotides. In certain embodiments, an antisense oligonucleotide is paired with a sense oligonucleotide to form an oligonucleotide duplex. In certain embodiments, an antisense oligonucleotide is unpaired and is a single-stranded antisense oligonucleotide. In certain embodiments, an antisense oligonucleotide comprises a conjugate group.

As used herein, “artificial mRNA compound” is a modified oligonucleotide, or portion thereof, having a nucleobase sequence comprising one or more codons.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety, and the bicyclic sugar moiety is a modified bicyclic furanosyl sugar moiety. In certain embodiments, the bicyclic sugar moiety does not comprise a furanosyl moiety.

As used herein, “cEt” or “constrained ethyl” or “cEt sugar moiety” means a bicyclic sugar moiety, wherein the first ring of the bicyclic sugar moiety is a ribosyl sugar moiety, the second ring of the bicyclic sugar is formed via a bridge connecting the 4′-carbon and the 2′-carbon, the bridge has the formula 4′-CH(CH₃)—O-2′, and the methyl group of the bridge is in the S configuration. A cEt bicyclic sugar moiety is in the β-D configuration.

As used herein, “complementary” in reference to an oligonucleotide means that at least 70% of the nucleobases of such oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases are nucleobase pairs that are capable of forming hydrogen bonds with one another. Complementary nucleobase pairs include adenine (A) and thymine (T), adenine (A) and uracil (U), cytosine (C) and guanine (G), 5-methyl cytosine (^{m}C) and guanine (G). Complementary oligonucleotides and/or nucleic acids need not have nucleobase complementarity at each nucleoside. Rather, some mismatches are tolerated. As used herein, “fully complementary” or “100% complementary” in reference to oligonucleotides means that such oligonucleotides are complementary to another oligonucleotide or nucleic acid at each nucleoside of the oligonucleotide.

As used herein, “conjugate group” means a group of atoms consisting of a conjugate moiety and a conjugate linker.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “conjugate linker” means a group of atoms comprising at least one bond.

As used herein, “CRISPR compound” means a modified oligonucleotide that comprises a DNA recognition portion and a tracrRNA recognition portion. As used herein, “DNA recognition portion” is nucleobase sequence that is complementary to a DNA target. As used herein, “tracrRNA recognition portion” is a nucleobase sequence that is bound to or is capable of binding to tracrRNA. The tracrRNA recognition portion of crRNA may bind to tracrRNA via hybridization or covalent attachment.

As used herein, “cytotoxic” or “cytotoxicity” in the context of an effect of an oligomeric compound or a parent oligomeric compound on cultured cells means an at least 2-fold increase in caspase activation following administration of 10 μM or less of the oligomeric compound or parent oligomeric compound to the cultured cells relative to cells cultured under the same conditions but that are not administered the oligomeric compound or parent oligomeric compound. In certain embodiments, cytotoxicity is measured using a standard in vitro cytotoxicity assay.

As used herein, “deoxy region” means a region of 5-12 contiguous nucleotides, wherein at least 70% of the nucleosides are stereo-standard DNA nucleosides. In certain embodiments, each nucleoside is selected from a stereo-standard DNA nucleoside (a nucleoside comprising a β-D-2′-deoxyribosyl sugar moiety), a stereo-non-standard nucleoside of Formula I-VII, a bicyclic nucleoside, and a substituted stereo-standard nucleoside. In certain embodiments, a deoxy region supports RNase H activity. In certain embodiments, a deoxy region is the gap of a gapmer.

As used herein, “double-stranded antisense compound” means an antisense compound comprising two oligomeric compounds that are complementary to each other and form a duplex, and wherein one of the two said oligomeric compounds comprises an antisense oligonucleotide.

As used herein, “expression” includes all the functions by which a gene's coded information is converted into structures present and operating in a cell. Such structures include, but are not limited to, the products of transcription and translation. As used herein, “modulation of expression” means any change in amount or activity of a product of transcription or translation of a gene. Such a change may be an increase or a reduction of any amount relative to the expression level prior to the modulation.

As used herein, “gapmer” means an oligonucleotide having a central region comprising a plurality of nucleosides that support RNase H cleavage positioned between a 5′-region and a 3′-region. Herein, the nucleosides of the 5′-region and 3′-region each comprise a 2′-substituted furanosyl sugar moiety or a bicyclic sugar moiety, and the 3′- and 5′-most nucleosides of the central region each comprise a sugar moiety independently selected from a 2′-deoxyfuranosyl sugar moiety or a sugar surrogate. The positions of the central region refer to the order of the nucleosides of the central region and are counted starting from the 5′-end of the central region. Thus, the 5′-most nucleoside of the central region is at position 1 of the central region. The “central region” may be referred to as a “gap”, and the “5′-region” and “3′-region” may be referred to as “wings”. Gaps of gapmers are deoxy regions.

As used herein, “hepatotoxic” in the context of a mouse means a plasma ALT level that is above 300 units per liter. Hepatotoxicity of an oligomeric compound or parent oligomeric compound that is administered to a mouse is determined by measuring the plasma ALT level of the mouse 24 hours to 2 weeks following at least one dose of 1-150 mg/kg of the compound.

As used herein, “hepatotoxic” in the context of a human means a plasma ALT level that is above 150 units per liter. Hepatotoxicity of an oligomeric compound or parent oligomeric compound that is administered to a human is determined by measuring the plasma ALT level of the human 24 hours to 2 weeks following at least one dose of 10-300 mg of the compound.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “inhibiting the expression or activity” refers to a reduction or blockade of the expression or activity relative to the expression or activity in an untreated or control sample and does not necessarily indicate a total elimination of expression or activity.

As used herein, “internucleoside linkage” or “internucleoside linking group” means a group or bond that forms a covalent linkage between adjacent nucleosides in an oligonucleotide. As used herein “modified internucleoside linkage” means any internucleoside linkage other than a naturally occurring, phosphodiester internucleoside linkage. “Phosphorothioate linkage” means a modified internucleoside linkage in which one of the non-bridging oxygen atoms of a phosphodiester is replaced with a sulfur atom. Modified internucleoside linkages may or may not contain a phosphorus atom. A “neutral internucleoside linkage” is a modified internucleoside linkage that does not have a negatively charged phosphate in a buffered aqueous solution at pH=7.0. A modified internucleoside linkage may optionally comprise a conjugate group.

As used herein, “linked nucleosides” are nucleosides that are connected in a continuous sequence (i.e. no additional nucleosides are present between those that are linked).

As used herein, “maximum tolerated dose” means the highest dose of a compound that does not cause unacceptable side effects. In certain embodiments, the maximum tolerated dose is the highest dose of a modified oligonucleotide that does not cause an ALT elevation of three times the upper limit of normal as measured by a standard assay.

As used herein, “mismatch” or “non-complementary” means a nucleobase of a first oligonucleotide that is not complementary with the corresponding nucleobase of a second oligonucleotide or target nucleic acid when the first and second oligomeric compound are aligned.

As used herein, “modulating” refers to changing or adjusting a feature in a cell, tissue, organ or organism.

As used herein, “MOE” means O-methoxyethyl. “2′-MOE” or “2′-O-methoxyethyl” means a 2′-OCH₂CH₂OCH₃ group at the 2′-position of a furanosyl ring. In certain embodiments, the 2′-OCH₂CH₂OCH₃ group is in place of the 2′-OH group of a ribosyl ring or in place of a 2′-H in a 2′-deoxyribosyl ring. A “2′-MOE sugar moiety” is a sugar moiety with a 2′-OCH₂CH₂OCH₃ group in place of the 2′-OH group of a furanosyl sugar moiety. Unless otherwise indicated, a 2′-MOE sugar moiety is in the β-D ribosyl configuration.

As used herein, a “2′-OMe sugar moiety” is a sugar moiety with a 2′-OCH₃ group in place of the 2′-OH group of a furanosyl sugar moiety. Unless otherwise indicated, a 2′-OMe sugar moiety is in the β-D ribosyl configuration and is a “stereo-standard 2′OMe sugar moiety”.

As used herein, a “2′-F sugar moiety” is a sugar moiety with a 2′-F group in place of the 2′-OH group of a furanosyl sugar moiety. Unless otherwise indicated, a 2′-F sugar moiety is in the β-D ribosyl configuration and is a “stereo-standard 2′-F sugar moiety”.

As used herein, “motif” means the pattern of unmodified and/or modified sugar moieties, nucleobases, and/or internucleoside linkages, in an oligonucleotide.

As used herein, “naturally occurring” means found in nature.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a modified nucleobase is a group of atoms capable of pairing with at least one unmodified nucleobase. A universal base is a nucleobase that can pair with any one of the five unmodified nucleobases. 5-methylcytosine (^{m}C) is one example of a modified nucleobase.

As used herein, “nucleobase sequence” means the order of contiguous nucleobases in a nucleic acid or oligonucleotide independent of any sugar moiety or internucleoside linkage modification.

As used herein, “nucleoside” means a moiety comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. A modified nucleoside may comprise a conjugate group.

As used herein, “oligomeric compound” means a compound consisting of (1) an oligonucleotide (a single-stranded oligomeric compound) or two oligonucleotides hybridized to one another (a double-stranded oligomeric compound); and (2) optionally one or more additional features, such as a conjugate group or terminal group which may be attached to the oligonucleotide of a single-stranded oligomeric compound or to one or both oligonucleotides of a double-stranded oligomeric compound.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 12-80 linked nucleosides, and optionally a conjugate group or terminal group. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, liquids, powders, or suspensions that can be aerosolized or otherwise dispersed for inhalation by a subject. In certain embodiments, a pharmaceutically acceptable carrier or diluent is sterile water; sterile saline; or sterile buffer solution.

As used herein “pharmaceutically acceptable salts” means physiologically and pharmaceutically acceptable salts of compounds, such as oligomeric compounds (including oligomeric compounds that are antisense agents or portions thereof), i.e., salts that retain the desired biological activity of the compound and do not impart undesired toxicological effects thereto.

As used herein “pharmaceutical composition” means a mixture of substances suitable for administering to a subject. For example, a pharmaceutical composition may comprise an antisense compound and an aqueous solution.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNA), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNAi oligonucleotide” means an RNAi antisense modified oligonucleotide or a RNAi sense modified oligonucleotide.

As used herein, “antisense RNAi oligonucleotide” means an oligonucleotide comprising a region that is complementary to a target sequence, and which includes at least one chemical modification suitable for RNAi.

As used herein, “antisense RNAi oligomeric compound” means a single-stranded oligomeric compound comprising a region that is complementary to a target sequence, and which includes at least one chemical modification suitable for RNAi.

As used herein, “sense RNAi oligonucleotide” means an oligonucleotide comprising a region that is complementary to a region of an RNAi antisense modified oligonucleotide, and which is capable of forming a duplex with such RNAi antisense modified oligonucleotide.

As used herein, “sense RNAi oligomeric compound” means a single-stranded oligomeric compound comprising a region that is complementary to a region of an RNAi antisense modified oligonucleotide and/or an RNAi antisense oligomeric compound, and which is capable of forming a duplex with such RNAi antisense modified oligonucleotide and/or RNAi antisense oligomeric compound.

A duplex formed by an antisense RNAi oligonucleotide and/or an antisense RNAi oligomeric compound with a sense RNAi oligonucleotide and/or a sense RNAi oligomeric compound is referred to as a double-stranded RNAi agent (dsRNAi) or a short interfering RNA (siRNA) or an RNAi duplex.

As used herein, “RNase H agent” means an antisense agent that acts, at least in part, through RNase H to modulate a target nucleic acid and/or protein encoded by a target nucleic acid. In certain embodiments, RNase H agents are single-stranded. In certain embodiments, RNase H agents are double-stranded. RNase H compounds may comprise conjugate groups and/or terminal groups. In certain embodiments, an RNase H agent modulates the amount or activity of a target nucleic acid. The term RNase H agent excludes antisense agents that act principally through RISC/Ago2.

As used herein, “RNase H antisense modified oligonucleotide” means an oligonucleotide comprising a region that is complementary to a target sequence, and which includes at least one chemical modification suitable for RNase H-mediated nucleic acid reduction.

As used herein, the term “single-stranded” in reference to an antisense compound means such a compound consisting of one oligomeric compound that is not paired with a second oligomeric compound to form a duplex. “Self-complementary” in reference to an oligonucleotide means an oligonucleotide that at least partially hybridizes to itself. A compound consisting of one oligomeric compound, wherein the oligonucleotide of the oligomeric compound is self-complementary, is a single-stranded compound. A single-stranded antisense or oligomeric compound may be capable of binding to a complementary oligomeric compound to form a duplex, in which case the compound would no longer be single-stranded.

As used herein, “stabilized phosphate group” refers to a 5′-chemical moiety that results in stabilization of a 5′-phosphate moiety of the 5′-terminal nucleoside of an oligonucleotide, relative to the stability of an unmodified 5′-phosphate of an unmodified nucleoside under biologic conditions. Such stabilization of a 5′-phophate group includes but is not limited to resistance to removal by phosphatases. Stabilized phosphate groups include, but are not limited to, 5′-vinyl phosphonates and 5′-cyclopropyl phosphonate.

As used herein, “stereo-standard nucleoside” means a nucleoside comprising a non-bicyclic furanosyl sugar moiety having the configuration of naturally occurring DNA and RNA as shown below. A “stereo-standard DNA nucleoside” is a nucleoside comprising a β-D-2′-deoxyribosyl sugar moiety. A “stereo-standard RNA nucleoside” is a nucleoside comprising a β-D-ribosyl sugar moiety. A “substituted stereo-standard nucleoside” is a stereo-standard nucleoside other than a stereo-standard DNA or stereo-standard RNA nucleoside. In certain embodiments, R₁ is a 2′-substituent and R₂-R₅ are each H. In certain embodiments, the 2′-substituent is selected from OMe, F, OCH₂CH₂OCH₃, O-alkyl, SMe, or NMA. In certain embodiments, R₁-R₄ are H and R₅ is a 5′-substituent selected from methyl, allyl, or ethyl. In certain embodiments, the heterocyclic base moiety Bx is selected from uracil, thymine, cytosine, 5-methyl cytosine, adenine or guanine. In certain embodiments, the heterocyclic base moiety Bx is other than uracil, thymine, cytosine, 5-methylcytosine, adenine or guanine.

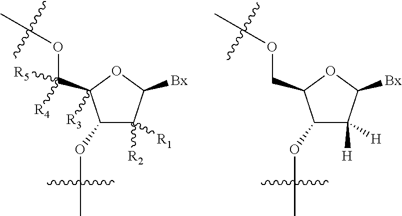

### Stereo-Standard Nucleoside Stereo-Standard DNA Nucleoside

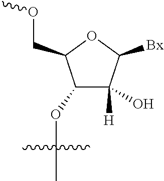

### Stereo-Standard RNA Nucleoside

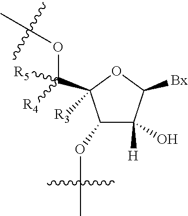

### Stereo-Standard 2′-Substituted Nucleoside

R₁ is a 2′-substituent other than H
R₂ is H

As used herein, “stereo-non-standard nucleoside” means a nucleoside comprising a non-bicyclic furanosyl sugar moiety having a configuration other than that of a stereo-standard sugar moiety. A stereo-non-standard nucleoside is a modified nucleoside. In certain embodiments, a “stereo-non-standard nucleoside” comprises a 2′-β-L-deoxyribosyl sugar moiety, 2′-α-D-deoxyribosyl sugar moiety, 2′-α-L-deoxyribosyl sugar moiety, a 2′-β-D-deoxyxylosyl sugar moiety, a 2′-β-L-deoxyxylosyl sugar moiety, a 2′-α-D-deoxyxylosyl sugar moiety, a 2′-α-L-deoxyxylosyl sugar moiety, a β-L-ribosyl sugar moiety, α-D-ribosyl sugar moiety, α-L-ribosyl sugar moiety, a β-D-xylosyl sugar moiety, β-L-xylosyl sugar moiety, a α-D-xylosyl sugar moiety, a 2′-α-L-xylosyl sugar moiety, a β-D-arabinosyl sugar moiety, β-L-arabinosyl sugar moiety, a α-D-arabinosyl sugar moiety, a 2′-α-L-arabinosyl sugar moiety, a β-D-lyxosyl sugar moiety, β-L-lyxosyl sugar moiety, a α-D-lyxosyl sugar moiety, a 2′-α-L-lyxosyl sugar moiety, a 2′-fluoro-β-D-arabinosyl sugar moiety, a 2′-fluoro-β-D-xylosyl sugar moiety, a 2′-fluoro-α-D-ribosyl sugar moiety, a 2′-fluoro-α-D-arabinosyl sugar moiety, a 2′-fluoro-α-D-xylosyl sugar moiety, a 2′-fluoro-α-L-ribosyl sugar moiety, a 2′-fluoro-β-L-xylosyl sugar moiety, a 2′-fluoro-α-L-arabinosyl sugar moiety, a 2′-fluoro-α-L-xylosyl sugar moiety, a 2′-fluoro-β-L-ribosyl sugar moiety, a 2′-fluoro-β-L-arabinosyl sugar moiety, a 2′-fluoro-β-D-lyxosyl sugar moiety, a 2′-fluoro-α-D-lyxosyl sugar moiety, a 2′-fluoro-α-L-lyxosyl sugar moiety, a 2′-fluoro-β-L-lyxosyl sugar moiety, a 2′-O-methyl-β-D-arabinosyl sugar moiety, a 2′-O-methyl-β-D-xylosyl sugar moiety, a 2′-O-methyl-α-D-ribosyl sugar moiety, a 2′-O-methyl-α-D-arabinosyl sugar moiety, a 2′-O-methyl-α-D-xylosyl sugar moiety, a 2′-O-methyl-α-L-ribosyl sugar moiety, a 2′-O-methyl-β-L-xylosyl sugar moiety, a 2′-O-methyl-α-L-arabinosyl sugar moiety, a 2′-O-methyl-α-L-xylosyl sugar moiety, a 2′-O-methyl-β-L-ribosyl sugar moiety, a 2′-O-methyl-β-L-arabinosyl sugar moiety, a 2′-O-methyl-β-D-lyxosyl sugar moiety, a 2′-O-methyl-α-D-lyxosyl sugar moiety, a 2′-O-methyl-α-L-lyxosyl sugar moiety, or a 2′-O-methyl-β-L-lyxosyl sugar moiety.

As used herein, “stereo-standard sugar moiety” means the sugar moiety of a stereo-standard nucleoside.

As used herein, “stereo-non-standard sugar moiety” means the sugar moiety of a stereo-non-standard nucleoside.

As used herein, “substituted stereo-non-standard nucleoside” means a stereo-non-standard nucleoside comprising a substituent other than the substituent corresponding to natural RNA or DNA. In certain embodiments, a substituted stereo-non-standard nucleoside comprises a 2′-fluoro-β-D-arabinosyl sugar moiety, a 2′-fluoro-β-D-xylosyl sugar moiety, a 2′-fluoro-α-D-ribosyl sugar moiety, a 2′-fluoro-α-D-arabinosyl sugar moiety, a 2′-fluoro-α-D-xylosyl sugar moiety, a 2′-fluoro-α-L-ribosyl sugar moiety, a 2′-fluoro-β-L-xylosyl sugar moiety, a 2′-fluoro-α-L-arabinosyl sugar moiety, a 2′-fluoro-α-L-xylosyl sugar moiety, a 2′-fluoro-β-L-ribosyl sugar moiety, a 2′-fluoro-β-L-arabinosyl sugar moiety, a 2′-fluoro-β-D-lyxosyl sugar moiety, a 2′-fluoro-α-D-lyxosyl sugar moiety, a 2′-fluoro-α-L-lyxosyl sugar moiety, a 2′-fluoro-β-L-lyxosyl sugar moiety, a 2′-O-methyl-β-D-arabinosyl sugar moiety, a 2′-O-methyl-β-D-xylosyl sugar moiety, a 2′-O-methyl-α-D-ribosyl sugar moiety, a 2′-O-methyl-α-D-arabinosyl sugar moiety, a 2′-O-methyl-α-D-xylosyl sugar moiety, a 2′-O-methyl-α-L-ribosyl sugar moiety, a 2′-O-methyl-β-L-xylosyl sugar moiety, a 2′-O-methyl-α-L-arabinosyl sugar moiety, a 2′-O-methyl-α-L-xylosyl sugar moiety, a 2′-O-methyl-β-L-ribosyl sugar moiety, a 2′-O-methyl-β-L-arabinosyl sugar moiety, a 2′-O-methyl-β-D-lyxosyl sugar moiety, a 2′-O-methyl-α-D-lyxosyl sugar moiety, a 2′-O-methyl-α-L-lyxosyl sugar moiety, or a 2′-O-methyl-β-L-lyxosyl sugar moiety.

As used herein, “subject” means a human or non-human animal selected for treatment or therapy.

As used herein, “sugar moiety” means an unmodified sugar moiety or a modified sugar moiety. As used herein, “unmodified sugar moiety” means a β-D-ribosyl moiety, as found in naturally occurring RNA, or a β-D-2′-deoxyribosyl sugar moiety as found in naturally occurring DNA. As used herein, “modified sugar moiety” or “modified sugar” means a sugar surrogate or a furanosyl sugar moiety other than a β-D-ribosyl or a β-D-2′-deoxyribosyl. Modified furanosyl sugar moieties may be modified or substituted at a certain position(s) of the sugar moiety, or unsubstituted, and they may or may not be stereo-non-standard sugar moieties. Modified furanosyl sugar moieties include bicyclic sugars and non-bicyclic sugars. As used herein, “sugar surrogate” means a modified sugar moiety that does not comprise a furanosyl or tetrahydrofuranyl ring (is not a “furanosyl sugar moiety”) and that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid,” “target RNA,” “target RNA transcript” and “nucleic acid target” means a nucleic acid that an oligomeric compound, such as an antisense compound, is designed to affect. In certain embodiments, an oligomeric compound comprises an oligonucleotide having a nucleobase sequence that is complementary to more than one RNA, only one of which is the target RNA of the oligomeric compound. In certain embodiments, the target RNA is an RNA present in the species to which an oligomeric compound is administered.

As used herein, “therapeutic index” means a comparison of the amount of a compound that causes a therapeutic effect to the amount that causes toxicity. Compounds having a high therapeutic index have strong efficacy and low toxicity. In certain embodiments, increasing the therapeutic index of a compound increases the amount of the compound that can be safely administered.

As used herein, “treat” refers to administering a compound or pharmaceutical composition to an animal in order to effect an alteration or improvement of a disease, disorder, or condition in the animal.

As used herein, “translation suppression element,” means any sequence and/or secondary structure in the 5′-UTR of a target transcript that reduces, inhibits, and/or suppresses translation of the target transcript. In certain embodiments, a translation suppression element comprises a uORF. In certain embodiments, a translation suppression element does not comprise a uORF. In certain embodiments, a translation suppression element comprises one or more stem-loops. In certain embodiments, a translation suppression element comprises greater than 60%, greater than 70%, or greater than 80% GC content. In certain embodiments, the translation suppression element is a uORF. In certain embodiments, the translation suppression element is a stem-loop.

### CERTAIN EMBODIMENTS

The present disclosure provides the following non-limiting embodiments:
Embodiment 1. An RNAi agent, comprising an antisense siRNA oligomeric compound comprising an antisense RNAi oligonucleotide consisting of 18-25 linked nucleosides, wherein
at least one internucleoside linking group of the antisense RNAi oligonucleotide is an internucleoside linking group of Formula I:

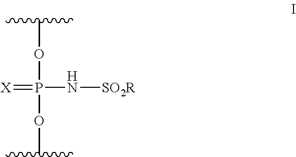

wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group.
Embodiment 2. The RNAi agent of embodiment 1, wherein the antisense RNAi oligonucleotide comprises a 5′-stabilized phosphate group.
Embodiment 3. The RNAi agent of embodiment 1 or embodiment 2, wherein the antisense RNAi oligonucleotide comprises at least one modified sugar moiety selected from a 2′-MOE sugar moiety and a stereo-non-standard 2′-F sugar moiety.
Embodiment 4. The RNAi agent of any of embodiments 1-3, wherein for at least one internucleoside linking group of Formula I, X is O and R is methyl.
Embodiment 5. The RNAi agent of any of embodiments 1-4, wherein for each internucleoside linking group of Formula I, X is O and R is methyl.
Embodiment 6. The RNAi agent of any of embodiments 1-5, wherein for at least one internucleoside linking group of Formula I, X is O and R is C₁₆ alkyl.
Embodiment 7. The RNAi agent of any of embodiments 1-6, further comprising a sense siRNA oligomeric compound comprising sense siRNA oligonucleotide consisting of 18-30 linked nucleosides, wherein the sense siRNA oligonucleotide has a complementary region of at least 18 consecutive nucleosides that are at least 85% complementary to an equal length region of the antisense siRNA oligonucleotide.
Embodiment 8. The RNAi agent of embodiment 7, wherein the complementary region of the sense siRNA oligonucleotide is at least 90, at least 95, or 100% complementary to the corresponding region of the antisense RNAi oligonucleotide.
Embodiment 9. The RNAi agent of any of embodiments 1-8, wherein the antisense RNAi oligonucleotide consists of 23 linked nucleosides.
Embodiment 10. The RNAi agent of any of embodiments 7-9, wherein the sense siRNA oligonucleotide consists of 21 linked nucleosides.
Embodiment 11. The RNAi agent of embodiment 10, wherein the complementary region of the sense siRNA consists of 21 nucleobases.
Embodiment 12. The RNAi agent of any of embodiments 1-11, wherein the antisense RNAi oligonucleotide has a target complementary region of at least 18 consecutive nucleosides that are at least 85% complementary to an equal length portion of a target nucleic acid.
Embodiment 13. The RNAi agent of embodiment 12, wherein the antisense RNAi oligonucleotide has a target complementary region that is at least 90%, at least 95%, or 100% complementary to an equal length portion of a target nucleic acid.
Embodiment 14. The RNAi agent of embodiment 12, wherein the target complementary region comprises 22 consecutive nucleosides.
Embodiment 15. The RNAi agent of embodiment 7, wherein the antisense RNAi oligonucleotide consists of 23 linked nucleosides, the sense siRNA oligonucleotide consists of 21 linked nucleosides, the sense siRNA oligonucleotide is 100% complementary to the antisense RNAi oligonucleotide, and the antisense RNAi oligonucleotide is at least 90% complementary to an equal length portion of a target nucleic acid.
Embodiment 16. The RNAi agent of embodiment 15, wherein the antisense RNAi oligonucleotide is at least 95% complementary to an equal length portion of a target nucleic acid.
Embodiment 17. The RNAi agent of any of embodiments 1-16, wherein the 5′-stabilized phosphate group is selected from vinyl phosphonate, mesyl phosphoramidate, and cyclopropyl phosphonate.
Embodiment 18. The RNAi agent of embodiment 17, wherein the 5′-stabilized phosphate group is vinyl phosphonate.
Embodiment 19. The RNAi agent of embodiment 17, wherein the 5′-stabilized phosphate group is mesyl phosphonate.
Embodiment 20. The RNAi agent of any of embodiments 1-19, wherein the first internucleoside linkage from the 5′-end of the antisense RNAi oligonucleotide is an internucleoside linkage of Formula I.
Embodiment 21. The RNAi agent of any of embodiments 1-20, wherein the second internucleoside linkage from the 5′-end of the antisense RNAi oligonucleotide is an internucleoside linkage of Formula I.
Embodiment 22. The RNAi agent of any of embodiments 9-21, wherein the 21^{st} internucleoside linkage from the 5′-end of the antisense RNAi oligonucleotide is an internucleoside linkage of Formula I.
Embodiment 23. The RNAi agent of any of embodiments 7-22, wherein the 22^{nd} internucleoside linkage from the 5′-end of the antisense RNAi oligonucleotide is an internucleoside linkage of Formula I.
Embodiment 24. The RNAi agent of any of embodiments 1-23, wherein the internucleoside linkage between the 6th and 7th nucleosides from the 5′-end of the antisense RNAi oligonucleotide is an internucleoside linkage of Formula I.
Embodiment 25. The RNAi agent of any of embodiments 1-24, wherein the internucleoside linkage between the 7th and 8th nucleosides from the 5′-end of the antisense RNAi oligonucleotide is an internucleoside linkage of Formula I.
Embodiment 26. The RNAi agent of embodiment 25, wherein the internucleoside linkage between the 7^{th} and 8^{th} nucleosides is an internucleoside linkage of Formula I wherein X is O and R is C₁₆ alkyl.
Embodiment 27. The RNAi agent of any of embodiments 5-26, wherein the first internucleoside linkage from the 5′-end of the sense RNAi oligonucleotide is an internucleoside linkage of Formula I.
Embodiment 28. The RNAi agent of any of embodiments 5-27, wherein the second internucleoside linkage from the 5′-end of the sense RNAi oligonucleotide is an internucleoside linkage of Formula I.
Embodiment 29. The RNAi agent of any of embodiments 10-28, wherein the 19^{th} internucleoside linkage from the 5′-end of the sense RNAi oligonucleotide is an internucleoside linkage of Formula I.
Embodiment 30. The RNAi agent of any of embodiments 10-29, wherein the 20^{th} internucleoside linkage from the 5′-end of the sense RNAi oligonucleotide is an internucleoside linkage of Formula I.
Embodiment 31. The RNAi agent of any of embodiments 7-30, wherein the 6^{th} internucleoside linkage from the 5′-end of the sense RNAi oligonucleotide is an internucleoside linkage of Formula I.
Embodiment 32. The RNAi agent of embodiment 31, wherein the 6^{th} internucleoside linkage from the 5′ end of the sense RNAi oligonucleotide is an internucleoside linkage of Formula I wherein X is O and R is C₁₆ alkyl.
Embodiment 33. The RNAi agent of any of embodiments 7-30, wherein the 7^{th} internucleoside linkage from the 5′-end of the sense RNAi oligonucleotide is an internucleoside linkage of Formula I.
Embodiment 34. The RNAi agent of any of embodiments 7-30, wherein the 9^{th} internucleoside linkage from the 5′-end of the sense RNAi oligonucleotide is an internucleoside linkage of Formula I.
Embodiment 35. The RNAi agent of any of embodiments 7-30, wherein the 10^{th} internucleoside linkage from the 5′-end of the sense RNAi oligonucleotide is an internucleoside linkage of Formula I.
Embodiment 36. The RNAi agent of any of embodiments 7-30, wherein the 11^{th} internucleoside linkage from the 5′-end of the sense RNAi oligonucleotide is an internucleoside linkage of Formula I.
Embodiment 37. The RNAi agent of any of embodiments 20-25 or 27-31 or 33-36, wherein for each internucleoside linkage of Formula I, X is O and R is methyl.
Embodiment 38. The RNAi agent of any of embodiments 20-37, wherein each internucleoside linkage that does not have Formula I is selected from a phosphodiester internucleoside linkage and a phosphorothioate internucleoside linkage.
Embodiment 39. The RNAi agent of any of embodiments 1-38, wherein the two 3′-terminal and 5′-terminal internucleoside linkages of the antisense RNAi oligonucleotide are selected from an internucleoside linkage of Formula I and a phosphorothioate internucleoside linkage, and the remaining internucleoside linkages are phosphodiester internucleoside linkages.
Embodiment 40. The RNAi agent of any of embodiments 7-39, wherein the two 3′-terminal and 5′-terminal internucleoside linkages of the sense RNAi oligonucleotide are selected from an internucleoside linkage of formula I and a phosphorothioate internucleoside linkage, and the remaining internucleoside linkages are phosphodiester internucleoside linkages.
Embodiment 41. The RNAi agent of embodiment 39 or 40, wherein X is O and R is methyl.
Embodiment 42. The RNAi agent of any of embodiments 1-41, wherein each nucleoside of the antisense RNAi oligonucleotide comprises a modified sugar moiety selected from 2′-OMe, 2′-MOE, 2′-F, and a sugar surrogate.
Embodiment 43. The RNAi agent of embodiment 42, wherein each 2′-F sugar moiety is independently selected from a stereo-standard 2′-F sugar moiety and a stereo-non-standard 2′-F sugar moiety.
Embodiment 44. The RNAi agent of embodiment 43, wherein each 2′-F sugar moiety is selected from 2′-fluoro-β-D-ribosyl and 2′-fluoro-β-D-xylosyl.
Embodiment 45. The RNAi agent of embodiment 44, wherein each 2′-F sugar moiety is a 2′-fluoro-β-D-xylosyl sugar moiety.
Embodiment 46. The RNAi agent of any of embodiments 42-45, wherein the antisense RNAi oligonucleotide consists of 23 linked nucleosides and the nucleosides at positions 2, 6, 14, and 16 from the 5′-end comprise 2′-F modified sugar moieties.
Embodiment 47. The RNAi agent of embodiment 46, wherein the first nucleoside from the 5′-end of the antisense RNAi oligonucleotide comprises a 2′-MOE sugar moiety.
Embodiment 48. The RNAi agent of any of embodiments 42-47, wherein each modified sugar moiety that is not a 2′-MOE sugar moiety or a 2′-F sugar moiety is a 2′-OMe sugar moiety.
Embodiment 49. The RNAi agent of embodiment 42, wherein the antisense RNAi oligonucleotide consists of 23 linked nucleosides and the nucleosides at positions 2, 6, 14, and 16 from the 5′-end comprise 2′-F sugar moieties or a sugar surrogate.
Embodiment 50. The RNAi agent of embodiment 42 or 49, wherein the sugar surrogate is fluoro hexitol nucleic acid (F-HNA).
Embodiment 51. The RNAi agent of any of embodiments 7-50, wherein each nucleoside of the sense RNAi oligonucleotide comprises a modified sugar moiety selected from 2′-OMe, 2′-F, and a sugar surrogate.
Embodiment 52. The RNAi agent of embodiment 51, wherein each 2′-F sugar moiety is independently selected from a stereo-standard 2′-F sugar moiety and a stereo-non-standard 2′-F sugar moiety.
Embodiment 53. The RNAi agent of embodiment 52, wherein each 2′-F sugar moiety is selected from 2′-fluoro-β-D-ribosyl and 2′-fluoro-β-D-xylosyl.
Embodiment 54. The RNAi agent of embodiment 53, wherein each 2′-F sugar moiety is a 2′-fluoro-β-D-xylosyl sugar moiety.
Embodiment 55. The RNAi agent of any of embodiments 51-54, wherein the sense RNAi oligonucleotide consists of 21 linked nucleosides and the nucleosides at positions 7, 9, 10, and 11 from the 5′-end comprise 2′-F modified sugar moieties.
Embodiment 56. The RNAi agent of embodiment 51, wherein the sense RNAi oligonucleotide consists of 21 linked nucleosides and the nucleosides at positions 7, 9, 10, and 11 from the 5′-end comprise 2′-F modified sugar moieties or a sugar surrogate.
Embodiment 57. The RNAi agent of embodiment 51 or 56, wherein the sugar surrogate is fluoro hexitol nucleic acid (F-HNA).
Embodiment 58. The RNAi agent of any of embodiments 7-50, wherein each nucleoside of the sense RNAi oligonucleotide comprises a sugar moiety selected from 2′-OMe, 2′-F, and 2′-β-D-deoxyribosyl.
Embodiment 59. The RNAi agent of embodiment 58, wherein the sense RNAi oligonucleotide consists of 30 linked nucleosides and the 3′-end comprises five to ten 2′-β-D-deoxyribosyl sugar moieties.
Embodiment 60. The RNAi agent of embodiment 59, wherein the internucleoside linkages between the nucleosides comprising 2′-β-D-deoxyribosyl sugar moieties are phosphorothioate internucleoside linkages.
Embodiment 61. The RNAi agent of any of embodiments 1-60, wherein the antisense siRNA oligomeric compound comprises a conjugate group.
Embodiment 62. The RNAi agent of embodiment 61, wherein the conjugate group comprises a conjugate moiety and a conjugate linker.
Embodiment 63. The RNAi agent of embodiment 62, wherein the conjugate moiety is a cell-targeting moiety.
Embodiment 64. The RNAi agent of embodiment 62, wherein the conjugate moiety is a lipid.
Embodiment 65. The RNAi agent of embodiment 62, wherein the conjugate moiety comprises C₁₂-C₂₀ alkyl.
Embodiment 66. The RNAi agent of embodiment 62, wherein the conjugate moiety is C₁₆ alkyl.
Embodiment 67. The RNAi agent of any of embodiments 61-66, wherein the conjugate moiety is part of an internucleoside linkage of Formula I.
Embodiment 68. The RNAi agent of any of embodiments 61-66, wherein the conjugate moiety is a 2′-modification of a sugar moiety.
Embodiment 69. The RNAi agent of any of embodiments 61-66, wherein the conjugate moiety is attached at the 3′-terminal of the antisense RNAi oligonucleotide.
Embodiment 70. The RNAi agent of any of embodiments 61-66, wherein the conjugate moiety is attached at the 5′-terminal of the antisense RNAi oligonucleotide.
Embodiment 71. The RNAi agent of any of embodiments 7-70, wherein the sense siRNA oligomeric compound comprises a conjugate group.
Embodiment 72. The RNAi agent of embodiment 71, wherein the conjugate group comprises a conjugate moiety and a conjugate linker.
Embodiment 73. The RNAi agent of embodiment 72, wherein the conjugate moiety is a cell-targeting moiety.
Embodiment 74. The RNAi agent of embodiment 72, wherein the conjugate moiety is a lipid.
Embodiment 75. The RNAi agent of embodiment 72, wherein the conjugate moiety comprises C₁₂-C₂₀ alkyl.
Embodiment 76. The RNAi agent of embodiment 72, wherein the conjugate moiety is C₁₆ alkyl.
Embodiment 77. The RNAi agent of embodiment 72, wherein the conjugate moiety is a carbohydrate.
Embodiment 78. The RNAi agent of embodiment 77, wherein the conjugate moiety comprises a GalNAc.
Embodiment 79. The RNAi agent of any of embodiments 71-78, wherein the conjugate moiety is part of an internucleoside linkage of Formula I.
Embodiment 80. The RNAi agent of any of embodiments 71-78, wherein the conjugate moiety is a 2′-modification of a sugar moiety.
Embodiment 81. The RNAi agent of any of embodiments 71-78, wherein the conjugate moiety is attached at the 3′-terminal of the sense RNAi oligonucleotide.
Embodiment 82. The RNAi agent of any of embodiments 71-78, wherein the conjugate moiety is attached at the 5′-terminal of the sense RNAi oligonucleotide.
Embodiment 83. A chirally enriched population of RNAi agents of any of embodiments 1-82, wherein the population is enriched for antisense RNAi oligonucleotides and/or sense RNAi oligonucleotides comprising at least one particular internucleoside linkage having a particular stereochemical configuration.
Embodiment 84. The chirally enriched population of embodiment 83, wherein the population is enriched for antisense RNAi oligonucleotides and/or sense RNAi oligonucleotides comprising at least one particular internucleoside of Formula I having the (Sp) or (Rp) configuration.
Embodiment 85. The chirally enriched population of embodiment 84, wherein the population is enriched for antisense RNAi oligonucleotides comprising at least one internucleoside linkage of Formula I having the (Sp) configuration.
Embodiment 86. The chirally enriched population of embodiment 84, wherein the population is enriched for antisense RNAi oligonucleotides comprising at least one internucleoside linkage of Formula I having the (Rp) configuration.
Embodiment 87. The chirally enriched population of embodiment 85, wherein the at least one internucleoside linkage of Formula I having the (Sp) configuration is the first internucleoside linkage from the 5′ end of the antisense RNAi oligonucleotide.
Embodiment 88. The chirally enriched population of embodiment 86, wherein the at least one internucleoside linkage of Formula I having the (Rp) configuration is the first internucleoside linkage from the 5′ end of the antisense RNAi oligonucleotide.
Embodiment 89. The chirally enriched population of embodiment 85, wherein the at least one internucleoside linkage of Formula I having the (Sp) configuration is the second internucleoside linkage from the 5′ end of the antisense RNAi oligonucleotide.
Embodiment 90. The chirally enriched population of embodiment 86, wherein the at least one internucleoside linkage of Formula I having the (Rp) configuration is the second internucleoside linkage from the 5′ end of the antisense RNAi oligonucleotide.
Embodiment 91. A method comprising administering at least two doses of an RNAi agent of any of embodiments 1-82 to an animal wherein:
the RNAi agent is administered to the animal at a dose frequency of once per 4 months, 5 months, 6 months, 7 months, 8 months, 9 months, 10 months, 11 months, or a year or more than a year.
Embodiment 92. An RNAi agent, comprising an antisense siRNA oligomeric compound comprising an antisense RNAi oligonucleotide consisting of 21-25 linked nucleosides and a sense RNAi oligomeric compound comprising a sense RNAi oligonucleotide consisting of 19-23 linked nucleosides, wherein the antisense RNAi oligonucleotide comprises a 5′-stabilized phosphate group; and at least one internucleoside linkage of Formula I:

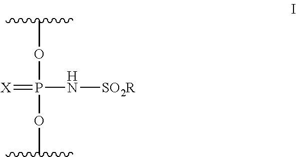

wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group; and
wherein each of at least three sugar moieties of the nucleosides of the antisense RNAi oligonucleotide is selected from a 2′-O-methyl-β-D-ribosyl sugar moiety, a 2′-O-methoxyethyl β-D-ribosyl sugar moiety, a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, and wherein each of at least three such sugar moieties are different from one another.
Embodiment 93. The RNAi agent of embodiment 93, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.
Embodiment 94. The RNAi agent of embodiment 93 or 94, wherein each sugar surrogate is independently selected from fluoro hexitol nucleic acid (F-HNA), hexitol nucleic acid (HNA) or altritol nucleic acid (ANA).
Embodiment 95. The RNAi agent of any of embodiments 92-94, wherein the antisense RNAi oligonucleotide comprises no more than three 2′-fluoro-β-D-ribosyl sugar moieties.
Embodiment 96. The RNAi agent of any of embodiments 92-95, wherein for at least one internucleoside linkage of Formula I, X is O and R is methyl.
Embodiment 97. The RNAi agent of any of embodiments 92-96, wherein for each internucleoside linkage of Formula I, X is O and R is methyl.
Embodiment 98. The RNAi agent of any of embodiments 92-97, wherein each internucleoside linkage of the antisense RNAi oligonucleotide is selected from an internucleoside linkage of Formula I, a phosphodiester internucleoside linkage, and a phosphorothioate internucleoside linkage.
Embodiment 99. The RNAi agent of embodiment 98, wherein the antisense RNAi oligonucleotide has an internucleoside linkage motif selected from sz(o)ₙzs, ss(o)ₙzs, and sz(o)ₙss, wherein each “s” is a phosphorothioate internucleoside linkage, each “z” is an internucleoside linkage of Formula I, each “o” is a phosphodiester internucleoside linkage, and n is from 16-20.
Embodiment 100. The RNAi agent of any of embodiments 92-99, wherein the antisense RNAi oligonucleotide consists of 23 linked nucleosides.
Embodiment 101. The RNAi agent of embodiment 100, wherein the antisense RNAi oligonucleotide has an internucleoside linkage motif selected from ssooooooooooooooooooss, zsooooooooooooooooooss, szooooooooooooooooooss, zzooooooooooooooooooss, ssooooooooooooooooooss, ssoooooooooooooooooozz, zsoooooooooooooooooozz, zsoooooooooooooooooozz, ssoooooooooooooooooosz, ssoooooooooooooooooozs, szoooooooooooooooooosz, zsoooooooooooooooooosz, zsoooooooooooooooooozs, szoooooooooooooooooozs, wherein each “s” is a phosphorothioate internucleoside linkage, each “z” is an internucleoside linkage of Formula I, and each “o” is a phosphodiester internucleoside linkage.
Embodiment 102. The RNAi agent of embodiment 101, wherein for each internucleoside linkage of Formula I, X is O and R is methyl.
Embodiment 103. The RNAi agent of any of embodiments 92-102, wherein the antisense RNAi oligonucleotide has a sugar motif of yxyyyxyyyyyyyxyxyyyyyyy or exyyyxyyyyyyyxyxyyyyyyy, from 5′ to 3′, wherein each “y” is a 2′-O-methyl-β-D-ribosyl sugar moiety, each “e” is a 2′-O-methoxyethyl-β-D-ribosyl sugar moiety, and each “x” is independently selected from a sugar surrogate, a stereo-non-standard sugar moiety, a 2′-β-D-deoxyribosyl sugar moiety, a β-D-ribosyl sugar moiety, and a 2′-fluoro-β-D-ribosyl sugar moiety, wherein at least one “x” is other-than a 2′-fluoro-β-D-ribosyl sugar moiety.
Embodiment 104. The RNAi agent of embodiment 103, wherein at least one “x” is a stereo-non-standard sugar moiety.
Embodiment 105. The RNAi agent of embodiment 103, wherein at least one “x” is a 2′-fluoro-β-D-xylosyl sugar moiety.
Embodiment 106. The RNAi agent of embodiment 103, wherein exactly one “x” is a stereo-non-standard sugar moiety.
Embodiment 107. The RNAi agent of embodiment 106, wherein exactly one “x” is a 2′-fluoro-β-D-xylosyl sugar moiety.
Embodiment 108. The RNAi agent of embodiment 103, wherein at least one “x” is a sugar surrogate.
Embodiment 109. The RNAi agent of embodiment 103, wherein exactly one “x” is a sugar surrogate.
Embodiment 110. The RNAi agent of embodiment 108 or 109, wherein the sugar surrogate is fluoro hexitol nucleic acid (F-HNA).
Embodiment 111. The RNAi agent of embodiment 110, wherein at least one “x” is a β-D-ribosyl sugar moiety.
Embodiment 112. The RNAi agent of any of embodiments 92-111, wherein the antisense RNAi oligonucleotide has a sugar motif selected from: yfyyyfyyyyyyyfyfyyyyyyy, y[f2bDx]yyyfyyyyyyyfyfyyyyyyy, yfyyy[f2bDx]yyyyyyyfyfyyyyyyy, yfyyyfyyyyyyy[f2bDx]yfyyyyyyy, yfyyyfyyyyyyyfy[f2bDx]yyyyyyy, y[f2bDx]yyy[f2bDx]yyyyyyy[f2bDx]y[f2bDx]yyyyyyy, yfyfyfyfyfyfyfyfyfyfyyy, efyfyfyfyfyfyfyfyfyfyyy, efyyyyyyyyyyyfyfyyyyyyy, efyyyfyyyyyyyfyfyyyyyyy, efyyyfy[16C2r]yyyyyfyfyyyyyyy, e[f2bDx]yyyfyyyyyyyfyfyyyyyyy, efyyy[f2bDx]yyyyyyyfyfyyyyyyy, efyyyyyyyyyy[f2bDx]yfyyyyyyy, efyyyfyyyyyyyfy[f2bDx]yyyyyyy, e[f2bDx]yyy[f2bDx]yyyyyyy[f2bDx]y[f2bDx]yyyyyyy, efyyy[F-HNA]yyyyyyyfyfyyyyyyy, efyyyfyyyyyyy[F-HNA]yfyyyyyyy, efyyyfyyyyyyyfy[F-HNA]yyyyyyy, yfyyydyyyyyyyfyfyyyyyyy, yfyyyfyyyyyyydyfyyyyyyy, yfyyyfyyyyyyyfydyyyyyyy, yfyyydyyyyyyydydyyyyyyy, yryyyfyyyyyyyfyfyyyyyyy, yfyyyryyyyyyyfyfyyyyyyy, yfyyyfyyyyyyyryfyyyyyyy, yfyyyfyyyyyyyfyryyyyyyy, yryyyryyyyyyyryryyyyyyy, y[bDdx]yyyfyyyyyyyfyfyyyyyyy, yfyyy[bDdx]yyyyyyyfyfyyyyyyy, yfyyyfyyyyyyy[bDdx]yfyyyyyyy, yfyyyfyyyyyyyfy[bDdx]yyyyyyy, y[bDdx]yyy[bDdx]yyyyyyy[bDdx]y[bDdx]yyyyyyy, y[F-HNA]yyyfyyyyyyyfyfyyyyyyy, yfyyy[F-HNA]yyyyyyyfyfyyyyyyy, yfyyyfyyyyyyy[F-HNA]yfyyyyyyy, yfyyyfyyyyyyyfy[F-HNA]yyyyyyy, y[F-HNA]yyy[F-HNA]yyyyyyy[F-HNA]y[F-HNA]yyyyyyy, yfyyyfyyyyyyy[2bDx]yfyyyyyyy, y[bDa]yyyfyyyyyyyfyfyyyyyyy, yfyyy[bDa]yyyyyyyfyfyyyyyyy, yfyyyfyyyyyyy[bDa]yfyyyyyyy, yfyyyfyyyyyyyfy[bDa]yyyyyyy, y[bDa]yyy[bDa]yyyyyyy[bDa]y[bDa]yyyyyyy, yfyyyfyyyyyyy[bDx]yfyyyyyyy, yfyyyfyffyyyyfyfyyyyydd, yfyyyfyffyyyyfyfyyyyy[bLdr][bLdr], yfyyyfyffyyyyfyfyyyyy[aDdr][aDdr], yfyyyfyffyyyyfyfyyyyy[bDdx][bDdx], yfyyyfyffyyyyfyfyyyyy[bLdx][bLdx], yfyyyfyffyyyyfyfyyyyy[aDdx][aDdx], yfyyyfyffyyyyfyfyyyyy[aLdx][aLdx]; wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C2r]” represents a 2′-O-hexadecyl-β-D-ribosyl sugar moiety, “[F-HNA]” represents the sugar surrogate 3′-fluoro-tetrahydropyran, “[bDdx]” represents a 2′-β-D-deoxyxylosyl sugar moiety, “[bDx]” represents a β-D-xylosyl sugar moiety, “[bDa]” represents a β-D-arabinosyl sugar moiety, “[bLdr]” represents a 2′-β-L-deoxyribosyl sugar moiety, “[aDdr]” represents a 2′-α-D-deoxyribosyl sugar moiety, “[aLdr]” represents a 2′-α-L-deoxyribosyl sugar moiety, “[bLdx]” represents a 2′-β-L-deoxyxylosyl sugar moiety, “[aDdx]” represents an 2′-α-D-deoxyxylosyl sugar moiety, and “[aLdx]” represents an 2′-α-L-deoxyxylosyl sugar moiety.
Embodiment 113. The RNAi agent of any of embodiments 92-112, wherein the antisense RNAi oligonucleotide has a sugar motif of dzyyyxyyyyyyyxyxyyyyyyy, dxyyyxyyyyyyyxyxyyyyydd, or ddyyyxyyyyyyyxyxyyyyydd, from 5′ to 3′, wherein each “y” is a 2′-O-methyl-β-D-ribosyl sugar moiety, each “d” is a 2′-β-D-deoxyribosyl sugar moiety, and each “x” is independently selected from a sugar surrogate, a stereo-non-standard sugar moiety, a 2′-β-D-deoxyribosyl sugar moiety, a β-D-ribosyl sugar moiety, and a 2′-fluoro-β-D-ribosyl sugar moiety, and “z” is independently selected from a sugar surrogate, a stereo-non-standard sugar moiety, a bicyclic sugar moiety, a 2′-β-D-deoxyribosyl sugar moiety, a β-D-ribosyl sugar moiety, and a 2′-fluoro-β-D-ribosyl sugar moiety.
Embodiment 114. The RNAi agent of embodiment 113, wherein at least one “x” is a stereo-non-standard sugar moiety.
Embodiment 115. The RNAi agent of embodiment 114, wherein at least one “x” is a 2′-fluoro-β-D-xylosyl sugar moiety.
Embodiment 116. The RNAi agent of embodiment 113, wherein at least one “x” is a sugar surrogate.
Embodiment 117. The RNAi agent of embodiment 116, wherein exactly one “x” is a sugar surrogate.
Embodiment 118. The RNAi agent of embodiment 116 or 117 wherein the sugar surrogate is fluoro hexitol nucleic acid (F-HNA).
Embodiment 119. The RNAi agent of embodiment 113, wherein at least one “x” is a bicyclic sugar moiety.
Embodiment 120. The RNAi agent of embodiment 119, wherein the bicyclic sugar moiety is selected from cEt and LNA.
Embodiment 121. The RNAi agent of any of embodiments 92-120, wherein the antisense RNAi oligonucleotide has a target complementary region of at least 18 consecutive nucleosides that are at least 85% complementary to an equal length portion of a target nucleic acid.
Embodiment 122. The RNAi agent of embodiment 121, wherein the antisense RNAi oligonucleotide has a target complementary region that is at least 90%, at least 95%, or 100% complementary to an equal length portion of a target nucleic acid.
Embodiment 123. The RNAi agent of embodiment 122, wherein the target complementary region comprises 21 consecutive nucleosides.
Embodiment 124. The RNAi agent of any of embodiments 92-123, wherein the sense RNAi oligonucleotide has a complementary region of at least 18 consecutive nucleosides that are at least 85% complementary to an equal length region of the antisense siRNA oligonucleotide.
Embodiment 125. The RNAi agent of embodiment 124, wherein the sense RNAi oligonucleotide consists of 2 fewer linked nucleosides than the antisense RNAi oligonucleotide.
Embodiment 126. The RNAi agent of embodiment 125, wherein the complementary region of the sense siRNA consists of 21 nucleobases.
Embodiment 127. The RNAi agent of embodiment 126, wherein the antisense RNAi oligonucleotide consists of 23 linked nucleosides, the sense siRNA oligonucleotide consists of 21 linked nucleosides, the sense siRNA oligonucleotide is 100% complementary to the antisense RNAi oligonucleotide, and the antisense RNAi oligonucleotide is at least 85% or at least 90% complementary to an equal length portion of a target nucleic acid.
Embodiment 128. The RNAi agent of any of embodiments 92-127, wherein the sense RNAi oligonucleotide comprises at least one internucleoside linkage of Formula I:

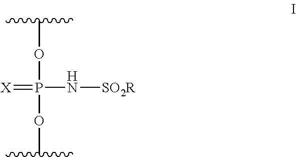

wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group.
Embodiment 129. The RNAi agent of embodiment 128, wherein each internucleoside linkage of the sense RNAi oligonucleotide is selected from an internucleoside linkage of Formula I, a phosphodiester internucleoside linkage, and a phosphorothioate internucleoside linkage.
Embodiment 130. The RNAi agent of embodiment 129, wherein the sense RNAi oligonucleotide has an internucleoside linkage motif selected of qq(o)ₙqq, wherein each “q” is independently selected from a phosphorothioate internucleoside linkage and an internucleoside linkage of Formula I, each “o” is a phosphodiester internucleoside linkage, and n is from 14-18.
Embodiment 131. The RNAi agent of embodiment 130, wherein the sense RNAi oligonucleotide has an internucleoside linkage motif selected of qq(o)ₙz(o)ₘqq, wherein each “q” is independently selected from a phosphorothioate internucleoside linkage and an internucleoside linkage of Formula I wherein X is O and R is methyl, each “o” is a phosphodiester internucleoside linkage, and “z” is an internucleoside linkage of Formula I wherein X is O and R is selected from C₁₀-C₂₀ alkyl, substituted C₁₀-C₂₀ alkyl, and a conjugate group, wherein n is from 2 to 5, m is from 8 to 15, and n+m is from 13 to 17.
Embodiment 132. The RNAi agent of any of embodiments 92-131, wherein the sense RNAi oligonucleotide consists of 21 linked nucleosides and has an internucleoside linkage motif selected from ssoooooooooooooooooo, ssoooooooooooooooooo, ssooooooooooooooooss, ssooozooooooooooooss, zzoooooooooooooooozz, ssoooozooozoooooooss, ssoooozozozoooooooss, ssoooozozzzoooooooss, zsoooooooooooooooosz, and zzoooooooooooooooooo, wherein each “s” is a phosphorothioate internucleoside linkage, each “z” is an internucleoside linkage of Formula I, and each “o” is a phosphodiester internucleoside linkage
Embodiment 133. The RNAi agent of any of embodiments 128-132, wherein for at least one internucleoside linkage of Formula I of the sense RNAi oligonucleotide, X is O and R is methyl.
Embodiment 134. The RNAi agent of any of embodiments 128-133, wherein for at least one internucleoside linkage of Formula I of the sense RNAi oligonucleotide, X is O and R is C₁₆.
Embodiment 135. The RNAi agent of any of embodiments 128-134, wherein for at least one internucleoside linkage of Formula I of the sense RNAi oligonucleotide, R is a conjugate group.
Embodiment 136. The RNAi agent of embodiment 135, wherein the conjugate group comprises a conjugate linker and a conjugate moiety.
Embodiment 137. The RNAi agent of embodiment 136, wherein the conjugate moiety is selected from a lipid, a carbohydrate, a GalNAc, an antibody or fragment thereof, or a peptide.
Embodiment 138. The RNAi agent of any of embodiments 92-137, wherein each of at least two sugar moieties of the sense RNAi is selected from a 2′-O-methyl-β-D-ribosyl sugar moiety, a 2′-O-methoxyethyl-β-D-ribosyl sugar moiety, a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, and wherein each of at least two such sugar moieties are different from each other.
Embodiment 139. The RNAi agent of any of embodiments 92-138, wherein the sense RNAi oligonucleotide consists of 21 linked nucleosides and has a sugar motif of yyyyyyxyxxxyyyyyyyyyy, from 5′ to 3′, wherein each “y” is a 2′-O-methyl-β-D-ribosyl sugar moiety and each “x” is independently selected from a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, wherein at least one “x” is other-than a 2′-fluoro-β-D-ribosyl sugar moiety.
Embodiment 140. The RNAi agent of embodiment 138 or 139, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.
Embodiment 141. The RNAi agent of embodiment 140, wherein at least one “x” is a 2′-β-D-deoxyribosyl sugar moiety.
Embodiment 142. The RNAi agent of embodiment 141, wherein each “x” is a 2′-β-D-deoxyribosyl sugar moiety.
Embodiment 143. The RNAi agent of embodiment 140, wherein at least one “x” is a 2′-fluoro-β-D-xylosyl sugar moiety.
Embodiment 144. The RNAi agent of embodiment 143, wherein each “x” is a 2′-fluoro-β-D-xylosyl sugar moiety.
Embodiment 145. The RNAi agent of embodiment 140, wherein at least one “x” is a sugar surrogate.
Embodiment 146. The RNAi agent of embodiment 145, wherein each sugar surrogate is independently selected from fluoro hexitol nucleic acid (F-HNA), hexitol nucleic acid (HNA) or altritol nucleic acid (ANA).
Embodiment 147. The RNAi agent of any of embodiments 92-146, wherein the sense RNAi oligonucleotide has a sugar motif selected from: yyyyyyfyff[f2bDx]yyyyyyyyyy, yyyyyyfyf[f2bDx]fyyyyyyyyyy, yyyyyyfy[f2bDx]ffyyyyyyyyyy, yyyyyy[f2bDx]yfffyyyyyyyyyy, yyyyyy[f2bDx]y[f2bDx][f2bDx][f2bDx]yyyyyyyyyy, yyyyy[16C2r]fyfffyyyyyyyyyy, yyyyy[C16A]fyfffyyyyyyyyyy, yyyyy[16C2r]fyfffyyyyyyyyyy, yyyyy[16C2r]fyff[f2bDx]yyyyyyyyyy, yyyyy[16C2r]fyf[f2bDx]fyyyyyyyyyy, yyyyy[16C2r]fy[f2bDx]ffyyyyyyyyyy, yyyyy[16C2r][f2bDx]yfffyyyyyyyyyy, yyyyy[16C2r][f2bDx]y[f2bDx][f2bDx][f2bDx]yyyyyyyyyy, yyyyyyfyfffyyyyyyyyyyddddddddd, yyyyy[16C2r]fyff[F-HNA]yyyyyyyyyy, yyyyy[16C2r]fyf[F-HNA]fyyyyyyyyyy, yyyyy[16C2r]fy[F-HNA]ffyyyyyyyyyy, yyyyy[16C2r][F-HNA]yfffyyyyyyyyyy, yyyyy[16C2r][F-HNA]yff[F-HNA]yyyyyyyyyy, yyyyy[16C2r][F-HNA]y[F-HNA][F-HNA][F-HNA]yyyyyyyyyy, yyyyyyyyyyyyyyyyyyyyy, yyyyyyyyfffyyyyyyyyyy, yyyyyyfyyffyyyyyyyyyy, yyyyyyfyfyfyyyyyyyyyy, yyyyyyfyffyyyyyyyyyyy, yyyyyyyyyffyyyyyyyyyy, yyyyyyfyyyfyyyyyyyyyy, yyyyyyfyfyyyyyyyyyyyy, yyyyyyyyfyfyyyyyyyyyy, yyyyyyyyffyyyyyyyyyyy, yyyyyyfyyfyyyyyyyyyyy, yyyyyyeyeeeyyyyyyyyyy, yyyyyyeyfffyyyyyyyyyy, yyyyyyfyeffyyyyyyyyyy, yyyyyyfyfefyyyyyyyyyy, yyyyyyfyffeyyyyyyyyyy, yyyyyyeyeffyyyyyyyyyy, yyyyyyfyeefyyyyyyyyyy, yyyyyyfyfeeyyyyyyyyyy, yyyyyyeyfefyyyyyyyyyy, yyyyyyeyffeyyyyyyyyyy, yyyyyyfyefeyyyyyyyyyy, yyyyyydyfffyyyyyyyyyy, yyyyyyfydffyyyyyyyyyy, yyyyyyfyfdfyyyyyyyyyy, yyyyyyfyffdyyyyyyyyyy, yyyyyydydffyyyyyyyyyy, yyyyyydyfdfyyyyyyyyyy, yyyyyydyffdyyyyyyyyyy, yyyyyydydfdyyyyyyyyyy, yyyyyyfydddyyyyyyyyyy, yyyyyydyddfyyyyyyyyyy, yyyyyydydddyyyyyyyyyy, yyyyydddddddddddyyyyy, yyyyyydddddddddyyyyyy, yyyyyyydddddddyyyyyyy, yyyyyyyydddddyyyyyyyy, eeeeedddddddddddeeeee, eeeeeedddddddddeeeeee, eeeeeeedddddddeeeeeee, eeeeeeeedddddeeeeeeee, yyyyyydydydydyddyyyyy, eeeeeedededededdeeeee, dydydydydydydydydydyd, dydydyfyfffydydydydyd, dedededededededededed, dededefefffededededed, ryryryryryryryryryryr, ryryryfyfffyryryryryr, rerererererererererer, rererefefffererererer, yyyyy[16C2r][f2bDx]yff[f2bDx]yyyyyyyyyy, yyyyyyryfffyyyyyyyyyy, yyyyyyfyrffyyyyyyyyyy, yyyyyyfyfrfyyyyyyyyyy, yyyyyyfyffryyyyyyyyyy, yyyyyyryrrryyyyyyyyyy, yyyyyyfyff[bDdx]yyyyyyyyyy, yyyyyyfyf[bDdx]fyyyyyyyyyy, yyyyyyfy[bDdx]ffyyyyyyyyyy, yyyyyy[bDdx]yfffyyyyyyyyyy, yyyyyy[bDdx]y[bDdx][bDdx][bDdx]yyyyyyyyyy, yyyyyyfyff[F-HNA]yyyyyyyyyy, yyyyyyfyf[F-HNA]fyyyyyyyyyy, yyyyyyfy[F-HNA]ffyyyyyyyyyy, yyyyyy[F-HNA]yfffyyyyyyyyyy, yyyyyy[F-HNA]y[F-HNA][F-HNA][F-HNA]yyyyyyyyyy, yyyyyyfyf[bDx]fyyyyyyyyyy, yyyyyyfyf[bDx]fyyyyyyyyyy; wherein]; wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C2r]” represents a 2′-O-hexadecyl-β-D-ribosyl sugar moiety, “[C16A]” represents 2′-O-hexylpalmitamide-β-D-ribosyl sugar moiety “[F-HNA]” represents a F-HNA sugar surrogate, “[bDdx]” represents a 2′-β-D-deoxyxylosyl sugar moiety, and “[bDx]” represents a β-D-xylosyl sugar moiety.
Embodiment 148. The RNAi agent of any of embodiments 92-147, wherein the sense RNAi oligonucleotide comprises a deoxy region consisting of 5 to 11 contiguous nucleosides flanked on the 5′ side by a 5′-region consisting of 5-8 linked 5′-region nucleosides and on the 3′ side by a 3′-region consisting of 5-8 linked 3′-region nucleosides; wherein
   each deoxy region nucleoside comprises a 2′-β-D-deoxyribosyl sugar moiety; and wherein
   each 5′-region nucleoside and each 3′-region nucleoside is selected from a 2′-O-methyl-β-D-ribosyl sugar moiety and a 2′-O-methoxyethyl-β-D-ribosyl sugar moiety.
Embodiment 149. The RNAi agent of embodiment 148, wherein each 5′-region nucleoside and each 3′-region nucleoside is a 2′-O-methyl-β-D-ribosyl sugar moiety.
Embodiment 150. The RNAi agent of any of embodiments 92-149, wherein the last two 3′-nucleosides of the antisense RNAi oligonucleotide are not paired with the sense RNAi oligonucleotide.
Embodiment 151. The RNAi agent of any of embodiments 92-150, wherein the last two 3′-nucleosides of the antisense RNAi oligonucleotide are not complementary to the target nucleic acid.
Embodiment 152. The RNAi agent of any of embodiments 92-151, wherein the last two 3′-nucleosides of the antisense RNAi oligonucleotide comprise a thymine nucleobase.
Embodiment 153. The RNAi agent of any of embodiments 92-152, wherein the 5′-stabilized phosphate group is selected from vinyl phosphonate, mesyl phosphoramidate, and cyclopropyl phosphonate.
Embodiment 154. The RNAi agent of any of embodiments 92-153, wherein the 5′-stabilized phosphate group is vinyl phosphonate.
Embodiment 155. The RNAi agent of any of embodiments 92-154, wherein the antisense siRNA oligomeric compound comprises a conjugate group.
Embodiment 156. The RNAi agent of embodiment 155, wherein the conjugate group comprises a conjugate moiety and a conjugate linker.
Embodiment 157. The RNAi agent of embodiment 156, wherein the conjugate moiety is a cell-targeting moiety.
Embodiment 158. The RNAi agent of embodiment 156, wherein the conjugate moiety is a lipid.
Embodiment 159. The RNAi agent of embodiment 156, wherein the conjugate moiety comprises C₁₂-C₂₀ alkyl.
Embodiment 160. The RNAi agent of embodiment 156, wherein the conjugate moiety is C₁₆ alkyl.
Embodiment 161. The RNAi agent of embodiment 156, wherein the conjugate moiety is a carbohydrate.
Embodiment 162. The RNAi agent of embodiment 156, wherein the conjugate moiety comprises a GalNAc.
Embodiment 163. The RNAi agent of embodiment 156, wherein the conjugate moiety comprises an antibody or an antibody fragment.
Embodiment 164. The RNAi agent of embodiment 156, wherein the conjugate moiety comprises a peptide.
Embodiment 165. The RNAi agent of any of embodiments 156-164, wherein the conjugate moiety is part of an internucleoside linkage of Formula I.
Embodiment 166. The RNAi agent of any of embodiments 156-164, wherein the conjugate moiety is a 2′-modification of a sugar moiety.
Embodiment 167. The RNAi agent of any of embodiments 156-164, wherein the conjugate moiety is attached at the 3′-terminal of the antisense RNAi oligonucleotide.
Embodiment 168. The RNAi agent of any of embodiments 156-164, wherein the conjugate moiety is attached at the 5′-terminal of the antisense RNAi oligonucleotide.
Embodiment 169. The RNAi agent of any of embodiments 92-168, wherein the sense siRNA oligomeric compound comprises a conjugate group.
Embodiment 170. The RNAi agent of embodiment 169, wherein the conjugate group comprises a conjugate moiety and a conjugate linker.
Embodiment 171. The RNAi agent of embodiment 170, wherein the conjugate moiety is a cell-targeting moiety.
Embodiment 172. The RNAi agent of embodiment 170, wherein the conjugate moiety is a lipid.
Embodiment 173. The RNAi agent of embodiment 170, wherein the conjugate moiety comprises C₁₂-C₂₀ alkyl.
Embodiment 174. The RNAi agent of embodiment 170, wherein the conjugate moiety is C₁₆ alkyl.
Embodiment 175. The RNAi agent of embodiment 170, wherein the conjugate moiety is a carbohydrate.
Embodiment 176. The RNAi agent of embodiment 170, wherein the conjugate moiety comprises a GalNAc.
Embodiment 177. The RNAi agent of embodiment 170, wherein the conjugate moiety comprises an antibody or an antibody fragment.
Embodiment 178. The RNAi agent of embodiment 170, wherein the conjugate moiety comprises a peptide.
Embodiment 179. The RNAi agent of any of embodiments 169-178, wherein the conjugate moiety is part of an internucleoside linkage of Formula I.
Embodiment 180. The RNAi agent of any of embodiments 169-179, wherein the conjugate moiety is a 2′-modification of a sugar moiety.
Embodiment 181. The RNAi agent of any of embodiments 169-179, wherein the conjugate moiety is attached at the 3′-terminal of the sense RNAi oligonucleotide.
Embodiment 182. The RNAi agent of any of embodiments 169-179, wherein the conjugate moiety is attached at the 5′-terminal of the sense RNAi oligonucleotide.
Embodiment 183. An RNAi agent, comprising an antisense siRNA oligomeric compound comprising an antisense RNAi oligonucleotide consisting of 21-25 linked nucleosides and a sense RNAi oligomeric compound comprising a sense RNAi oligonucleotide consisting of 19-23 linked nucleosides, wherein the 5′-terminus of the antisense RNAi oligonucleotide has Structure A:

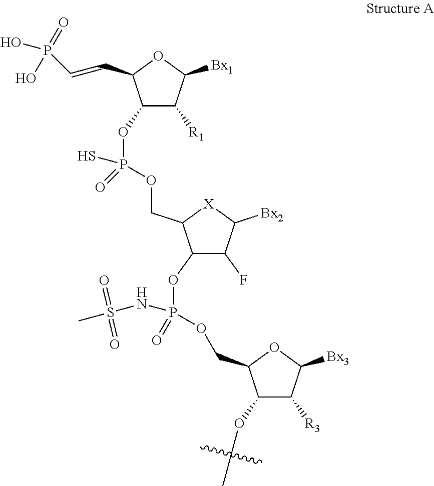

wherein
X is selected from O and O—C(H₂);
each Bx is an independently selected heterocyclic base moiety;
each of R₁ and R₃ is independently O[C(R₄)(R₅)]ₙ—[(C═O)ₘ—Z]ⱼ—R₆; wherein
each R₄ and R₅ is, independently, H, halogen, C₁-C₆ alkyl or substituted C₁-C₆ alkyl;
each Z is O, S or N(E₁);
R₆ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
Q is O, S or NJ₃;
each J₁, J₂ and J₃ is, independently, H or C₁-C₆ alkyl.
Embodiment 184. The RNAi agent of embodiment 183, wherein the 5′-terminus of the antisense RNAi oligonucleotide has Structure Aᵣ:

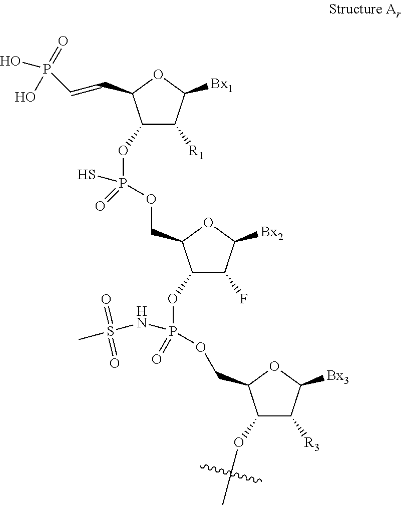

wherein
each Bx is an independently selected heterocyclic base moiety;
each of R₁ and R₃ is independently O[C(R₄)(R₅)]ₙ—[(C═O)ₘ—Z]ⱼ—R₆; wherein
each R₄ and R₅ is, independently, H, halogen, C₁-C₆ alkyl or substituted C₁-C₆ alkyl;
each Z is O, S or N(E₁);
R₆ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
Q is O, S or NJ₃;
each J₁, J₂ and J₃ is, independently, H or C₁-C₆ alkyl.
Embodiment 185. The RNAi agent of embodiment 183, wherein the 5′-terminus of the antisense RNAi oligonucleotide has Structure Aₓ:

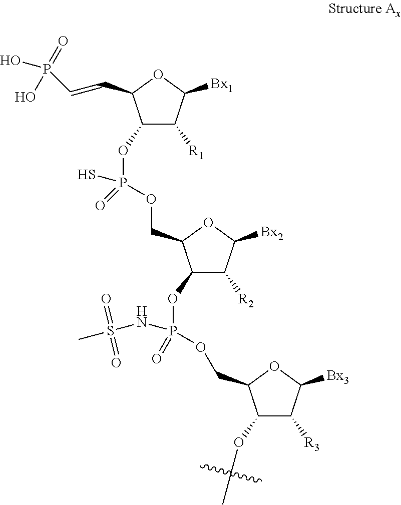

wherein
each Bx is an independently selected heterocyclic base moiety;
each of R₁ and R₃ is independently O[C(R₄)(R₅)]ₙ—[(C═O)ₘ—Z]ⱼ—R₆; wherein
each R₄ and R₅ is, independently, H, halogen, C₁-C₆ alkyl or substituted C₁-C₆ alkyl;
each Z is O, S or N(E₁);
R₆ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
Q is O, S or NJ₃;
each J₁, J₂ and J₃ is, independently, H or C₁-C₆ alkyl.
Embodiment 186. The RNAi agent of embodiment 183, wherein the 5′-terminus of the antisense RNAi oligonucleotide has Structure Aₕ:

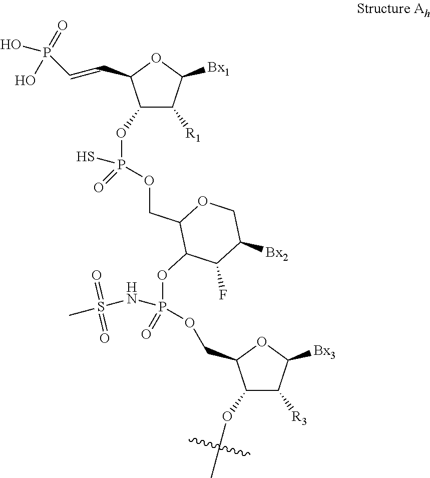

wherein
each Bx is an independently selected heterocyclic base moiety;
each of R₁ and R₃ is independently O[C(R₄)(R₅)]ₙ—[(C═O)ₘ—Z]ⱼ—R₆; wherein
each R₄ and R₅ is, independently, H, halogen, C₁-C₆ alkyl or substituted C₁-C₆ alkyl;
each Z is O, S or N(E₁);
R₆ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
Q is O, S or NJ₃;
each J₁, J₂ and J₃ is, independently, H or C₁-C₆ alkyl.
Embodiment 187. The RNAi agent of any of embodiments 183-186, wherein R₁ is selected from OCH₃ and OCH₂CH₂OCH₃.
Embodiment 188. The RNAi agent of any of embodiments 183-187, wherein R₃ is OCH₃.
Embodiment 189. The RNAi agent of any of embodiments 183-188, wherein each Bx is selected from adenine, cytosine, uracil, thymine, guanine, and 5-methyl cytosine.
Embodiment 190. The RNAi agent of any of embodiments 183-189, wherein Bx₁ is thymine.
Embodiment 191. The RNAi agent of any of embodiments 183-190, wherein the antisense RNAi oligonucleotide comprises at least one sugar moiety selected from 2′-O-methyl-β-D-ribosyl sugar moiety, a 2′-O-methoxyethyl-β-D-ribosyl sugar moiety, a sugar surrogate, a stereo-non-standard sugar moiety, a 2′-β-D-deoxyribosyl sugar moiety, and a β-D-ribosyl sugar moiety.
Embodiment 192. The RNAi agent of embodiment 191, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.
Embodiment 193. The RNAi agent of embodiment 191 or 192, wherein each sugar surrogate is independently selected from fluoro hexitol nucleic acid (F-HNA), hexitol nucleic acid (HNA), or altritol nucleic acid (ANA).
Embodiment 194. The RNAi agent of any of embodiments 183-193, wherein the antisense RNAi oligonucleotide comprises no more than three 2′-fluoro-β-D-ribosyl sugar moieties.
Embodiment 195. The RNAi agent of any of embodiments 183-194, wherein each internucleoside linkage of the antisense RNAi oligonucleotide is selected from mesyl phosphoramidate internucleoside linkage, a phosphodiester internucleoside linkage, and a phosphorothioate internucleoside linkage.
Embodiment 196. The RNAi agent of any of embodiments 183-195, wherein the antisense RNAi oligonucleotide has an internucleoside linkage motif selected from sz(o)ₙzs and sz(o)ₙss, wherein each “s” is a phosphorothioate internucleoside linkage, each “z” is an internucleoside linkage of Formula I, each “o” is a phosphodiester internucleoside linkage, and n is from 16-20.
Embodiment 197. The RNAi agent of embodiment 196, wherein for each internucleoside linkage of Formula I, R is methyl.
Embodiment 198. The RNAi agent of any of embodiments 183-197, wherein the antisense RNAi oligonucleotide consists of 23 linked nucleosides.
Embodiment 199. The RNAi agent of any of embodiments 183-198, wherein the antisense RNAi oligonucleotide has a sugar motif selected from: yfyyyfyyyyyyyfyfyyyyyyy, y[f2bDx]yyyfyyyyyyyfyfyyyyyyy, yfyyy[f2bDx]yyyyyyyfyfyyyyyyy, yfyyyfyyyyyyy[f2bDx]yfyyyyyyy, yfyyyfyyyyyyyfy[f2bDx]yyyyyyy, y[f2bDx]yyy[f2bDx]yyyyyyy[f2bDx]y[f2bDx]yyyyyyy, yfyfyfyfyfyfyfyfyfyfyyy, efyfyfyfyfyfyfyfyfyfyyy, efyyyyyyyyyyyfyfyyyyyyy, efyyyfyyyyyyyfyfyyyyyyy, efyyyfy[16C2r]yyyyyfyfyyyyyyy, e[f2bDx]yyyfyyyyyyyfyfyyyyyyy, efyyy[f2bDx]yyyyyyyfyfyyyyyyy, efyyyfyyyyyyy[f2bDx]yfyyyyyyy, efyyyfyyyyyyyfy[f2bDx]yyyyyyy, e[f2bDx]yyy[f2bDx]yyyyyyy[f2bDx]y[f2bDx]yyyyyyy, efyyy[F-HNA]yyyyyyyfyfyyyyyyy, efyyyfyyyyyyy[F-HNA]yfyyyyyyy, efyyyfyyyyyyyfy[F-HNA]yyyyyyy, yfyyydyyyyyyyfyfyyyyyyy, yfyyyfyyyyyyydyfyyyyyyy, yfyyyfyyyyyyyfydyyyyyyy, yfyyydyyyyyyydydyyyyyyy, yryyyfyyyyyyyfyfyyyyyyy, yfyyyryyyyyyyfyfyyyyyyy, yfyyyfyyyyyyyryfyyyyyyy, yfyyyfyyyyyyyfyryyyyyyy, yfyyy[bDdx]yyyyyyyfyfyyyyyyy, yfyyyfyyyyyyy[bDdx]yfyyyyyyy, yfyyyfyyyyyyyfy[bDdx]yyyyyyy, y[F-HNA]yyyfyyyyyyyfyfyyyyyyy, yfyyy[F-HNA]yyyyyyyfyfyyyyyyy, yfyyyfyyyyyyy[F-HNA]yfyyyyyyy, yfyyyfyyyyyyyfy[F-HNA]yyyyyyy, y[F-HNA]yyy[F-HNA]yyyyyyy[F-HNA]y[F-HNA]yyyyyyy, yfyyyfyyyyyyy[2bDx]yfyyyyyyy, yfyyy[bDa]yyyyyyyfyfyyyyyyy, yfyyyfyyyyyyy[bDa]yfyyyyyyy, yfyyyfyyyyyyyfy[bDa]yyyyyyy, yfyyyfyyyyyyy[bDx]yfyyyyyyy, yfyyyfyffyyyyfyfyyyyydd, yfyyyfyffyyyyfyfyyyyy[bLdr][bLdr], yfyyyfyffyyyyfyfyyyyy[aDdr][aDdr], yfyyyfyffyyyyfyfyyyyy[bDdx][bDdx], yfyyyfyffyyyyfyfyyyyy[bLdx][bLdx], yfyyyfyffyyyyfyfyyyyy[aDdx][aDdx], yfyyyfyffyyyyfyfyyyyy[aLdx][aLdx]; wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C2r]” represents a 2′-O-hexadecyl-β-D-ribosyl sugar moiety, “[F-HNA]” represents a F-HNA sugar surrogate, “[bDdx]” represents a 2′-β-D-deoxyxylosyl sugar moiety, “[bDx]” represents a β-D-xylosyl sugar moiety, “[bDa]” represents a β-D-arabinosyl sugar moiety, “[bLdr]” represents a 2′-β-L-deoxyribosyl sugar moiety, “[aDdr]” represents a 2′-α-D-deoxyribosyl sugar moiety, “[aLdr]” represents a 2′-α-L-deoxyribosyl sugar moiety, “[bLdx]” represents a 2′-β-L-deoxyxylosyl sugar moiety, “[aDdx]” represents an 2′-α-D-deoxyxylosyl sugar moiety, and “[aLdx]” represents an 2′-α-L-deoxyxylosyl sugar moiety.
Embodiment 200. The RNAi agent of any of embodiments 183-199, wherein the antisense RNAi oligonucleotide has a target complementary region of at least 18 consecutive nucleosides that are at least 85% complementary to an equal length portion of a target nucleic acid.
Embodiment 201. The RNAi agent of embodiment 200, wherein the antisense RNAi oligonucleotide has a target complementary region that is at least 90%, at least 95%, or 100% complementary to an equal length portion of a target nucleic acid.
Embodiment 202. The RNAi agent of embodiment 201, wherein the target complementary region comprises 21 consecutive nucleosides.
Embodiment 203. The RNAi agent of any of embodiments 183-202, wherein the sense RNAi oligonucleotide has a complementary region of at least 18 consecutive nucleosides that are at least 85% complementary to an equal length region of the antisense siRNA oligonucleotide.
Embodiment 204. The RNAi agent of embodiment 203, wherein the sense RNAi oligonucleotide consists of 2 fewer linked nucleosides than the antisense RNAi oligonucleotide.
Embodiment 205. The RNAi agent of embodiment 203, wherein the complementary region of the sense siRNA consists of 21 nucleobases.
Embodiment 206. The RNAi agent of embodiment 203, wherein the antisense RNAi oligonucleotide consists of 23 linked nucleosides, the sense siRNA oligonucleotide consists of 21 linked nucleosides, the sense siRNA oligonucleotide is 100% complementary to the antisense RNAi oligonucleotide, and the antisense RNAi oligonucleotide is at least 85% complementary to an equal length portion of a target nucleic acid.
Embodiment 207. The RNAi agent of any of embodiments 183-206, each internucleoside linkage of the sense RNAi oligonucleotide is selected from an internucleoside linkage of Formula I, a phosphodiester internucleoside linkage, and a phosphorothioate internucleoside linkage.
Embodiment 208. The RNAi agent of embodiment 207, wherein the sense RNAi oligonucleotide has an internucleoside linkage motif selected of qq(o)ₙqq, wherein each “q” is independently selected from a phosphorothioate internucleoside linkage and an internucleoside linkage of Formula I, each “o” is a phosphodiester internucleoside linkage, and n is from 14-18.
Embodiment 209. The RNAi agent of embodiment 207 or 208, wherein for each internucleoside linkage of Formula I, R is methyl.
Embodiment 210. The RNAi agent of embodiment 219, wherein the sense RNAi oligonucleotide has an internucleoside linkage motif selected of qq(o)ₙz(o)ₘqq, wherein each “q” is independently selected from a phosphorothioate internucleoside linkage and an internucleoside linkage of Formula I wherein X is O and R is methyl, each “o” is a phosphodiester internucleoside linkage, and “z” is an internucleoside linkage of Formula I wherein X is O and R is selected from C₁₀-C₂₀ alkyl, substituted C₁₀-C₂₀ alkyl, and a conjugate group, wherein
   n is from 2 to 5, m is from 8 to 15, and n+m is from 13 to 17.
Embodiment 211. The RNAi agent of any of embodiments 183-210, wherein the sense RNAi oligonucleotide consists of 21 linked nucleosides and has an internucleoside linkage motif selected from ssoooooooooooooooooo, ssoooooooooooooooooo, ssooooooooooooooooss, ssooooooooooooooooss, zzoooooooooooooooozz, ssoooozooozoooooooss, ssoooozozozoooooooss, ssoooozozzzoooooooss, zsoooooooooooooooosz, and zzoooooooooooooooooo, wherein each “s” is a phosphorothioate internucleoside linkage, each “z” is an internucleoside linkage of Formula I, and each “o” is a phosphodiester internucleoside linkage.
Embodiment 212. The RNAi agent of any of embodiments 183-211, wherein for at least one internucleoside linkage of Formula I of the sense RNAi oligonucleotide, X is O and R is methyl.
Embodiment 213. The RNAi agent of any of embodiments 183-211, wherein for at least one internucleoside linkage of Formula I of the sense RNAi oligonucleotide, X is O and R is C₁₆.
Embodiment 214. The RNAi agent of any of embodiments 183-211, wherein for at least one internucleoside linkage of Formula I of the sense RNAi oligonucleotide, R is a conjugate group.
Embodiment 215. The RNAi agent of embodiment 214, wherein the conjugate group comprises a conjugate linker and a conjugate moiety.
Embodiment 216. The RNAi agent of embodiment 215, wherein the conjugate moiety is selected from a lipid, a carbohydrate, a GalNAc, an antibody or fragment thereof, or a peptide.
Embodiment 217. The RNAi agent of any of embodiments 183-216, wherein each of at least two sugar moieties of the sense RNAi is selected from a 2′-O-methyl-β-D-ribosyl sugar moiety, a 2′-O-methoxyethyl-β-D-ribosyl sugar moiety, a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, and wherein each of at least two such sugar moieties are different from each other.
Embodiment 218. The RNAi agent of any of embodiments 183-217, wherein the sense RNAi oligonucleotide consists of 21 linked nucleosides and has a sugar motif of yyyyyyxyxxxyyyyyyyyyy, from 5′ to 3′, wherein each “y” is a 2′-O-methyl β-D-ribosyl sugar moiety and each “x” is independently selected from a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, wherein at least one “x” is other-than a 2′-fluoro-β-D-ribosyl sugar moiety.
Embodiment 219. The RNAi agent of embodiment 217 or 218, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.
Embodiment 220. The RNAi agent of embodiment 219, wherein at least one “x” is a 2′-β-D-deoxyribosyl sugar
Embodiment 221. The RNAi agent of embodiment 220, wherein each “x” is a 2′-β-D-deoxyribosyl sugar moiety.
Embodiment 222. The RNAi agent of embodiment 219, wherein at least one “x” is a 2′-fluoro-β-D-xylosyl sugar moiety.
Embodiment 223. The RNAi agent of embodiment 222, wherein each “x” is a 2′-fluoro-β-D-xylosyl sugar moiety.
Embodiment 224. The RNAi agent of embodiment 219, wherein at least one “x” is a sugar surrogate.
Embodiment 225. The RNAi agent of embodiment 224, wherein each sugar surrogate is independently selected from fluoro hexitol nucleic acid (F-HNA), hexitol nucleic acid (HNA) or altritol nucleic acid (ANA).
Embodiment 226. The RNAi agent of any of embodiments 183-225, wherein the sense RNAi oligonucleotide has a sugar motif selected from: yyyyyyfyff[f2bDx]yyyyyyyyyy, yyyyyyfyf[f2bDx]fyyyyyyyyyy, yyyyyyfy[f2bDx]ffyyyyyyyyyy, yyyyyy[f2bDx]yfffyyyyyyyyyy, yyyyyy[f2bDx]y[f2bDx][f2bDx][f2bDx]yyyyyyyyyy, yyyyy[16C2r]fyfffyyyyyyyyyy, yyyyy[C16A]fyfffyyyyyyyyyy, yyyyy[16C2r]fyfffyyyyyyyyyy, yyyyy[16C2r]fyff[f2bDx]yyyyyyyyyy, yyyyy[16C2r]fyf[f2bDx]fyyyyyyyyyy, yyyyy[16C2r]fy[f2bDx]ffyyyyyyyyyy, yyyyy[16C2r][f2bDx]yfffyyyyyyyyyy, yyyyy[16C2r][f2bDx]y[f2bDx][f2bDx][f2bDx]yyyyyyyyyy, yyyyyyfyfffyyyyyyyyyyddddddddd, yyyyy[16C2r]fyff[F-HNA]yyyyyyyyyy, yyyyy[16C2r]fyf[F-HNA]fyyyyyyyyyy, yyyyy[16C2r]fy[F-HNA]ffyyyyyyyyyy, yyyyy[16C2r][F-HNA]yfffyyyyyyyyyy, yyyyy[16C2r][F-HNA]yff[F-HNA]yyyyyyyyyy, yyyyy[16C2r][F-HNA]y[F-HNA][F-HNA][F-HNA]yyyyyyyyyy, yyyyyyyyyyyyyyyyyyyyy, yyyyyyyyfffyyyyyyyyyy, yyyyyyfyyffyyyyyyyyyy, yyyyyyfyfyfyyyyyyyyyy, yyyyyyfyffyyyyyyyyyyy, yyyyyyyyyffyyyyyyyyyy, yyyyyyfyyyfyyyyyyyyyy, yyyyyyfyfyyyyyyyyyyyy, yyyyyyyyfyfyyyyyyyyyy, yyyyyyyyffyyyyyyyyyyy, yyyyyyfyyfyyyyyyyyyyy, yyyyyyeyeeeyyyyyyyyyy, yyyyyyeyfffyyyyyyyyyy, yyyyyyfyeffyyyyyyyyyy, yyyyyyfyfefyyyyyyyyyy, yyyyyyfyffeyyyyyyyyyy, yyyyyyeyeffyyyyyyyyyy, yyyyyyfyeefyyyyyyyyyy, yyyyyyfyfeeyyyyyyyyyy, yyyyyyeyfefyyyyyyyyyy, yyyyyyeyffeyyyyyyyyyy, yyyyyyfyefeyyyyyyyyyy, yyyyyydyfffyyyyyyyyyy, yyyyyyfydffyyyyyyyyyy, yyyyyyfyfdfyyyyyyyyyy, yyyyyyfyffdyyyyyyyyyy, yyyyyydydffyyyyyyyyyy, yyyyyydyfdfyyyyyyyyyy, yyyyyydyffdyyyyyyyyyy, yyyyyydydfdyyyyyyyyyy, yyyyyyfydddyyyyyyyyyy, yyyyyydyddfyyyyyyyyyy, yyyyyydydddyyyyyyyyyy, yyyyydddddddddddyyyyy, yyyyyydddddddddyyyyyy, yyyyyyydddddddyyyyyyy, yyyyyyyydddddyyyyyyyy, eeeeedddddddddddeeeee, eeeeeedddddddddeeeeee, eeeeeeedddddddeeeeeee, eeeeeeeedddddeeeeeeee, yyyyyydydydydyddyyyyy, eeeeeedededededdeeeee, dydydydydydydydydydyd, dydydyfyfffydydydydyd, dedededededededededed, dededefefffededededed, ryryryryryryryryryryr, ryryryfyfffyryryryryr, rerererererererererer, rererefefffererererer, yyyyy[16C2r][f2bDx]yff[f2bDx]yyyyyyyyyy, yyyyyyryfffyyyyyyyyyy, yyyyyyfyrffyyyyyyyyyy, yyyyyyfyfrfyyyyyyyyyy, yyyyyyfyffryyyyyyyyyy, yyyyyyryrrryyyyyyyyyy, yyyyyyfyff[bDdx]yyyyyyyyyy, yyyyyyfyf[bDdx]fyyyyyyyyyy, yyyyyyfy[bDdx]ffyyyyyyyyyy, yyyyyy[bDdx]yfffyyyyyyyyyy, yyyyyy[bDdx]y[bDdx][bDdx][bDdx]yyyyyyyyyy, yyyyyyfyff[F-HNA]yyyyyyyyyy, yyyyyyfyf[F-HNA]fyyyyyyyyyy, yyyyyyfy[F-HNA]ffyyyyyyyyyy, yyyyyy[F-HNA]yfffyyyyyyyyyy, yyyyyy[F-HNA]y[F-HNA][F-HNA][F-HNA]yyyyyyyyyy, yyyyyyfyf[bDx]fyyyyyyyyyy, yyyyyyfyf[bDx]fyyyyyyyyyy; wherein]; wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C2r]” represents a 2′-O-hexadecyl-β-D-ribosyl sugar moiety, “[C16A]” represents 2′-O-hexylpalmitamide-β-D-ribosyl sugar moiety “[F-HNA]” represents a F-HNA sugar surrogate, “[bDdx]” represents a 2′-β-D-deoxyxylosyl sugar moiety, and “[bDx]” represents a β-D-xylosyl sugar moiety.
Embodiment 227. The RNAi agent of any of embodiments 183-226, wherein the sense RNAi oligonucleotide comprises a deoxy region consisting of 5 to 11 contiguous nucleosides flanked on the 5′ side by a 5′-region consisting of 5-8 linked 5′-region nucleosides and on the 3′ side by a 3′-region consisting of 5-8 linked 3′-region nucleosides; wherein
   each deoxy region nucleoside comprises a 2′-β-D-deoxyribosyl sugar moiety; and wherein
   each 5′-region nucleoside and each 3′-region nucleoside is selected from a 2′-O-methyl-β-D-ribosyl sugar moiety and a 2′-O-methoxyethyl-β-D-ribosyl sugar moiety.
Embodiment 228. The RNAi agent of embodiment 227, wherein each 5′-region nucleoside and each 3′-region nucleoside is a 2′-O-methyl-β-D-ribosyl sugar moiety.
Embodiment 229. The RNAi agent of any of embodiments 183-228, wherein the last two 3′-nucleosides of the antisense RNAi oligonucleotide are not paired with the sense RNAi oligonucleotide.
Embodiment 230. The RNAi agent of any of embodiments 183-229, wherein the last two 3′-nucleosides of the antisense RNAi oligonucleotide are not complementary to the target nucleic acid.
Embodiment 231. The RNAi agent of any of embodiments 283-229, wherein the last two 3′-nucleosides of the antisense RNAi oligonucleotide comprise a thymine nucleobase.
Embodiment 232. The RNAi agent of any of embodiments 183-231, wherein the antisense siRNA oligomeric compound comprises a conjugate group.
Embodiment 233. The RNAi agent of embodiment 232, wherein the conjugate group comprises a conjugate moiety and a conjugate linker.
Embodiment 234. The RNAi agent of embodiment 233, wherein the conjugate moiety is selected from a lipid, a carbohydrate, a peptide, and antibody, or an antibody fragment.
Embodiment 235. The RNAi agent of embodiment 233, wherein the conjugate group comprises a C₁₂-C₂₀ alkyl, C₁₆ alkyl, or a GalNAc.
Embodiment 236. The RNAi agent of any of embodiments 183-235, wherein the sense siRNA oligomeric compound comprises a conjugate group.
Embodiment 237. The RNAi agent of embodiment 236, wherein the conjugate group comprises a conjugate moiety and a conjugate linker.
Embodiment 238. The RNAi agent of embodiment 237, wherein the conjugate moiety is a cell-targeting moiety.
Embodiment 239. The RNAi agent of embodiment 237, wherein the conjugate moiety is a lipid.
Embodiment 240. The RNAi agent of embodiment 237, wherein the conjugate moiety comprises C₁₂-C₂₀ alkyl.
Embodiment 241. The RNAi agent of embodiment 237, wherein the conjugate moiety is C₁₆ alkyl.
Embodiment 242. The RNAi agent of embodiment 237, wherein the conjugate moiety is a carbohydrate.
Embodiment 243. The RNAi agent of embodiment 237, wherein the conjugate moiety comprises a GalNAc.
Embodiment 244. The RNAi agent of embodiment 237, wherein the conjugate moiety comprises an antibody or an antibody fragment.
Embodiment 245. The RNAi agent of embodiment 237, wherein the conjugate moiety comprises a peptide.
Embodiment 246. The RNAi agent of any of embodiments 236-245, wherein the conjugate moiety is part of an internucleoside linkage of Formula I.
Embodiment 247. The RNAi agent of any of embodiments 236-245, wherein the conjugate moiety is a 2′-modification of a sugar moiety.
Embodiment 248. The RNAi agent of any of embodiments 236-245, wherein the conjugate moiety is attached at the 3′-terminal of the sense RNAi oligonucleotide.
Embodiment 249. The RNAi agent of any of embodiments 236-245, wherein the conjugate moiety is attached at the 5′-terminal of the sense RNAi oligonucleotide.
Embodiment 250. A chirally enriched population of RNAi agents of any of embodiments 92-249, wherein the population is enriched for antisense RNAi oligonucleotides and/or sense RNAi oligonucleotides comprising at least one particular internucleoside linkage having a particular stereochemical configuration.
Embodiment 251. The chirally enriched population of embodiment 250, wherein the population is enriched for antisense RNAi oligonucleotides and/or sense RNAi oligonucleotides comprising at least one particular internucleoside of Formula I having the (Sp) or (Rp) configuration.
Embodiment 252. The chirally enriched population of embodiment 250, wherein the population is enriched for antisense RNAi oligonucleotides comprising at least one internucleoside linkage of Formula I having the (Sp) configuration.
Embodiment 253. The chirally enriched population of embodiment 250, wherein the population is enriched for antisense RNAi oligonucleotides comprising at least one internucleoside linkage of Formula I having the (Rp) configuration.
Embodiment 254. The chirally enriched population of embodiment 252 wherein the at least one internucleoside linkage of Formula I having the (Sp) configuration is the first internucleoside linkage from the 5′ end of the antisense RNAi oligonucleotide.
Embodiment 255. The chirally enriched population of embodiment 253, wherein the at least one internucleoside linkage of Formula I having the (Rp) configuration is the first internucleoside linkage from the 5′ end of the antisense RNAi oligonucleotide.
Embodiment 256. The chirally enriched population of embodiment 252, wherein the at least one internucleoside linkage of Formula I having the (Sp) configuration is the second internucleoside linkage from the 5′ end of the antisense RNAi oligonucleotide.
Embodiment 257. The chirally enriched population of embodiment 253, wherein the at least one internucleoside linkage of Formula I having the (Rp) configuration is the second internucleoside linkage from the 5′ end of the antisense RNAi oligonucleotide.
Embodiment 258. A method comprising administering at least two doses of an RNAi agent of any of embodiments 92-257 to an animal wherein:
   the RNAi agent is administered to the animal at a dose frequency of once per 4 months, 5 months, 6 months, 7 months, 8 months, 9 months, 10 months, 11 months, or a year or more than a year.
Embodiment 259. An RNAi agent, comprising an antisense RNAi oligomeric compound comprising an antisense RNAi oligonucleotide consisting of 21-25 linked nucleosides and a sense RNAi oligomeric compound comprising a sense RNAi oligonucleotide consisting of 19-23 linked nucleosides, wherein the antisense RNAi oligonucleotide comprises a 5′-stabilized phosphate group; wherein the antisense RNAi oligonucleotide comprises at least one nucleoside comprising a sugar moiety selected from a stereo-non-standard sugar moiety, a sugar surrogate, a 2′-β-D-deoxyribosyl sugar moiety, or a β-D-ribosyl sugar moiety; and wherein the antisense RNAi oligonucleotide comprises at least one internucleoside linkage of Formula XIV:

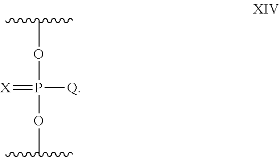

Q is R^{A} or R^{B}; wherein independently for each internucleoside linkage of Formula XIV:
X is selected from O or S;
R^{A} is —NHSO₂R; R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl, substituted C₁-C₆ alkynyl, N(C₁-C₆ alkyl)₂, and a conjugate group;
R^{B} is —N═CR¹⁰R¹¹; wherein R¹⁰ and R¹¹ are each independently alkyl, or optionally wherein R¹⁰ and R¹¹, along with the intervening atoms, join to form a heterocyclic ring.
Embodiment 260. The RNAi agent of embodiment 259, wherein the antisense RNAi oligonucleotide has an internucleoside linkage motif selected from sq(o)ₙqs, qq(o)ₙqs, ss(o)ₙqs, and sq(o)ₙss, wherein each “s” is a phosphorothioate internucleoside linkage, each “q” is an internucleoside linkage of Formula XIV, each “o” is a phosphodiester internucleoside linkage, and n is from 16-20.
Embodiment 261. The RNAi agent of embodiment 259, wherein the antisense RNAi oligonucleotide comprises at least one nucleoside comprising a 2′-fluoro-β-D-ribosyl sugar moiety, and at least one internucleoside linkage of Formula XIV is adjacent to the at least one nucleoside comprising a 2′-fluoro-β-D-ribosyl sugar moiety.
Embodiment 262. The RNAi agent of embodiment 261, wherein the internucleoside linkage of Formula XIV is to the 3′ of the at least one nucleoside comprising a 2′-fluoro-β-D-ribosyl sugar moiety.
Embodiment 263. The RNAi agent of embodiment 261, wherein the internucleoside linkage of Formula XIV is to the 5′ of the at least one nucleoside comprising a 2′-fluoro-β-D-ribosyl sugar moiety.
Embodiment 264. The RNAi agent of any of embodiments 259-263, wherein the sense RNAi oligonucleotide comprises at least one internucleoside linkage of Formula XIV:

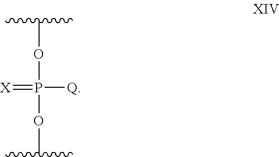

Q is R^{A} or R^{B}; wherein independently for each internucleoside linkage of Formula XIV:
X is selected from O or S;
R^{A} is —NHSO₂R; R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl, substituted C₁-C₆ alkynyl, N(C₁-C₆ alkyl)₂, and a conjugate group;
R^{B} is —N═CR¹⁰R¹¹; wherein R¹⁰ and R¹¹ are each independently alkyl, or optionally wherein R¹⁰ and R¹¹, along with the intervening atoms, join to form a heterocyclic ring.
Embodiment 265. The RNAi agent of any of embodiments 259-264, wherein the sense RNAi oligonucleotide has an internucleoside linkage motif selected from sq(o)ₙqs, qq(o)ₙqs, ss(o)ₙqs, and sq(o)ₙss, wherein each “s” is a phosphorothioate internucleoside linkage, each “q” is an internucleoside linkage of Formula XIV, each “o” is a phosphodiester internucleoside linkage, and n is from 14-18.
Embodiment 266. The RNAi agent of any of embodiments 259-265, wherein the sense RNAi oligonucleotide comprises at least one nucleoside comprising a 2′-fluoro-β-D-ribosyl sugar moiety, and at least one internucleoside linkage of Formula XIV is adjacent to the at least one nucleoside comprising a 2′-fluoro-β-D-ribosyl sugar moiety.
Embodiment 267. The RNAi agent of embodiment 266, wherein the internucleoside linkage of Formula XIV is to the 3′ of the at least one nucleoside comprising a 2′-fluoro-β-D-ribosyl sugar moiety.
Embodiment 268. The RNAi agent of embodiment 267, wherein the internucleoside linkage of Formula XIV is to the 5′ of the at least one nucleoside comprising a 2′-fluoro-β-D-ribosyl sugar moiety.
Embodiment 269. The RNAi agent of any of embodiments 259-268, wherein the sense RNAi oligonucleotide comprises at least one nucleoside comprising a 2′-β-D-deoxyribosyl sugar moiety, and at least one internucleoside linkage of Formula XIV is adjacent to the at least one nucleoside comprising a 2′-β-D-deoxyribosyl sugar moiety.
Embodiment 270. The RNAi agent of embodiment 269, wherein the internucleoside linkage of Formula XIV is to the 3′ of the at least one nucleoside comprising a 2′-β-D-deoxyribosyl sugar moiety.
Embodiment 271. The RNAi agent of embodiment 270, wherein the internucleoside linkage of Formula XIV is to the 5′ of the at least one nucleoside comprising a 2′-β-D-deoxyribosyl sugar moiety.
Embodiment 272. The RNAi agent of any of embodiments 259-271, wherein for at least one internucleoside linkage of Formula XIV, Q is R^{A}.
Embodiment 273. The RNAi agent of any of embodiments 259-271, wherein for each internucleoside linkage of Formula XIV, Q is R^{A}.
Embodiment 274. The RNAi agent of any of embodiments 259-271, wherein for at least one internucleoside linkage of Formula XIV, Q is R^{B}.
Embodiment 275. The RNAi agent of any of embodiments 259-271, wherein for each internucleoside linkage of Formula XIV, Q is R^{B}.
Embodiment 276. The RNAi agent of embodiment 272 or 273, wherein for at least one internucleoside linkage of Formula XIV, Q is R^{A} and R is C₁-C₂₀ alkyl.
Embodiment 277. The RNAi agent of embodiment 272 or 273, wherein for each internucleoside linkage of Formula XIV, Q is R^{A} and R is C₁-C₂₀ alkyl.
Embodiment 278. The RNAi agent of embodiment 276 or 277, wherein for at least one internucleoside linkage of Formula XIV, Q is R^{A} and R is C₁₆ alkyl.
Embodiment 279. The RNAi agent of embodiment 276 or 277, wherein for at least one internucleoside linkage of Formula XIV, Q is R^{A} and R is methyl.
Embodiment 280. The RNAi agent of embodiment 276 or 277, wherein for each internucleoside linkage of Formula XIV, Q is R^{A} and R is methyl.
Embodiment 281. The RNAi agent of any of embodiments 259-280, wherein X is O.
Embodiment 282. The RNAi agent of embodiment of embodiments 259-280, wherein X is S.
Embodiment 283. The RNAi agent of any of embodiments 259-282, wherein each remaining internucleoside linkage that does not have Formula XIV is selected from a phosphodiester internucleoside linkage and a phosphorothioate internucleoside linkage.
Embodiment 284. An RNAi agent, comprising an antisense RNAi oligomeric compound comprising an antisense RNAi oligonucleotide consisting of 21-25 linked nucleosides and a sense RNAi oligomeric compound comprising a sense RNAi oligonucleotide consisting of 19-23 linked nucleosides, wherein the antisense RNAi oligonucleotide comprises a 5′-stabilized phosphate group; and at least one internucleoside linkage of Formula I:

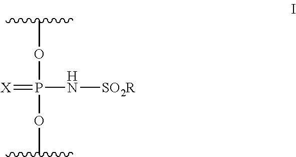

wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group; and
wherein each of at least three sugar moieties of the nucleosides of the antisense RNAi oligonucleotide is selected from a 2′-O-methyl-β-D-ribosyl sugar moiety, a 2′-O-methoxyethyl β-D-ribosyl sugar moiety, a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, and wherein each of at least three such sugar moieties are different from one another.
Embodiment 285. The RNAi agent of embodiment 284, wherein for at least one internucleoside linkage of Formula I, X is O and R is methyl.
Embodiment 286. The RNAi agent of any of embodiment 284, wherein for each internucleoside linkage of Formula I, X is O and R is methyl.
Embodiment 287. The RNAi agent of any of embodiments 284-286, wherein each internucleoside linkage of the antisense RNAi oligonucleotide is selected from an internucleoside linkage of Formula I, a phosphodiester internucleoside linkage, and a phosphorothioate internucleoside linkage.
Embodiment 288. The RNAi agent of embodiment 286, wherein the antisense RNAi oligonucleotide has an internucleoside linkage motif selected from sz(o)nzs, ss(o)nzs, and sz(o)nss, wherein each “s” is a phosphorothioate internucleoside linkage, each “z” is an internucleoside linkage of Formula I, each “o” is a phosphodiester internucleoside linkage, and n is from 16-20.
Embodiment 289. The RNAi agent of any of embodiments 259-288, wherein the antisense RNAi oligonucleotide consists of 23 linked nucleosides.
Embodiment 290. The RNAi agent of embodiment 289, wherein the antisense RNAi oligonucleotide has an internucleoside linkage motif selected from zsooooooooooooooooooss, szooooooooooooooooooss, zzooooooooooooooooooss, zzoooooooooooooooooozs, ssooooooooooooooooooss, ssoooooooooooooooooozz, zsoooooooooooooooooozz, zsoooooooooooooooooozz, ssoooooooooooooooooosz, ssoooooooooooooooooozs, szoooooooooooooooooosz, zsoooooooooooooooooosz, zsoooooooooooooooooozs, szoooooooooooooooooozs, szooozooooooozozooooss, ssooozooooooozozooooss, ssooooooooooozozooooss, szooooooooooozooooooss, zoooooooooooooooooooss, szoooooooooooooooooozz, ssooooooooooooooooooss, ssooozooooooooooooooss, ssooooooooooooozooooss, ssooooooooooozooooooss wherein each “s” is a phosphorothioate internucleoside linkage, each “z” is an internucleoside linkage of Formula I or of Formula XIV, and each “o” is a phosphodiester internucleoside linkage.
Embodiment 291. The RNAi agent of embodiment 290, wherein for each internucleoside linkage of Formula I, X is O and R is methyl.
Embodiment 292. The RNAi agent of any of embodiments 259-291, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.
Embodiment 293. The RNAi agent of any of embodiments 259-292, wherein each sugar surrogate is independently selected from fluoro hexitol nucleic acid (F-HNA), hexitol nucleic acid (HNA) or altritol nucleic acid (ANA).
Embodiment 294. The RNAi agent of any of embodiments 259-293, wherein the antisense RNAi oligonucleotide comprises no more than three 2′-fluoro-β-D-ribosyl sugar moieties.
Embodiment 295. The RNAi agent of any of embodiments 289-294, wherein the antisense RNAi oligonucleotide has a sugar motif of yxyyyxyyyyyyyxyxyyyyyyy or exyyyxyyyyyyyxyxyyyyyyy, from 5′ to 3′, wherein each “y” is a 2′-O-methyl-β-D-ribosyl sugar moiety, each “e” is a 2′-O-methoxyethyl-β-D-ribosyl sugar moiety, and each “x” is independently selected from a sugar surrogate, a stereo-non-standard sugar moiety, a 2′-β-D-deoxyribosyl sugar moiety, a β-D-ribosyl sugar moiety, and a 2′-fluoro-β-D-ribosyl sugar moiety, wherein at least one “x” is other-than a 2′-fluoro-β-D-ribosyl sugar moiety.
Embodiment 296. The RNAi agent of embodiment 295, wherein at least one “x” is a stereo-non-standard sugar
Embodiment 297. The RNAi agent of embodiment 295, wherein at least one “x” is a 2′-fluoro-β-D-xylosyl sugar moiety.
Embodiment 298. The RNAi agent of embodiment 297, wherein exactly one “x” is a stereo-non-standard sugar
Embodiment 299. The RNAi agent of embodiment 298, wherein exactly one “x” is a 2′-fluoro-β-D-xylosyl sugar moiety.
Embodiment 300. The RNAi agent of embodiment 295, wherein at least one “x” is a sugar surrogate.
Embodiment 301. The RNAi agent of embodiment 300, wherein exactly one “x” is a sugar surrogate.
Embodiment 302. The RNAi agent of embodiment 300 or 301, wherein the sugar surrogate is fluoro hexitol nucleic acid (F-HNA).
Embodiment 303. The RNAi agent of embodiment 295, wherein at least one “x” is a β-D-ribosyl sugar moiety.
Embodiment 304. The RNAi agent of any of embodiments 289-303, wherein the antisense RNAi oligonucleotide has a sugar motif selected from: y[f2bDx]yyyfyyyyyyyfyfyyyyyyy, yfyyy[f2bDx]yyyyyyyfyfyyyyyyy, yfyyyfyyyyyyy[f2bDx]yfyyyyyyy, yfyyyfyyyyyyyfy[f2bDx]yyyyyyy, y[f2bDx]yyy[f2bDx]yyyyyyy[f2bDx]y[f2bDx]yyyyyyy, efyyyfy[16C2r]yyyyyfyfyyyyyyy, e[f2bDx]yyyfyyyyyyyfyfyyyyyyy, efyyy[f2bDx]yyyyyyyfyfyyyyyyy, efyyyfyyyyyyy[f2bDx]yfyyyyyyy, efyyyfyyyyyyyfy[f2bDx]yyyyyyy, e[f2bDx]yyy[f2bDx]yyyyyyy[f2bDx]y[f2bDx]yyyyyyy, efyyy[F-HNA]yyyyyyyfyfyyyyyyy, efyyyfyyyyyyy[F-HNA]yfyyyyyyy, efyyyfyyyyyyyfy[F-HNA]yyyyyyy, yfyyydyyyyyyyfyfyyyyyyy, yfyyyfyyyyyyydyfyyyyyyy, yfyyyfyyyyyyyfydyyyyyyy, yfyyydyyyyyyydydyyyyyyy, yryyyfyyyyyyyfyfyyyyyyy, yfyyyryyyyyyyfyfyyyyyyy, yfyyyfyyyyyyyryfyyyyyyy, yfyyyfyyyyyyyfyryyyyyyy, yryyyryyyyyyyryryyyyyyy, y[bDdx]yyyfyyyyyyyfyfyyyyyyy, yfyyy[bDdx]yyyyyyyfyfyyyyyyy, yfyyyfyyyyyyy[bDdx]yfyyyyyyy, yfyyyfyyyyyyyfy[bDdx]yyyyyyy, y[bDdx]yyy[bDdx]yyyyyyy[bDdx]y[bDdx]yyyyyyy, y[F-HNA]yyyfyyyyyyyfyfyyyyyyy, yfyyy[F-HNA]yyyyyyyfyfyyyyyyy, yfyyyfyyyyyyy[F-HNA]yfyyyyyyy, yfyyyfyyyyyyyfy[F-HNA]yyyyyyy, y[F-HNA]yyy[F-HNA]yyyyyyy[F-HNA]y[F-HNA]yyyyyyy, yfyyyfyyyyyyy[2bDx]yfyyyyyyy, y[bDa]yyyfyyyyyyyfyfyyyyyyy, yfyyy[bDa]yyyyyyyfyfyyyyyyy, yfyyyfyyyyyyy[bDa]yfyyyyyyy, yfyyyfyyyyyyyfy[bDa]yyyyyyy, y[bDa]yyy[bDa]yyyyyyy[bDa]y[bDa]yyyyyyy, yfyyyfyyyyyyy[bDx]yfyyyyyyy, yfyyyfyffyyyyfyfyyyyydd, yfyyyfyffyyyyfyfyyyyy[bLdr][bLdr], yfyyyfyffyyyyfyfyyyyy[aDdr][aDdr], yfyyyfyffyyyyfyfyyyyy[bDdx][bDdx], yfyyyfyffyyyyfyfyyyyy[bLdx][bLdx], yfyyyfyffyyyyfyfyyyyy[aDdx][aDdx], yfyyyfyffyyyyfyfyyyyy[aLdx][aLdx], yfyyyfyyyyyyyfyfyyyyyee, yfyyyfyyyyyyyfyfyyyyykk, yfyyyfyyyyyyyfyfyyyyy[LNA][LNA], yfyyyfyyyyyyyfyfyyyyy[F-HNA][F-HNA], [ANA]fyyyfyyyyyyyfyfyyyyyyy, y[ANA]yyyfyyyyyyyfyfyyyyyyy, yfyyyf[ANA]yyyyyyfyfyyyyyyy, yfyyyfyy[ANA]yyyyfyfyyyyyyy, yfyyyfyyyyyyy[ANA]yfyyyyyyy, yfyyyfyyyyyyyfyf[ANA]yyyyyy, yfyyyfyy[ANA]yyyy[ANA]yf[ANA]yyyyy[ANA], yfyyyfyyyyyyyfyfyyyyyyk, yfyyyfyyyyyyyfyfyyyyyke, yfyyyfyyyyyyyfyfyyyyyky, efyyydyyyyyyydydyyyyyyy, yfyyyfyyyyyyy[ANA]yf[ANA]yyyyyy, yfyyyfyyyyyyyfyfyyyyy[ANA]y, yfyyyfyyyyyyyfyfyyyyy[f2bDa][f2bDa], yfyyyfyyyyyyyfyfyyyyynn, yfyyyfyyyyyyyfyfyyyyy[DMAEOE][DMAEOE], yfyyyfyyyyyyyfyfyyyyy[HNA][HNA], yfyyyfyyyyyyyfyfyyyyy[SM5LNA][SM5LNA], yfyyyfyyyyyyyfyfyyyyy[DMAOE][DMAOE], yfyyyfyyyyyyyfyfyyyyydd, yfyyyfyyyyyyyfyfyyyyy[aLdr][aLdr], [HNA]fyyyfyyyyyyyfyfyyyyyyy, dfyyyfyyyyyyyfyfyyyyyyy, y[HNA]yyyfyyyyyyyfyfyyyyyyy, e[HNA]yyyfyyyyyyyfyfyyyyyyy, yfyyyf[HNA]yyyyyyfyfyyyyyyy, yfyyyfyy[HNA]yyyyfyfyyyyyyy, yfyyyfyyyyyyy[HNA]yfyyyyyyy, yfyyyfyyyyyyyfyf[HNA]yyyyyy, yfyyyfyy[HNA]yyyy[HNA]yf[HNA]yyyyy[HNA], yfyyyfyyyyyyy[HNA]yf[HNA]yyyyyy, yfyyyfyyyyyyyfyfyyyyy[HNA]y, kfyyyfyyyyyyyfyfyyyyyyy, e[F-HNA]yyyfyyyyyyyfyfyyyyyyy, e[LNA]yyyfyyyyyyyfyfyyyyyyy, edyyyfyyyyyyyfyfyyyyyyy, edyyydyyyyyyydydyyyyyyy, ydyyyfyyyyyyyfyfyyyyyyy, ydyyydyyyyyyydydyyyyyyy, efyyfyfyyyyfyfyyyyyyyyy, edyydydyyyydyfyyyyyyyyy, efyyyfyyyyyyyfyfyyyyydd, [F-HNA]fyyyfyyyyyyyfyfyyyyyyy, eyyyyfyyyyyyyfyfyyyyyyy, eryyyryyyyyyyryryyyyyyy, efyyydyyyyyyyfyfyyyyyyy, efyyyfyyyyyyyfydyyyyyyy, efyyyfyyyyyyydyfyyyyyyy; wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C2r]” represents a 2′-O-hexadecyl-β-D-ribosyl sugar moiety, “[F-HNA]” represents 3′-fluoro-hexitol sugar surrogate, “[bDdx]” represents a 2′-β-D-deoxyxylosyl sugar moiety, “[bDx]” represents a β-D-xylosyl sugar moiety, “[bDa]” represents a β-D-arabinosyl sugar moiety, “[bLdr]” represents a 2′-β-L-deoxyribosyl sugar moiety, “[aDdr]” represents a 2′-α-D-deoxyribosyl sugar moiety, “[aLdr]” represents a 2′-α-L-deoxyribosyl sugar moiety, “[bLdx]” represents a 2′-β-L-deoxyxylosyl sugar moiety, “[aDdx]” represents an 2′-α-D-deoxyxylosyl sugar moiety, “[aLdx]” represents an 2′-α-L-deoxyxylosyl sugar moiety, “[LNA]” represents a β-D-LNA sugar moiety, “[f2bDa]” represents a 2′-fluoro-β-D-arabionsyl sugar moiety, “n” represents a 2′-O—(N-methylacetamide) ribosyl sugar moiety, “[DMAEOE]” represents a 2′-O-(2-(2-(N,N-dimethyl)aminoethoxy)ethyl)-β-D-ribosyl sugar moiety, “[SM5LNA]” represents a (5'S)-5′methyl-LNA sugar moiety, “[ANA]” represents an ANA sugar surrogate, and “[HNA]” represents an HNA sugar surrogate.
Embodiment 305. The RNAi agent of embodiment 304, wherein the antisense RNAi oligonucleotide has a sugar motif selected from: y[f2bDx]yyyfyyyyyyyfyfyyyyyyy, y[f2bDx]yyyyyyyyyyfyfyyyyyyy, efyyyfy[16C2r]yyyyyfyfyyyyyyy, e[f2bDx]yyyfyyyyyyyfyfyyyyyyy, efyyy[F-HNA]yyyyyyyfyfyyyyyyy, efyyyfyyyyyyy[F-HNA]yfyyyyyyy, efyyyfyyyyyyyfy[F-HNA]yyyyyyy, y[F-HNA]yyyfyyyyyyyfyfyyyyyyy, yfyyy[F-HNA]yyyyyyyfyfyyyyyyy, yfyyyfyyyyyyy[F-HNA]yfyyyyyyy, or yfyyyfyyyyyyyfy[F-HNA]yyyyyyy.
Embodiment 306. The RNAi agent of any of embodiments 289-305, wherein the antisense RNAi oligonucleotide has a sugar motif of dxyyyxyyyyyyyxyxyyyyyyy, dxyyyxyyyyyyyxyxyyyyydd, or ddyyyxyyyyyyyxyxyyyyydd, from 5′ to 3′, wherein each “y” is a 2′-O-methyl-β-D-ribosyl sugar moiety, each “d” is a 2′-β-D-deoxyribosyl sugar moiety, and each “x” is independently selected from a sugar surrogate, a stereo-non-standard sugar moiety, a 2′-β-D-deoxyribosyl sugar moiety, a β-D-ribosyl sugar moiety, and a 2′-fluoro-β-D-ribosyl sugar moiety, and “z” is independently selected from a sugar surrogate, a stereo-non-standard sugar moiety, a bicyclic sugar moiety, a 2′-β-D-deoxyribosyl sugar moiety, a β-D-ribosyl sugar moiety, and a 2′-fluoro-β-D-ribosyl sugar moiety.
Embodiment 307. The RNAi agent of embodiment 306, wherein at least one “x” is a stereo-non-standard sugar
Embodiment 308. The RNAi agent of embodiment 307, wherein at least one “x” is a 2′-fluoro-β-D-xylosyl sugar moiety.
Embodiment 309. The RNAi agent of embodiment 306, wherein at least one “x” is a sugar surrogate.
Embodiment 310. The RNAi agent of embodiment 306, wherein exactly one “x” is a sugar surrogate.
Embodiment 311. The RNAi agent of embodiment 309 or 310 wherein the sugar surrogate is fluoro hexitol nucleic acid (F-HNA).
Embodiment 312. An RNAi agent, comprising an antisense RNAi oligomeric compound comprising an antisense RNAi oligonucleotide consisting of 21-25 linked nucleosides and a sense RNAi oligomeric compound comprising a sense RNAi oligonucleotide consisting of 19-23 linked nucleosides, wherein the antisense RNAi oligonucleotide has the formula (from 5′ to 3′):

G-N_{z}—X¹_{z}—(Yₒ)ₙ—Yᵥ—X²ᵥ—(Yₒ)ₚ—Yᵥ—X³ᵥ—Yᵥ—X⁴ᵥ—(Yₒ)_{q}—Y_{z}-Q¹_{z}-Q², wherein:
G is a stabilized phosphate moiety;
N is a nucleoside comprising a sugar moiety selected from a 2′-O-methyl-β-D-ribosyl sugar moiety, a 2′-O-methoxyethyl-β-D-ribosyl sugar moiety, and a 2′-β-D-deoxyribosyl sugar moiety;
X¹ is a nucleoside comprising a sugar moiety independently selected from a 2′-fluoro-β-D-ribosyl sugar moiety, 2′-β-D-deoxyribosyl sugar moiety, a 2′-fluoro-β-D-xylosyl sugar moiety, an ANA sugar surrogate and an F-HNA sugar surrogate;
each X², X³, and X⁴ is a nucleoside comprising a sugar moiety independently selected from a 2′-fluoro-β-D-ribosyl sugar moiety, 2′-β-D-deoxyribosyl sugar moiety, a 2′-fluoro-β-D-xylosyl sugar moiety, and a sugar surrogate;
provided that at least one X¹, X², X³, or X⁴ comprises a sugar moiety other-than a 2′-fluoro-β-D-ribosyl sugar moiety;
each Y is a nucleoside comprising a 2′-O-methyl-β-D-ribosyl sugar moiety;
each Q¹ and Q² is independently a nucleoside;
each “z” is independently selected from a mesyl phosphoramidate internucleoside linkage and a phosphorothioate internucleoside linkage;
each “v” is independently selected from a mesyl phosphoramidate internucleoside linkage and a phosphodiester internucleoside linkage;
provided that at least one internucleoside linkage “z” or “v” is a mesyl phosphoramidate internucleoside linkage;
each internucleoside linkage “o” is a phosphodiester internucleoside linkage;
n is from 1-3,
p is from 5-7, and
q is from 3-5.
Embodiment 313. The RNAi agent of embodiment 312, wherein at least one of X², X³, or X⁴ comprises a sugar surrogate.
Embodiment 314. The RNAi agent of embodiment 313, wherein exactly one of X², X³, or X⁴ comprises a sugar surrogate.
Embodiment 315. The RNAi agent of any one of embodiments 312 to 314, wherein the sugar surrogate is selected from ANA and F-HNA.
Embodiment 316. The RNAi agent of any of embodiments 313-315, wherein each remaining X², X³, and X⁴ comprises a 2′-fluoro-β-D-ribosyl sugar moiety.
Embodiment 317. The RNAi agent of embodiment 313, wherein at least one of X², X³, or X⁴ comprises a 2′-fluoro-β-D-xylosyl sugar moiety.
Embodiment 318. The RNAi agent of embodiment 313, wherein exactly one of X², X³, or X⁴ comprises a 2′-fluoro-β-D-xylosyl sugar moiety
Embodiment 319. The RNAi agent of any of embodiments 317 or 318, wherein each remaining X², X³, and X⁴ comprises a 2′-fluoro-β-D-ribosyl sugar moiety.
Embodiment 320. The RNAi agent of embodiment 313, wherein at least one of X², X³, or X⁴ comprises a 2′-β-D-deoxyribosyl sugar moiety.
Embodiment 321. The RNAi agent of embodiment 313, wherein exactly one of X², X³, or X⁴ comprises a 2′-β-D-deoxyribosyl sugar moiety.
Embodiment 322. The RNAi agent of embodiment 320 or 321, wherein each remaining X², X³, and X⁴ comprises a 2′-fluoro-β-D-ribosyl sugar moiety.
Embodiment 323. The RNAi agent of embodiment 312, wherein each X², X³, and X⁴ comprises a 2′-fluoro-β-D-ribosyl sugar moiety.
Embodiment 324. The RNAi agent of any one of embodiments 312 to 323, wherein X¹ comprises a 2′-fluoro-β-D-xylosyl sugar moiety.
Embodiment 325. The RNAi agent of any one of embodiments 312 to 323, wherein X¹ comprises an F-HNA sugar surrogate.
Embodiment 325a. The RNAi agent of any one of embodiments 312 to 323, wherein X¹ comprises a 2′-β-D-deoxyribosyl sugar moiety.
Embodiment 326. The RNAi agent of any of embodiments 312-325, wherein N comprises a 2′-O-methoxyethyl-β-D-ribosyl sugar moiety.
Embodiment 327. The RNAi agent of any of embodiments 312-326, wherein N comprises a thymine nucleobase.
Embodiment 328. The RNAi agent of any of embodiments 312-327, wherein each of Q¹ and Q² is a nucleoside comprising a sugar moiety selected from a 2′-fluoro-β-D-ribosyl sugar moiety, a 2′-O-methyl-β-D-ribosyl sugar moiety, a 2′-β-D-deoxyribosyl sugar moiety, 2′-α-D-deoxyribosyl sugar moiety, 2′-β-L-deoxyribosyl sugar moiety, 2′-α-L-deoxyribosyl sugar moiety, a 2′-β-D-deoxyxylosyl sugar moiety, 2′-α-D-deoxyxylosyl sugar moiety, 2′-β-L-deoxyxylosyl sugar moiety, 2′-α-L-deoxyxylosyl sugar moiety, a 2′-MOE sugar moiety, a cEt sugar moiety, an LNA sugar moiety, an F-HNA sugar surrogate, an ANA sugar surrogate, an HNA sugar surrogate, 2′-O—(N-methylacetamide) ribosyl sugar moiety, a 2′-O-(2-(2-(N,N-dimethyl)aminoethoxy)ethyl)-β-D-ribosyl sugar moiety, and a (5'S)-5′methyl-LNA sugar moiety.
Embodiment 329. The RNAi agent of any of embodiments 312-328, wherein each of Q¹ and Q² comprises a sugar moiety independently selected from a 2′-O-methoxyethyl-β-D-ribosyl sugar moiety, a 2′-O-methyl-β-D-ribosyl sugar moiety, and a cEt sugar moiety.
Embodiment 330. The RNAi agent of any of embodiments 312-329, wherein each of Q¹ and Q² comprises a thymine nucleobase.
Embodiment 331. The RNAi agent of any of embodiments 312-330, wherein the antisense RNAi oligonucleotide contains no more than 3 or no more than 4 mesyl phosphoramidate internucleoside linkages.
Embodiment 332. The RNAi agent of any of embodiments 312-331, wherein n is 2, p is 6, and q is 4.
Embodiment 333. The RNAi agent of any of embodiments 259-332, wherein the antisense RNAi oligonucleotide has a target complementary region of at least 18 consecutive nucleosides that are at least 85% complementary to an equal length portion of a target nucleic acid.
Embodiment 334. The RNAi agent of embodiment 333, wherein the antisense RNAi oligonucleotide has a target complementary region that is at least 90%, at least 95%, or 100% complementary to an equal length portion of a target nucleic acid.
Embodiment 335. The RNAi agent of embodiment 334, wherein the target complementary region consists of 21 consecutive nucleosides.
Embodiment 336. The RNAi agent of any of embodiments 259-335, wherein the sense RNAi oligonucleotide has a complementary region of at least 18 consecutive nucleosides that are at least 85% complementary to an equal length region of the antisense RNAi oligonucleotide.
Embodiment 337. The RNAi agent of embodiment 336, wherein the sense RNAi oligonucleotide consists of 2 fewer linked nucleosides than the antisense RNAi oligonucleotide.
Embodiment 338. The RNAi agent of embodiment 337, wherein the complementary region of the sense RNAi consists of 21 nucleobases.
Embodiment 339. The RNAi agent of embodiment 338, wherein the antisense RNAi oligonucleotide consists of 23 linked nucleosides, the sense RNAi oligonucleotide consists of 21 linked nucleosides, the sense RNAi oligonucleotide is 100% complementary to the antisense RNAi oligonucleotide, and the antisense RNAi oligonucleotide is at least 85% or at least 90% complementary to an equal length portion of a target nucleic acid.
Embodiment 340. The RNAi agent of any of embodiments 229-339, wherein the sense RNAi oligonucleotide comprises at least one internucleoside linkage of Formula I:

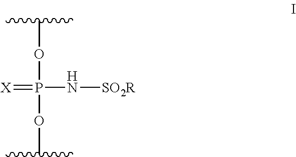

wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group.
Embodiment 341. The RNAi agent of embodiment 340, wherein each internucleoside linkage of the sense RNAi oligonucleotide is selected from an internucleoside linkage of Formula I, a phosphodiester internucleoside linkage, and a phosphorothioate internucleoside linkage.
Embodiment 342. The RNAi agent of embodiment 341, wherein the sense RNAi oligonucleotide has an internucleoside linkage motif selected of qq(o)ₙqq, wherein each “q” is independently selected from a phosphorothioate internucleoside linkage and an internucleoside linkage of Formula I, each “o” is a phosphodiester internucleoside linkage, and n is from 14-18.
Embodiment 343. The RNAi agent of embodiment 342, wherein the sense RNAi oligonucleotide has an internucleoside linkage motif selected of qq(o)ₙz(o)ₘqq, wherein each “q” is independently selected from a phosphorothioate internucleoside linkage and an internucleoside linkage of Formula I wherein X is O and R is methyl, each “o” is a phosphodiester internucleoside linkage, and “z” is an internucleoside linkage of Formula I wherein X is O and R is selected from C₁₀-C₂₀ alkyl, substituted C₁₀-C₂₀ alkyl, and a conjugate group, wherein n is from 2 to 5, m is from 8 to 15, and n+m is from 13 to 17.
Embodiment 344. The RNAi agent of any of embodiments 259-343, wherein the sense RNAi oligonucleotide consists of 21 linked nucleosides and has an internucleoside linkage motif selected from ssoooooooooooooooooo, ssoooooooooooooooooo, ssooooooooooooooooss, ssooooooooooooooooss, zzoooooooooooooooozz, ssoooozooozoooooooss, ssoooozozozoooooooss, ssoooozozzzoooooooss, zsoooooooooooooooosz, and zzoooooooooooooooooo, wherein each “s” is a phosphorothioate internucleoside linkage, each “z” is an internucleoside linkage of Formula I, and each “o” is a phosphodiester internucleoside linkage.
Embodiment 345. The RNAi agent of any of embodiments 341-344, wherein for at least one internucleoside linkage of Formula I of the sense RNAi oligonucleotide, X is O and R is methyl.
Embodiment 346. The RNAi agent of any of embodiments 341-345, wherein for at least one internucleoside linkage of Formula I of the sense RNAi oligonucleotide, X is O and R is C₁₆.
Embodiment 347. The RNAi agent of any of embodiments 341-346, wherein for at least one internucleoside linkage of Formula I of the sense RNAi oligonucleotide, R is a conjugate group.
Embodiment 348. The RNAi agent of embodiment 347, wherein the conjugate group comprises a conjugate linker and a conjugate moiety.
Embodiment 349. The RNAi agent of embodiment 348, wherein the conjugate moiety is selected from a lipid, a carbohydrate, a GalNAc, an antibody or fragment thereof, or a peptide.
Embodiment 350. The RNAi agent of any of embodiments 259-349, wherein each of at least two sugar moieties of the sense RNAi is selected from a 2′-O-methyl-β-D-ribosyl sugar moiety, a 2′-O-methoxyethyl-β-D-ribosyl sugar moiety, a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, and wherein each of at least two such sugar moieties are different from each other.
Embodiment 351. The RNAi agent of any of embodiments 259-350, wherein the sense RNAi oligonucleotide consists of 21 linked nucleosides and has a sugar motif of yyyyyyxyxxxyyyyyyyyyy, from 5′ to 3′, wherein each “y” is a 2′-O-methyl β-D-ribosyl sugar moiety and each “x” is independently selected from a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, wherein at least one “x” is other-than a 2′-fluoro-β-D-ribosyl sugar moiety.
Embodiment 352. The RNAi agent of embodiment 350 or 351, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.
Embodiment 353. The RNAi agent of embodiment 351, wherein at least one “x” is a 2′-β-D-deoxyribosyl sugar moiety.
Embodiment 354. The RNAi agent of embodiment 353, wherein each “x” is a 2′-β-D-deoxyribosyl sugar moiety.
Embodiment 355. The RNAi agent of embodiment 351, wherein at least one “x” is a 2′-fluoro-β-D-xylosyl sugar moiety.
Embodiment 356. The RNAi agent of embodiment 355, wherein each “x” is a 2′-fluoro-β-D-xylosyl sugar moiety.
Embodiment 357. The RNAi agent of embodiment 351, wherein at least one “x” is a sugar surrogate.
Embodiment 358. The RNAi agent of embodiment 357, wherein each sugar surrogate is independently selected from fluoro hexitol nucleic acid (F-HNA), hexitol nucleic acid (HNA) or altritol nucleic acid (ANA).
Embodiment 359. The RNAi agent of any of embodiments 259-358, wherein the sense RNAi oligonucleotide has a sugar motif selected from: yyyyyyfyff[f2bDx]yyyyyyyyyy, yyyyyyfyf[f2bDx]fyyyyyyyyyy, yyyyyyfy[f2bDx]ffyyyyyyyyyy, yyyyyy[f2bDx]yfffyyyyyyyyyy, yyyyyy[f2bDx]y[f2bDx][f2bDx][f2bDx]yyyyyyyyyy, yyyyy[16C2r]fyfffyyyyyyyyyy, yyyyy[C16A]fyfffyyyyyyyyyy, yyyyy[16C2r]fyfffyyyyyyyyyy, yyyyy[16C2r]fyff[f2bDx]yyyyyyyyyy, yyyyy[16C2r]fyf[f2bDx]fyyyyyyyyyy, yyyyy[16C2r]fy[f2bDx]ffyyyyyyyyyy, yyyyy[16C2r][f2bDx]yfffyyyyyyyyyy, yyyyy[16C2r][f2bDx]y[f2bDx][f2bDx][f2bDx]yyyyyyyyyy, yyyyyyfyfffyyyyyyyyyyddddddddd, yyyyy[16C2r]fyff[F-HNA]yyyyyyyyyy, yyyyy[16C2r]fyf[F-HNA]fyyyyyyyyyy, yyyyy[16C2r]fy[F-HNA]ffyyyyyyyyyy, yyyyy[16C2r][F-HNA]yfffyyyyyyyyyy, yyyyy[16C2r][F-HNA]yff[F-HNA]yyyyyyyyyy, yyyyy[16C2r][F-HNA]y[F-HNA][F-HNA][F-HNA]yyyyyyyyyy, yyyyyyyyyyyyyyyyyyyyy, yyyyyyyyfffyyyyyyyyyy, yyyyyyfyyffyyyyyyyyyy, yyyyyyfyfyfyyyyyyyyyy, yyyyyyfyffyyyyyyyyyyy, yyyyyyyyyffyyyyyyyyyy, yyyyyyfyyyfyyyyyyyyyy, yyyyyyfyfyyyyyyyyyyyy, yyyyyyyyfyfyyyyyyyyyy, yyyyyyyyffyyyyyyyyyyy, yyyyyyfyyfyyyyyyyyyyy, yyyyyyeyeeeyyyyyyyyyy, yyyyyyeyfffyyyyyyyyyy, yyyyyyfyeffyyyyyyyyyy, yyyyyyfyfefyyyyyyyyyy, yyyyyyfyffeyyyyyyyyyy, yyyyyyeyeffyyyyyyyyyy, yyyyyyfyeefyyyyyyyyyy, yyyyyyfyfeeyyyyyyyyyy, yyyyyyeyfefyyyyyyyyyy, yyyyyyeyffeyyyyyyyyyy, yyyyyyfyefeyyyyyyyyyy, yyyyyydyfffyyyyyyyyyy, yyyyyyfydffyyyyyyyyyy, yyyyyyfyfdfyyyyyyyyyy, yyyyyyfyffdyyyyyyyyyy, yyyyyydydffyyyyyyyyyy, yyyyyydyfdfyyyyyyyyyy, yyyyyydyffdyyyyyyyyyy, yyyyyydydfdyyyyyyyyyy, yyyyyyfydddyyyyyyyyyy, yyyyyydyddfyyyyyyyyyy, yyyyyydydddyyyyyyyyyy, yyyyydddddddddddyyyyy, yyyyyydddddddddyyyyyy, yyyyyyydddddddyyyyyyy, yyyyyyyydddddyyyyyyyy, eeeeedddddddddddeeeee, eeeeeedddddddddeeeeee, eeeeeeedddddddeeeeeee, eeeeeeeedddddeeeeeeee, yyyyyydydydydyddyyyyy, eeeeeedededededdeeeee, dydydydydydydydydydyd, dydydyfyfffydydydydyd, dedededededededededed, dededefefffededededed, ryryryryryryryryryryr, ryryryfyfffyryryryryr, rerererererererererer, rererefefffererererer, yyyyy[16C2r][f2bDx]yff[f2bDx]yyyyyyyyyy, yyyyyyryfffyyyyyyyyyy, yyyyyyfyrffyyyyyyyyyy, yyyyyyfyfrfyyyyyyyyyy, yyyyyyfyffryyyyyyyyyy, yyyyyyryrrryyyyyyyyyy, yyyyyyfyff[bDdx]yyyyyyyyyy, yyyyyyfyf[bDdx]fyyyyyyyyyy, yyyyyyfy[bDdx]ffyyyyyyyyyy, yyyyyy[bDdx]yfffyyyyyyyyyy, yyyyyy[bDdx]y[bDdx][bDdx][bDdx]yyyyyyyyyy, yyyyyyfyff[F-HNA]yyyyyyyyyy, yyyyyyfyf[F-HNA]fyyyyyyyyyy, yyyyyyfy[F-HNA]ffyyyyyyyyyy, yyyyyy[F-HNA]yfffyyyyyyyyyy, yyyyyy[F-HNA]y[F-HNA][F-HNA][F-HNA]yyyyyyyyyy, yyyyyyfyf[bDx]fyyyyyyyyyy, yyyyyyfyf[bDx]fyyyyyyyyyy, yyyyyyfyf[ANA]fyyyyyyyyyy, [ANA]yyyyyfyf[ANA]fyyyyyy[ANA]yyy, [ANA]yy[ANA]yyfyf[ANA]fyyyyyyyyyy, yyy[ANA]y[ANA]fyf[ANA]fyyyyyyyyyy, yyyyyyfyf[ANA]f[ANA]yyyyy[ANA]yyy, [ANA]yyyy[ANA]fyf[ANA]f[ANA]yyyyyyyyy, [ANA]yyyy[ANA]fyf[ANA]fyyyy[ANA]yyyyy, [ANA]yyyy[ANA]fyf[ANA]f[ANA]yyy[ANA]yyyyy, yyyyyyfyf[HNA]fyyyyyyyyyy, [HNA]yyyyyfyf[HNA]fyyyyyy[HNA]yyy, [HNA]yy[HNA]yyfyf[HNA]fyyyyyyyyyy, yyy[HNA]y[HNA]fyf[HNA]fyyyyyyyyyy, yyyyyyfyf[HNA]f[HNA]yyyyy[HNA]yyy, [HNA]yyyy[HNA]fyf[HNA]fyyyy[HNA]yyyyy, [HNA]yyyy[HNA]fyf[HNA]f[HNA]yyyyyyyyy, [HNA]yyyy[HNA]fyf[HNA]f[HNA]yyy[HNA]yyyyy, yyyyyydydydydydyyyyyy, yyyyyy[bDa]yfffyyyyyyyyyy, yyyyyyfy[bDa]ffyyyyyyyyyy, yyyyyyfyf[bDa]fyyyyyyyyyy, yyyyyyfyff[bDa]yyyyyyyyyy, yyyyyy[bDa]y[bDa][bDa][bDa]yyyyyyyyyy, yyyyyyyyyyfyyyyyyyyyy, yyyyyyyyfyyyyyyyyyyyy, yyyyyyfyyyyyyyyyyyyyy, yyyyyyyydddyyyyyyyyyy, wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C2r]” represents a 2′-O-hexadecyl-β-D-ribosyl sugar moiety, “[C16A]” represents 2′-O-hexylpalmitamide-β-D-ribosyl sugar moiety “[F-HNA]” represents a F-HNA sugar surrogate, “[HNA]” represents an HNA sugar surrogate; “[ANA]” represents an ANA sugar surrogate; “[bDdx]” represents a 2′-β-D-deoxyxylosyl sugar moiety, “[bDx]” represents a β-D-xylosyl sugar moiety, “[bDa]” represents a β-D-arabinosyl sugar moiety.
Embodiment 360. The RNAi agent of any of embodiments 259-359, wherein the sense RNAi oligonucleotide has the formula (from 5′ to 3′):

Y_{z}—Y_{z}—(Yₒ)ₙ—Yᵥ—X¹ᵥ—Yᵥ—X²ᵥ—X³ᵥ—X⁴ᵥ—(Yₒ)ₚ—Y_{z}—Y_{z},—Y, wherein:
each X¹, X², X³, and X⁴ is a nucleoside comprising a sugar moiety independently selected from a 2′-fluoro-β-D-ribosyl sugar moiety, 2′-β-D-deoxyribosyl sugar moiety, a 2′-fluoro-β-D-xylosyl sugar moiety, and a sugar surrogate;
provided that at least one X¹, X², X³, or X⁴ comprises a sugar moiety other-than a 2′-fluoro-β-D-ribosyl sugar moiety;
each Y is a nucleoside comprising a 2′-O-methyl-β-D-ribosyl sugar moiety;
each “z” is independently selected from a mesyl phosphoramidate internucleoside linkage and a phosphorothioate internucleoside linkage;
each “v” is independently selected from a mesyl phosphoramidate internucleoside linkage and a phosphodiester internucleoside linkage;
provided that at least one internucleoside linkage “z” or “v” is a mesyl phosphoramidate internucleoside linkage;
each internucleoside linkage “o” is a phosphodiester internucleoside linkage;
n is from 2-4,
p is from 6-8.
Embodiment 361. An RNAi agent, comprising an antisense RNAi oligomeric compound comprising an antisense RNAi oligonucleotide consisting of 21-25 linked nucleosides and a sense RNAi oligomeric compound comprising a sense RNAi oligonucleotide consisting of 19-23 linked nucleosides, wherein the sense RNAi oligonucleotide has the formula (from 5′ to 3′):

Y_{z}—Y_{z}—(Yₒ)ₙ—Yᵥ—X¹ᵥ—Yᵥ—X²ᵥ—X³ᵥ—X⁴ᵥ—(Yₒ)ₚ—Y_{z}—Y_{z}—Y, wherein:
each X¹, X², X³, and X⁴ is a nucleoside comprising a sugar moiety independently selected from a 2′-fluoro-β-D-ribosyl sugar moiety, 2′-β-D-deoxyribosyl sugar moiety, a 2′-fluoro-β-D-xylosyl sugar moiety, 2′-O-methyl-β-D-ribosyl sugar moiety, and a sugar surrogate;
provided that at least one X¹, X², X³, or X⁴ comprises a sugar moiety other-than a 2′-fluoro-β-D-ribosyl sugar moiety;
each Y is a nucleoside comprising a 2′-O-methyl-β-D-ribosyl sugar moiety;
each “z” is independently selected from a mesyl phosphoramidate internucleoside linkage and a phosphorothioate internucleoside linkage;
each “v” is independently selected from a mesyl phosphoramidate internucleoside linkage and a phosphodiester internucleoside linkage;
provided that at least one internucleoside linkage “z” or “v” is a mesyl phosphoramidate internucleoside linkage;
each internucleoside linkage “o” is a phosphodiester internucleoside linkage;
n is from 2-4,
p is from 6-8.
Embodiment 362. The RNAi agent of embodiment 360 or 361, wherein at least one of X¹, X², X³, or X⁴ comprises a sugar surrogate.
Embodiment 363. The RNAi agent of embodiment 362, wherein X¹ and/or X⁴ comprises a sugar surrogate.
Embodiment 364. The RNAi agent of embodiment 362, wherein both X¹ and X⁴ comprises a sugar surrogate.
Embodiment 365. The RNAi agent of any of embodiments 363-365, wherein the sugar surrogate is F-HNA.
Embodiment 366. The RNAi agent of embodiment 360 or 361, wherein at least one of X¹, X², X³, or X⁴ comprises a 2′-β-D-deoxyribosyl sugar moiety.
Embodiment 367. The RNAi agent of embodiment 366, wherein X¹ comprises a 2′-β-D-deoxyribosyl sugar moiety.
Embodiment 368. The RNAi agent of embodiment 366, wherein X² or X⁴ comprises a 2′-β-D-deoxyribosyl sugar moiety.
Embodiment 369. The RNAi agent of embodiment 360 or 361, wherein at least one of X¹, X², X³, or X⁴ comprises a 2′-O-methyl-β-D-ribosyl sugar moiety.
Embodiment 370. The RNAi agent of embodiment 360 or 361, wherein each of X¹, X², X³, or X⁴ comprises a 2′-O-methyl-β-D-ribosyl sugar moiety.
Embodiment 371. The RNAi agent of any of embodiments 360-370, wherein n is 3 and p is 7.
Embodiment 372. The RNAi agent of any of embodiments 259-317, wherein the last two 3′-nucleosides of the antisense RNAi oligonucleotide are not paired with the sense RNAi oligonucleotide.
Embodiment 373. The RNAi agent of any of embodiments 259-372, wherein the last two 3′-nucleosides of the antisense RNAi oligonucleotide are not complementary to the target nucleic acid.
Embodiment 374. The RNAi agent of any of embodiments 259-373, wherein the last two 3′-nucleosides of the antisense RNAi oligonucleotide comprise a thymine nucleobase.
Embodiment 375. The RNAi agent of any of embodiments 259-374, wherein the 5′-stabilized phosphate group is selected from vinyl phosphonate, mesyl phosphoramidate, methylene phosphonate, and cyclopropyl phosphonate.
Embodiment 376. The RNAi agent of any of embodiments 259-374, wherein the 5′-stabilized phosphate group is vinyl phosphonate.
Embodiment 377. The RNAi agent of any of embodiments 259-376, wherein the antisense RNAi oligomeric compound comprises a conjugate group.
Embodiment 378. The RNAi agent of embodiment 377, wherein the conjugate group comprises a conjugate moiety and a conjugate linker.
Embodiment 379. The RNAi agent of embodiment 378, wherein the conjugate moiety is a cell-targeting moiety.
Embodiment 380. The RNAi agent of embodiment 378, wherein the conjugate moiety is a lipid.
Embodiment 381. The RNAi agent of embodiment 378, wherein the conjugate moiety comprises C₁₂-C₂₀ alkyl.
Embodiment 382. The RNAi agent of embodiment 378, wherein the conjugate moiety is C₁₆ alkyl.
Embodiment 383. The RNAi agent of embodiment 378, wherein the conjugate moiety is a carbohydrate.
Embodiment 384. The RNAi agent of embodiment 378, wherein the conjugate moiety comprises a GalNAc.
Embodiment 385. The RNAi agent of embodiment 378, wherein the conjugate moiety comprises an antibody or an antibody fragment.
Embodiment 386. The RNAi agent of embodiment 378, wherein the conjugate moiety comprises a peptide.
Embodiment 387. The RNAi agent of any of embodiments 378-386, wherein the conjugate moiety is part of an internucleoside linkage of Formula I.
Embodiment 388. The RNAi agent of any of embodiments 378-386, wherein the conjugate moiety is a 2′-modification of a sugar moiety.
Embodiment 389. The RNAi agent of any of embodiments 378-386, wherein the conjugate moiety is attached at the 3′-terminal of the antisense RNAi oligonucleotide.
Embodiment 390. The RNAi agent of any of embodiments 378-386, wherein the conjugate moiety is attached at the 5′-terminal of the antisense RNAi oligonucleotide.
Embodiment 391. The RNAi agent of any of embodiments 259-390, wherein the sense RNAi oligomeric compound comprises a conjugate group.
Embodiment 392. The RNAi agent of embodiment 391, wherein the conjugate group comprises a conjugate moiety and a conjugate linker.
Embodiment 393. The RNAi agent of embodiment 392, wherein the conjugate moiety is a cell-targeting moiety.
Embodiment 394. The RNAi agent of embodiment 392, wherein the conjugate moiety is a lipid.
Embodiment 395. The RNAi agent of embodiment 392, wherein the conjugate moiety comprises C₁₂-C₂₀ alkyl.
Embodiment 396. The RNAi agent of embodiment 392, wherein the conjugate moiety is C₁₆ alkyl.
Embodiment 397. The RNAi agent of embodiment 392, wherein the conjugate moiety is a carbohydrate.
Embodiment 398. The RNAi agent of embodiment 392, wherein the conjugate moiety comprises a GalNAc.
Embodiment 399. The RNAi agent of embodiment 392, wherein the conjugate moiety comprises an antibody or an antibody fragment.
Embodiment 400. The RNAi agent of embodiment 392, wherein the conjugate moiety comprises a peptide.
Embodiment 401. The RNAi agent of any of embodiments 392-400, wherein the conjugate moiety is part of an internucleoside linkage of Formula I.
Embodiment 402. The RNAi agent of any of embodiments 392-400, wherein the conjugate moiety is a 2′-modification of a sugar moiety.
Embodiment 403. The RNAi agent of any of embodiments 392-400, wherein the conjugate moiety is attached at the 3′-terminus of the sense RNAi oligonucleotide.
Embodiment 404. The RNAi agent of any of embodiments 392-400, wherein the conjugate moiety is attached at the 5′-terminus of the sense RNAi oligonucleotide.
Embodiment 405. An RNAi agent, comprising an antisense RNAi oligomeric compound comprising an antisense RNAi oligonucleotide consisting of 21-25 linked nucleosides and a sense RNAi oligomeric compound comprising a sense RNAi oligonucleotide consisting of 19-23 linked nucleosides, wherein the 5′-terminus of the antisense RNAi oligonucleotide has Structure A:

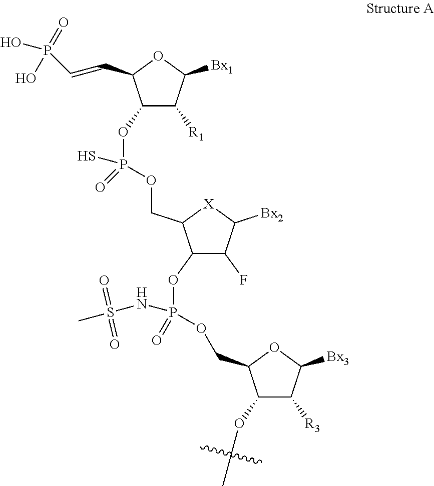

wherein
X is selected from O and O—C(H₂);
each Bx is an independently selected heterocyclic base moiety;
each of R₁ and R₃ is independently O[C(R₄)(R₅)]ₙ—[(C═O)ₘ—Z]ⱼ—R₆; wherein
each R₄ and R₅ is, independently, H, halogen, C₁-C₆ alkyl or substituted C₁-C₆ alkyl;
each Z is O, S or N(E₁);
R₆ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
   Q is O, S or NJ₃;
   each J₁, J₂ and J₃ is, independently, H or C₁-C₆ alkyl.
Embodiment 406. The RNAi agent of embodiment 405, wherein the 5′-terminus of the antisense RNAi oligonucleotide has Structure Aᵣ:

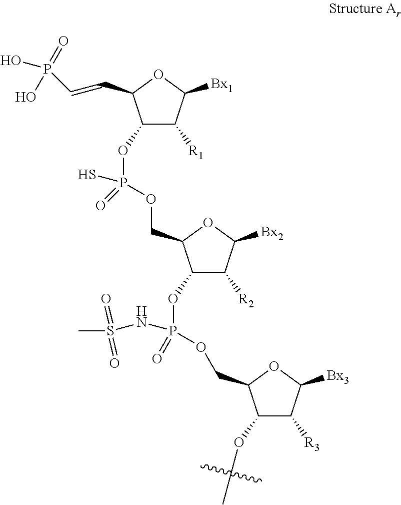

wherein
each Bx is an independently selected heterocyclic base moiety;
each of R₁ and R₃ is independently O[C(R₄)(R₅)]ₙ—[(C═O)ₘ—Z]ⱼ—R₆; wherein
each R₄ and R₅ is, independently, H, halogen, C₁-C₆ alkyl or substituted C₁-C₆ alkyl;
each Z is O, S or N(E₁);
R₆ is H, halogen, C₁-C₆ alkyl, substituted C₁-C₆ alkyl, C₂-C₆ alkenyl, substituted C₂-C₆ alkenyl, C₂-C₆
alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
Q is O, S or NJ₃;
each J₁, J₂ and J₃ is, independently, H or C₁-C₆ alkyl.
Embodiment 407. The RNAi agent of embodiment 405, wherein the 5′-terminus of the antisense RNAi oligonucleotide has Structure Aₓ:

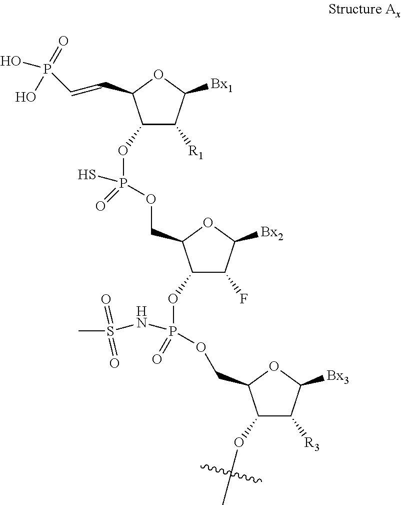

wherein
each Bx is an independently selected heterocyclic base moiety;
each of R₁ and R₃ is independently O[C(R₄)(R₅)]ₙ—[(C═O)ₘ—Z]ⱼ—R₆; wherein
each R₄ and R₅ is, independently, H, halogen, C₁-C₆ alkyl or substituted C₁-C₆ alkyl;
each Z is O, S or N(E₁);
R₆ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
   Q is O, S or NJ₃;
   each J₁, J₂ and J₃ is, independently, H or C₁-C₆ alkyl.
Embodiment 408. The RNAi agent of embodiment 405, wherein the 5′-terminus of the antisense RNAi oligonucleotide has Structure Aₕ:

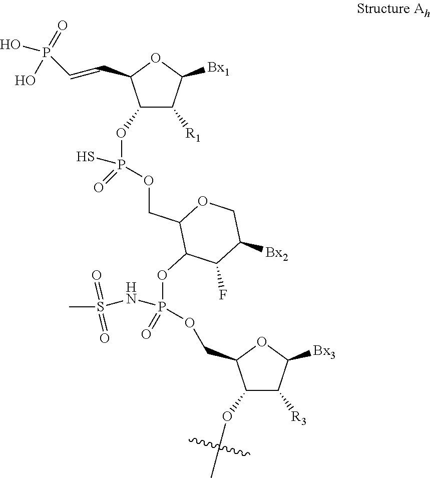

wherein
each Bx is an independently selected heterocyclic base moiety;
each of R₁ and R₃ is independently O[C(R₄)(R₅)]ₙ—[(C═O)ₘ—Z]ⱼ—R₆; wherein
each R₄ and R₅ is, independently, H, halogen, C₁-C₆ alkyl or substituted C₁-C₆ alkyl;
each Z is O, S or N(E₁);
R₆ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
E₁, E₂ and E₃ are each, independently, H, C₁-C₆ alkyl or substituted C₁-C₆ alkyl;
n is from 1 to 6;
m is 0 or 1;
j is 0 or 1;
each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
   Q is O, S or NJ₃;
   each J₁, J₂ and J₃ is, independently, H or C₁-C₆ alkyl.
Embodiment 409. The RNAi agent of any of embodiments 405-408, wherein R₁ is selected from OCH₃ and OCH₂CH₂OCH₃.
Embodiment 410. The RNAi agent of any of embodiments 405-408, wherein R₃ is OCH₃.
Embodiment 411. The RNAi agent of any of embodiments 405-410, wherein each Bx is selected from adenine, cytosine, uracil, thymine, guanine, and 5-methyl cytosine.
Embodiment 412. The RNAi agent of any of embodiments 405-411, wherein Bx₁ is thymine.
Embodiment 413. The RNAi agent of any of embodiments 405-412, wherein the antisense RNAi oligonucleotide comprises at least one sugar moiety selected from 2′-O-methyl-β-D-ribosyl sugar moiety, a 2′-O-methoxyethyl-β-D-ribosyl sugar moiety, a sugar surrogate, a stereo-non-standard sugar moiety, a 2′-β-D-deoxyribosyl sugar moiety, and a β-D-ribosyl sugar moiety.
Embodiment 414. The RNAi agent of embodiment 413, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.
Embodiment 415. The RNAi agent of embodiment 413, wherein each sugar surrogate is independently selected from fluoro hexitol nucleic acid (F-HNA), hexitol nucleic acid (HNA), or altritol nucleic acid (ANA).
Embodiment 416. The RNAi agent of any of embodiments 405-415, wherein the antisense RNAi oligonucleotide comprises no more than three 2′-fluoro-β-D-ribosyl sugar moieties.
Embodiment 417. The RNAi agent of any of embodiments 405-416, wherein each internucleoside linkage of the antisense RNAi oligonucleotide is selected from mesyl phosphoramidate internucleoside linkage, a phosphodiester internucleoside linkage, and a phosphorothioate internucleoside linkage.
Embodiment 418. The RNAi agent of any of embodiments 405-417, wherein the antisense RNAi oligonucleotide has an internucleoside linkage motif selected from sz(o)ₙzs and sz(o)ₙss, wherein each “s” is a phosphorothioate internucleoside linkage, each “z” is an internucleoside linkage of Formula I, each “o” is a phosphodiester internucleoside linkage, and n is from 16-20.
Embodiment 419. The RNAi agent of embodiment 418, wherein for each internucleoside linakge of Formula I, R is methyl.
Embodiment 420. The RNAi agent of any of embodiments 405-419, wherein the antisense RNAi oligonucleotide consists of 23 linked nucleosides.
Embodiment 421. The RNAi agent of any of embodiments 405-420, wherein the antisense RNAi oligonucleotide has a sugar motif selected from: y[f2bDx]yyyfyyyyyyyfyfyyyyyyy, yfyyy[f2bDx]yyyyyyyfyfyyyyyyy, yfyyyfyyyyyyy[f2bDx]yfyyyyyyy, yfyyyyyyyyyyfy[f2bDx]yyyyyyy, y[f2bDx]yyy[f2bDx]yyyyyyy[f2bDx]y[f2bDx]yyyyyyy, efyyyfy[16C2r]yyyyyfyfyyyyyyy, e[f2bDx]yyyfyyyyyyyfyfyyyyyyy, efyyy[f2bDx]yyyyyyyfyfyyyyyyy, efyyyfyyyyyyy[f2bDx]yfyyyyyyy, efyyyfyyyyyyyfy[f2bDx]yyyyyyy, e[f2bDx]yyy[f2bDx]yyyyyyy[f2bDx]y[f2bDx]yyyyyyy, efyyy[F-HNA]yyyyyyyfyfyyyyyyy, efyyyfyyyyyyy[F-HNA]yfyyyyyyy, efyyyfyyyyyyyfy[F-HNA]yyyyyyy, yfyyydyyyyyyyfyfyyyyyyy, yfyyyfyyyyyyydyfyyyyyyy, yfyyyfyyyyyyyfydyyyyyyy, yfyyydyyyyyyydydyyyyyyy, yryyyfyyyyyyyfyfyyyyyyy, yfyyyryyyyyyyfyfyyyyyyy, yfyyyfyyyyyyyryfyyyyyyy, yfyyyfyyyyyyyfyryyyyyyy, yryyyryyyyyyyryryyyyyyy, y[bDdx]yyyfyyyyyyyfyfyyyyyyy, yfyyy[bDdx]yyyyyyyfyfyyyyyyy, yfyyyfyyyyyyy[bDdx]yfyyyyyyy, yfyyyfyyyyyyyfy[bDdx]yyyyyyy, y[bDdx]yyy[bDdx]yyyyyyy[bDdx]y[bDdx]yyyyyyy, y[F-HNA]yyyfyyyyyyyfyfyyyyyyy, yfyyy[F-HNA]yyyyyyyfyfyyyyyyy, yfyyyfyyyyyyy[F-HNA]yfyyyyyyy, yfyyyfyyyyyyyfy[F-HNA]yyyyyyy, y[F-HNA]yyy[F-HNA]yyyyyyy[F-HNA]y[F-HNA]yyyyyyy, yfyyyfyyyyyyy[2bDx]yfyyyyyyy, y[bDa]yyyfyyyyyyyfyfyyyyyyy, yfyyy[bDa]yyyyyyyfyfyyyyyyy, yfyyyfyyyyyyy[bDa]yfyyyyyyy, yfyyyfyyyyyyyfy[bDa]yyyyyyy, y[bDa]yyy[bDa]yyyyyyy[bDa]y[bDa]yyyyyyy, yfyyyfyyyyyyy[bDx]yfyyyyyyy, yfyyyfyffyyyyfyfyyyyydd, yfyyyfyffyyyyfyfyyyyy[bLdr][bLdr], yfyyyfyffyyyyfyfyyyyy[aDdr][aDdr], yfyyyfyffyyyyfyfyyyyy[bDdx][bDdx], yfyyyfyffyyyyfyfyyyyy[bLdx][bLdx], yfyyyfyffyyyyfyfyyyyy[aDdx][aDdx], yfyyyfyffyyyyfyfyyyyy[aLdx][aLdx], yfyyyfyyyyyyyfyfyyyyyee, yfyyyfyyyyyyyfyfyyyyykk, yfyyyfyyyyyyyfyfyyyyy[LNA][LNA], yfyyyfyyyyyyyfyfyyyyy[F-HNA][F-HNA], [ANA]fyyyfyyyyyyyfyfyyyyyyy, y[ANA]yyyfyyyyyyyfyfyyyyyyy, yfyyyf[ANA]yyyyyyfyfyyyyyyy, yfyyyfyy[ANA]yyyyfyfyyyyyyy, yfyyyfyyyyyyy[ANA]yfyyyyyyy, yfyyyfyyyyyyyfyf[ANA]yyyyyy, yfyyyfyy[ANA]yyyy[ANA]yf[ANA]yyyyy[ANA], yfyyyfyyyyyyyfyfyyyyyyk, yfyyyfyyyyyyyfyfyyyyyke, yfyyyfyyyyyyyfyfyyyyyky, efyyydyyyyyyydydyyyyyyy, yfyyyfyyyyyyy[ANA]yf[ANA]yyyyyy, yfyyyfyyyyyyyfyfyyyyy[ANA]y, yfyyyfyyyyyyyfyfyyyyy[f2bDa][f2bDa], yfyyyfyyyyyyyfyfyyyyynn, yfyyyfyyyyyyyfyfyyyyy[DMAEOE][DMAEOE], yfyyyfyyyyyyyfyfyyyyy[HNA][HNA], yfyyyfyyyyyyyfyfyyyyy[SM5LNA][SM5LNA], yfyyyfyyyyyyyfyfyyyyy[DMAOE][DMAOE], yfyyyfyyyyyyyfyfyyyyydd, yfyyyfyyyyyyyfyfyyyyy[aLdr][aLdr], [HNA]fyyyfyyyyyyyfyfyyyyyyy, dfyyyfyyyyyyyfyfyyyyyyy, y[HNA]yyyfyyyyyyyfyfyyyyyyy, e[HNA]yyyfyyyyyyyfyfyyyyyyy, yfyyyf[HNA]yyyyyyfyfyyyyyyy, yfyyyfyy[HNA]yyyyfyfyyyyyyy, yfyyyfyyyyyyy[HNA]yfyyyyyyy, yfyyyfyyyyyyyfyf[HNA]yyyyyy, yfyyyfyy[HNA]yyyy[HNA]yf[HNA]yyyyy[HNA], yfyyyfyyyyyyy[HNA]yf[HNA]yyyyyy, yfyyyfyyyyyyyfyfyyyyy[HNA]y, kfyyyfyyyyyyyfyfyyyyyyy, e[F-HNA]yyyfyyyyyyyfyfyyyyyyy, e[LNA]yyyfyyyyyyyfyfyyyyyyy, edyyyfyyyyyyyfyfyyyyyyy, edyyydyyyyyyydydyyyyyyy, ydyyyfyyyyyyyfyfyyyyyyy, ydyyydyyyyyyydydyyyyyyy, efyyfyfyyyyfyfyyyyyyyyy, edyydydyyyydyfyyyyyyyyy, efyyyfyyyyyyyfyfyyyyydd, [F-HNA]fyyyfyyyyyyyfyfyyyyyyy, eyyyyfyyyyyyyfyfyyyyyyy, eryyyryyyyyyyryryyyyyyy, efyyydyyyyyyyfyfyyyyyyy, efyyyfyyyyyyyfydyyyyyyy, efyyyfyyyyyyydyfyyyyyyy; wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C2r]” represents a 2′-O-hexadecyl-β-D-ribosyl sugar moiety, “[F-HNA]” represents the sugar surrogate 3′-fluoro-hexitol sugar surrogate, “[bDdx]” represents a 2′-β-D-deoxyxylosyl sugar moiety, “[bDx]” represents a β-D-xylosyl sugar moiety, “[bDa]” represents a β-D-arabinosyl sugar moiety, “[bLdr]” represents a 2′-β-L-deoxyribosyl sugar moiety, “[aDdr]” represents a 2′-α-D-deoxyribosyl sugar moiety, “[aLdr]” represents a 2′-α-L-deoxyribosyl sugar moiety, “[bLdx]” represents a 2′-β-L-deoxyxylosyl sugar moiety, “[aDdx]” represents an 2′-α-D-deoxyxylosyl sugar moiety, “[aLdx]” represents an 2′-α-L-deoxyxylosyl sugar moiety, “[LNA]” represents a β-D-LNA sugar moiety, “[f2bDa]” represents a 2′-fluoro-β-D-arabionsyl sugar moiety, “n” represents a 2′-O—(N-methylacetamide) ribosyl sugar moiety, “[DMAEOE]” represents a 2′-O-(2-(2-(N,N-dimethyl)aminoethoxy)ethyl)-β-D-ribosyl sugar moiety, “[SM5LNA]” represents a (5'S)-5′methyl-LNA sugar moiety, “[ANA]” represents an ANA sugar surrogate, and “[HNA]” an HNA sugar surrogate.
Embodiment 422. The RNAi agent of any of embodiments 405-421, wherein the antisense RNAi oligonucleotide has a target complementary region of at least 18 consecutive nucleosides that are at least 85% complementary to an equal length portion of a target nucleic acid.
Embodiment 423. The RNAi agent of embodiment 422, wherein the antisense RNAi oligonucleotide has a target complementary region that is at least 90%, at least 95%, or 100% complementary to an equal length portion of a target nucleic acid.
Embodiment 424. The RNAi agent of embodiment 423, wherein the target complementary region consists of 21 consecutive nucleosides.
Embodiment 425. The RNAi agent of any of embodiments 405-424, wherein the sense RNAi oligonucleotide has a complementary region of at least 18 consecutive nucleosides that are at least 85% complementary to an equal length region of the antisense RNAi oligonucleotide.
Embodiment 426. The RNAi agent of embodiment 425, wherein the sense RNAi oligonucleotide consists of 2 fewer linked nucleosides than the antisense RNAi oligonucleotide.
Embodiment 427. The RNAi agent of embodiment 426, wherein the complementary region of the sense RNAi consists of 21 nucleobases.
Embodiment 428. The RNAi agent of embodiment 427, wherein the antisense RNAi oligonucleotide consists of 23 linked nucleosides, the sense RNAi oligonucleotide consists of 21 linked nucleosides, the sense RNAi oligonucleotide is 100% complementary to the antisense RNAi oligonucleotide, and the antisense RNAi oligonucleotide is at least 85% complementary to an equal length portion of a target nucleic acid.
Embodiment 429. The RNAi agent of any of embodiments 405-428, each internucleoside linkage of the sense RNAi oligonucleotide is selected from an internucleoside linkage of Formula I, a phosphodiester internucleoside linkage, and a phosphorothioate internucleoside linkage.
Embodiment 430. The RNAi agent of embodiment 429, wherein the sense RNAi oligonucleotide has an internucleoside linkage motif selected of qq(o)ₙqq, wherein each “q” is independently selected from a phosphorothioate internucleoside linkage and an internucleoside linkage of Formula I, each “o” is a phosphodiester internucleoside linkage, and n is from 14-18.
Embodiment 431. The RNAi agent of embodiment 429 or 430, wherein for each internucleoside linkage of Formula I, R is methyl.
Embodiment 432. The RNAi agent of embodiment 431, wherein the sense RNAi oligonucleotide has an internucleoside linkage motif selected of qq(o)ₙz(o)ₘqq, wherein each “q” is independently selected from a phosphorothioate internucleoside linkage and an internucleoside linkage of Formula I wherein X is O and R is methyl, each “o” is a phosphodiester internucleoside linkage, and “z” is an internucleoside linkage of Formula I wherein X is O and R is selected from C₁₀-C₂₀ alkyl, substituted C₁₀-C₂₀ alkyl, and a conjugate group, wherein n is from 2 to 5, m is from 8 to 15, and n+m is from 13 to 17.
Embodiment 433. The RNAi agent of any of embodiments 405-432, wherein the sense RNAi oligonucleotide consists of 21 linked nucleosides and has an internucleoside linkage motif selected from ssoooooooooooooooooo, ssoooooooooooooooooo, ssooooooooooooooooss, ssooooooooooooooooss, zzoooooooooooooooozz, ssoooozooozoooooooss, ssoooozozozoooooooss, ssoooozozzzoooooooss, zsoooooooooooooooosz, and zzoooooooooooooooooo, wherein each “s” is a phosphorothioate internucleoside linkage, each “z” is an internucleoside linkage of Formula I and each “o” is a phosphodiester internucleoside linkage.
Embodiment 434. The RNAi agent of any of embodiments 405-433, wherein for at least one internucleoside linkage of Formula I of the sense RNAi oligonucleotide, X is O and R is methyl.
Embodiment 435. The RNAi agent of any of embodiments 405-434, wherein for at least one internucleoside linkage of Formula I of the sense RNAi oligonucleotide, X is O and R is C₁₆.
Embodiment 436. The RNAi agent of any of embodiments 405-435, wherein for at least one internucleoside linkage of Formula I of the sense RNAi oligonucleotide, R is a conjugate group.
Embodiment 437. The RNAi agent of embodiment 438, wherein the conjugate group comprises a conjugate linker and a conjugate moiety.
Embodiment 438. The RNAi agent of embodiment 437, wherein the conjugate moiety is selected from a lipid, a carbohydrate, a GalNAc, an antibody or fragment thereof, or a peptide.
Embodiment 439. The RNAi agent of any of embodiments 405-438, wherein each of at least two sugar moieties of the sense RNAi is selected from a 2′-O-methyl-β-D-ribosyl sugar moiety, a 2′-O-methoxyethyl-β-D-ribosyl sugar moiety, a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, and wherein each of at least two such sugar moieties are different from each other.
Embodiment 440. The RNAi agent of any of embodiments 405-439, wherein the sense RNAi oligonucleotide consists of 21 linked nucleosides and has a sugar motif of yyyyyyxyxxxyyyyyyyyyy, from 5′ to 3′, wherein each “y” is a 2′-O-methyl β-D-ribosyl sugar moiety and each “x” is independently selected from a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, wherein at least one “x” is other-than a 2′-fluoro-β-D-ribosyl sugar moiety.
Embodiment 441. The RNAi agent of embodiment 439 or 440, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.
Embodiment 442. The RNAi agent of embodiment 440, wherein at least one “x” is a 2′-β-D-deoxyribosyl sugar moiety.
Embodiment 443. The RNAi agent of embodiment 442, wherein each “x” is a 2′-β-D-deoxyribosyl sugar moiety.
Embodiment 444. The RNAi agent of embodiment 440, wherein at least one “x” is a 2′-fluoro-β-D-xylosyl sugar moiety.
Embodiment 445. The RNAi agent of embodiment 444, wherein each “x” is a 2′-fluoro-β-D-xylosyl sugar moiety.
Embodiment 446. The RNAi agent of embodiment 440, wherein at least one “x” is a sugar surrogate.
Embodiment 447. The RNAi agent of embodiment 446, wherein each sugar surrogate is independently selected from fluoro hexitol nucleic acid (F-HNA), hexitol nucleic acid (HNA) or altritol nucleic acid (ANA).
Embodiment 448. The RNAi agent of any of embodiments 405-447, wherein the sense RNAi oligonucleotide has a sugar motif selected from: yyyyyyfyff[f2bDx]yyyyyyyyyy, yyyyyyfyf[f2bDx]fyyyyyyyyyy, yyyyyyfy[f2bDx]ffyyyyyyyyyy, yyyyyy[f2bDx]yfffyyyyyyyyyy, yyyyyy[f2bDx]y[f2bDx][f2bDx][f2bDx]yyyyyyyyyy, yyyyy[16C2r]fyfffyyyyyyyyyy, yyyyy[C16A]fyfffyyyyyyyyyy, yyyyy[16C2r]fyfffyyyyyyyyyy, yyyyy[16C2r]fyff[f2bDx]yyyyyyyyyy, yyyyy[16C2r]fyf[f2bDx]fyyyyyyyyyy, yyyyy[16C2r]fy[f2bDx]ffyyyyyyyyyy, yyyyy[16C2r][f2bDx]yfffyyyyyyyyyy, yyyyy[16C2r][f2bDx]y[f2bDx][f2bDx][f2bDx]yyyyyyyyyy, yyyyyyfyfffyyyyyyyyyyddddddddd, yyyyy[16C2r]fyff[F-HNA]yyyyyyyyyy, yyyyy[16C2r]fyf[F-HNA]fyyyyyyyyyy, yyyyy[16C2r]fy[F-HNA]ffyyyyyyyyyy, yyyyy[16C2r][F-HNA]yfffyyyyyyyyyy, yyyyy[16C2r][F-HNA]yff[F-HNA]yyyyyyyyyy, yyyyy[16C2r][F-HNA]y[F-HNA][F-HNA][F-HNA]yyyyyyyyyy, yyyyyyyyyyyyyyyyyyyyy, yyyyyyyyfffyyyyyyyyyy, yyyyyyfyyffyyyyyyyyyy, yyyyyyfyfyfyyyyyyyyyy, yyyyyyfyffyyyyyyyyyyy, yyyyyyyyyffyyyyyyyyyy, yyyyyyfyyyfyyyyyyyyyy, yyyyyyfyfyyyyyyyyyyyy, yyyyyyyyfyfyyyyyyyyyy, yyyyyyyyffyyyyyyyyyyy, yyyyyyfyyfyyyyyyyyyyy, yyyyyyeyeeeyyyyyyyyyy, yyyyyyeyfffyyyyyyyyyy, yyyyyyfyeffyyyyyyyyyy, yyyyyyfyfefyyyyyyyyyy, yyyyyyfyffeyyyyyyyyyy, yyyyyyeyeffyyyyyyyyyy, yyyyyyfyeefyyyyyyyyyy, yyyyyyfyfeeyyyyyyyyyy, yyyyyyeyfefyyyyyyyyyy, yyyyyyeyffeyyyyyyyyyy, yyyyyyfyefeyyyyyyyyyy, yyyyyydyfffyyyyyyyyyy, yyyyyyfydffyyyyyyyyyy, yyyyyyfyfdfyyyyyyyyyy, yyyyyyfyffdyyyyyyyyyy, yyyyyydydffyyyyyyyyyy, yyyyyydyfdfyyyyyyyyyy, yyyyyydyffdyyyyyyyyyy, yyyyyydydfdyyyyyyyyyy, yyyyyyfydddyyyyyyyyyy, yyyyyydyddfyyyyyyyyyy, yyyyyydydddyyyyyyyyyy, yyyyydddddddddddyyyyy, yyyyyydddddddddyyyyyy, yyyyyyydddddddyyyyyyy, yyyyyyyydddddyyyyyyyy, eeeeedddddddddddeeeee, eeeeeedddddddddeeeeee, eeeeeeedddddddeeeeeee, eeeeeeeedddddeeeeeeee, yyyyyydydydydyddyyyyy, eeeeeedededededdeeeee, dydydydydydydydydydyd, dydydyfyfffydydydydyd, dedededededededededed, dededefefffededededed, ryryryryryryryryryryr, ryryryfyfffyryryryryr, rerererererererererer, rererefefffererererer, yyyyy[16C2r][f2bDx]yff[f2bDx]yyyyyyyyyy, yyyyyyryfffyyyyyyyyyy, yyyyyyfyrffyyyyyyyyyy, yyyyyyfyfrfyyyyyyyyyy, yyyyyyfyffryyyyyyyyyy, yyyyyyryrrryyyyyyyyyy, yyyyyyfyff[bDdx]yyyyyyyyyy, yyyyyyfyf[bDdx]fyyyyyyyyyy, yyyyyyfy[bDdx]ffyyyyyyyyyy, yyyyyy[bDdx]yfffyyyyyyyyyy, yyyyyy[bDdx]y[bDdx][bDdx][bDdx]yyyyyyyyyy, yyyyyyfyff[F-HNA]yyyyyyyyyy, yyyyyyfyf[F-HNA]fyyyyyyyyyy, yyyyyyfy[F-HNA]ffyyyyyyyyyy, yyyyyy[F-HNA]yfffyyyyyyyyyy, yyyyyy[F-HNA]y[F-HNA][F-HNA][F-HNA]yyyyyyyyyy, yyyyyyfyf[bDx]fyyyyyyyyyy, yyyyyyfyf[bDx]fyyyyyyyyyy, yyyyyyfyf[ANA]fyyyyyyyyyy, [ANA]yyyyyfyf[ANA]fyyyyyy[ANA]yyy, [ANA]yy[ANA]yyfyf[ANA]fyyyyyyyyyy, yyy[ANA]y[ANA]fyf[ANA]fyyyyyyyyyy, yyyyyyfyf[ANA]f[ANA]yyyyy[ANA]yyy, [ANA]yyyy[ANA]fyf[ANA]f[ANA]yyyyyyyyy, [ANA]yyyy[ANA]fyf[ANA]fyyyy[ANA]yyyyy, [ANA]yyyy[ANA]fyf[ANA]f[ANA]yyy[ANA]yyyyy, yyyyyyfyf[HNA]fyyyyyyyyyy, [HNA]yyyyyfyf[HNA]fyyyyyy[HNA]yyy, [HNA]yy[HNA]yyfyf[HNA]fyyyyyyyyyy, yyy[HNA]y[HNA]fyf[HNA]fyyyyyyyyyy, yyyyyyfyf[HNA]f[HNA]yyyyy[HNA]yyy, [HNA]yyyy[HNA]fyf[HNA]fyyyy[HNA]yyyyy, [HNA]yyyy[HNA]fyf[HNA]f[HNA]yyyyyyyyy, [HNA]yyyy[HNA]fyf[HNA]f[HNA]yyy[HNA]yyyyy, yyyyyydydydydydyyyyyy, yyyyyy[bDa]yfffyyyyyyyyyy, yyyyyyfy[bDa]ffyyyyyyyyyy, yyyyyyfyf[bDa]fyyyyyyyyyy, yyyyyyfyff[bDa]yyyyyyyyyy, yyyyyy[bDa]y[bDa][bDa][bDa]yyyyyyyyyy, yyyyyyyyyyyyyyyyyyyy, yyyyyyyyfyyyyyyyyyyyy, yyyyyyfyyyyyyyyyyyyyy, yyyyyyyydddyyyyyyyyyy, wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C2r]” represents a 2′-O-hexadecyl-β-D-ribosyl sugar moiety, “[C16A]” represents 2′-O-hexylpalmitamide-β-D-ribosyl sugar moiety “[F-HNA]” represents the sugar surrogate 3′-fluoro-hexitol sugar surrogate, “[HNA]” represents an HNA sugar surrogate; “[ANA]” represents an ANA sugar surrogate; “[bDdx]” represents a 2′-β-D-deoxyxylosyl sugar moiety, “[bDx]” represents a β-D-xylosyl sugar moiety, “[bDa]” represents a β-D-arabinosyl sugar moiety.
Embodiment 449. The RNAi agent of any of embodiments 405-449, wherein the last two 3′-nucleosides of the antisense RNAi oligonucleotide are not paired with the sense RNAi oligonucleotide.
Embodiment 450. The RNAi agent of any of embodiments 405-449, wherein the last two 3′-nucleosides of the antisense RNAi oligonucleotide are not complementary to the target nucleic acid.
Embodiment 451. The RNAi agent of any of embodiments 405-449, wherein the last two 3′-nucleosides of the antisense RNAi oligonucleotide comprise a thymine nucleobase.
Embodiment 452. The RNAi agent of any of embodiments 405-451, wherein the antisense RNAi oligomeric compound comprises a conjugate group.
Embodiment 453. The RNAi agent of embodiment 452, wherein the conjugate group comprises a conjugate moiety and a conjugate linker.
Embodiment 454. The RNAi agent of embodiment 452, wherein the conjugate moiety is selected from a lipid, a carbohydrate, a peptide, and antibody, or an antibody fragment.
Embodiment 455. The RNAi agent of embodiment 452, wherein the conjugate group comprises a C₁₂-C₂₀ alkyl, C₁₆ alkyl, or a GalNAc.
Embodiment 456. The RNAi agent of any of embodiments 405-455, wherein the sense RNAi oligomeric compound comprises a conjugate group.
Embodiment 457. The RNAi agent of embodiment 456, wherein the conjugate group comprises a conjugate moiety and a conjugate linker.
Embodiment 458. The RNAi agent of embodiment 457, wherein the conjugate moiety is a cell-targeting moiety.
Embodiment 459. The RNAi agent of embodiment 457, wherein the conjugate moiety is a lipid.
Embodiment 460. The RNAi agent of embodiment 457, wherein the conjugate moiety comprises C₁₂-C₂₀ alkyl.
Embodiment 461. The RNAi agent of embodiment 457, wherein the conjugate moiety is C₁₆ alkyl.
Embodiment 462. The RNAi agent of embodiment 457, wherein the conjugate moiety is a carbohydrate.
Embodiment 463. The RNAi agent of embodiment 457, wherein the conjugate moiety comprises a GalNAc.
Embodiment 464. The RNAi agent of embodiment 457, wherein the conjugate moiety comprises an antibody or an antibody fragment.
Embodiment 465. The RNAi agent of embodiment 457, wherein the conjugate moiety comprises a peptide.
Embodiment 466. The RNAi agent of any of embodiments 452-465, wherein the conjugate moiety is part of an internucleoside linkage of Formula I.
Embodiment 467. The RNAi agent of any of embodiments 452-462, wherein the conjugate moiety is a 2′-modification of a sugar moiety.
Embodiment 468. The RNAi agent of any of embodiments 452-465, wherein the conjugate moiety is attached at the 3′-terminal of the sense RNAi oligonucleotide.
Embodiment 469. The RNAi agent of any of embodiments 452-465, wherein the conjugate moiety is attached at the 5′-terminal of the sense RNAi oligonucleotide.
Embodiment 470. The RNAi agent of any of embodiments 259-469, wherein each nucleobase of the antisense RNAi oligonucleotide is selected from uracil, thymine, guanine, adenine, cytosine, and 5-methylcytosine.
Embodiment 471. The RNAi agent of any of embodiments 259-470, wherein each nucleobase of the sense RNAi oligonucleotide is selected from uracil, thymine, guanine, adenine, cytosine, and 5-methylcytosine.
Embodiment 472. A chirally enriched population of RNAi agents of any of embodiments 259-471, wherein the population is enriched for antisense RNAi oligonucleotides and/or sense RNAi oligonucleotides comprising at least one particular internucleoside linkage having a particular stereochemical configuration.
Embodiment 473. The chirally enriched population of embodiment 472, wherein the population is enriched for antisense RNAi oligonucleotides and/or sense RNAi oligonucleotides comprising at least one particular internucleoside of Formula I having the (Sp) or (Rp) configuration.
Embodiment 474. The chirally enriched population of embodiment 472, wherein the population is enriched for antisense RNAi oligonucleotides comprising at least one internucleoside linkage of Formula I having the (Sp) configuration.
Embodiment 475. The chirally enriched population of embodiment 472, wherein the population is enriched for antisense RNAi oligonucleotides comprising at least one internucleoside linkage of Formula I having the (Rp) configuration.
Embodiment 476. The chirally enriched population of embodiment 473, wherein the at least one internucleoside linkage of Formula I having the (Sp) configuration is the first internucleoside linkage from the 5′ end of the antisense RNAi oligonucleotide.
Embodiment 477. The chirally enriched population of embodiment 474, wherein the at least one internucleoside linkage of Formula I having the (Rp) configuration is the first internucleoside linkage from the 5′ end of the antisense RNAi oligonucleotide.
Embodiment 478. The chirally enriched population of embodiment 473, wherein the at least one internucleoside linkage of Formula I having the (Sp) configuration is the second internucleoside linkage from the 5′ end of the antisense RNAi oligonucleotide.
Embodiment 479. The chirally enriched population of embodiment 474, wherein the at least one internucleoside linkage of Formula I having the (Rp) configuration is the second internucleoside linkage from the 5′ end of the antisense RNAi oligonucleotide.
Embodiment 480. A population of RNAi agents of any of embodiments 259-471, wherein each internucleoside linkage of the antisense RNAi oligonucleotide and the sense RNAi oligonucleotide is stereorandom.
Embodiment 481. A method comprising administering at least two doses of an RNAi agent of any of embodiments 259-480 to an animal wherein:
   the RNAi agent is administered to the animal at a dose frequency of once per 4 months, 5 months, 6 months, 7 months, 8 months, 9 months, 10 months, 11 months, or a year or more than a year.

### Certain Compounds

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula I:

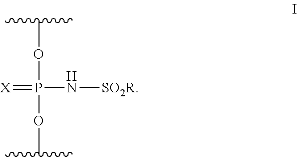

X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl, substituted C₁-C₆ alkynyl, N(C₁-C₆ alkyl)₂, and a conjugate group.

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula II.

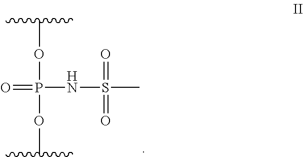 [SMILES: *OP(=O)(NS(C)(=O)=O)O*]

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula III.

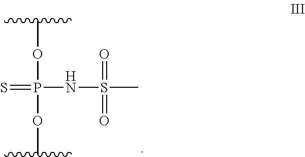 [SMILES: *OP(=S)(NS(C)(=O)=O)O*]

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula IV.

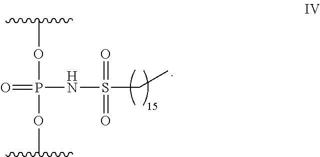

In certain embodiments, compounds described herein are oligomeric compounds (including oligomeric compounds that are antisense agents or portions thereof) comprising or consisting of oligonucleotides consisting of linked nucleosides and having at least one internucleoside linking group of Formula XIV: Q is R^{A} or R^{B}; wherein independently for each internucleoside linkage of Formula XIV:

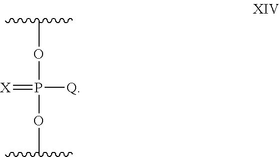

X is selected from O or S;
R^{A} is —NHSO₂R; R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group;
R^{B} is —N═CR¹⁰R¹¹, wherein R¹⁰ and R¹¹ are each independently alkyl, or optionally wherein R¹⁰ and R¹¹, along with the intervening atoms, join to form a heterocyclic ring.

Oligonucleotides may be unmodified oligonucleotides or may be modified oligonucleotides. Modified oligonucleotides comprise at least one modification relative to an unmodified oligonucleotide (i.e., comprise at least one modified nucleoside (comprising a modified sugar moiety, a stereo-non-standard nucleoside, and/or a modified nucleobase) and/or at least one modified internucleoside linkage). In certain embodiments, the modified internucleoside linkage is a modified internucleoside linking group having Formula I or Formula XIV. In certain embodiments, compounds described herein are oligomeric compounds (including oligomeric compounds that are antisense agents or portions thereof) having at least one modified internucleoside linking group having Formula I or Formula XIV.

In certain embodiments, compounds described herein comprise an antisense RNAi oligomeric compound comprising an antisense RNAi oligonucleotide having a 5′-terminus having Structure A, Structure Aᵣ, Structure Aₓ, or Structure Aₕ

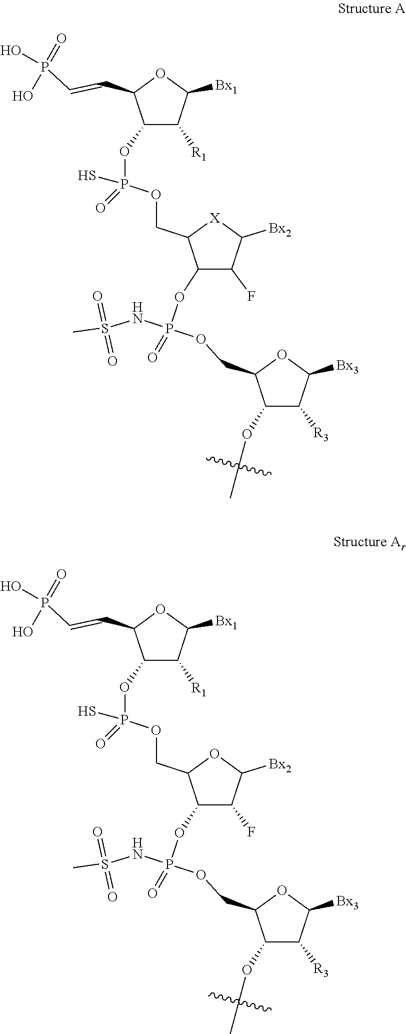

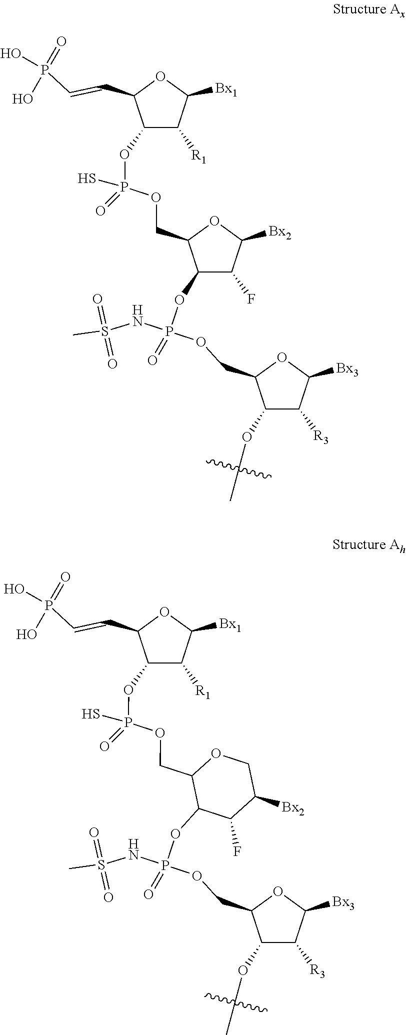

wherein
each Bx is an independently selected heterocyclic base moiety;
each of R₁ and R₃ is independently O[C(R₄)(R₅)]ₙ—[(C═O)ₘ—Z]ⱼ—R₆; wherein
   each R₄ and R₅ is, independently, H, halogen, C₁-C₆ alkyl or substituted C₁-C₆ alkyl;
   each Z is O, S or N(E₁);
   R₆ is H, halogen, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or N(E₂)(E₃);
   E₁, E₂ and E₃ are each, independently, H, C₁-C₆ alkyl or substituted C₁-C₆ alkyl;
   n is from 1 to 6;
   m is 0 or 1;
   j is 0 or 1;
   each substituted group comprises one or more optionally protected substituent groups independently selected from halogen, OJ₁, N(J₁)(J₂), =NJ₁, SJ₁, N₃, CN, OC(=Q)J₁, OC(=Q)N(J₁)(J₂) and C(=Q)N(J₁)(J₂);
      Q is O, S or NJ₃;
      each J₁, J₂ and J₃ is, independently, H or C₁-C₆ alkyl.

### I. Modifications

### A. Modified Nucleosides

Modified nucleosides comprise a stereo-non-standard nucleoside, or a modified sugar moiety, or a modified nucleobase, or any combination thereof.

### 1. Certain Modified Sugar Moieties

In certain embodiments, modified sugar moieties are stereo-non-standard sugar moieties. In certain embodiments, sugar moieties are substituted furanosyl stereo-standard sugar moieties. In certain embodiments, modified sugar moieties are bicyclic or tricyclic furanosyl sugar moieties. In certain embodiments, modified sugar moieties are sugar surrogates. Such sugar surrogates may comprise one or more substitutions corresponding to those of other types of modified sugar moieties.

a. Stereo-Non-Standard Sugar Moieties

In certain embodiments, modified sugar moieties are stereo-non-standard sugar moieties shown in Formulas V-XI below:

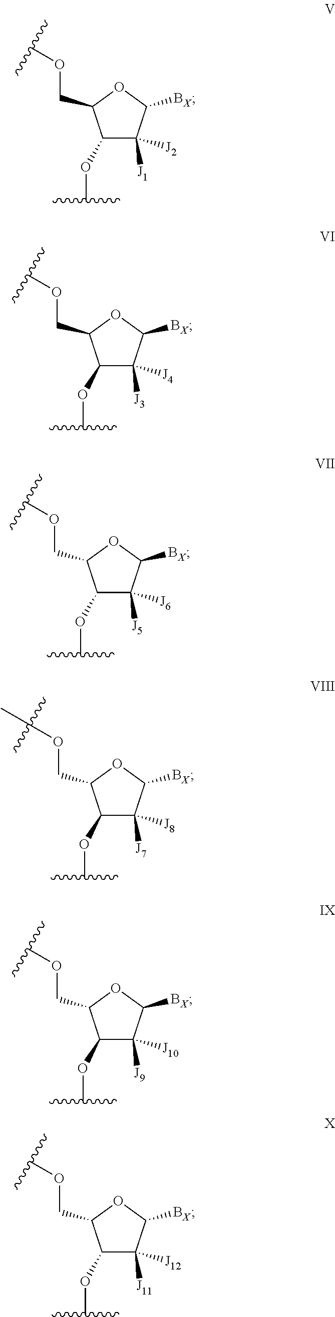

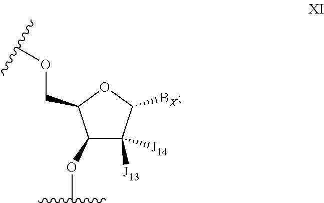

wherein
one of J₁ and J₂ is H and the other of J₁ and J₂ is selected from H, OH, F, OCH₃, OCH₂CH₂OCH₃, O—C₁-C₆ alkoxy, and SCH₃;
one of J₃ and J₄ is H and the other of J₃ and J₄ is selected from H, OH, F, OCH₃, OCH₂CH₂OCH₃, O—C₁-C₆ alkoxy, and SCH₃; and wherein
one of J₅ and J₆ is H and the other of J₅ and J₆ is selected from H, OH, F, OCH₃, OCH₂CH₂OCH₃, O—C₁-C₆ alkoxy, and SCH₃; and wherein
one of J₇ and J₈ is H and the other of J₇ and J₈ is selected from H, OH, F, OCH₃, OCH₂CH₂OCH₃, O—C₁-C₆ alkoxy, and SCH₃; and wherein
one of J₉ and J₁₀ is H and the other of J₉ and J₁₀ is selected from H, OH, F, OCH₃, OCH₂CH₂OCH₃, O—C₁-C₆ alkoxy, and SCH₃; and wherein
one of J₁₁ and J₁₂ is H and the other of J₁₁ and J₁₂ is selected from H, OH, F, OCH₃, OCH₂CH₂OCH₃, O—C₁-C₆ alkoxy, and SCH₃; and wherein
one of J₁₃ and J₁₄ is H and the other of J₁₃ and J₁₄ is selected from H, OH, F, OCH₃, OCH₂CH₂OCH₃, O—C₁-C₆ alkoxy, and SCH₃; and
Bx is a is a heterocyclic base moiety.

The chemical structure, name, and shorthand associated with various stereo-non-standard sugar moieties are shown in the table below.

TABLE 1
Stereo-non-standard Sugar Moieties

| Chemical | | | | |
|---|---|---|---|---|
| Structure | J_{odd} | Jₑᵥₑₙ | Sugar moiety name | Shorthand |
| V | H | H | 2′-α-D-deoxyribosyl | aDdr |
| VI | H | H | 2′-β-D-deoxyxylosyl | bDdx |
| VII | H | H | 2′-α-L-deoxyxylosyl | aLdx |
| VIII | H | H | 2′-β-L-deoxyribosyl | bLdr |
| IX | H | H | 2′-α-L-deoxyribosyl | aLdr |
| X | H | H | 2′-β-L-deoxyxylosyl | bLdx |
| XI | H | H | 2′-α-D-deoxyxylosyl | aDdx |
| V | H | OH | α-D-ribosyl | aDr |
| VI | H | OH | β-D-xylosyl | bDx |
| VII | H | OH | α-L-lyxosyl | aLl |
| VIII | H | OH | β-L-arabinosyl | bLa |
| IX | H | OH | α-L-arabinosyl | aLa |
| X | H | OH | β-L-lyxosyl | bLl |
| XI | H | OH | α-D-xylosyl | aDx |
| V | OH | H | α-D-arabinosyl | aDa |
| VI | OH | H | ß-D-lyxosyl | bDl |
| VII | OH | H | α-L-xylosyl | aLx |
| VIII | OH | H | β-L-ribosyl | bLr |
| IX | OH | H | α-L-ribosyl | aLr |
| X | OH | H | β-L-xylosyl | bLx |
| XI | OH | H | α-D-lyxosyl | aDl |
| V | H | F | 2′-fluoro-α-D-ribosyl | f2aDr |
| VI | H | F | 2′-fluoro-β-D-xylosyl | f2bDx |
| VII | H | F | 2′-fluoro-α-L-lyxosyl | f2aLl |
| VIII | H | F | 2′-fluoro-β-L-arabinosyl | f2bLa |
| IX | H | F | 2′-fluoro-α-L-arabinosyl | f2aLa |
| X | H | F | 2′-fluoro-β-L-lyxosyl | f2bLl |
| XI | H | F | 2′-fluoro-α-D-xylosyl | f2aDx |
| V | F | H | 2′-fluoro-α-D-arabinosyl | f2aDa |
| VI | F | H | 2′-fluoro-β-D-lyxosyl | f2bDl |
| VII | F | H | 2′-fluoro-α-L-xylosyl | f2aLx |
| VIII | F | H | 2′-fluoro-β-L-ribosyl | f2bLr |
| IX | F | H | 2′-fluoro-α-L-ribosyl | f2aLr |
| X | F | H | 2′-fluoro-β-L-xylosyl | f2bLx |
| XI | F | H | 2′-fluoro-α-D-lyxosyl | f2aDl |

Certain stereo-non-standard sugar moieties have been previously described in, e.g., Seth et al., WO2020/072991, Seth et al., WO2021/030763, and Seth et al., WO2019/157531, both of which are incorporated by reference herein in their entirety.

b. Substituted Stereo-Standard Sugar Moieties

In certain embodiments, modified sugar moieties are substituted stereo-standard furanosyl sugar moieties comprising one or more acyclic substituent, including but not limited to substituents at the 2′, 3′, 4′, and/or 5′ positions. In certain embodiments, the furanosyl sugar moiety is a ribosyl sugar moiety. In certain embodiments one or more acyclic substituent of substituted stereo-standard sugar moieties is branched. Examples of 2′-substituent groups suitable for substituted stereo-standard sugar moieties include but are not limited to: 2′-F, 2′-OCH₃ (“2′-OMe” or “2′-O-methyl”), and 2′-O(CH₂)₂OCH₃ (“2′-MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, C₁-C₁₀ alkyl, C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO2), thiol, thioalkoxy, thioalkyl, halogen, alkyl, aryl, alkenyl and alkynyl. Examples of 3′-substituent groups include 3′-methyl (see Frier, et al., The ups and downs of nucleic acid duplex stability: structure-stability studies on chemically-modified DNA:RNA duplexes. Nucleic Acids Res., 25, 4429-4443, 1997.) Examples of 4′-substituent groups suitable for substituted stereo-standard sugar moieties include but are not limited to alkoxy (e.g., methoxy), alkyl, and those described in Manoharan et al., WO 2015/106128. Examples of 5′-substituent groups suitable for substituted stereo-standard sugar moieties include but are not limited to: 5′-methyl (R or S), 5′-allyl, 5′-ethyl, 5′-vinyl, and 5′-methoxy. In certain embodiments, non-bicyclic modified sugars comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836. 2′,4′-difluoro modified sugar moieties have been described in Martinez-Montero, et al., Rigid 2′,4′-difluororibonucleosides: synthesis, conformational analysis, and incorporation into nascent RNA by HCV polymerase. J. Org. Chem., 2014, 79:5627-5635. Modified sugar moieties comprising a 2′-modification (OMe or F) and a 4′-modification (OMe or F) have also been described in Malek-Adamian, et al., J. Org. Chem, 2018, 83:9839-9849.

In certain embodiments, a 2′-substituted stereo-standard nucleoside comprises a sugar moiety comprising a non-bridging 2′-substituent group selected from: F, NH₂, N₃, OCF₃, OCH₃, SCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted stereo-standard nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted stereo-standard nucleoside comprises a sugar moiety comprising a 2′-substituent group selected from: F, OCH₃, and OCH₂CH₂OCH₃.

In certain embodiments, the 4′ O of 2′-deoxyribose can be substituted with a S to generate 4′-thio DNA (see Takahashi, et al., Nucleic Acids Research 2009, 37:1353-1362). This modification can be combined with other modifications detailed herein. In certain such embodiments, the sugar moiety is further modified at the 2′ position. In certain embodiments the sugar moiety comprises a 2′-fluoro. A thymidine with this sugar moiety has been described in Watts, et al., J. Org. Chem. 2006, 71 (3): 921-925 (4′-S-fluoro5-methylarauridine or FAMU).

c. Bicyclic Nucleosides

Certain nucleosides comprise modified sugar moieties that comprise a bridging sugar substituent that forms a second ring resulting in a bicyclic sugar moiety. In certain such embodiments, the bicyclic sugar moiety comprises a 4′ to 2′ bridge between the 4′ and the 2′ furanose ring atoms. In certain such embodiments, the furanose ring is a ribose ring. Examples of sugar moieties comprising such 4′ to 2′ bridging sugar substituents include but are not limited to bicyclic sugars comprising: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt” when in the S configuration), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672), 4′-C(═O)—N(CH₃)₂-2′, 4′-C(═O)—N(R)₂-2′, 4′-C(═S)—N(R)₂-2′ and analogs thereof (see, e.g., Obika et al., WO2011052436A1, Yusuke, WO2017018360A1).

Additional bicyclic sugar moieties are known in the art, see, for example: Freier et al., Nucleic Acids Research, 1997, 25 (22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Elayadi et al.; Christiansen, et al., J. Am. Chem. Soc. 1998, 120, 5458-5463; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

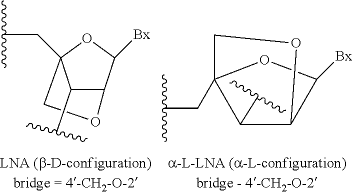

α-L-methyleneoxy (4′-CH₂—O-2′) or α-L-LNA bicyclic nucleosides have been incorporated into antisense oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

The term “substituted” following a position of the furanosyl ring, such as “2′-substituted” or “2′-4′-substituted”, indicates that is the only position(s) having a substituent other than those found in unmodified sugar moieties in oligonucleotides.

d. Sugar Surrogates

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), altritol nucleic acid (“ANA”), mannitol nucleic acid (“MNA”) (see, e.g., Leumann, CJ. Bioorg. & Med. Chem. 2002, 10, 841-854), fluoro HNA (“F-HNA” or “fluoro hexitol nucleic acid”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran. For F-HNA, the corresponding sugar surrogate can be referred to as “3′-fluoro-hexitol sugar surrogate” or “F-HNA sugar surrogate”; for ANA, the corresponding sugar moiety can be referred to as “altritol nucleic acid sugar surrogate” or “ANA sugar surrogate”, and for HNA, the corresponding sugar surrogate can be referred to as “hexitol nucleic acid sugar surrogate” or “HNA sugar surrogate”. In certain embodiments, sugar surrogates comprise rings having no heteroatoms. For example, nucleosides comprising bicyclo[3.1.0]-hexane have been described (see, e.g., Marquez, et al., J. Med. Chem. 1996, 39:3739-3749).

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate comprising the following structure:

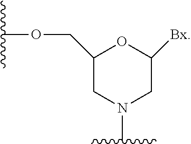

In certain embodiments, morpholinos may be modified, for example by adding or altering various substituent groups from the above morpholino structure. Such sugar surrogates are refered to herein as “modified morpholinos.” In certain embodiments, morpholino residues replace a full nucleotide, including the internucleoside linkage, and have the structures shown below, wherein Bx is a heterocyclic base moiety.

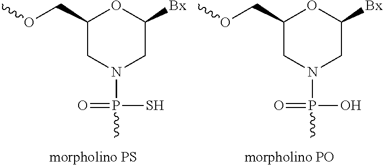

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), glycol nucleic acid (“GNA”, see Schlegel, et al., J. Am. Chem. Soc. 2017, 139:8537-8546) and nucleosides and oligonucleotides described in Manoharan et al., WO2011/133876. In certain embodiments, acyclic sugar surrogates are selected from:

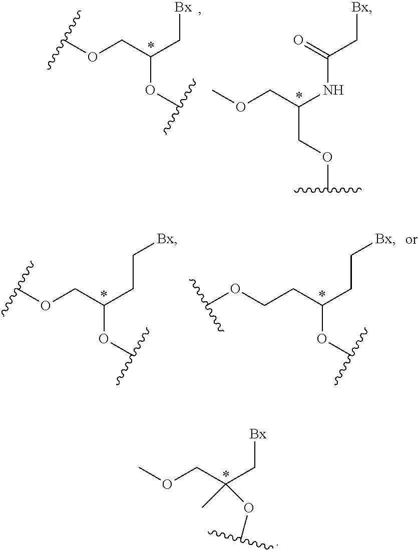

Many other bicyclic and tricyclic sugar and sugar surrogate ring systems are known in the art that can be used in modified nucleosides. Certain such ring systems are described in Hanessian, et al., J. Org. Chem., 2013, 78:9051-9063 and include bcDNA and tcDNA. Modifications to bcDNA and teDNA, such as 6′-fluoro, have also been described (Dogovic and Leumann, J. Org. Chem., 2014, 79:1271-1279).

e. Conjugated Nucleosides and Terminal Groups

In certain embodiments, modified sugar moieties comprise a conjugate group and/or a terminal group. Modified sugar moieties are linked to conjugate groups through a conjugate linker. In certain embodiments, modified furanosyl sugar moieties comprise conjugate groups attached at the 2′, 3′, or 5′ positions. In certain embodiments, the 3′-most sugar moiety of the nucleoside is modified with a conjugate group or a terminal group. In certain embodiments, the 5′-most sugar moiety of the nucleoside is modified with a conjugate group or a terminal group. In certain embodiments, a sugar moiety near the 3′ end of the nucleoside is modified with a conjugate group. In certain embodiments, a sugar moiety near the 5′ end of the nucleoside is modified with a conjugate group.

Examples of terminal groups include but are not limited to conjugate groups, capping groups, phosphate group, protecting groups, modified or unmodified nucleosides, and two or more nucleosides that are independently modified or unmodified.

In certain embodiments, terminal groups at the 5′-terminus comprise a stabilized phosphate group. In certain such embodiments, the phosphorus atom of the stabilized phosphate group is attached to the 5′-terminal nucleoside through a phosphorus-carbon bond. In certain embodiments, the carbon of that phosphorus-carbon bond is in turn bound to the 5′-position of the nucleoside.

In certain embodiments, the oligonucleotide comprises a 5′-stabilized phosphate group having the following formula:

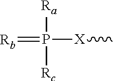

wherein:
   Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
   R_{b} is O or S;
   X is substituted or unsubstituted C; and wherein X is attached to the 5′-terminal nucleoside. In certain embodiments, X is bound to an atom at the 5′-position of the 5′-terminal nucleoside. In certain such embodiments, the 5′-atom is a carbon and the bond between X and the 5′-carbon of the 5′-terminal nucleoside is a carbon-carbon single bond. In certain embodiments, it is a carbon-carbon double bond. In certain embodiments, it is a carbon-carbon triple bond. In certain embodiments, the 5′-carbon is substituted. In certain embodiments, X is substituted. In certain embodiments, X is unsubstituted.

In certain embodiments, the oligonucleotide comprises a 5′-stabilized phosphate group having the following formula:

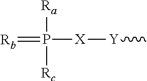

wherein:
   Rₐ and R_{c} are each, independently, OH, SH, C₁-C₆ alkyl, substituted C₁-C₆ alkyl, C₁-C₆ alkoxy, substituted C₁-C₆ alkoxy, amino or substituted amino;
   R_{b} is O or S;
   X is substituted or unsubstituted C;
   Y is selected from C, S, and N. In certain embodiments, Y is substituted or unsubstituted C. The bond between X and Y may be a single-, double-, or triple-bond.

Certain 5′-stabilized phosphate groups have been previously described; see, e.g., Prakash et al., WO2011/139699 and Prakash et al., WO2011/139702, hereby incorporated by reference herein in their entirety.

In certain embodiments, the stabilized phosphate group is 5′-vinyl phosphonate, 5′-methylene phosphonate or 5′-cyclopropyl phosphonate.

In certain embodiments, a terminal group at the 5′-terminus is a 5′-mesyl phosphoramidate, having formula XII:

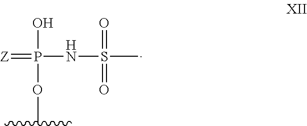

wherein Z is O or S.

In certain embodiments, a terminal group at the 5′-terminus is a 5′-mesyl phosphoramidate, having formula XIII:

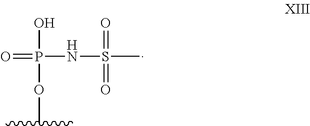 [SMILES: *OP(=O)(O)NS(C)(=O)=O]

### 2. Modified Nucleobases

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443. In certain embodiments, modified nucleosides comprise double-headed nucleosides having two nucleobases. Such compounds are described in detail in Sorinas et al., J. Org. Chem, 2014 79:8020-8030.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

In certain embodiments, compounds comprise or consist of a modified oligonucleotide complementary to a target nucleic acid comprising one or more modified nucleobases. In certain embodiments, the modified nucleobase is 5-methylcytosine. In certain embodiments, each cytosine is a 5-methylcytosine.

### B. Modified Internucleoside Linkages

a. Internucleoside Linkages of Formula I

In certain embodiments, antisense agents, oligomeric compounds, and modified oligonucleotides described herein having one or more modified internucleoside linkages having Formula I are selected over compounds lacking such internucleoside linkages having Formula I because of one or more desirable properties. In certain embodiments, antisense agents, oligomeric compounds, and modified oligonucleotides described herein having one or more modified internucleoside linkages having Formula I have enhanced cellular uptake. In certain embodiments, antisense agents, oligomeric compounds, and modified oligonucleotides described herein having one or more modified internucleoside linkages having Formula I have enhanced affinity for target nucleic acids. In certain embodiments, antisense agents, oligomeric compounds, and modified oligonucleotides described herein having one or more modified internucleoside linkages having Formula I have increased stability in the presence of nucleases. In certain embodiments, antisense agents, oligomeric compounds, and modified oligonucleotides described herein having one or more modified internucleoside linkages having Formula I have enhanced cellular uptake, enhanced affinity for target nucleic acids, and increased stability in the presence of nucleases. In certain embodiments, antisense agents, oligomeric compounds, and modified oligonucleotides described herein having one or more modified internucleoside linkages having Formula I have enhanced bioavailability. In certain embodiments, antisense agents, oligomeric compounds, and modified oligonucleotides described herein having one or more modified internucleoside linkages having Formula I have enhanced RNase H activity. In certain embodiments, antisense agents, oligomeric compounds, and modified oligonucleotides described herein having one or more modified internucleoside linkages having Formula I have enhanced RNAi activity. In certain embodiments, antisense agents, oligomeric compounds, and modified oligonucleotides described herein having one or more modified internucleoside linkages having Formula I have enhanced CRISPR activity. In certain embodiments, antisense agents, oligomeric compounds, and modified oligonucleotides described herein having one or more modified internucleoside linkages having Formula I have reduced interactions with certain proteins. In certain embodiments, antisense agents, oligomeric compounds, and modified oligonucleotides described herein having one or more modified internucleoside linkages having Formula I have increased interactions with certain proteins.

In certain embodiments, oligomeric compounds (including oligomeric compounds that are antisense agents or portions thereof) comprise or consist of a modified oligonucleotide complementary to a target nucleic acid comprising one or more modified internucleoside linkages having Formula I:

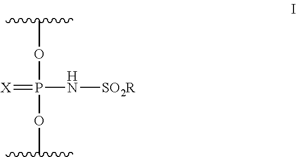

wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₁-C₆ alkenyl, C₁-C₆ alkynyl, substituted C₁-C₂₀ alkyl, substituted C₁-C₆ alkenyl substituted C₁-C₆ alkynyl, and a conjugate group.

### Other Internucleoside Linkages

In certain embodiments, antisense agents, oligomeric compounds, and modified oligonucleotides comprise or consist of a modified oligonucleotide complementary to a target nucleic acid comprising one or more modified internucleoside linkages. In certain embodiments, the modified internucleoside linkages are phosphorothioate linkages. In certain embodiments, each internucleoside linkage of an antisense compound is a phosphorothioate internucleoside linkage.

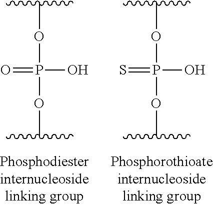 [SMILES: *OP(=O)(O)O* ; *OP(O)(=S)O*]

In certain embodiments, nucleosides of modified oligonucleotides may be linked together using any internucleoside linkage. The two main classes of internucleoside linkages are defined by the presence or absence of a phosphorus atom. Representative phosphorus-containing internucleoside linkages include unmodified phosphodiester internucleoside linkages, modified phosphotriesters such as THP phosphotriester and isopropyl phosphotriester, phosphonates such as methylphosphonate, isopropyl phosphonate, isobutyl phosphonate, and phosphonoacetate, phosphoramidates, phosphorothioate, and phosphorodithioate (“HS—P═S”). Representative non-phosphorus containing internucleoside linkages include but are not limited to methylenemethylimino (—CH₂—N(CH₃)—O—CH₂—), thiodiester, thionocarbamate (—O—C(═O)(NH)—S—); siloxane (—O—SiH₂—O—); formacetal, thioacetamido (TANA), alt-thioformacetal, glycine amide, and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. Methods of preparation of phosphorous-containing and non-phosphorous-containing internucleoside linkages are well known to those skilled in the art.

Neutral internucleoside linkages include, without limitation, phosphotriesters, phosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH2 component parts.

b. Chiral Internucleoside Linkages

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. All phosphorothioate linkages described herein are stereorandom unless otherwise specified. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

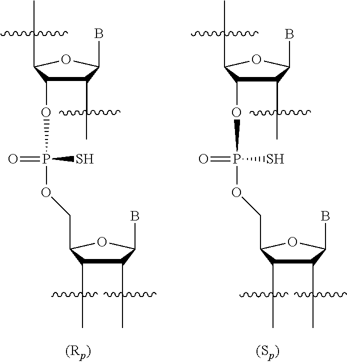

Unless otherwise indicated, chiral internucleoside linkages of modified oligonucleotides described herein can be stereorandom or in a particular stereochemical configuration.

In certain embodiments, an internucleoside linkage of Formula I may comprise a chiral center. In certain embodiments, modified oligonucleotides comprise chiral linkages of Formula II, as shown below.

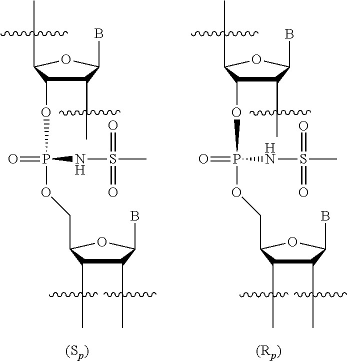

c. Alternatives to 5′ to 3′ Internucleoside Linkages

In certain embodiments, nucleic acids can be linked 2′ to 5′ rather than the standard 3′ to 5′ linkage. Such a linkage is illustrated below.

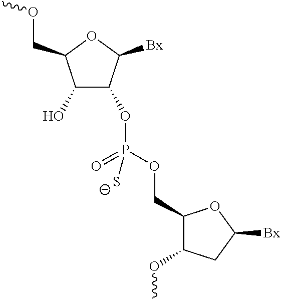

In certain embodiments, nucleosides can be linked by 2′, 3′-phosphodiester bonds. In certain such embodiments, the nucleosides are threofuranosyl nucleosides (TNA; see Bala, et al., J Org. Chem. 2017, 82:5910-5916). A TNA linkage is shown below.

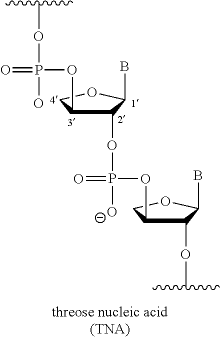

Additional modified linkages include α,β-D-CNA type linkages and related conformationally-constrained linkages, shown below. Synthesis of such molecules has been described previously (see Dupouy, et al., Angew. Chem. Int. Ed. Engl., 2014, 45:3623-3627; Borsting, et al. Tetrahedron, 2004, 60:10955-10966; Ostergaard, et al., ACS Chem. Biol. 2014, 9:1975-1979; Dupouy, et al., Eur. J. Org. Chem., 2008, 1285-1294; Martinez, et al., PLOS One, 2011, 6:e25510; Dupouy, et al., Eur. J. Org. Chem., 2007, 5256-5264; Boissonnet, et al., New J. Chem., 2011, 35: 1528˜1533.)

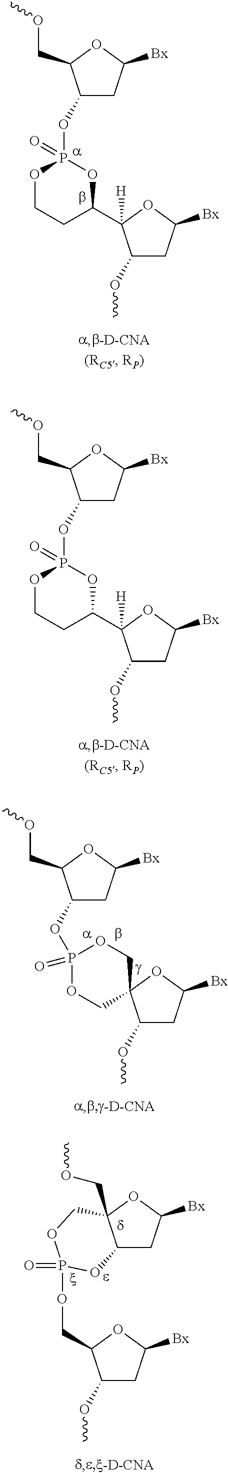

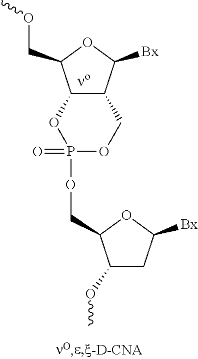

d. Linkages Having Conjugate Groups

In certain embodiments, an internucleoside linking group may comprise a conjugate group. In certain embodiments, an internucleoside linking group of Formula I comprises a conjugate group. In certain embodiments, the conjugate group of a modified oligonucleotide may be attached to the remainder of the modified oligonucleotide through a modified internucleoside having Formula I:

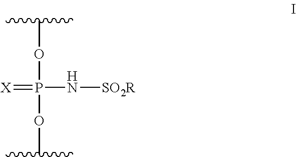

wherein R comprises a conjugate group. In certain embodiments, the conjugate group comprises a cell-targeting moiety. In certain embodiments, the conjugate group comprises a carbohydrate or carbohydrate cluster. In certain embodiments, the conjugate group comprises GalNAc. In certain embodiments, the conjugate group comprises a lipid. In certain embodiments, the conjugate group comprises C₁₀-C₂₀ alkyl. In certain embodiments, the conjugate group comprises C₁₆ alkyl.

In certain embodiments, the internucleoside linking group comprising a conjugate group has Formula IV:

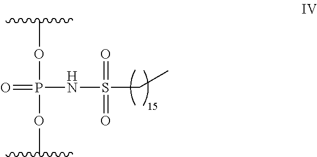

### II. Certain Motifs

In certain embodiments, antisense agents, oligomeric compounds, and modified oligonucleotides described herein comprise or consist of oligonucleotides. Modified oligonucleotides can be described by their motif, e.g. a pattern of unmodified and/or modified sugar moieties, nucleobases, and/or internucleoside linkages. In certain embodiments, modified oligonucleotides comprise one or more stereo-non-standard nucleosides. In certain embodiments, modified oligonucleotides comprise one or more stereo-standard nucleosides. In certain embodiments, modified oligonucleotides comprise one or more modified nucleoside comprising a modified sugar. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns or motifs of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### A. Certain Sugar Motifs

In certain embodiments, antisense agents, oligomeric compounds, and modified oligonucleotides described herein comprise or consist of oligonucleotides. In certain embodiments, oligonucleotides comprise one or more type of modified sugar and/or unmodified sugar moiety arranged along the oligonucleotide or region thereof in a defined pattern or sugar motif. In certain instances, such sugar motifs include without limitation any of the sugar modifications discussed herein.

In certain embodiments, each nucleoside of a modified oligonucleotide, or portion thereof, comprises a 2′-substituted sugar moiety, a bicyclic sugar moiety, a sugar surrogate, or a 2′-deoxyribosyl sugar moiety. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, PNA, THP, and F-HNA.

In certain embodiments, modified oligonucleotides comprise at least 12, at least 13, at least 14, at least 15, at least 16, at least 17, at least 18, at least 19, or at least 20 nucleosides comprising a modified sugar moiety. In certain embodiments, the modified sugar moiety is selected independently from a 2′-substituted sugar moiety, a bicyclic sugar moiety, or a sugar surrogate. In certain embodiments, the 2′-substituted sugar moiety is selected from a 2′-MOE sugar moiety, a 2′-NMA sugar moiety, a 2′-OMe sugar moiety, and a 2′-F sugar moiety. In certain embodiments, the bicyclic sugar moiety is selected from a cEt sugar moiety and an LNA sugar moiety. In certain embodiments, the sugar surrogate is selected from morpholino, modified morpholino, THP, and F-HNA.

In certain embodiments, modified oligonucleotides comprise at least 3 differently-modified nucleosides. In certain embodiments, the differently-modified nucleosides comprise sugar moieties selected from a 2′-O-methyl-β-D-ribosyl sugar moiety, a 2′-O-methoxyethyl β-D-ribosyl sugar moiety, a sugar surrogate, a stereo-non-standard sugar moiety, a 2′-β-D-deoxyribosyl sugar moiety, a β-D-ribosyl sugar moiety, and a 2′-fluoro-β-D-ribosyl sugar moiety. In certain embodiments, the sugar surrogate is selected from morpholino, modified morpholino, THP, HNA, and F-HNA. In certain embodiments, the sugar surrogate is F-HNA or HNA. In certain embodiments, the stereo-non-standard sugar moiety is selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.

In certain embodiments, the sense RNAi oligonucleotide consists of 21 linked nucleosides and has a sugar motif of yyyyyyxyxxxyyyyyyyyyy, from 5′ to 3′, wherein each “y” is a 2′-O-methyl-β-D-ribosyl sugar moiety and each “x” is independently selected from a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, wherein at least one “x” is other-than a 2′-fluoro-β-D-ribosyl sugar moiety. In certain embodiments, the sugar surrogate is selected from morpholino, modified morpholino, THP, HNA, and F-HNA. In certain embodiments, the sugar surrogate is F-HNA or HNA. In certain embodiments, the stereo-non-standard sugar moiety is selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.

In certain embodiments, the antisense RNAi oligonucleotide has a sugar motif of yxyyyxyyyyyyyxyxyyyyyyy or exyyyxyyyyyyyxyxyyyyyyy, from 5′ to 3′, wherein each “y” is a 2′-O-methyl-β-D-ribosyl sugar moiety, each “e” is a 2′-O-methoxyethyl-β-D-ribosyl sugar moiety, and each “x” is independently selected from a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, wherein at least one “x” is other-than a 2′-fluoro-β-D-ribosyl sugar moiety.

In certain embodiments, a sense RNAi oligonucleotide has any of the sugar motifs described in the table below.

TABLE 2a
Sense RNAi oligonucleotide sugar motifs

yyyyyyfyff[f2bDx]yyyyyyyyyy
yyyyyyfyf[f2bDx]fyyyyyyyyyy
yyyyyyfy[f2bDx]ffyyyyyyyyyy
yyyyyy[f2bDx]yfffyyyyyyyyyy
yyyyyy[f2bDx]y[f2bDx][f2bDx][f2bDx]yyyyyyyyyy
fyfyfyfyfyfyfyfyfyfyf
yyyyyyfyfffyyyyyyyyyy
yyyyy[16C2r]fyfffyyyyyyyyyy
yyyyy[C16A]fyfffyyyyyyyyyy
yyyyy[16C2r]fyfffyyyyyyyyyy
yyyyy[16C2r]fyff[f2bDx]yyyyyyyyyy
yyyyy[16C2r]fyf[f2bDx]fyyyyyyyyyy
yyyyy[16C2r]fy[f2bDx]ffyyyyyyyyyy
yyyyy[16C2r][f2bDx]yfffyyyyyyyyyy
yyyyy[16C2r][f2bDx]y[f2bDx][f2bDx][f2bDx]yyyyyyyyyy
yyyyyyfyfffyyyyyyyyyyddddddddd
yyyyy[16C2r]fyff[F-HNA]yyyyyyyyyy
yyyyy[16C2r]fyf[F-HNA]fyyyyyyyyyy
yyyyy[16C2r]fy[F-HNA]ffyyyyyyyyyy
yyyyy[16C2r][F-HNA]yfffyyyyyyyyyy
yyyyy[16C2r][F-HNA]yff[F-HNA]yyyyyyyyyy
yyyyy[16C2r][F-HNA]y[F-HNA][F-HNA][F-HNA]yyyyyyyyyy
yyyyyyyyyyyyyyyyyyyyy
yyyyyyyyfffyyyyyyyyyy
yyyyyyfyyffyyyyyyyyyy
yyyyyyfyfyfyyyyyyyyyy
yyyyyyfyffyyyyyyyyyyy
yyyyyyyyyffyyyyyyyyyy
yyyyyyfyyyfyyyyyyyyyy
yyyyyyfyfyyyyyyyyyyyy
yyyyyyyyfyfyyyyyyyyyy
yyyyyyyyffyyyyyyyyyyy
yyyyyyfyyfyyyyyyyyyyy
yyyyyyeyeeeyyyyyyyyyy
yyyyyyeyfffyyyyyyyyyy
yyyyyyfyeffyyyyyyyyyy
yyyyyyfyfefyyyyyyyyyy
yyyyyyfyffeyyyyyyyyyy
yyyyyyeyeffyyyyyyyyyy
yyyyyyfyeefyyyyyyyyyy
yyyyyyfyfeeyyyyyyyyyy
yyyyyyeyfefyyyyyyyyyy
yyyyyyeyffeyyyyyyyyyy
yyyyyyfyefeyyyyyyyyyy
yyyyyydyfffyyyyyyyyyy
yyyyyyfydffyyyyyyyyyy
yyyyyyfyfdfyyyyyyyyyy
yyyyyyfyffdyyyyyyyyyy
yyyyyydydffyyyyyyyyyy
yyyyyydyfdfyyyyyyyyyy
yyyyyydyffdyyyyyyyyyy
yyyyyydydfdyyyyyyyyyy
yyyyyyfydddyyyyyyyyyy
yyyyyydyddfyyyyyyyyyy
yyyyyydydddyyyyyyyyyy
yyyyydddddddddddyyyyy
yyyyyydddddddddyyyyyy
yyyyyyydddddddyyyyyyy
yyyyyyyydddddyyyyyyyy
eeeeedddddddddddeeeee
eeeeeedddddddddeeeeee
eeeeeeedddddddeeeeeee
eeeeeeeedddddeeeeeeee
yyyyyydydydydyddyyyyy
eeeeeedededededdeeeee
dydydydydydydydydydyd
dydydyfyfffydydydydyd
dedededededededededed
dededefefffededededed
ryryryryryryryryryryr
ryryryfyfffyryryryryr
rerererererererererer
rererefefffererererer
yyyyy[16C2r][f2bDx]yff[f2bDx]yyyyyyyyyy
yyyyyyryfffyyyyyyyyyy
yyyyyyfyrffyyyyyyyyyy
yyyyyyfyfrfyyyyyyyyyy
yyyyyyfyffryyyyyyyyyy
yyyyyyryrrryyyyyyyyyy
yyyyyyfyff[bDdx]yyyyyyyyyy
yyyyyyfyf[bDdx]fyyyyyyyyyy
yyyyyyfy[bDdx]ffyyyyyyyyyy
yyyyyy[bDdx]yfffyyyyyyyyyy
yyyyyy[bDdx]y[bDdx][bDdx][bDdx]yyyyyyyyyy
yyyyyyfyff[F-HNA]yyyyyyyyyy
yyyyyyfyf[F-HNA]fyyyyyyyyyy
yyyyyyfy[F-HNA]ffyyyyyyyyyy
yyyyyy[F-HNA]yfffyyyyyyyyyy
yyyyyy[F-HNA]y[F-HNA][F-HNA][F-HNA]yyyyyyyyyy
yyyyyyfyf[2bDx]fyyyyyyyyyy
yyyyyyfyf[bDx]fyyyyyyyyyy
yyyyyyfyf[ANA]fyyyyyyyyyy
[ANA]yyyyyfyf[ANA]fyyyyyy[ANA]yyy
[ANA]yy[ANA]yyfyf[ANA]fyyyyyyyyyy
yyy[ANA]y[ANA]fyf[ANA]fyyyyyyyyyy
yyyyyyfyf[ANA]f[ANA]yyyyy[ANA]yyy
[ANA]yyyy[ANA]fyf[ANA]f[ANA]yyyyyyyyy
[ANA]yyyy[ANA]fyf[ANA]fyyyy[ANA]yyyyy
[ANA]yyyy[ANA]fyf[ANA]f[ANA]yyy[ANA]yyyyy
yyyyyyfyf[HNA]fyyyyyyyyyy
[HNA]yyyyyfyf[HNA]fyyyyyy[HNA]yyy
[HNA]yy[HNA]yyfyf[HNA]fyyyyyyyyyy
yyy[HNA]y[HNA]fyf[HNA]fyyyyyyyyyy
yyyyyyfyf[HNA]f[HNA]yyyyy[HNA]yyy
[HNA]yyyy[HNA]fyf[HNA]fyyyy[HNA]yyyyy
[HNA]yyyy[HNA]fyf[HNA]f[HNA]yyyyyyyyy
[HNA]yyyy[HNA]fyf[HNA]f[HNA]yyy[HNA]yyyyy
yyyyyydydydydydyyyyyy
yyyyyy[bDa]yfffyyyyyyyyyy
yyyyyyfy[bDa]ffyyyyyyyyyy
yyyyyyfyf[bDa]fyyyyyyyyyy
yyyyyyfyff[bDa]yyyyyyyyyy
yyyyyy[bDa]y[bDa][bDa][bDa]yyyyyyyyyy
yyyyyyyyyyfyyyyyyyyyy
yyyyyyyyfyyyyyyyyyyyy
yyyyyyfyyyyyyyyyyyyyy
yyyyyyyydddyyyyyyyyyy

In the table above, “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[C16A]” represents 2′-O-hexylpalmitamide-β-D-ribosyl sugar moiety, “[16C2r]” represents a 2′-O-hexadecyl-β-D-ribosyl sugar moiety, “[F-HNA]” represents the sugar surrogate 3′-fluoro-tetrahydropyran, “[bDdx]” represents a 2′-β-D-deoxyxylosyl sugar moiety, “[bDx]” represents a 2′-β-D-xylosyl sugar moiety, “[bDa]” represents a 2′-β-D-arabinosyl sugar moiety, “[ANA]” represents an ANA sugar surrogate, “[HNA]” represents an HNA sugar surrogate.

In certain embodiments, an antisense RNAi oligonucleotide has any of the sugar motifs described in the table below.

TABLE 2b
Antisense RNAi oligonucleotide sugar motifs

yfyyyfyyyyyyyfyfyyyyyyy
y[f2bDx]yyyfyyyyyyyfyfyyyyyyy
yfyyy[f2bDx]yyyyyyyfyfyyyyyyy
yfyyyfyyyyyyy[f2bDx]yfyyyyyyy
yfyyyfyyyyyyyfy[f2bDx]yyyyyyy
y[f2bDx]yyy[f2bDx]yyyyyyy[f2bDx]y[f2bDx]yyyyyyy
yfyfyfyfyfyfyfyfyfyfyyy
efyfyfyfyfyfyfyfyfyfyyy
efyyyyyyyyyyyfyfyyyyyyy
efyyyfyyyyyyyfyfyyyyyyy
efyyyfy[16C2r]yyyyyfyfyyyyyyy
e[f2bDx]yyyfyyyyyyyfyfyyyyyyy
efyyy[f2bDx]yyyyyyyfyfyyyyyyy
efyyyfyyyyyyy[f2bDx]yfyyyyyyy
efyyyfyyyyyyyfy[f2bDx]yyyyyyy
e[f2bDx]yyy[f2bDx]yyyyyyy[f2bDx]y[f2bDx]yyyyyyy
efyyy[F-HNA]yyyyyyyfyfyyyyyyy
efyyyfyyyyyyy[F-HNA]yfyyyyyyy
efyyyfyyyyyyyfy[F-HNA]yyyyyyy
yfyyydyyyyyyyfyfyyyyyyy
yfyyyfyyyyyyydyfyyyyyyy
yfyyyfyyyyyyyfydyyyyyyy
yfyyydyyyyyyydydyyyyyyy
yryyyfyyyyyyyfyfyyyyyyy
yfyyyryyyyyyyfyfyyyyyyy
yfyyyfyyyyyyyryfyyyyyyy
yfyyyfyyyyyyyfyryyyyyyy
yryyyryyyyyyyryryyyyyyy
y[bDdx]yyyfyyyyyyyfyfyyyyyyy
yfyyy[bDdx]yyyyyyyfyfyyyyyyy
yfyyyfyyyyyyy[bDdx]yfyyyyyyy
yfyyyfyyyyyyyfy[bDdx]yyyyyyy
y[bDdx]yyy[bDdx]yyyyyyy[bDdx]y[bDdx]yyyyyyy
y[F-HNA]yyyfyyyyyyyfyfyyyyyyy
yfyyy[F-HNA]yyyyyyyfyfyyyyyyy
yfyyyfyyyyyyy[F-HNA]yfyyyyyyy
yfyyyfyyyyyyyfy[F-HNA]yyyyyyy
y[F-HNA]yyy[F-HNA]yyyyyyy[F-HNA]y[F-HNA]yyyyyyy
yfyyyfyyyyyyy[2bDx]yfyyyyyyy
y[bDa]yyyfyyyyyyyfyfyyyyyyy
yfyyy[bDa]yyyyyyyfyfyyyyyyy
yfyyyfyyyyyyy[bDa]yfyyyyyyy
yfyyyfyyyyyyyfy[bDa]yyyyyyy
y[bDa]yyy[bDa]yyyyyyy[bDa]y[bDa]yyyyyyy
yfyyyfyyyyyyy[bDx]yfyyyyyyy
yfyyyfyffyyyyfyfyyyyydd
yfyyyfyffyyyyfyfyyyyy[bLdr][bLdr]
yfyyyfyffyyyyfyfyyyyy[aLdr][aLdr]
yfyyyfyffyyyyfyfyyyyy[aDdr][aDdr]
yfyyyfyffyyyyfyfyyyyy[bDdx][bDdx]
yfyyyfyffyyyyfyfyyyyy[bLdx][bLdx]
yfyyyfyffyyyyfyfyyyyy[aDdx][aDdx]
yfyyyfyffyyyyfyfyyyyy[aLdx][aLdx]
yfyyyfyyyyyyyfyfyyyyyee
yfyyyfyyyyyyyfyfyyyyykk
yfyyyfyyyyyyyfyfyyyyy[LNA][LNA]
yfyyyfyyyyyyyfyfyyyyy[F-HNA][F-HNA]
[ANA]fyyyfyyyyyyyfyfyyyyyyy
y[ANA]yyyfyyyyyyyfyfyyyyyyy
yfyyyf[ANA]yyyyyyfyfyyyyyyy
yfyyyfyy[ANA]yyyyfyfyyyyyyy
yfyyyfyyyyyyy[ANA]yfyyyyyyy
yfyyyfyyyyyyyfyf[ANA]yyyyyy
yfyyyfyy[ANA]yyyy[ANA]yf[ANA]yyyyy[ANA]
yfyyyfyyyyyyyfyfyyyyyyk
yfyyyfyyyyyyyfyfyyyyyke
yfyyyfyyyyyyyfyfyyyyyky
efyyydyyyyyyydydyyyyyyy
yfyyyfyyyyyyy[ANA]yf[ANA]yyyyyy
yfyyyfyyyyyyyfyfyyyyy[ANA]y
yfyyyfyyyyyyyfyfyyyyy[f2bDa][f2bDa]
yfyyyfyyyyyyyfyfyyyyynn
yfyyyfyyyyyyyfyfyyyyy[DMAEOE][DMAEOE]
yfyyyfyyyyyyyfyfyyyyy[HNA][HNA]
yfyyyfyyyyyyyfyfyyyyy[SM5LNA][SM5LNA]
yfyyyfyyyyyyyfyfyyyyy[DMAOE][DMAOE]
yfyyyfyyyyyyyfyfyyyyydd
yfyyyfyyyyyyyfyfyyyyy[aLdr][aLdr]
[HNA]fyyyfyyyyyyyfyfyyyyyyy
dfyyyfyyyyyyyfyfyyyyyyy
y[HNA]yyyfyyyyyyyfyfyyyyyyy
e[HNA]yyyfyyyyyyyfyfyyyyyyy
yfyyyf[HNA]yyyyyyfyfyyyyyyy
yfyyyfyy[HNA]yyyyfyfyyyyyyy
yfyyyfyyyyyyy[HNA]yfyyyyyyy
yfyyyfyyyyyyyfyf[HNA]yyyyyy
yfyyyfyy[HNA]yyyy[HNA]yf[HNA]yyyyy[HNA]
yfyyyfyyyyyyy[HNA]yf[HNA]yyyyyy
yfyyyfyyyyyyyfyfyyyyy[HNA]y
kfyyyfyyyyyyyfyfyyyyyyy
e[F-HNA]yyyfyyyyyyyfyfyyyyyyy
e[LNA]yyyfyyyyyyyfyfyyyyyyy
edyyyfyyyyyyyfyfyyyyyyy
edyyydyyyyyyydydyyyyyyy
ydyyyfyyyyyyyfyfyyyyyyy
ydyyydyyyyyyydydyyyyyyy
efyyfyfyyyyfyfyyyyyyyyy
edyydydyyyydyfyyyyyyyyy
efyyyfyyyyyyyfyfyyyyydd
[F-HNA]fyyyfyyyyyyyfyfyyyyyyy
eyyyyfyyyyyyyfyfyyyyyyy
eryyyryyyyyyyryryyyyyyy
efyyydyyyyyyyfyfyyyyyyy
efyyyfyyyyyyyfydyyyyyyy
efyyyfyyyyyyydyfyyyyyyy

In the table above, “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[C16A]” represents 2′-O-hexylpalmitamide-β-D-ribosyl sugar moiety, “[16C2r]” represents a 2′-O-hexadecyl-β-D-ribosyl sugar moiety, “[F-HNA]” represents the sugar surrogate 3′-fluoro-tetrahydropyran, “[bDdx]” represents a 2′-β-D-deoxyxylosyl sugar moiety, “[bDx]” represents a 2′-β-D-xylosyl sugar moiety, “[bDa]” represents a β-D-arabinosyl sugar moiety, “[bLdr]” represents a 2′-β-L-deoxyribosyl sugar moiety, “[aDdr]” represents a 2′-α-D-deoxyribosyl sugar moiety, “[aLdr]” represents a 2′-α-L-deoxyribosyl sugar moiety, “[bLdx]” represents a 2′-β-L-deoxyxylosyl sugar moiety, a subscript “[aDdx]” represents a 2′-α-D-deoxyxylosyl sugar moiety, a subscript “[aLdx]” represents an 2′-α-L-deoxyxylosyl sugar moiety, “[LNA]” represents a β-D-LNA sugar moiety, “[f2bDa]” represents a 2′-fluoro-β-D-arabionsyl sugar moiety, “[DMAEOE]” represents a 2′-O-(2-(2-(N,N-dimethyl)aminoethoxy)ethyl)-β-D-ribosyl sugar moiety, “[SM5LNA]” represents a (5'S)-5′methyl-LNA sugar moiety, “[ANA]” represents an ANA sugar surrogate, and “[HNA]” represents an HNA sugar surrogate.

### B. Certain Nucleobase Motifs

In certain embodiments antisense agents, oligomeric compounds, and modified oligonucleotides described herein comprise or consist of oligonucleotides. In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, one nucleoside comprising a modified nucleobase is in the central region of a modified oligonucleotide. In certain such embodiments, the sugar moiety of said nucleoside is a 2′-β-D-deoxyribosyl moiety. In certain such embodiments, the modified nucleobase is selected from: 5-methyl cytosine, 2-thiopyrimidine, 2-thiothymine, 6-methyladenine, inosine, pseudouracil, or 5-propynepyrimidine.

### C. Certain Internucleoside Linkage Motifs

In certain embodiments, antisense agents, oligomeric compounds, and modified oligonucleotides described herein comprise or consist of oligonucleotides. In certain embodiments, oligonucleotides comprise modified and/or unmodified internucleoside linkages arranged along the oligonucleotide or region thereof in a defined pattern or motif.

In certain embodiments, the one or two 5′-most internucleoside linkages are internucleoside linkages of Formula I. In certain embodiments, the one or two 3′-most internucleoside linkages are internucleoside linkages of Formula I. In certain embodiments, each internucleoside linkage is selected from an internucleoside linkage of Formula I, a phosphorothioate internucleoside linkage, and a phosphodiester internucleoside linkage. In certain embodiments, each internucleoside linkage is selected from an internucleoside linkage of Formula I and a phosphodiester internucleoside linkage.

In certain embodiments, each phosphorothioate internucleoside linkage is independently selected from a stereorandom phosphorothioate, a (Sp) phosphorothioate, and a (Rp) phosphorothioate. In certain embodiments, the internucleoside linkages within the central region of a modified oligonucleotide are all modified. In certain such embodiments, all of the phosphorothioate linkages are stereorandom. In certain embodiments, all of the phosphorothioate linkages in the 5′-region and 3′-region are (Sp) phosphorothioates, and the central region comprises at least one Sp, Sp, Rp motif. In certain embodiments, populations of modified oligonucleotides are enriched for modified oligonucleotides comprising such internucleoside linkage motifs.

In certain embodiments, a double-stranded antisense agent is a double-stranded RNAi duplex comprising an antisense RNAi oligomeric compound and a sense RNAi oligomeric compound, wherein one or both of the RNAi antisense RNAi oligonucleotide and/or sense RNAi oligomeric compound have one or more modified internucleoside linking groups having Formula I. In certain embodiments, the RNAi antisense modified oligonucleotide comprises at least two, at least three, at least four, at least five, or at least six modified internucleoside linking groups having Formula I. In certain embodiments, the Sense RNAi oligonucleotide comprises at least two, at least three, at least four, at least five, or at least six modified internucleoside linking groups having Formula I.

In certain embodiments, the antisense RNAi oligonucleotide comprises exactly one modified internucleoside linking group having Formula I. In certain embodiments, the antisense RNAi oligonucleotide comprises exactly two modified internucleoside linking groups having Formula I. In certain embodiments, the antisense RNAi oligonucleotide comprises exactly three modified internucleoside linking groups having Formula I. In certain embodiments, the antisense RNAi oligonucleotide comprises exactly four modified internucleoside linking groups having Formula I.

In certain embodiments, the sense RNAi oligonucleotide comprises exactly one modified internucleoside linking group having Formula I. In certain embodiments, the sense RNAi oligonucleotide comprises exactly two modified internucleoside linking groups having Formula I. In certain embodiments, the sense RNAi oligonucleotide comprises exactly three modified internucleoside linking groups having Formula I. In certain embodiments, the sense RNAi oligonucleotide comprises exactly four modified internucleoside linking groups having Formula I. In certain embodiments, the sense RNAi oligonucleotide comprises exactly five modified internucleoside linking groups having Formula I.

In certain embodiments, at least one of the five 3′-most internucleoside linking groups of the antisense RNAi oligonucleotide is a modified internucleoside linking group having Formula I. In certain embodiments, at least two of the five 3′-most internucleoside linking groups of the antisense RNAi oligonucleotide are modified internucleoside linking groups having Formula I.

### D. Certain Modified Oligonucleotides

In certain embodiments, antisense agents, oligomeric compounds, and modified oligonucleotides described herein comprise or consist of modified oligonucleotides. In certain embodiments, the above modifications (sugar, nucleobase, internucleoside linkage) are incorporated into a modified oligonucleotide. In certain embodiments, modified oligonucleotides are characterized by their modifications, motifs, and overall lengths. In certain embodiments, such parameters are each independent of one another. Thus, unless otherwise indicated, each internucleoside linkage of a modified oligonucleotide may be modified or unmodified and may or may not follow the modification pattern of the sugar moieties. Likewise, such modified oligonucleotides may comprise one or more modified nucleobase independent of the pattern of the sugar modifications. Furthermore, in certain instances, a modified oligonucleotide is described by an overall length or range and by lengths or length ranges of two or more regions (e.g., a region of nucleosides having specified sugar modifications), in such circumstances it may be possible to select numbers for each range that result in an oligonucleotide having an overall length falling outside the specified range. In such circumstances, both elements must be satisfied. For example, in certain embodiments, a modified oligonucleotide consists of 15-20 linked nucleosides and has a sugar motif consisting of three regions or segments, A, B, and C, wherein region or segment A consists of 2-6 linked nucleosides having a specified sugar moiety, region or segment B consists of 6-10 linked nucleosides having a specified sugar moiety, and region or segment C consists of 2-6 linked nucleosides having a specified sugar moiety. Such embodiments do not include modified oligonucleotides where A and C each consist of 6 linked nucleosides and B consists of 10 linked nucleosides (even though those numbers of nucleosides are permitted within the requirements for A, B, and C) because the overall length of such oligonucleotide is 22, which exceeds the upper limit of 20 for the overall length of the modified oligonucleotide. Unless otherwise indicated, all modifications are independent of nucleobase sequence except that the modified nucleobase 5-methylcytosine is necessarily a “C” in an oligonucleotide sequence. In certain embodiments, when a DNA nucleoside or DNA-like nucleoside that comprises a T in a DNA sequence is replaced with a RNA-like nucleoside, the nucleobase T is replaced with the nucleobase U. Each of these compounds has an identical target RNA.

In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that XSY. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments oligonucleotides have a nucleobase sequence that is complementary to a second oligonucleotide or an identified reference nucleic acid, such as a target nucleic acid. In certain embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 70%, at least 80%, at least 90%, at least 95%, or 100% complementary to the second oligonucleotide or nucleic acid, such as a target nucleic acid.

### III. Certain Conjugated Compounds

In certain embodiments, antisense agents, oligomeric compounds, and modified oligonucleotides described herein comprise or consist of a modified oligonucleotide that optionally comprises a conjugate group. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate moieties or terminal groups are attached at the 3′ and/or 5′-end of oligonucleotides. In certain such embodiments, conjugate moieties (or terminal groups) are attached at the 3′-end of oligonucleotides. In certain embodiments, conjugate moieties are attached near the 3′-end of oligonucleotides. In certain embodiments, conjugate moieties (or terminal groups) are attached at the 5′-end of oligonucleotides. In certain embodiments, conjugate moieties are attached near the 5′-end of oligonucleotides.

In certain embodiments, at least one internucleoside linkage has formula I:

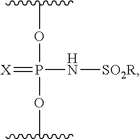

wherein R comprises a conjugate group. In certain embodiments, R is C₁₆.

### A. Certain Conjugate Groups and Conjugate Moieties

In certain embodiments, modified oligonucleotides comprise one or more conjugate moieties or conjugate groups. In certain embodiments, conjugate groups modify one or more properties of the molecule, including but not limited to pharmacodynamics, pharmacokinetics, stability, binding, absorption, tissue distribution, cellular distribution, cellular uptake, charge and clearance. In certain embodiments, conjugate moieties impart a new property on the molecule, e.g., fluorophores or reporter groups that enable detection of the molecule.

Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic, a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, i, 923-937), a tocopherol group (Nishina et al., Molecular Therapy Nucleic Acids, 2015, 4, e220; doi: 10.1038/mtna.2014.72 and Nishina et al., Molecular Therapy, 2008, 16, 734-740), or a GalNAc cluster (e.g., WO2014/179620).

a. Conjugate Moieties

Conjugate moieties include, without limitation, intercalators, reporter molecules, polyamines, polyamides, peptides, carbohydrates (e.g., GalNAc), vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

b. Conjugate Linkers

In certain embodiments, conjugate groups comprise a conjugate linker that attaches a conjugate moiety to the remainder of the modified oligonucleotide. In certain embodiments, a conjugate linker is a single chemical bond (i.e. conjugate moiety is attached to the remainder of the modified oligonucleotide via a conjugate linker through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to oligomeric compounds, such as the oligonucleotides provided herein. In general, a bifunctional linking moiety comprises at least two functional groups. One of the functional groups is selected to bind to a particular site on an oligomeric compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group or conjugate moiety to be cleaved from the remainder of the oligonucleotide. For example, in certain circumstances oligomeric compounds (including oligomeric compounds that are antisense agents or portions thereof) or modified oligonucleotides comprising a particular conjugate moiety are better taken up by a particular cell type, but once the compound has been taken up, it is desirable that the conjugate group be cleaved to release an unconjugated oligonucleotide. Thus, certain conjugate moieties may comprise one or more cleavable moieties, typically within the conjugate linker. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a nucleoside comprising a 2′-deoxyfuranosyl that is attached to either the 3′ or 5′-terminal nucleoside of an oligonucleotide by a phosphodiester internucleoside linkage and covalently attached to the remainder of the conjugate linker or conjugate moiety by a phosphodiester or phosphorothioate linkage. In certain such embodiments, the cleavable moiety is a nucleoside comprising a 2′-β-D-deoxyribosyl sugar moiety. In certain such embodiments, the cleavable moiety is 2′-deoxyadenosine.

c. Certain Cell-Targeting Conjugate Moieties

In certain embodiments, a conjugate group comprises a cell-targeting conjugate moiety. In certain embodiments, a conjugate group has the general formula:

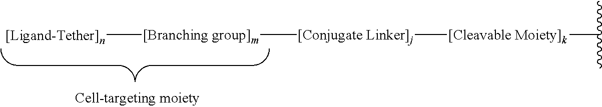

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, the cell-targeting moiety comprises a branching group comprising one or more groups selected from alkyl, amino, oxo, amide, disulfide, polyethylene glycol, ether, thioether and hydroxylamino groups. In certain embodiments, the branching group comprises a branched aliphatic group comprising groups selected from alkyl, amino, oxo, amide, disulfide, polyethylene glycol, ether, thioether and hydroxylamino groups. In certain such embodiments, the branched aliphatic group comprises groups selected from alkyl, amino, oxo, amide and ether groups. In certain such embodiments, the branched aliphatic group comprises groups selected from alkyl, amino and ether groups. In certain such embodiments, the branched aliphatic group comprises groups selected from alkyl and ether groups. In certain embodiments, the branching group comprises a mono or polycyclic ring system.

In certain embodiments, each tether of a cell-targeting moiety comprises one or more groups selected from alkyl, substituted alkyl, ether, thioether, disulfide, amino, oxo, amide, phosphodiester, and polyethylene glycol, in any combination. In certain embodiments, each tether is a linear aliphatic group comprising one or more groups selected from alkyl, ether, thioether, disulfide, amino, oxo, amide, and polyethylene glycol, in any combination. In certain embodiments, each tether is a linear aliphatic group comprising one or more groups selected from alkyl, phosphodiester, ether, amino, oxo, and amide, in any combination. In certain embodiments, each tether is a linear aliphatic group comprising one or more groups selected from alkyl, ether, amino, oxo, and amid, in any combination. In certain embodiments, each tether is a linear aliphatic group comprising one or more groups selected from alkyl, amino, and oxo, in any combination. In certain embodiments, each tether is a linear aliphatic group comprising one or more groups selected from alkyl and oxo, in any combination. In certain embodiments, each tether is a linear aliphatic group comprising one or more groups selected from alkyl and phosphodiester, in any combination. In certain embodiments, each tether comprises at least one phosphorus linking group or neutral linking group. In certain embodiments, each tether comprises a chain from about 6 to about 20 atoms in length. In certain embodiments, each tether comprises a chain from about 10 to about 18 atoms in length. In certain embodiments, each tether comprises about 10 atoms in chain length.

In certain embodiments, each ligand of a cell-targeting moiety has an affinity for at least one type of receptor on a target cell. In certain embodiments, each ligand has an affinity for at least one type of receptor on the surface of a mammalian lung cell.

In certain embodiments, each ligand of a cell-targeting moiety is a carbohydrate, carbohydrate derivative, modified carbohydrate, polysaccharide, modified polysaccharide, or polysaccharide derivative. In certain such embodiments, the conjugate group comprises a carbohydrate cluster (see, e.g., Maier et al., “Synthesis of Antisense Oligonucleotides Conjugated to a Multivalent Carbohydrate Cluster for Cellular Targeting,” Bioconjugate Chemistry, 2003, 14, 18-29, or Rensen et al., “Design and Synthesis of Novel N-Acetylgalactosamine-Terminated Glycolipids for Targeting of Lipoproteins to the Hepatic Asiaglycoprotein Receptor,” J. Med. Chem. 2004, 47, 5798-5808, which are incorporated herein by reference in their entirety). In certain such embodiments, each ligand is an amino sugar or a thio sugar. For example, amino sugars may be selected from any number of compounds known in the art, such as sialic acid, α-D-galactosamine, β-muramic acid, 2-deoxy-2-methylamino-L-glucopyranose, 4,6-dideoxy-4-formamido-2,3-di-O-methyl-D-mannopyranose, 2-deoxy-2-sulfoamino-D-glucopyranose and N-sulfo-D-glucosamine, and N-glycolyl-α-neuraminic acid. For example, thio sugars may be selected from 5-Thio-β-D-glucopyranose, methyl 2,3,4-tri-O-acetyl-1-thio-6-O-trityl-α-D-glucopyranoside, 4-thio-β-D-galactopyranose, and ethyl 3,4,6,7-tetra-O-acetyl-2-deoxy-1,5-dithio-α-D-gluco-heptopyranoside.

In certain embodiments, oligomeric compounds (including oligomeric compounds that are antisense agents or portions thereof) or modified oligonucleotides described herein comprise a conjugate group found in any of the following references: Lee, Carbohydr Res, 1978, 67, 509-514; Connolly et al., J Biol Chem, 1982, 257, 939-945; Pavia et al., Int J Pep Protein Res, 1983, 22, 539-548; Lee et al., Biochem, 1984, 23, 4255-4261; Lee et al., Glycoconjugate J, 1987, 4, 317-328; Toyokuni et al., Tetrahedron Lett, 1990, 31, 2673-2676; Biessen et al., J Med Chem, 1995, 38, 1538-1546; Valentijn et al., Tetrahedron, 1997, 53, 759-770; Kim et al., Tetrahedron Lett, 1997, 38, 3487-3490; Lee et al., Bioconjug Chem, 1997, 8, 762-765; Kato et al., Glycobiol, 2001, 11, 821-829; Rensen et al., J Biol Chem, 2001, 276, 37577-37584; Lee et al., Methods Enzymol, 2003, 362, 38-43; Westerlind et al., Glycoconj J, 2004, 21, 227-241; Lee et al., Bioorg Med Chem Lett, 2006, 16 (19), 5132-5135; Maierhofer et al., Bioorg Med Chem, 2007, 15, 7661-7676; Khorev et al., Bioorg Med Chem, 2008, 16, 5216-5231; Lee et al., Bioorg Med Chem, 2011, 19, 2494-2500; Kornilova et al., Analyt Biochem, 2012, 425, 43-46; Pujol et al., Angew Chemie Int Ed Engl, 2012, 51, 7445-7448; Biessen et al., J Med Chem, 1995, 38, 1846-1852; Sliedregt et al., J Med Chem, 1999, 42, 609-618; Rensen et al., J Med Chem, 2004, 47, 5798-5808; Rensen et al., Arterioscler Thromb Vasc Biol, 2006, 26, 169-175; van Rossenberg et al., Gene Ther, 2004, 11, 457-464; Sato et al., J Am Chem Soc, 2004, 126, 14013-14022; Lee et al., J Org Chem, 2012, 77, 7564-7571; Biessen et al., FASEB J, 2000, 14, 1784-1792; Rajur et al., Bioconjug Chem, 1997, 8, 935-940; Duff et al., Methods Enzymol, 2000, 313, 297-321; Maier et al., Bioconjug Chem, 2003, 14, 18-29; Jayaprakash et al., Org Lett, 2010, 12, 5410-5413; Manoharan, Antisense Nucleic Acid Drug Dev, 2002, 12, 103-128; Merwin et al., Bioconjug Chem, 1994, 5, 612-620; Tomiya et al., Bioorg Med Chem, 2013, 21, 5275-5281; International applications WO1998/013381; WO2011/038356; WO1997/046098; WO2008/098788; WO2004/101619; WO2012/037254; WO2011/120053; WO2011/100131; WO2011/163121; WO2012/177947; WO2013/033230; WO2013/075035; WO2012/083185; WO2012/083046; WO2009/082607; WO2009/134487; WO2010/144740; WO2010/148013; WO1997/020563; WO2010/088537; WO2002/043771; WO2010/129709; WO2012/068187; WO2009/126933; WO2004/024757; WO2010/054406; WO2012/089352; WO2012/089602; WO2013/166121; WO2013/165816; U.S. Pat. Nos. 4,751,219; 8,552,163; 6,908,903; 7,262,177; 5,994,517; 6,300,319; 8,106,022; 7,491,805; 7,491,805; 7,582,744; 8,137,695; 6,383,812; 6,525,031; 6,660,720; 7,723,509; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; Published U.S. Patent Application Publications US2011/0097264; US2011/0097265; US2013/0004427; US2005/0164235; US2006/0148740; US2008/0281044; US2010/0240730; US2003/0119724; US2006/0183886; US2008/0206869; US2011/0269814; US2009/0286973; US2011/0207799; US2012/0136042; US2012/0165393; US2008/0281041; US2009/0203135; US2012/0035115; US2012/0095075; US2012/0101148; US2012/0128760; US2012/0157509; US2012/0230938; US2013/0109817; US2013/0121954; US2013/0178512; US2013/0236968; US2011/0123520; US2003/0077829; US2008/0108801; and US2009/0203132.

### Compositions and Methods for Formulating Pharmaceutical Compositions

Antisense agents, oligomeric compounds, and modified oligonucleotides described herein may be admixed with pharmaceutically acceptable active or inert substances for the preparation of pharmaceutical compositions. Compositions and methods for the formulation of pharmaceutical compositions are dependent upon a number of criteria, including, but not limited to, route of administration, extent of disease, or dose to be administered.

Certain embodiments provide pharmaceutical compositions comprising one or more oligomeric compounds (including oligomeric compounds that are antisense agents or portions thereof) or a salt thereof. In certain such embodiments, the pharmaceutical composition comprises a suitable pharmaceutically acceptable diluent or carrier. In certain embodiments, a pharmaceutical composition comprises a sterile saline solution and one or more oligomeric compound. In certain embodiments, such pharmaceutical composition consists of a sterile saline solution and one or more oligomeric compound. In certain embodiments, the sterile saline is pharmaceutical grade saline. In certain embodiments, a pharmaceutical composition comprises one or more oligomeric compound and sterile water. In certain embodiments, a pharmaceutical composition consists of one oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, a pharmaceutical composition consists of one or more oligomeric compound and sterile PBS. In certain embodiments, the sterile PBS is pharmaceutical grade PBS. Compositions and methods for the formulation of pharmaceutical compositions are dependent upon a number of criteria, including, but not limited to, route of administration, extent of disease, or dose to be administered.

An oligomeric compound described herein complementary to a target nucleic acid can be utilized in pharmaceutical compositions by combining the oligomeric compound with a suitable pharmaceutically acceptable diluent or carrier and/or additional components such that the pharmaceutical composition is suitable for injection. In certain embodiments, a pharmaceutically acceptable diluent is phosphate buffered saline. Accordingly, in one embodiment, employed in the methods described herein is a pharmaceutical composition comprising an oligomeric compound complementary to a target nucleic acid and a pharmaceutically acceptable diluent. In certain embodiments, the pharmaceutically acceptable diluent is phosphate buffered saline. In certain embodiments, the oligomeric compound comprises or consists of a modified oligonucleotide provided herein.

Pharmaceutical compositions comprising oligomeric compounds (including oligomeric compounds that are antisense agents or portions thereof) provided herein encompass any pharmaceutically acceptable salts, esters, or salts of such esters, or any other oligonucleotide which, upon administration to an animal, including a human, is capable of providing (directly or indirectly) the biologically active metabolite or residue thereof. In certain embodiments, the oligomeric compound comprises or consists of a modified oligonucleotide. Accordingly, for example, the disclosure is also drawn to pharmaceutically acceptable salts of compounds, prodrugs, pharmaceutically acceptable salts of such prodrugs, and other bioequivalents. Suitable pharmaceutically acceptable salts include, but are not limited to, sodium and potassium salts.

### Certain Mechanisms

In certain embodiments, oligomeric compounds (including oligomeric compounds that are antisense agents or portions thereof) described herein comprise or consist of modified oligonucleotides. In certain such embodiments, the oligomeric compounds described herein are capable of hybridizing to a target nucleic acid, resulting in at least one antisense activity. In certain embodiments, compounds described herein selectively affect one or more target nucleic acid. Such compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in a significant undesired antisense activity.

In certain antisense activities, hybridization of a compound described herein to a target nucleic acid results in recruitment of a protein that cleaves the target nucleic acid. For example, certain compounds described herein result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, compounds described herein are sufficiently “DNA-like” to elicit RNase H activity. Nucleosides that are sufficiently “DNA-like” to elicit RNase H activity are referred to as DNA mimics herein. Further, in certain embodiments, one or more non-DNA-like nucleoside in in the RNA:DNA duplex is tolerated.

In certain antisense activities, hybridization of an antisense agent, oligomeric compound, or modified oligonucleotide described herein to a target nucleic acid results in modulation of the splicing of a target pre-mRNA. For example, in certain embodiments, hybridization of a compound described herein will increase exclusion of an exon. For example, in certain embodiments, hybridization of a compound described herein will increase inclusion of an exon.

In certain antisense activities, antisense agents described herein or a portion of the antisense agent is loaded into an RNA-induced silencing complex (RISC), ultimately resulting in cleavage of the target nucleic acid. For example, certain compounds described herein result in cleavage of the target nucleic acid by Argonaute. Compounds that are loaded into RISC are RNAi agents. RNAi agents may be double-stranded (siRNA) or single-stranded (ssRNA).

In certain antisense activities, antisense agents, oligomeric compounds, or modified oligonucleotides described herein result in a CRISPR system cleaving a target DNA. In certain antisense activities, compounds described herein result in a CRISPR system editing a target DNA.

In certain antisense activities, hybridization of an antisense agent, oligomeric compound, or modified oligonucleotide described herein to a target nucleic acid results in disruption of secondary structural elements, such as stem-loops and hairpins. For example, in certain embodiments, hybridization of a compound described herein to a stem-loop that is part of a translation suppression element leads to an increase in protein expression.

In certain antisense activities, hybridization of an antisense agent, oligomeric compound, or modified oligonucleotide described herein to a target nucleic acid leads to no-go decay mediated mRNA degradation.

In certain antisense activities, hybridization of an antisense agent, oligomeric compound, or modified oligonucleotide described herein to a target nucleic acid leads to activation of nonsense-mediated decay mRNA degradation.

In certain embodiments, antisense agents, oligomeric compounds, or modified oligonucleotides described herein are artificial mRNA compounds, the nucleobase sequence of which encodes for a protein.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein, and/or a phenotypic change in a cell or animal.

### Certain RNAi Agents

In certain embodiments, oligomeric compounds described herein having one or more internucleoside linkages of Formula I are RNAi agents. In certain embodiments, internucleoside linkages having Formula I can replace one or more phosphorothioate or phosphodiester internucleoside linkages in any RNAi motif. Certain RNAi motifs are described in, e.g., Freier, et al., WO2020/160163, incorporated by reference herein in its entirety; as well as, e.g., Rajeev, et al., WO2013/075035; Maier, et al., WO2016/028649; Theile, et al., WO2018/098328; Nair, et al., WO2019/217459; each of which is incorporated by reference herein.

### Target Nucleic Acids, Target Regions and Nucleotide Sequences

In certain embodiments, antisense agents, oligomeric compounds, or modified oligonucleotides described herein comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: an mRNA and a pre-mRNA, including intronic, exonic and untranslated regions. In certain embodiments, the target RNA is an mRNA. In certain embodiments, the target nucleic acid is a pre-mRNA. In certain embodiments, a pre-mRNA and corresponding mRNA are both target nucleic acids of a single compound. In certain such embodiments, the target region is entirely within an intron of a target pre-mRNA. In certain embodiments, the target region spans an intron/exon junction. In certain embodiments, the target region is at least 50% within an intron. In certain embodiments, the target nucleic acid is a microRNA. In certain embodiments, the target region is in the 5′ UTR of a gene. In certain embodiments, the target region is within a translation suppression element region of a target nucleic acid.

### Certain Compounds

Certain compounds described herein (e.g., antisense agents, oligomeric compounds, and modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms. All tautomeric forms of the compounds provided herein are included unless otherwise indicated.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples are intended to illustrate certain aspects of the invention and are not intended to limit the invention in any way.

### Example 1: Design of siRNA to HPRT1 Having Chiral Mesyl Phosphoramidate Internucleoside Linkages

Double-stranded siRNA comprising modified oligonucleotides having mesyl phosphoramidate internucleoside linkages (Formula II) in the antisense RNAi oligonucleotides were synthesized using standard techniques. Each internucleoside linkage is either a phosphorothioate internucleoside linkage (“s”), a phosphodiester internucleoside linkage (“o”), or a mesyl phosphoramidate internucleoside linkage (“z”), indicated by Formula II below.

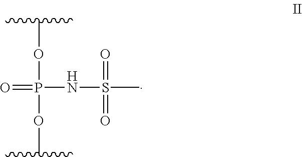 [SMILES: *OP(=O)(NS(C)(=O)=O)O*]

A subscript “[zS]” indicates a mesyl phosphoramidate linkage in a chiral(S) configuration as shown below:

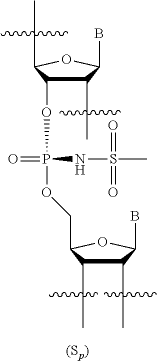

A subscript “[zR]” indicates a mesyl phosphoramidate linkage in a chiral (R) configuration as shown below:

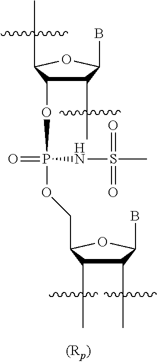

Each antisense RNAi oligonucleotide described in the table below has the sequence AUAAAAUCUACAGUCAUAGGAAU (SEQ ID NO: 3) and is 100% complementary to GenBank Accession No. NM_000194.2 (SEQ ID NO: 1) from nucleosides 444 to 466. Each antisense RNAi oligonucleotide has a 5′-phosphate.

TABLE 3
Design of antisense RNAi oligonucleotides
targeted to human/mouse HPRT1 containing
chiral mesyl phosphoramidate linkages

| | | SEQ |
|---|---|---|
| Compound | Chemistry Notation | ID |
| No. | (5′ to 3′) | NO. |
| 1337111 | p.A_{ys}U_{fs}A_{yo}A_{fo}A_{yo}A_{fo}U_{yo} | 3 |
| | C_{fo}U_{yo}A_{fo}C_{yo}A_{fo}G_{yo}U_{fo}C_{yo} | |
| | A_{fo}U_{yo}A_{fo}G_{yo}G_{fo}A_{ys}A_{fs}U_{y} | |
| 1465680 | p.AyzU_{fs}A_{yo}A_{fo}A_{yo}A_{fo}U_{yo} | 3 |
| | C_{fo}U_{yo}A_{fo}C_{yo}A_{fo}G_{yo}U_{fo}C_{yo} | |
| | A_{fo}U_{yo}A_{fo}G_{yo}G_{fo}A_{ys}A_{fs}U_{y} | |
| 1465681 | p.Ay_{[}zS_{]}U_{fs}A_{yo}A_{fo}A_{yo}A_{fo} | 3 |
| | U_{yo}C_{fo}U_{yo}A_{fo}C_{yo}A_{fo}G_{yo}U_{fo} | |
| | C_{yo}A_{fo}U_{yo}A_{fo}G_{yo}G_{fo}A_{ys}A_{fs} | |
| | U_{y} | |
| 1590251 | p.Ay_{[}zR_{]}U_{fs}A_{yo}A_{fo}A_{yo}A_{fo} | 3 |
| | U_{yo}C_{fo}U_{yo}A_{fo}C_{yo}A_{fo}G_{yo}U_{fo} | |
| | C_{yo}A_{fo}U_{yo}A_{fo}G_{yo}G_{fo}A_{ys}A_{fs} | |
| | U_{y} | |
| 1590252 | p.A_{ys}UfzA_{yo}A_{fo}A_{yo}A_{fo}U_{yo} | 3 |
| | C_{fo}U_{yo}A_{fo}C_{yo}A_{fo}G_{yo}U_{fo}C_{yo} | |
| | A_{fo}U_{yo}A_{fo}G_{yo}G_{fo}A_{ys}A_{fs}U_{y} | |
| 1590253 | p.A_{ys}Uf_{[}zS_{]}A_{yo}A_{fo}A_{yo}A_{fo} | 3 |
| | U_{yo}C_{fo}U_{yo}A_{fo}C_{yo}A_{fo}G_{yo}U_{fo} | |
| | C_{yo}A_{fo}U_{yo}A_{fo}G_{yo}G_{fo}A_{ys}A_{fs} | |
| | U_{y} | |
| 1590254 | p.A_{ys}Uf_{[}zR_{]}A_{yo}A_{fo}A_{yo}A_{fo} | 3 |
| | U_{yo}C_{fo}U_{yo}A_{fo}C_{yo}A_{fo}G_{yo}U_{fo} | |
| | C_{yo}A_{fo}U_{yo}A_{fo}G_{yo}G_{fo}A_{ys}A_{fs} | |
| | U_{y} | |
| 1590255 | p.AyzUfzA_{yo}A_{fo}A_{yo}A_{fo}U_{yo} | 3 |
| | C_{fo}U_{yo}A_{fo}C_{yo}A_{fo}G_{yo}U_{fo}C_{yo} | |
| | A_{fo}U_{yo}A_{fo}G_{yo}G_{fo}A_{ys}A_{fs}U_{y} | |
| 1590256 | p.Ay_{[}zS_{]}Uf_{[}zS_{]}A_{yo}A_{fo}A_{yo} | 3 |
| | A_{fo}U_{yo}C_{fo}U_{yo}A_{fo}C_{yo}A_{fo}G_{yo} | |
| | U_{fo}C_{yo}A_{fo}U_{yo}A_{fo}G_{yo}G_{fo}A_{ys} | |
| | A_{fs}U_{y} | |
| 1590257 | p.Ay_{[}zR_{]}Uf_{[}zS_{]}A_{yo}A_{fo}A_{yo} | 3 |
| | A_{fo}U_{yo}C_{fo}U_{yo}A_{fo}C_{yo}A_{fo}G_{yo} | |
| | U_{fo}C_{yo}A_{fo}U_{yo}A_{fo}G_{yo}G_{fo}A_{ys} | |
| | A_{fs}U_{y} | |
| 1590258 | p.Ay_{[}zS_{]}Uf_{[}zR_{]}A_{yo}A_{fo}A_{yo} | 3 |
| | A_{fo}U_{yo}C_{fo}U_{yo}A_{fo}C_{yo}A_{fo}G_{yo} | |
| | U_{fo}C_{yo}A_{fo}U_{yo}A_{fo}G_{yo}G_{fo}A_{ys} | |
| | A_{fs}U_{y} | |
| 1590259 | p.Ay_{[}zR_{]}Uf_{[}zR_{]}A_{yo}A_{fo}A_{yo} | 3 |
| | A_{fo}U_{yo}C_{fo}U_{yo}A_{fo}C_{yo}A_{fo}G_{yo} | |
| | U_{fo}C_{yo}A_{fo}U_{yo}A_{fo}G_{yo}G_{fo}A_{ys} | |
| | A_{fs}U_{y} | |

In the table above, a “p.” represents a 5′-phosphate, a subscript “f” represents a 2′-fluoro-β-D-ribosyl sugar moiety, a subscript “y” represents a 2′-OMe-β-D-ribosyl sugar moiety, a subscript “s” indicates a phosphorothioate internucleoside linkage, a subscript “o” represents a phosphodiester internucleoside linkage, a subscript “z” represents a mesyl phosphoramidate internucleoside linkage, a subscript “[zS]” represents a mesyl phosphoramidate linkage in chiral(S) configuration, and a subscript “[zR]” represents a mesyl phosphoramidate linkage in chiral (R) configuration. Subscripts of nucleotides having a phosphoramidate internucleoside linkage of generic Formula II are bold and underlined.

The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Compound No. 1337113 further comprises a 3′-linked C7 amino modifier (Glen Research), shown below:

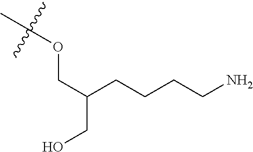 [SMILES: *OCC(CO)CCCCN]

TABLE 4
Design of sense RNAi oligonucleotides targeted
to human/mouse HPRT1 containing chiral mesyl
phosphoramidate linkages

| | | SEQ |
|---|---|---|
| Compound | Chemistry Notation | ID |
| No. | (5′ to 3′) | NO. |
| 1337113 | U_{fs}C_{ys}C_{fo}U_{yo}A_{fo}U_{yo}G_{fo}A_{yo} | 4 |
| | C_{fo}U_{yo}G_{fo}U_{yo}A_{fo}G_{yo}A_{fo}U_{yo} | |
| | U_{fo}U_{yo}U_{fo}A_{yo}U_{fo}- | |
| | [3′-amino C7 tag] | |

A subscript “f” represents a 2′-fluoro-β-D-ribosyl sugar moiety, a subscript “y” represents a 2′-OMe-β-D-ribosyl sugar moiety, a subscript “s” indicates a phosphorothioate internucleoside linkage, and a subscript “o” represents a phosphodiester internucleoside linkage.

TABLE 5
Design of siRNA targeted to human/mouse HPRT1 containing
chiral mesyl phosphoramidate linkages

| Duplex | | |
|---|---|---|
| Compound | Antisense Strand | Sense Strand |
| No. | Compound No. | Compound No. |
| 1590260 | 1337111 | 1337113 |
| 1590261 | 1465680 | 1337113 |
| 1590262 | 1590251 | 1337113 |
| 1590263 | 1590252 | 1337113 |
| 1590264 | 1465681 | 1337113 |
| 1590265 | 1590253 | 1337113 |
| 1590266 | 1590254 | 1337113 |
| 1590267 | 1590255 | 1337113 |
| 1590268 | 1590256 | 1337113 |
| 1590269 | 1590257 | 1337113 |
| 1590270 | 1590258 | 1337113 |
| 1590271 | 1590259 | 1337113 |

### Example 2: Design of siRNA to HPRT1 with Stereo-Standard Nucleosides and Stereo-Non-Standard Nucleosides

Modified oligonucleotides in the table below having either stereo-standard nucleosides or stereo-non-standard nucleosides in the antisense RNAi oligonucleotides and/or the sense RNAi oligonucleotide were synthesized using standard techniques.

Each antisense RNAi oligonucleotide described in the table below has the sequence AUAAAAUCUACAGUCAUAGGAAU (SEQ ID NO: 3) and is 100% complementary to GenBank Accession No. NM_000194.2 (SEQ ID NO: 1) from nucleosides 444 to 466.

TABLE 6
Design of antisense RNAi oligonucleotides
targeted to human/mouse HPRT1 containing
stereo-standard nucleosides and stereo-non-
standard nucleosides

| | | SEQ |
|---|---|---|
| Compound | Chemistry Notation | ID |
| No. | (5′ to 3′) | NO. |
| 1453015 | A_{ys}U_{fs}A_{yo}A_{yo}A_{yo}A_{fo}U_{yo}C_{yo} | 3 |
| | U_{yo}A_{yo}C_{yo}A_{yo}G_{yo}U_{fo}C_{yo}A_{fo} | |
| | U_{yo}A_{yo}G_{yo}G_{yo}A_{ys}A_{ys}U_{y} | |
| 1590189 | A_{ys}U_{[f2bDx]s}A_{yo}A_{yo}A_{yo}A_{fo} | 3 |
| | U_{yo}C_{yo}U_{yo}A_{yo}C_{yo}A_{yo}G_{yo}U_{fo} | |
| | C_{yo}A_{fo}U_{yo}A_{yo}G_{yo}G_{yo}A_{ys}A_{ys} | |
| | U_{y} | |
| 1590190 | A_{ys}U_{fs}A_{yo}A_{yo}A_{yo}A_{[f2bDx]o} | 3 |
| | U_{yo}C_{yo}U_{yo}A_{yo}C_{yo}A_{yo}G_{yo}U_{fo} | |
| | C_{yo}A_{fo}U_{yo}A_{yo}G_{yo}G_{yo}A_{ys}A_{ys} | |
| | U_{y} | |
| 1590191 | A_{ys}U_{fs}A_{yo}A_{yo}A_{yo}A_{fo}U_{yo}C_{yo} | 3 |
| | U_{yo}A_{yo}C_{yo}A_{yo}G_{yo}U_{[f2bDx]o} | |
| | C_{yo}A_{fo}U_{yo}A_{yo}G_{yo}G_{yo}A_{ys}A_{ys} | |
| | U_{y} | |
| 1590192 | A_{ys}U_{fs}A_{yo}A_{yo}A_{yo}A_{fo}U_{yo}C_{yo} | 3 |
| | U_{yo}A_{yo}C_{yo}A_{yo}G_{yo}U_{fo}C_{yo} | |
| | A_{[f2bDx]o}U_{yo}A_{yo}G_{yo}G_{yo}A_{ys} | |
| | A_{ys}U_{y} | |
| 1590193 | A_{ys}U_{[f2bDx]s}A_{yo}A_{yo}A_{yo} | 3 |
| | A_{[f2bDx]o}U_{yo}C_{yo}U_{yo}A_{yo}C_{yo} | |
| | A_{yo}G_{yo}U_{[f2bDx]o}C_{yo} | |
| | A_{[f2bDx]o}U_{yo}A_{yo}G_{yo}G_{yo}A_{ys} | |
| | A_{ys}U_{y} | |

In the table above, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, and a subscript “o” represents a phosphodiester internucleoside linkage.

The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Each sense RNAi oligomeric compound further comprises a GalNAc conjugated at the 3′-oxygen of the oligonucleotide via a THA linker as shown below:

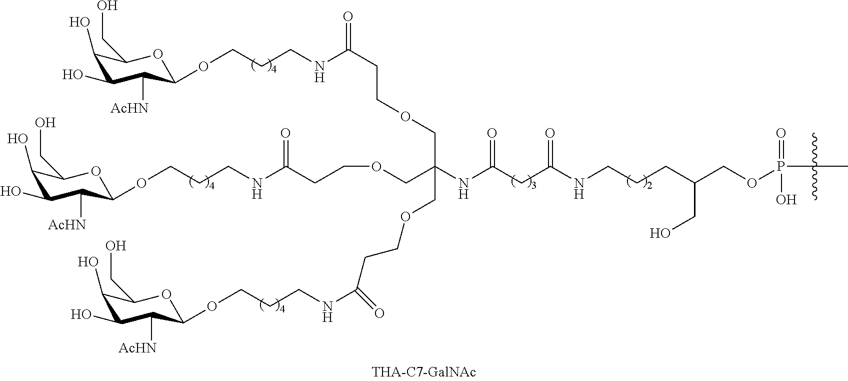

TABLE 7
Design of sense RNAi oligomeric compounds
targeted to human/mouse HPRT1 containing
stereo-standard nucleosides and stereo-
non-standard nucleosides

| | | SEQ |
|---|---|---|
| Compound | Chemistry Notation | ID |
| No. | (5′ to 3′) | NO. |
| 1448688 | U_{ys}C_{ys}C_{yo}U_{yo}A_{yo}U_{yo}G_{fo}A_{yo} | 4 |
| | C_{fo}U_{fo}G_{fo}U_{yo}A_{yo}G_{yo}A_{yo}U_{yo} | |
| | U_{yo}U_{yo}U_{yo}A_{yo}U_{y}-THA-C7- | |
| | GalNAc | |
| 1590194 | U_{ys}C_{ys}C_{yo}UyoA_{yo}U_{yo}G_{fo}A_{yo} | 4 |
| | C_{fo}U_{fo}G_{[f2bDx]o}U_{yo}A_{yo}G_{yo} | |
| | A_{yo}U_{yo}U_{yo}U_{yo}U_{yo}A_{yo}U_{y}-THA- | |
| | C7-GalNAc | |
| 1590195 | U_{ys}C_{ys}C_{yo}U_{yo}A_{yo}U_{yo}G_{fo}A_{yo} | 4 |
| | C_{fo}U_{[f2bDx]o}G_{fo}U_{yo}A_{yo}G_{yo} | |
| | A_{yo}U_{yo}U_{yo}U_{yo}U_{yo}A_{yo}U_{y}-THA- | |
| | C7-GalNAc | |
| 1590197 | U_{ys}C_{ys}C_{yo}U_{yo}A_{yo}U_{yo}G_{fo}A_{yo} | 4 |
| | C_{[f2bDx]o}U_{fo}G_{fo}U_{yo}A_{yo}G_{yo} | |
| | A_{yo}U_{yo}U_{yo}U_{yo}U_{yo}A_{yo}U_{y}-THA- | |
| | C7-GalNAc | |
| 1590198 | U_{ys}C_{ys}C_{yo}U_{yo}A_{yo}U_{yo}G_{[f2bDx]o} | 4 |
| | A_{yo}C_{fo}U_{fo}G_{fo}U_{yo}A_{yo}G_{yo}A_{yo} | |
| | U_{yo}U_{yo}U_{yo}U_{yo}A_{yo}U_{y}-THA-C7- | |
| | GalNAc | |
| 1590200 | U_{ys}C_{ys}C_{yo}U_{yo}A_{yo}U_{yo}G_{[f2bDx]o} | 4 |
| | A_{yo}C_{[f2bDx]o}U_{[f2bDx]o}G_{[f2bDx]o} | |
| | U_{yo}A_{yo}G_{yo}A_{yo}U_{yo}U_{yo}U_{yo}U_{yo}A_{yo} | |
| | U_{y}-THA-C7-GalNAc | |

In the table above, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, and a subscript “o” represents a phosphodiester internucleoside linkage.

### Example 3: Design of siRNA to Human APOE Having Modified Phosphoramidate Internucleoside Linkages

Double-stranded siRNA comprising modified oligonucleotides having mesyl phosphoramidate internucleoside linkages in the antisense RNAi oligonucleotides and/or the sense RNAi oligonucleotide were synthesized using standard techniques. Each internucleoside linkage is either a phosphorothioate internucleoside linkage (“s”), a phosphodiester internucleoside linkage (“o”), or a modified phosphoramidate internucleoside linkage (“IV”), as shown below.

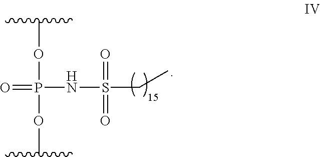

Compound No. 1518275 in the table below is 100% complementary to GenBank Accession No. NM_001302688.1 (SEQ ID NO: 2) from nucleosides 1030 to 1052 (SEQ ID NO: 38). Compound Nos. 1590434, 1590437, and 1590442 are 100% complementary to SEQ ID NO: 2 aside from a single mismatch at position 1 on the 5′-end.

TABLE 8
Design of antisense RNAi oligonucleo-
tidestargeted to human APOE containing
modified phosphoramidate linkages

| | | SEQ |
|---|---|---|
| Compound | Chemistry Notation | ID |
| No. | (5′ to 3′) | NO. |
| 1518275 | G_{ys}G_{fs}C_{yo}U_{fo}C_{yo}G_{fo}A_{yo}A_{fo} | 5 |
| | C_{yo}C_{fo}A_{yo}G_{fo}C_{yo}U_{fo}C_{yo}U_{fo} | |
| | U_{yo}G_{fo}A_{yo}G_{fo}G_{ys}C_{ys}G_{y} | |
| 1590434 | vP-TₑₛG_{fs}C_{yo}U_{fo}C_{yo}G_{fo}A_{yo} | 6 |
| | A_{fo}C_{yo}C_{fo}A_{yo}G_{fo}C_{yo}U_{fo}C_{yo} | |
| | U_{fo}U_{yo}G_{fo}A_{yo}G_{fo}G_{ys}C_{ys}G_{y} | |
| 1590437 | vP-TₑₛG_{fs}C_{yo}U_{yo}C_{yo}G_{yo}A_{yo} | 6 |
| | A_{yo}C_{yo}C_{yo}A_{yo}G_{yo}C_{yo}U_{fo}C_{yo} | |
| | U_{fo}U_{yo}G_{yo}A_{yo}G_{yo}G_{ys}C_{ys}G_{y} | |
| 1590442 | vP-TₑₛG_{fs}C_{yo}U_{yo}C_{yo}G_{yo}A_{yo} | 6 |
| | Ay_{[}IV_{]}C_{yo}C_{yo}A_{yo}G_{yo}C_{yo}U_{fo} | |
| | C_{yo}U_{fo}U_{yo}G_{yo}A_{yo}G_{yo}G_{ys}C_{ys}G_{y} | |

In the table above, a “vP-” represents a vinyl phosphonate (vP) moiety on the 5′-end, a subscript “e” represents a 2′-O-methyoxyethyl-β-D-ribosyl sugar moiety, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, a subscript “o” represents a phosphodiester internucleoside linkage, and a subscript “[IV]” represents an internucleoside linkage of Formula IV. Subscripts of nucleotides having a phosphoramidate internucleoside linkage of generic Formula I are bold and underlined.

The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

TABLE 9
Design of sense RNAi oligonucleotides
targeted to human APOE containing
modified phosphoramidate linkages

| | | SEQ |
|---|---|---|
| Compound | Chemistry Notation | ID |
| No. | (5′ to 3′) | NO. |
| 1518269 | C_{fs}C_{ys}U_{fo}C_{yo}A_{fo}A_{yo}G_{fo}A_{yo} | 7 |
| | G_{fo}C_{yo}U_{fo}G_{yo}G_{fo}U_{yo}U_{fo}C_{yo} | |
| | G_{fo}A_{yo}G_{fs}C_{ys}C_{f} | |
| 1590435 | C_{fs}C_{ys}U_{fo}C_{yo}A_{fo}A_{yo}G_{fo}A_{yo} | 8 |
| | G_{fo}C_{yo}U_{fo}G_{yo}G_{fo}U_{yo}U_{fo}C_{yo} | |
| | G_{fo}A_{yo}G_{fs}C_{ys}A_{f} | |
| 1590438 | C_{ys}C_{ys}U_{yo}C_{yo}A_{yo}A_{yo}G_{fo}A_{yo} | 8 |
| | G_{fo}C_{fo}U_{fo}G_{yo}G_{yo}U_{yo}U_{yo}C_{yo} | |
| | G_{yo}A_{yo}G_{ys}C_{ys}A_{y} | |
| 1590440 | C_{ys}C_{ys}U_{yo}C_{yo}A_{yo}Ay_{[}IV_{]}G_{fo} | 8 |
| | A_{yo}G_{fo}C_{fo}U_{fo}G_{yo}G_{yo}U_{yo}U_{yo} | |
| | C_{yo}G_{yo}A_{yo}G_{ys}C_{ys}A_{y} | |

In the table above, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, a subscript “o” represents a phosphodiester internucleoside linkage, and a subscript “[IV]” represents an internucleoside linkage of Formula IV. Subscripts of nucleotides having a phosphoramidate internucleoside linkage of generic Formula I are bold and underlined.

TABLE 10
Design of siRNA targeted to human APOE containing
modified phosphoramidate linkages

| | Antisense | Sense |
|---|---|---|
| Duplex | Strand | Strand |
| Compound | Compound | Compound |
| No. | No. | No. |
| 1518266 | 1518275 | 1518269 |
| 1590436 | 1590434 | 1590435 |
| 1590439 | 1590437 | 1590438 |
| 1590441 | 1590437 | 1590440 |
| 1590443 | 1590442 | 1590438 |

### Example 4: Design of siRNA to HPRT1 Having C16-Modified Nucleosides

Modified oligonucleotides in the table below having either standard nucleosides or C16-modified nucleosides in the antisense RNAi oligonucleotides and/or the sense RNAi oligonucleotide were synthesized using standard techniques.

Compound No. 1449196 in the table below is 100% complementary to GenBank Accession No. NM_000194.2 (SEQ ID NO: 1) from nucleosides 444 to 466. Compound Nos. 1586322 and 1590779 are 100% complementary to SEQ ID NO: 1 aside from a single mismatch at position 1 on the 5′-end.

TABLE 11
Design of antisense RNAi oligomeric compounds
targeted to human/mouse HPRT1 containing
C16-modified nucleosides

| | | SEQ |
|---|---|---|
| Compound | Chemistry Notation | ID |
| No. | (5′ to 3′) | NO. |
| 1449196 | p.A_{ys}U_{fs}A_{yo}A_{yo}A_{yo}A_{fo}U_{yo} | 3 |
| | C_{yo}U_{yo}A_{yo}C_{yo}A_{yo}G_{yo}U_{fo}C_{yo} | |
| | A_{fo}U_{yo}A_{yo}G_{yo}G_{yo}A_{ys}A_{ys}U_{y} | |
| 1586322 | vP-TₑₛU_{fs}A_{yo}A_{yo}A_{yo}A_{fo}U_{yo} | 9 |
| | C_{yo}U_{yo}A_{yo}C_{yo}A_{yo}G_{yo}U_{fo}C_{yo} | |
| | A_{fo}U_{yo}A_{yo}G_{yo}G_{yo}A_{ys}A_{ys}U_{y} | |
| 1590779 | vP-TₑₛU_{fs}A_{yo}A_{yo}A_{yo}A_{fo}U_{yo} | 9 |
| | C_{[16C2r]o}U_{yo}A_{yo}C_{yo}A_{yo}G_{yo} | |
| | U_{fo}C_{yo}A_{fo}U_{yo}A_{yo}G_{yo}G_{yo}A_{ys} | |
| | A_{ys}U_{y} | |

In the table above, a “p.” represents a 5′-phosphate, a “vP-” represents a vinyl phosphonate (vP) moiety on the 5′-end, a subscript “e” represents a 2′-O-methyoxyethyl-β-D-ribosyl sugar moiety, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, and a subscript “o” represents a phosphodiester internucleoside linkage. A subscript “[16C2r]” represents the sugar moiety of a 2′-O-hexadecyl modified nucleoside as shown below:

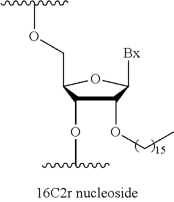

The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

TABLE 12
Design of sense RNAi oligomeric compounds
targeted to human/mouse HPRT1 containing
C16-modified nucleosides

| | | SEQ |
|---|---|---|
| Compound | Chemistry Notation | ID |
| No. | (5′ to 3′) | NO. |
| 1505889 | U_{ys}C_{ys}C_{yo}U_{yo}A_{yo}U_{yo}G_{fo}A_{yo} | 4 |
| | C_{fo}U_{fo}G_{fo}U_{yo}A_{yo}G_{yo}A_{yo}U_{yo} | |
| | U_{yo}U_{yo}U_{ys}A_{ys}U_{y} | |
| 1586323 | U_{ys}C_{ys}C_{yo}U_{yo}A_{yo}U_{yo}G_{fo}A_{yo} | 10 |
| | C_{fo}U_{fo}G_{fo}U_{yo}A_{yo}G_{yo}A_{yo}U_{yo} | |
| | U_{yo}U_{yo}U_{ys}A_{ys}A_{y} | |
| 1586324 | U_{ys}C_{ys}C_{yo}U_{yo}A_{yo}U_{[16C2r]o} | 10 |
| | G_{fo}A_{yo}C_{fo}U_{fo}G_{fo}U_{yo}A_{yo}G_{yo} | |
| | A_{yo}U_{yo}U_{yo}U_{yo}U_{ys}A_{ys}A_{y} | |
| 1590179 | [C16-HA]ₒU_{ys}C_{ys}C_{yo}U_{yo}A_{yo} | 10 |
| | U_{yo}G_{fo}A_{yo}C_{fo}U_{fo}G_{fo}U_{yo}A_{yo} | |
| | G_{yo}A_{yo}U_{yo}U_{yo}U_{yo}U_{ys}A_{ys}A_{y} | |
| 1590461 | U_{ys}C_{ys}C_{yo}U_{yo}A_{yo}Uy_{[}IV_{]}G_{fo} | 10 |
| | A_{yo}C_{fo}U_{fo}G_{fo}U_{yo}A_{yo}G_{yo}A_{yo} | |
| | U_{yo}U_{yo}U_{yo}U_{ys}A_{ys}A_{y} | |
| 1591095 | U_{ys}C_{ys}C_{yo}U_{yo}A_{yo}U_{yo}G_{fo}A_{yo} | 10 |
| | C_{fo}U_{fo}G_{fo}U_{yo}A_{yo}G_{yo}A_{yo}U_{yo} | |
| | U_{yo}U_{yo}U_{ys}A_{ys}A_{yo}[3nC7-C16] | |
| 1591114 | U_{ys}C_{ys}C_{yo}U_{yo}A_{yo}U_{[C16Am]o} | 10 |
| | G_{fo}A_{yo}C_{fo}U_{fo}G_{fo}U_{yo}A_{yo}G_{yo} | |
| | A_{yo}U_{yo}U_{yo}U_{yo}U_{ys}A_{ys}A_{y} | |

In the table above, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, a subscript “o” represents a phosphodiester internucleoside linkage, and a subscript “[IV]” represents an internucleoside linkage of Formula IV as shown in Example 3. Subscripts of nucleotides having a phosphoramidate internucleoside linkage of generic Formula XVII are bold and underlined. A subscript “[16C2r]” represents the sugar moiety of a 2′-O-hexadecyl modified nucleoside as shown below:

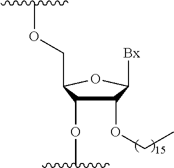

A subscript “[C16Am]” represents the sugar moiety of 2′-O-hexylpalmitamide modified nucleosideas shown below:

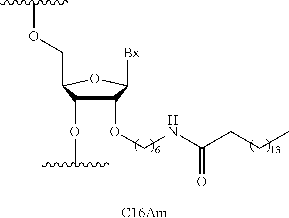

“[C16-HA]” represents a hexylaminopalmitate moiety, as shown below, which is attached to the 5′-nucleoside via a phosphodiester linkage.

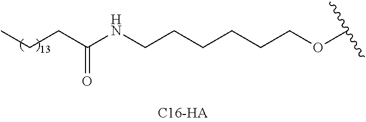

“[3nC7-C16]” represents a palmitate moiety linked to a 3′-C7 amino modifier, as shown below, which is attached to the 3′-nucleoside via a phosphodiester linkage.

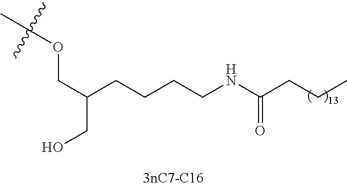

TABLE 13
Design of siRNA targeted to human/mouse
HPRT1 containing C16-modified nucleosides

| Duplex | Antisense Strand | Sense Strand |
|---|---|---|
| Compound No. | Compound No. | Compound No. |
| 1679408 | 1449196 | 1505889 |
| 1588821 | 1586322 | 1586323 |
| 1588822 | 1586322 | 1586324 |
| 1590462 | 1586322 | 1590461 |
| 1679399 | 1586322 | 1591114 |
| 1599465 | 1590779 | 1586323 |
| 1599475 | 1586322 | 1590179 |
| 1599476 | 1586322 | 1591095 |

### Example 5: Design of siRNA to HPRT1 Having Mesyl Phosphoramidate Internucleoside Linkages

Double-stranded siRNA comprising modified oligonucleotides having mesyl phosphoramidate internucleoside linkages (Formula II) in the antisense RNAi oligonucleotides and/or the sense RNAi oligonucleotide were synthesized using standard techniques. Each internucleoside linkage is either a phosphorothioate internucleoside linkage (“s”), a phosphodiester internucleoside linkage (“o”), or a mesyl phosphoramidate internucleoside linkage (“z”), indicated by Formula II below.

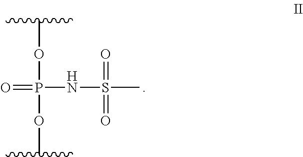 [SMILES: *OP(=O)(NS(C)(=O)=O)O*]

Compound No. 1449196 in the table below is 100% complementary to GenBank Accession No. NM_000194.2 (SEQ ID NO: 1) from nucleosides 444 to 466. All other compound IDs in the table below are 100% complementary to SEQ ID NO: 1 aside from a single mismatch at position 1 on the 5′-end.

TABLE 14
Design of antisense RNAi oligonucleotides
targeted to human/mouse HPRT1 containing
mesyl phosphoramidate linkages

| | | SEQ |
|---|---|---|
| Compound | Chemistry Notation | ID |
| No. | (5′ to 3′) | NO. |
| 1449196 | p. A_{ys}U_{fs}A_{yo}A_{yo}A_{yo}A_{fo}U_{yo} | 3 |
| | C_{yo}U_{yo}A_{yo}C_{yo}A_{yo}G_{yo}U_{fo}C_{yo} | |
| | A_{fo}U_{yo}A_{yo}G_{yo}G_{yo}A_{ys}A_{ys}U_{y} | |
| 1586322 | vP-TₑₛU_{fs}A_{yo}A_{yo}A_{yo}A_{fo}U_{yo} | 9 |
| | C_{yo}U_{yo}A_{yo}C_{yo}A_{yo}G_{yo}U_{fo}C_{yo} | |
| | A_{fo}U_{yo}A_{yo}G_{yo}G_{yo}A_{ys}A_{ys}U_{y} | |
| 1591115 | vP-TₑₛU_{fs}A_{yo}A_{yo}AyₒA_{fo}U_{yo} | 9 |
| | C_{yo}U_{yo}A_{yo}C_{yo}A_{yo}G_{yo}U_{fo}C_{yo} | |
| | A_{fo}U_{yo}A_{yo}G_{yo}G_{yo}AyzAyzU_{y} | |
| 1591230 | vP-TezU_{fs}A_{yo}A_{yo}A_{yo}A_{fo}U_{yo} | 9 |
| | C_{yo}U_{yo}A_{yo}C_{yo}A_{yo}G_{yo}U_{fo}C_{yo} | |
| | A_{fo}U_{yo}A_{yo}G_{yo}G_{yo}AyzAyzU_{y} | |
| 1591231 | vP-TezU_{fs}A_{yo}A_{yo}A_{yo}AfzU_{yo} | 9 |
| | C_{yo}U_{yo}A_{yo}C_{yo}A_{yo}G_{yo}U_{fo}C_{yo} | |
| | A_{fo}U_{yo}A_{yo}G_{yo}G_{yo}AyzAyzU_{y} | |
| 1591241 | z.TₑₛU_{fs}A_{yo}A_{yo}A_{yo}A_{fo}U_{yo} | 9 |
| | C_{yo}U_{yo}A_{yo}C_{yo}A_{yo}G_{yo}U_{fo}C_{yo} | |
| | A_{fo}U_{yo}A_{yo}G_{yo}G_{yo}A_{ys}A_{ys}U_{y} | |

In the table above, a “p.” represents a 5′-phosphate, a “z.” represents a 5′-mesyl phosphoramidate terminal group (shown in Formula XIII below), a “vP-” represents a vinyl phosphonate (vP) moiety on the 5′-end, a subscript “e” represents a 2′-O-methyoxyethyl-β-D-ribosyl sugar moiety, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, and a subscript “o” represents a phosphodiester internucleoside linkage, and a subscript “z” represents a mesyl phosphoramidate internucleoside linkage or a 5′-mesyl phosphoramidate (Formula XIII). Subscripts of nucleotides having a phosphoramidate internucleoside linkage of generic Formula I are bold and underlined.

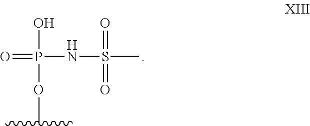 [SMILES: *OP(=O)(O)NS(C)(=O)=O]

The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

TABLE 15
Design of sense RNAi oligonucleotides
targeted to human/mouse HPRT1 containing
mesyl phosphoramidate linkages

| | | SEQ |
|---|---|---|
| Compound | Chemistry Notation | ID |
| No. | (5′ to 3′) | NO. |
| 1505889 | U_{ys}C_{ys}C_{yo}U_{yo}A_{yo}U_{yo}G_{fo}A_{yo} | 4 |
| | C_{fo}U_{fo}G_{fo}U_{yo}A_{yo}G_{yo}A_{yo}U_{yo} | |
| | U_{yo}U_{yo}U_{ys}A_{ys}U_{y} | |
| 1586323 | U_{ys}C_{ys}C_{yo}U_{yo}A_{yo}U_{yo}G_{fo}A_{yo} | 10 |
| | C_{fo}U_{fo}G_{fo}U_{yo}A_{yo}G_{yo}A_{yo}U_{yo} | |
| | U_{yo}U_{yo}U_{ys}A_{ys}A_{y} | |
| 1591116 | UyzCyzC_{yo}U_{yo}A_{yo}U_{yo}G_{fo}A_{yo} | 10 |
| | C_{fo}U_{fo}G_{fo}U_{yo}A_{yo}G_{yo}A_{yo}U_{yo} | |
| | U_{yo}U_{yo}UyzAyzA_{y} | |
| 1591233 | U_{ys}C_{ys}C_{yo}U_{yo}A_{yo}U_{yo}GfzA_{yo} | 10 |
| | C_{fo}U_{fo}GfzU_{yo}A_{yo}G_{yo}A_{yo}U_{yo} | |
| | U_{yo}U_{yo}U_{ys}A_{ys}A_{y} | |
| 1591239 | U_{ys}C_{ys}C_{yo}U_{yo}A_{yo}U_{yo}GfzA_{yo} | 10 |
| | CfzU_{fo}GfzU_{yo}A_{yo}G_{yo}A_{yo}U_{yo} | |
| | U_{yo}U_{yo}U_{ys}A_{ys}A_{y} | |
| 1591240 | U_{ys}C_{ys}C_{yo}U_{yo}A_{yo}U_{yo}GfzA_{yo} | 10 |
| | CfzUfzGfzU_{yo}A_{yo}G_{yo}A_{yo}U_{yo} | |
| | U_{yo}U_{yo}U_{ys}A_{ys}A_{y} | |

In the table above, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, a subscript “o” represents a phosphodiester internucleoside linkage, and a subscript “z” represents a mesyl phosphoramidate internucleoside linkage. Subscripts of nucleotides having a phosphoramidate internucleoside linkage of generic Formula I are bold and underlined.

TABLE 16
Design of siRNA targeted to human/mouse HPRT1
containing mesyl phosphoramidate linkages

| Duplex | Antisense Strand | Sense Strand |
|---|---|---|
| Compound No. | Compound No. | Compound No. |
| 1679408 | 1449196 | 1505889 |
| 1588821 | 1586322 | 1586323 |
| 1679400 | 1591115 | 1591116 |
| 1679401 | 1591230 | 1591116 |
| 1679402 | 1591231 | 1591116 |
| 1679403 | 1586322 | 1591233 |
| 1679404 | 1586322 | 1591239 |
| 1679405 | 1586322 | 1591240 |
| 1679406 | 1591115 | 1591240 |
| 1679407 | 1591241 | 1586323 |

### Example 6: Design of siRNA to HPRT1 Having Mesyl Phosphoramidate Internucleoside Linkages

Double-stranded siRNA comprising modified oligonucleotides having mesyl phosphoramidate internucleoside linkages (Formula II) in the antisense RNAi oligonucleotides and/or sense RNAi oligonucleotide were synthesized using standard techniques. Each internucleoside linkage is either a phosphorothioate internucleoside linkage (“s”), a phosphodiester internucleoside linkage (“o”), or a mesyl phosphoramidate internucleoside linkage (“z”), indicated by Formula II below.

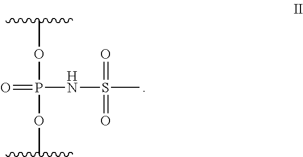 [SMILES: *OP(=O)(NS(C)(=O)=O)O*]

The antisense RNAi oligonucleotides are described as in Table 12 above, and the sense RNAi oligomeric compounds are described in the table below. The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Certain sense RNAi oligomeric compounds contain a C16 conjugate, as indicated in the table below.

TABLE 17
Design of sense RNAi oligomeric compounds
targeted to human/mouse HPRT1 containing
mesyl phosphoramidate linkages

| | | SEQ |
|---|---|---|
| Compound | Chemistry Notation | ID |
| No. | (5′ to 3′) | NO. |
| 1505889 | U_{ys}C_{ys}C_{yo}U_{yo}A_{yo}U_{yo}G_{fo}A_{yo} | 4 |
| | C_{fo}U_{fo}G_{fo}U_{yo}A_{yo}G_{yo}A_{yo}U_{yo} | |
| | U_{yo}U_{yo}U_{ys}A_{ys}U_{y} | |
| 1586323 | U_{ys}C_{ys}C_{yo}U_{yo}A_{yo}U_{yo}G_{fo}A_{yo} | 10 |
| | C_{fo}U_{fo}G_{fo}U_{yo}A_{yo}G_{yo}A_{yo}U_{yo} | |
| | U_{yo}U_{yo}U_{ys}A_{ys}A_{y} | |
| 1590179 | [C16-HA]ₒU_{ys}C_{ys}C_{yo}U_{yo}A_{yo} | 10 |
| | U_{yo}G_{fo}A_{yo}C_{fo}U_{fo}G_{fo}U_{yo}A_{yo} | |
| | G_{yo}A_{yo}U_{yo}U_{yo}U_{yo}U_{ys}A_{ys}A_{y} | |
| 1591242 | [C16-HA]ₒUyzCyzC_{yo}U_{yo}A_{yo} | 10 |
| | U_{yo}G_{fo}A_{yo}C_{fo}U_{fo}G_{fo}U_{yo}A_{yo} | |
| | G_{yo}A_{yo}U_{yo}U_{yo}U_{yo}UyzAyzA_{y} | |
| 1591243 | [C16-HA]ₒU_{ys}C_{ys}C_{yo}U_{yo}A_{yo} | 10 |
| | U_{yo}GfzA_{yo}C_{fo}U_{fo}GfzU_{yo}A_{yo} | |
| | G_{yo}A_{yo}U_{yo}U_{yo}U_{yo}U_{ys}A_{ys}A_{y} | |
| 1591244 | [C16-HA]ₒU_{ys}C_{ys}C_{yo}U_{yo}A_{yo} | 10 |
| | U_{yo}GfzA_{yo}CfzU_{fo}GfzU_{yo}A_{yo} | |
| | G_{yo}A_{yo}U_{yo}U_{yo}U_{yo}U_{ys}A_{ys}A_{y} | |
| 1591245 | [C16-HA]ₒU_{ys}C_{ys}C_{yo}U_{yo}A_{yo} | 10 |
| | U_{yo}GfzA_{yo}CfzUfzGfzU_{yo}A_{yo} | |
| | G_{yo}A_{yo}U_{yo}U_{yo}U_{yo}U_{ys}A_{ys}A_{y} | |

In the table above, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, a subscript “o” represents a phosphodiester internucleoside linkage, and a subscript “z” represents a mesyl phosphoramidate internucleoside linkage. Subscripts of nucleotides having a phosphoramidate internucleoside linkage of generic Formula XVII are bold and underlined.

“[C16-HA]” represents a hexylaminopalmitate moiety, as shown below, which is attached to the 5′-nucleoside via a phosphodiester linkage.

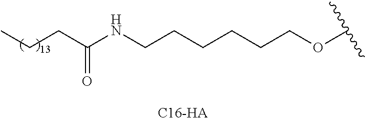

TABLE 18
Design of siRNA targeted to human/mouse HPRT1
containing mesyl phosphoramidate linkages

| Duplex | Antisense Strand | Sense Strand |
|---|---|---|
| Compound No. | Compound No. | Compound No. |
| 1679408 | 1449196 | 1505889 |
| 1588821 | 1586322 | 1586323 |
| 1599465 | 1586322 | 1590179 |
| 1679409 | 1591115 | 1591242 |
| 1679410 | 1591230 | 1591242 |
| 1679411 | 1591231 | 1591242 |
| 1679414 | 1586322 | 1591243 |
| 1679415 | 1586322 | 1591244 |
| 1679416 | 1586322 | 1591245 |
| 1679417 | 1591115 | 1591245 |
| 1679418 | 1591241 | 1590179 |

### Example 7: Design of siRNA to HPRT1 with Stereo-Standard Nucleosides and Stereo-Non-Standard Nucleosides

Modified oligonucleotides in the table below having either stereo-standard nucleosides or stereo-non-standard nucleosides in the antisense RNAi oligonucleotides and/or the sense RNAi oligomeric compounds were synthesized using standard techniques. The sense oligomeric compounds contain a C16 conjugate, as indicated in the table below. Each antisense RNAi oligonucleotide described in the table below has the sequence TUAAAAUCUACAGUCAUAGGAAU (SEQ ID NO: 9) and, aside from a mismatch at position 1 on the 5′-end of the antisense RNAi oligonucleotide, is 100% complementary to GenBank Accession No. NM_000194.2 (SEQ ID NO: 1) from nucleosides 444 to 466.

TABLE 19
Design of antisense RNAi oligonucleotides
targeted to human/mouse HPRT1 containing
stereo-standard nucleosides and stereo-
non-standard nucleosides

| | | SEQ |
|---|---|---|
| Compound | Chemistry Notation | ID |
| No. | (5′ to 3′) | NO. |
| 1586322 | vP-TₑₛU_{fs}A_{yo}A_{yo}A_{yo}A_{fo}U_{yo}C_{yo}U_{yo} | 9 |
| | A_{yo}C_{yo}A_{yo}G_{yo}U_{fo}C_{yo}A_{fo}U_{yo}A_{yo}G_{yo} | |
| | G_{yo}A_{ys}A_{ys}U_{y} | |
| 1591246 | vP-TₑₛU_{[f2bDx]s}A_{yo}A_{yo}A_{yo}A_{fo}U_{yo} | 9 |
| | C_{yo}U_{yo}A_{yo}C_{yo}A_{yo}G_{yo}U_{fo}C_{yo}A_{fo}U_{yo} | |
| | A_{yo}G_{yo}G_{yo}A_{ys}A_{ys}U_{y} | |
| 1591247 | vP-TₑₛU_{fs}A_{yo}A_{yo}A_{yo}A_{[f2bDx]o}U_{yo} | 9 |
| | C_{yo}U_{yo}A_{yo}C_{yo}A_{yo}G_{yo}U_{fo}C_{yo}A_{fo}U_{yo} | |
| | A_{yo}G_{yo}G_{yo}A_{ys}A_{ys}U_{y} | |
| 1591248 | vP-TₑₛU_{fs}A_{yo}A_{yo}A_{yo}A_{fo}U_{yo}C_{yo}U_{yo} | 9 |
| | A_{yo}C_{yo}A_{yo}G_{yo}U_{[f2bDx]o}C_{yo}A_{fo}U_{yo} | |
| | A_{yo}G_{yo}G_{yo}A_{ys}A_{ys}U_{y} | |
| 1591249 | vP-TₑₛU_{fs}A_{yo}A_{yo}A_{yo}A_{fo}U_{yo}C_{yo}U_{yo} | 9 |
| | A_{yo}C_{yo}A_{yo}G_{yo}U_{fo}C_{yo}A_{[f2bDx]o}U_{yo} | |
| | A_{yo}G_{yo}G_{yo}A_{ys}A_{ys}U_{y} | |
| 1591250 | vP-TₑₛU_{[f2bDx]s}A_{yo}A_{yo}A_{yo} | 9 |
| | A_{[f2bDx]o}U_{yo}C_{yo}U_{yo}A_{yo}C_{yo}AyₒG_{yo} | |
| | U_{[f2bDx]o}C_{yo}A_{[f2bDx]o}U_{yo}A_{yo}G_{yo} | |
| | G_{yo}A_{ys}A_{ys}U_{y} | |

In the table above, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-OMe-β-D-ribosyl sugar moiety, a subscript “e” represents a 2′-O-methyoxyethyl-β-D-ribosyl sugar moiety, a subscript “s” represents a phosphorothioate internucleoside linkage, and a subscript “o” represents a phosphodiester internucleoside linkage. Each antisense RNAi oligonucleotide contains a vinyl phosphonate (vP) moiety on the 5′-end.

TABLE 20
Design of sense RNAi oligomeric compounds
targeted to human/mouse HPRT1 containing
stereo-standard nucleosides and stereo-
non-standard nucleosides

| | | SEQ |
|---|---|---|
| Compound | Chemistry Notation | ID |
| No. | (5′ to 3′) | NO. |
| 1586324 | U_{ys}C_{ys}C_{yo}U_{yo}A_{yo}U_{[16C2r]o}G_{fo} | 10 |
| | A_{yo}C_{fo}U_{fo}G_{fo}U_{yo}A_{yo}G_{yo}A_{yo}U_{yo} | |
| | U_{yo}U_{yo}U_{ys}A_{ys}A_{y} | |
| 1591251 | U_{ys}C_{ys}C_{yo}U_{yo}A_{yo}U_{[16C2r]o}G_{fo} | 4 |
| | A_{yo}C_{fo}U_{fo}G_{[f2bDx]o}U_{yo}A_{yo}G_{yo} | |
| | A_{yo}U_{yo}U_{yo}U_{yo}U_{ys}A_{ys}U_{y} | |
| 1591252 | U_{ys}C_{ys}C_{yo}U_{yo}A_{yo}U_{[16C2r]o}G_{fo} | 4 |
| | A_{yo}C_{fo}U_{[f2bDx]o}G_{fo}U_{yo}A_{yo}G_{yo} | |
| | A_{yo}U_{yo}U_{yo}U_{yo}U_{ys}A_{ys}U_{y} | |
| 1591253 | U_{ys}C_{ys}C_{yo}U_{yo}A_{yo}U_{[16C2r]o}G_{fo} | 4 |
| | A_{yo}C_{[f2bDx]o}U_{fo}G_{fo}U_{yo}A_{yo}G_{yo} | |
| | A_{yo}U_{yo}U_{yo}U_{yo}U_{ys}A_{ys}U_{y} | |
| 1591254 | U_{ys}C_{ys}C_{yo}U_{yo}A_{yo}U_{[16C2r]o} | 4 |
| | G_{[f2bDx]o}A_{yo}C_{fo}U_{fo}G_{fo}U_{yo}A_{yo} | |
| | G_{yo}A_{yo}U_{yo}U_{yo}U_{yo}U_{ys}A_{ys}U_{y} | |
| 1591255 | U_{ys}C_{ys}C_{yo}U_{yo}A_{yo}U_{[16C2r]o} | 4 |
| | G_{[f2bDx]o}A_{yo}C_{[f2bDx]o} | |
| | U_{[f2bDx]o}G_{[f2bDx]o}U_{yo}A_{yo} | |
| | G_{yo}A_{yo}U_{yo}U_{yo}U_{yo}U_{ys}A_{ys}U_{y} | |

In the table above, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, and a subscript “o” represents a phosphodiester internucleoside linkage. A subscript “[16C2r]” represents the sugar moiety of a 2′-O-hexadecyl modified nucleoside as shown below:

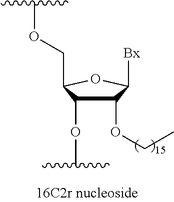

### Example 8: Design of siRNA to HPRT1 Having Mesyl Phosphoramidate Internucleoside Linkages

Double-stranded siRNA comprising modified oligonucleotides having mesyl phosphoramidate internucleoside linkages (Formula II) in the antisense RNAi oligonucleotides and/or sense RNAi oligonucleotide were synthesized using standard techniques. Each internucleoside linkage is either a phosphorothioate internucleoside linkage (“s”), a phosphodiester internucleoside linkage (“o”), or a mesyl phosphoramidate internucleoside linkage (“z”), indicated by Formula II below.

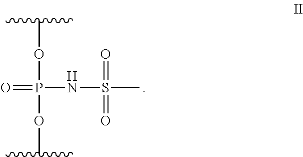 [SMILES: *OP(=O)(NS(C)(=O)=O)O*]

Each compound in the table below is 100% complementary to GenBank Accession No. NM_000194.2 (SEQ ID NO: 1) from nucleosides 444 to 466 aside from a single mismatch at position 1 on the 5′-end.

TABLE 21
Design of antisense RNAi oligonucleotides
targeted to human/mouse HPRT1 containing
mesyl phosphoramidate linkages

| | | SEQ |
|---|---|---|
| Compound | Chemistry Notation | ID |
| No. | (5′ to 3′) | NO. |
| 1595969 | vP-TₑₛU_{fs}A_{yo}A_{yo}A_{yo}A_{fo}U_{yo}C_{yo} | 9 |
| | U_{yo}A_{yo}C_{yo}A_{yo}G_{yo}U_{fo}C_{yo}A_{fo}U_{yo} | |
| | A_{yo}G_{yo}G_{yo}A_{ys}AyzU_{y} | |
| 1595970 | vP-TₑₛU_{fs}A_{yo}A_{yo}A_{yo}A_{fo}U_{yo}C_{yo} | 9 |
| | U_{yo}A_{yo}C_{yo}A_{yo}G_{yo}U_{fo}C_{yo}A_{fo}U_{yo} | |
| | A_{yo}G_{yo}G_{yo}AyzA_{ys}U_{y} | |
| 1595971 | vP-TₑₛUfzA_{yo}A_{yo}A_{yo}A_{fo}U_{yo}C_{yo} | 9 |
| | U_{yo}A_{yo}C_{yo}A_{yo}G_{yo}U_{fo}C_{yo}A_{fo}U_{yo} | |
| | A_{yo}G_{yo}G_{yo}A_{ys}AyzU_{y} | |
| 1595973 | vP-TezU_{fs}A_{yo}A_{yo}A_{yo}A_{fo}U_{yo}C_{yo} | 9 |
| | U_{yo}A_{yo}C_{yo}A_{yo}G_{yo}U_{fo}C_{yo}A_{fo}U_{yo} | |
| | A_{yo}G_{yo}G_{yo}A_{ys}AyzU_{y} | |

In the table above, a “vP-” represents a vinyl phosphonate (vP) moiety on the 5′-end, a subscript “e” represents a 2′-O-methyoxyethyl-β-D-ribosyl sugar moiety, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, and a subscript “o” represents a phosphodiester internucleoside linkage, and a subscript “z” represents a mesyl phosphoramidate internucleoside linkage. Subscripts of nucleotides having a phosphoramidate internucleoside linkage of generic Formula I are bold and underlined.

The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). The sense RNAi oligomeric compounds contain a C16 conjugate, as indicated in the table below.

TABLE 22
Design of sense RNAi oligomeric compounds
targeted to human/mouse HPRT1

| | | SEQ |
|---|---|---|
| Compound | Chemistry Notation | ID |
| No. | (5′ to 3′) | NO. |
| 1590179 | [C16-HA]ₒU_{ys}C_{ys}C_{yo}U_{yo}A_{yo} | 10 |
| | U_{yo}G_{fo}A_{yo}C_{fo}U_{fo}G_{fo}U_{yo}A_{yo} | |
| | G_{yo}A_{yo}U_{yo}U_{yo}U_{yo}U_{ys}A_{ys}A_{y} | |
| 1595972 | [C16-HA]ₒUyzC_{ys}C_{yo}U_{yo}A_{yo} | 10 |
| | U_{yo}G_{fo}A_{yo}C_{fo}U_{fo}G_{fo}U_{yo}A_{yo} | |
| | G_{yo}A_{yo}U_{yo}U_{yo}U_{yo}U_{ys}AyzA_{y} | |

In the table above, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, a subscript “o” represents a phosphodiester internucleoside linkage, and a subscript “z” represents a mesyl phosphoramidate internucleoside linkage. Subscripts of nucleotides having a phosphoramidate internucleoside linkage of generic Formula I are bold and underlined. “[C16-HA]” represents a hexylaminopalmitate moiety, as shown below, which is attached to the 5′-nucleoside via a phosphodiester linkage.

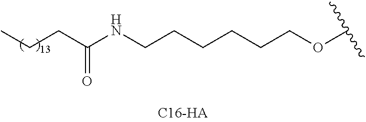

TABLE 23
Design of siRNA targeted to human/mouse HPRT1
containing mesyl phosphoramidate linkages

| Duplex | Antisense Strand | Sense Strand |
|---|---|---|
| Compound No. | Compound No. | Compound No. |
| 1679419 | 1595969 | 1590179 |
| 1679420 | 1595970 | 1590179 |
| 1679421 | 1595973 | 1590179 |
| 1679422 | 1595971 | 1590179 |
| 1679424 | 1595973 | 1595972 |
| 1679437 | 1595971 | 1595972 |

### Example 9: Design of siRNA to HPRT1 Having Modified and Unmodified Nucleobases

Double-stranded siRNA were synthesized using standard techniques. Compound No. 1586322 in the table below is 100% complementary to GenBank Accession No. NM_000194.2 (SEQ ID NO: 1) from nucleosides 444 to 466 aside from a single mismatch at position 1 on the 5′-end.

TABLE 24
Design of antisense RNAi oligonucleotides
targeted to human/mouse HPRT1

| | | SEQ |
|---|---|---|
| Compound | Chemistry Notation | ID |
| No. | (5′ to 3′) | NO. |
| 1586322 | vP-TesUfsAyoAyoAyoAfoUyo | 9 |
| | CyoUyoAyoCyoAyoGyoUfoCyo | |
| | AfoUyoAyoGyoGyoAysAysUy | |

In the table above, a “vP-” represents a vinyl phosphonate (vP) moiety on the 5′-end, a subscript “e” represents a 2′-O-methyoxyethyl-β-D-ribosyl sugar moiety, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, and a subscript “o” represents a phosphodiester internucleoside linkage.

The sense RNAi oligonucleotide is complementary to the first of the 21 nucleosides of the antisense RNAi oligonucleotide (from 5′ to 3′), and the last two 3′-nucleosides of the antisense RNAi oligonucleotides are not paired with the sense RNAi oligonucleotide (are overhanging nucleosides). The last nine 3′-nucleosides of the sense RNAi oligonucleotide are not paired with the antisense RNAi oligonucleotide, nor are they complementary to the complement of GenBank Accession No. NM_000194.2 (SEQ ID NO: 1).

TABLE 25
Design of sense RNAi oligonucleotides
targeted to human/mouse HPRT1

| | | SEQ |
|---|---|---|
| Compound | Chemistry Notation | ID |
| No. | (5′ to 3′) | NO. |
| 1595977 | U_{ys}C_{ys}C_{yo}U_{yo}A_{yo}U_{yo}G_{fo}A_{yo} | 11 |
| | C_{fo}U_{fo}G_{fo}U_{yo}A_{yo}G_{yo}A_{yo}U_{yo} | |
| | U_{yo}U_{yo}U_{ys}A_{ys}A_{ys}T_{ds}A_{ds}^{m}C_{ds} | |
| | T_{ds}A_{ds}^{m}C_{ds}T_{ds}A_{ds}^{m}C_{d} | |

In the table above, a subscript “f” represents a 2′-fluoro-β-D-ribosyl sugar moiety, a subscript “y” represents a 2′-OMe-β-D-ribosyl sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

TABLE 26
Design of siRNA targeted to human/mouse HPRT1

| Duplex | Antisense Strand | Sense Strand |
|---|---|---|
| Compound No. | Compound No. | Compound No. |
| 1679438 | 1586322 | 1595977 |

### Example 10: Design of siRNA to HPRT1 Containing 2′-Fluoro-β-D-Xylosyl Nucleosides

Modified oligonucleotides in the table below having either stereo-standard nucleosides or stereo-non-standard nucleosides in the antisense RNAi oligonucleotides and/or the sense RNAi oligomeric compound were synthesized using standard techniques. The following structure shows a 2′-fluoro-β-D-xylosyl nucleoside (f2bDx), wherein Bx is a heterocyclic base moiety:

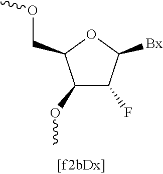

The sense RNAi oligomeric compounds contain a C16 conjugate, as indicated in the table below.

TABLE 27
Design of sense RNAi oligomeric compounds
targeted to human/mouse HPRT1 containing
2′-fluoro-ß-D-xylosyl nucleosides

| | | SEQ |
|---|---|---|
| Compound | Chemistry Notation | ID |
| No. | (5′ to 3′) | NO. |
| 1600514 | U_{ys}C_{ys}C_{yo}U_{yo}A_{yo}U_{[16C2r]o} | 10 |
| | G_{fo}A_{yo}C_{fo}U_{[f2bDx]o}G_{fo}U_{yo} | |
| | A_{yo}G_{yo}A_{yo}U_{yo}U_{yo}U_{yo}U_{ys}A_{ys}A_{y} | |
| 1600515 | U_{ys}C_{ys}C_{yo}U_{yo}A_{yo}U_{[16C2r]o} | 10 |
| | G_{fo}A_{yo}C_{[f2bDx]o}U_{fo}G_{fo}U_{yo} | |
| | A_{yo}G_{yo}A_{yo}U_{yo}U_{yo}U_{yo}U_{ys}A_{ys}A_{y} | |

In the table above, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage. A subscript “[16C2r]” represents a 2′-O-hexadecylribosyl sugar moiety.

The antisense RNAi oligonucleotides of the designed RNAi agents described below are described herein above. Compound No. 1586324 is described herein above. The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). The sense RNAi oligomeric compounds contain a C16 conjugate, as indicated in the table below.

TABLE 28
Design of siRNA targeted to human/mouse HPRT1 containing
2′-fluoro-β-D-xylosyl nucleosides

| Duplex | Antisense Strand | Sense Strand |
|---|---|---|
| Compound No. | Compound No. | Compound No. |
| 1588822 | 1586322 | 1586324 |
| 1612512 | 1591246 | 1586324 |
| 1612513 | 1591248 | 1586324 |
| 1612514 | 1586322 | 1600514 |
| 1612515 | 1586322 | 1600515 |
| 1612516 | 1591246 | 1600514 |
| 1616032 | 1591248 | 1600515 |

### Example 11: Design of siRNA to HPRT1 Containing 3′-Fluoro-Hexitolnucleosides (F-HNA)

Modified oligonucleotides in the table below having either stereo-standard nucleosides or stereo-non-standard nucleosides in the antisense RNAi oligonucleotides and/or the sense RNAi oligomeric compound were synthesized using standard techniques. The following structure shows a 3′-fluoro-hexitol nucleoside (F-HNA), a nucleoside comprising a 3′-fluoro-tetrahydropyranose sugar surrogate), wherein Bx is a heterocyclic base moiety:

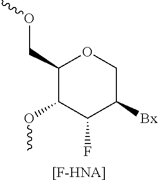

Each antisense RNAi oligonucleotide described in the table below is 100% complementary to SEQ ID NO: 14 (ENSEMBL Gene ID ENSMUSG00000025630.9, from ENSEMBL release 104: May 2021) from nucleosides 14094 to 14116, aside from a single mismatch at position 1 on the 5′ end of the antisense RNAi oligonucleotide.

TABLE 29
Design of antisense RNAi oligonucleotides
targeted to human/mouse HPRT1 containing
3′-fluoro-hexitolnucleosides

| | | SEQ |
|---|---|---|
| Compound | Chemistry Notation | ID |
| No. | (5′ to 3′) | NO. |
| 1586322 | vP-TₑₛU_{fs}A_{yo}A_{yo}A_{yo}A_{fo}U_{yo} | 9 |
| | C_{yo}U_{yo}A_{yo}C_{yo}A_{yo}G_{yo}U_{fo}C_{yo} | |
| | A_{fo}U_{yo}A_{yo}G_{yo}G_{yo}A_{ys}A_{ys}U_{y} | |
| 1591256 | vP-TₑₛU_{fs}A_{yo}A_{yo}A_{yo}A_{[F-HNA]o} | 9 |
| | U_{yo}C_{yo}U_{yo}A_{yo}C_{yo}A_{yo}G_{yo}U_{fo} | |
| | C_{yo}A_{fo}U_{yo}A_{yo}G_{yo}G_{yo}A_{ys}A_{ys}U_{y} | |
| 1591257 | vP-TₑₛU_{fs}A_{yo}A_{yo}A_{yo}A_{fo}U_{yo} | 9 |
| | C_{yo}U_{yo}A_{yo}C_{yo}A_{yo}G_{yo}U_{[F-HNA]o} | |
| | C_{yo}A_{fo}U_{yo}A_{yo}G_{yo}G_{yo}A_{ys}A_{ys}U_{y} | |
| 1591258 | vP-TₑₛU_{fs}A_{yo}A_{yo}A_{yo}A_{fo}U_{yo} | 9 |
| | C_{yo}U_{yo}A_{yo}C_{yo}A_{yo}G_{yo}U_{fo}C_{yo} | |
| | A_{[F-HNA]o}U_{yo}A_{yo}G_{yo}G_{yo}A_{ys} | |
| | A_{ys}U_{y} | |
| 1609650 | vP-TₑₛU_{fs}A_{yo}A_{yo}A_{yo}A_{fo}U_{yo} | 12 |
| | C_{yo}U_{yo}A_{yo}C_{yo}A_{yo}G_{yo}T_{[F-HNA]o} | |
| | C_{yo}A_{fo}U_{yo}A_{yo}G_{yo}G_{yo}A_{ys}A_{ys}U_{y} | |

In the table above, a subscript “[f2bDx]” represents a 2′-fluoro-β-D-xylosyl sugar moiety, a subscript “[F-HNA]” represents a 3′-fluoro-hexitol sugar surrogate, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “s” represents a phosphorothioate internucleoside linkage, and a subscript “o” represents a phosphodiester internucleoside linkage. Each antisense RNAi oligonucleotide contains a vinyl phosphonate (vP-) moiety on the 5′-end.

TABLE 30
Design of sense RNAi oligomeric compounds
targeted to human/mouse HPRT1 containing
3′-fluoro-hexitolnucleosides

| | | SEQ |
|---|---|---|
| Compound | Chemistry Notation | ID |
| No. | (5′ to 3′) | NO. |
| 1586324 | U_{ys}C_{ys}C_{yo}U_{yo}A_{yo}U_{[16C2r]o} | 10 |
| | G_{fo}A_{yo}C_{fo}U_{fo}G_{fo}U_{yo}A_{yo}G_{yo} | |
| | A_{yo}U_{yo}U_{yo}U_{yo}U_{ys}A_{ys}A_{y} | |
| 1591254 | U_{ys}C_{ys}C_{yo}U_{yo}A_{yo}U_{[16C2r]o} | 4 |
| | G_{[f2bDx]o}A_{yo}C_{fo}U_{fo}G_{fo}U_{yo} | |
| | A_{yo}G_{yo}A_{yo}U_{yo}U_{yo}U_{yo}U_{ys}A_{ys}U_{y} | |
| 1591255 | U_{ys}C_{ys}C_{yo}U_{yo}A_{yo}U_{[16C2r]o} | 4 |
| | G_{[f2bDx]o}A_{yo}C_{[f2bDx]o} | |
| | U_{[f2bDx]o}G_{[f2bDx]o}U_{yo}A_{yo} | |
| | G_{yo}A_{yo}U_{yo}U_{yo}U_{yo}U_{ys}A_{ys}U_{y} | |
| 1591260 | U_{ys}C_{ys}C_{yo}U_{yo}A_{yo}U_{[16C2r]o}G_{fo} | 4 |
| | A_{yo}C_{fo}U_{fo}G_{[F-HNA]o}U_{yo}A_{yo}G_{yo} | |
| | A_{yo}U_{yo}U_{yo}U_{yo}U_{ys}A_{ys}U_{y} | |
| 1591261 | U_{ys}C_{ys}C_{yo}U_{yo}A_{yo}U_{[16C2r]o}G_{fo} | 4 |
| | A_{yo}C_{fo}U_{[F-HNA]o}G_{fo}U_{yo}A_{yo}G_{yo} | |
| | A_{yo}U_{yo}U_{yo}U_{yo}U_{ys}A_{ys}U_{y} | |
| 1591262 | U_{ys}C_{ys}C_{yo}U_{yo}A_{yo}U_{[16C2r]o}G_{fo} | 4 |
| | A_{yo}C_{[F-HNA]o}U_{fo}G_{fo}U_{yo}A_{yo}G_{yo} | |
| | A_{yo}U_{yo}U_{yo}U_{yo}U_{ys}A_{ys}U_{y} | |
| 1601235 | U_{ys}C_{ys}C_{yo}U_{yo}A_{yo}U_{[16C2r]o}G_{fo} | 10 |
| | A_{yo}C_{fo}U_{fo}G_{[F-HNA]o}U_{yo}A_{yo}G_{yo} | |
| | A_{yo}U_{yo}U_{yo}U_{yo}U_{ys}A_{ys}A_{y} | |
| 1601238 | U_{ys}C_{ys}C_{yo}U_{yo}A_{yo}U_{[16C2r]o} | 10 |
| | G_{[F-HNA]o}A_{yo}C_{fo}U_{fo}G_{fo}U_{yo}A_{yo} | |
| | G_{yo}A_{yo}U_{yo}U_{yo}U_{yo}U_{ys}A_{ys}A_{y} | |
| 1601239 | U_{ys}C_{ys}C_{yo}U_{yo}A_{yo}U_{[16C2r]o} | 10 |
| | G_{[F-HNA]o}A_{yo}C_{fo}U_{fo}G_{[F-HNA]o} | |
| | U_{yo}A_{yo}G_{yo}A_{yo}U_{yo}U_{yo}U_{yo}U_{ys}A_{ys}A_{y} | |
| 1615237 | U_{ys}C_{ys}C_{yo}U_{yo}A_{yo}U_{[16C2r]o} | 13 |
| | G_{[F-HNA]o}A_{yo}^{m}C_{[F-HNA]o} | |
| | T_{[F-HNA]o}G_{[F-HNA]o}U_{yo}A_{yo}G_{yo} | |
| | A_{yo}U_{yo}U_{yo}U_{yo}U_{ys}A_{ys}A_{y} | |

In the table above, a subscript “[f2bDx]” represents a 2′-fluoro-β-D-xylosyl sugar moiety, a subscript “[F-HNA]” represents a 3′-fluoro-hexitol sugar surrogate, a subscript “[16C2r]” represents a 2′-O-hexadecylribosyl sugar moiety, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage. “^{m}C” in the table above represents a 5-methylcytosine.

TABLE 31
Design of siRNA targeted to human/mouse HPRT1
containing 3′-fluoro-hexitolnucleosides

| Duplex | Antisense Strand | Sense Strand |
|---|---|---|
| Compound No. | Compound No. | Compound No. |
| 1588822 | 1586322 | 1586324 |
| 1615555 | 1591256 | 1586324 |
| 1615556 | 1609650 | 1586324 |
| 1615557 | 1591258 | 1586324 |
| 1615558 | 1586322 | 1601235 |
| 1615559 | 1586322 | 1601238 |
| 1615560 | 1586322 | 1601239 |
| 1615561 | 1586322 | 1615237 |
| 1615562 | 1591256 | 1601239 |

### Example 12: In Vivo Activity of siRNA with Stereo-Standard Nucleosides and Stereo-Non-Standard Nucleosides in Wild-Type Mice

### In Vivo Study Design

The RNAi agents described above were tested in C57Bl6/J female mice. The mice were divided into groups of 2 mice each. Each mouse received a single ICV bolus of compound at 1, 10, 100, and 700 μg of RNAi agent and sacrificed two weeks later. A group of 4 mice received PBS as a negative control.

### RNA Analysis

After two weeks, mice were sacrificed, and RNA was extracted from cortex, thoracic cord, and liver for real-time PCR analysis of measurement of RNA expression of HPRT1 using primer-probe set RTS43125 (forward sequence CTCCTCAGACCGCTTTTTGC, designated herein as SEQ ID NO: 15; reverse sequence TAACCTGGTTCATCATCGCTAATC, designated herein as SEQ ID NO: 16; probe sequence CCGTCATGCCGACCCGCAGT, designated herein as SEQ ID NO: 17). Results are presented as percent mouse HPRT1 RNA relative to the amount in PBS treated mice (% ctrl), normalized to mouse cyclophilin A, measured by primer-probe set m_cyclo24 (forward sequence TCGCCGCTTGCTGCA, designated herein as SEQ ID NO: 18; reverse sequence ATCGGCCGTGATGTCGA, designated herein as SEQ ID NO: 19; probe sequence CCATGGTCAACCCCACCGTGTTC, designated herein as SEQ ID NO: 20).

N.D. in the table below refers to instances that no data was available. In the cases where there was not a significant dose-response effect, the ED₅₀ was not calculated (N.C.).

TABLE 32
Reduction of mouse HPRT1 RNA by siRNA containing 2′-fluoro-β-D-xylosyl nucleosides

| | | Cortex | | Thoracic Cord | | Liver | |
|---|---|---|---|---|---|---|---|
| | | HPRT1 | | HPRT1 | | HPRT1 | |
| Compound | Dose | RNA | ED₅₀ | RNA | ED₅₀ | RNA | ED₅₀ |
| No. | (μg) | (% ctrl) | (μg) | (% ctrl) | (μmol) | (% ctrl) | (μg) |
| PBS | N/A | 100 | N/A | 100 | N/A | 100 | N/A |
| 1588822 | 1 | 99 | 31 | 95 | 11 | 104 | 35 |
| | 10 | 75‡ | | 44 | | 79 | |
| | 100 | 23 | | 21 | | 23 | |
| | 700 | 7 | | 11 | | 9 | |
| 1612512 | 1 | 92 | 32 | 103 | 15 | 96 | 66 |
| | 10 | 87 | | 50 | | 92 | |
| | 100 | 14 | | 23 | | 37 | |
| | 700 | N.D. | | N.D. | | N.D. | |
| 1612513 | 1 | 97 | 41 | 105 | 75 | 104 | N.C. |
| | 10 | 78 | | 94 | | 103 | |
| | 100 | 31 | | 40 | | 62 | |
| | 700 | N.D. | | N.D. | | N.D. | |
| 1612514 | 1 | 104 | 47 | 96 | 20 | 98 | 52 |
| | 10 | 82 | | 57 | | 86 | |
| | 100 | 31 | | 25 | | 31 | |
| | 700 | 9 | | 13 | | 13 | |
| 1612515 | 1 | 97 | 32 | 100 | 50 | 116 | 51 |
| | 10 | 72 | | 83 | | 93 | |
| | 100 | 26 | | 32 | | 25 | |
| | 700 | 14 | | 14 | | 10 | |
| 1612516 | 1 | 75 | 21 | 80 | 14 | 102 | 117 |
| | 10 | 70 | | 57 | | 96 | |
| | 100 | 27 | | 24 | | 53 | |
| | 700 | 9 | | 11 | | 15 | |
| 1616032 | 1 | 87 | 98 | 109 | 65 | 109 | 76 |
| | 10 | 92 | | 81 | | 98 | |
| | 100 | 50 | | 40 | | 39 | |
| | 700 | 11‡ | | 17‡ | | 15‡ | |

‡indicates that fewer than 2 samples were available

### Example 13: In Vivo Activity of siRNA with Nucleosides Having F-HNA in Wild-Type Mice

### In Vivo Study Design

The RNAi agents described above were tested in C57Bl6/J female mice. The mice were divided into groups of 2 mice each. Each mouse received a single ICV bolus of compound at 1, 10, 100, and 700 μg of RNAi agent and sacrificed two weeks later. A group of 4 mice received PBS as a negative control.

### RNA Analysis

After two weeks, mice were sacrificed, and RNA was extracted from cortex, thoracic cord, and liver for real-time PCR analysis of measurement of RNA expression of HPRT1 using primer-probe set RTS43125 (described herein above). Results are presented as percent mouse HPRT1 RNA relative to the amount in PBS treated mice (% ctrl), normalized to mouse cyclophilin A, measured by primer-probe set m_cyclo24 (described herein above).

TABLE 33
Reduction of mouse HPRT1 RNA by siRNA containing 3′-fluoro-hexitolnucleosides

| | | Cortex | | Thoracic Cord | | Liver | |
|---|---|---|---|---|---|---|---|
| | | HPRT1 | | HPRT1 | | HPRT1 | |
| Compound | Dose | RNA | ED₅₀ | RNA | ED₅₀ | RNA | ED₅₀ |
| No. | (μg) | (% ctrl) | (μg) | (% ctrl) | (μmol) | (% ctrl) | (μg) |
| PBS | N/A | 100 | N/A | 100 | N/A | 100 | N/A |
| 1588822 | 1 | 99 | 31 | 95 | 11 | 104 | 35 |
| | 10 | 75‡ | | 44 | | 79 | |
| | 100 | 23 | | 21 | | 23 | |
| | 700 | 7 | | 11 | | 9 | |
| 1615555 | 1 | 98 | 63 | 104 | 40 | 98 | 74 |
| | 10 | 83 | | 80 | | 97 | |
| | 100 | 39 | | 25 | | 38 | |
| | 700 | 15 | | 17 | | 12 | |
| 1615556 | 1 | 86 | 27 | 87 | 29 | 91 | 41 |
| | 10 | 70 | | 68 | | 76 | |
| | 100 | 26 | | 29 | | 33 | |
| | 700 | 12 | | 17 | | 12 | |
| 1615557 | 1 | 82 | 73 | 96 | 67 | 97 | 57 |
| | 10 | 93 | | 75 | | 93 | |
| | 100 | 36 | | 31 | | 28 | |
| | 700 | 21 | | 36 | | 21 | |
| 1615558 | 1 | 66 | 22 | 73 | 20 | 94 | 52 |
| | 10 | 78 | | 72 | | 85 | |
| | 100 | 25 | | 20 | | 33 | |
| | 700 | 9 | | 13 | | 12 | |
| 1615559 | 1 | 104 | 56 | 104 | 15 | 107 | 74 |
| | 10 | 77 | | 50 | | 94 | |
| | 100 | 41 | | 20 | | 39 | |
| | 700 | 8 | | 13 | | 12 | |
| 1615560 | 1 | 99 | 19 | 76 | 17 | 104 | 103 |
| | 10 | 57 | | 64 | | 96 | |
| | 100 | 25 | | 24 | | 49 | |
| | 700 | 9 | | 14 | | 13 | |
| 1615561 | 1 | 109 | 236 | 76 | 124 | 104 | 248 |
| | 10 | 103 | | 79 | | 102 | |
| | 100 | 66 | | 49 | | 67 | |
| | 700 | 28 | | 35 | | 29 | |
| 1615562 | 1 | 123 | 211 | 108 | 156 | 117 | 343 |
| | 10 | 98 | | 88 | | 103 | |
| | 100 | 57 | | 47 | | 74 | |
| | 700 | 33 | | 36 | | 34 | |

‡indicates that fewer than 2 samples were available

### Example 14: Dose-Dependent Inhibition of Human/Mouse HPRT1 in Hela Cells by siRNA Containing 2′-Fluoro-β-D-Xylosyl Nucleosides

The RNAi agents described above were tested at various doses in HeLa cells. Cultured HeLa cells at a density of 8,000 cells per well were treated by RNAiMAX with various concentrations of siRNA as specified in the tables below. After a treatment period of approximately 6 hours, total RNA was isolated from the cells and HPRT1 RNA levels were measured by quantitative real-time RTPCR. Human HPRT1 primer-probe set RTS35336 (forward sequence TTGTTGTAGGATATGCCCTTGA, designated herein as SEQ ID NO: 21; reverse sequence GCGATGTCAATAGGACTCCAG, designated herein as SEQ ID NO: 22; probe sequence AGCCTAAGATGAGAGTTCAAGTTGAGTTTGG, designated herein as SEQ ID NO: 23) was used to measure RNA levels. HPRT1 RNA levels were normalized to total RNA content, as measured by RIBOGREEN®. Reduction of HPRT1 RNA is presented in the tables below as percent HPRT1 RNA, relative to untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+ (Top−Bottom)/(1+10\hat{ }((Log IC50−X)*HillSlope)), with the following constraints: Bottom=0, Top=100.

TABLE 34
Dose-dependent reduction of human HPRT1 RNA in Hela cells by siRNA containing
2′-fluoro-β-D-xylosyl nucleosides

| | HPRT1 RNA (% UTC) | | | | | | | | | | | | |
|---|---|---|---|---|---|---|---|---|---|---|---|---|---|
| Compound | 0.0006 | 0.0017 | 0.0051 | 0.0152 | 0.0457 | \|0.1372 | 0.4115 | 1.2346 | 3.7037 | 11.111 | 33.333 | 100 | IC₅₀ |
| No. | nM | nM | nM | nM | nM | nM | nM | nM | nM | nM | nM | nM | (nM) |
| 1588822 | 98 | 98 | 104 | 93 | 77 | 71 | 44 | 33 | 17 | 13 | 8 | 8 | 0.40 |
| 1612512 | 109 | 100 | 91 | 92 | 94 | 80 | 59 | 42 | 27 | 17 | 13 | 7 | 0.89 |
| 1612513 | 94 | 99 | 106 | 106 | 98 | 80 | 62 | 43 | 26 | 18 | 9 | 6 | 0.97 |
| 1612514 | 94 | 105 | 101 | 102 | 99 | 90 | 62 | 41 | 26 | 17 | 9 | 14 | 1.02 |
| 1612515 | 102 | 99 | 100 | 90 | 85 | 66 | 50 | 37 | 27 | 19 | 13 | 11 | 0.58 |
| 1612516 | 101 | 96 | 104 | 90 | 85 | 74 | 58 | 44 | 34 | 25 | 14 | 13 | 1.00 |
| 1616032 | 87 | 104 | 108 | 74 | 102 | 67 | 62 | 43 | 31 | 22 | 16 | 10 | 0.97 |

### Example 15: Dose-Dependent Inhibition of Human HPRT1 in Hela Cells by siRNA Containing 3′-fluoro-hexitolnucleosides

The RNAi agents described above were tested at various doses in HeLa cells. Cultured HeLa cells at a density of 8,000 cells per well were treated by RNAiMAX with various concentrations of siRNA as specified in the tables below. After a treatment period of approximately 6 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to untreated control cells (% UTC). N.D. in the table below refers to instance(s) where the value was Not Defined.

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100.

TABLE 35
Dose-dependent reduction of human HPRT1 RNA in HeLa cells by siRNA containing 3′-fluoro-hexitolnucleosides

| | HPRT1 RNA (% UTC) | | | | | | | | | | | | |
|---|---|---|---|---|---|---|---|---|---|---|---|---|---|
| Compound | 0.0006 | 0.0017 | 0.0051 | 0.0152 | 0.0457 | 0.1372 | 0.4115 | 1.2346 | 3.7037 | 11.111 | 33.333 | 100 | IC₅₀ |
| No. | nM | nM | nM | nM | nM | nM | nM | nM | nM | nM | nM | nM | (nM) |
| 1588822 | 98 | 98 | 104 | 93 | 77 | 71 | 44 | 33 | 17 | 13 | 8 | 8 | 0.40 |
| 1615555 | 101 | 107 | 92 | 97 | 98 | 75 | 72 | 51 | 30 | 22 | 15 | 10 | 1.39 |
| 1615556 | 92 | 99 | 109 | 90 | 91 | 81 | 68 | 51 | 37 | 24 | 19 | 6 | 1.57 |
| 1615557 | 109 | 95 | 96 | 96 | 91 | 86 | 67 | 52 | 36 | 26 | 17 | 11 | 1.68 |
| 1615558 | 95 | 102 | 103 | 103 | 92 | 80 | 68 | 48 | 36 | 24 | 15 | 22 | 1.59 |
| 1615559 | 97 | 102 | 101 | 103 | 80 | 67 | 64 | 43 | 30 | 21 | 14 | 16 | 0.92 |
| 1615560 | 99 | 102 | 99 | 103 | 86 | 83 | 70 | 61 | 43 | 38 | 28 | N.D. | 3.17 |
| 1615561 | 131 | 85 | 85 | 82 | 74 | 76 | 67 | 71 | 64 | 61 | 61 | 69 | >100 |
| 1615562 | 128 | 83 | 89 | 87 | 84 | 77 | 78 | 76 | 71 | 65 | 65 | 54 | >100 |

### Example 16: Dose-Dependent Inhibition of Human HPRT1 in Hela Cells by siRNA Containing Chiral Mesyl Phosphoramidate Linkages

RNAi agents described above were tested at various doses in HeLa cells. Cultured HeLa cells at a density of 8,000 cells per well were treated by RNAiMAX with various concentrations of siRNA as specified in the tables below. After a treatment period of approximately 6 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100.

TABLE 36
Dose-dependent reduction of human HPRT1 RNA in Hela cells by siRNA containing chiral mesyl
phosphoramidate linkages

| | HPRT1 RNA (% UTC) | | | | | | | | | | | | |
|---|---|---|---|---|---|---|---|---|---|---|---|---|---|
| Compound | 0.0006 | 0.0017 | 0.0051 | 0.0152 | 0.0457 | 0.1372 | 0.4115 | 1.2346 | 3.7037 | 11.111 | 33.333 | 100 | IC₅₀ |
| No. | nM | nM | nM | nM | nM | nM | nM | nM | nM | nM | nM | nM | (nM) |
| 1590260 | 93 | 100 | 107 | 112 | 84 | 80 | 69 | 56 | 49 | 27 | 8 | 5 | 1.98 |
| 1590261 | 90 | 97 | 113 | 90 | 84 | 81 | 74 | 54 | 72 | 42 | 11 | 7 | 3.70 |
| 1590262 | 110 | 97 | 94 | 96 | 87 | 80 | 79 | 85 | 53 | 40 | 14 | 7 | 4.66 |
| 1590263 | 115 | 89 | 96 | 94 | 82 | 68 | 60 | 46 | 37 | 22 | 7 | 5 | 0.89 |
| 1590264 | 95 | 111 | 94 | 96 | 96 | 72 | 67 | 46 | 40 | 26 | 7 | 5 | 1.30 |
| 1590265 | 89 | 109 | 101 | 105 | 111 | 90 | 72 | 70 | 49 | 33 | 9 | 6 | 3.29 |
| 1590266 | 91 | 110 | 99 | 107 | 71 | 59 | 70 | 71 | 39 | 30 | 12 | 6 | 1.70 |
| 1590267 | 92 | 112 | 96 | 106 | 88 | 90 | 102 | 77 | 48 | 41 | 22 | 11 | 5.68 |
| 1590268 | 94 | 109 | 97 | 112 | 95 | 93 | 79 | 67 | 55 | 31 | 9 | 4 | 3.49 |
| 1590269 | 100 | 106 | 94 | 92 | 100 | 69 | 58 | 49 | 37 | 25 | 8 | 6 | 1.13 |
| 1590270 | 99 | 107 | 95 | 98 | 90 | 82 | 76 | 76 | 58 | 42 | 18 | 7 | 4.84 |
| 1590271 | 91 | 105 | 104 | 99 | 100 | 88 | 78 | 62 | 45 | 33 | 12 | 7 | 2.86 |

### Example 17: Design of siRNA to Mouse FXII with Mesyl Phosphoramidate Internucleoside Linkages

Modified oligonucleotides in the table below having mesyl phosphoramidate internucleoside linkages in the antisense RNAi oligonucleotides and/or the sense RNAi oligonucleotide were synthesized using standard techniques.

Each antisense RNAi oligonucleotide described in the table below has the sequence UAAAGCACUUUAUUGAGUUUCUG (SEQ ID NO: 24). Aside from a single mismatch at position 1 on the 5′-end, each antisense RNAi oligonucleotide is 100% complementary to the complement of GenBank Accession No. NC_000079.6, truncated from nucleosides 55415001 to 55430000, (SEQ ID NO: 25) from nucleosides 12005 to 12026 (SEQ ID NO: 39).

TABLE 37
Design of antisense RNAi oligonucleotides
targeted to mouse FXII containing mesyl
phosphoramidate internucleoside linkages

| | | SEQ |
|---|---|---|
| Compound | Chemistry Notation | ID |
| No. | (5′ to 3′) | NO. |
| 1523579 | U_{ys}A_{fs}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{ys}U_{ys}G_{y} | |
| 1601822 | U_{yz}A_{fs}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{yz}U_{ys}G_{y} | |
| 1601823 | U_{yz}A_{fs}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{ys}U_{yz}G_{y} | |
| 1601824 | U_{ys}A_{fz}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{yz}U_{ys}G_{y} | |
| 1601962 | U_{ys}A_{fz}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{ys}U_{yz}G_{y} | |
| 1528440 | Z.U_{ys}A_{fs}A_{yo}A_{yo}G_{yo}C_{fo}A_{yo} | 24 |
| | C_{yo}U_{yo}U_{yo}U_{yo}A_{yo}U_{yo}U_{fo}G_{yo} | |
| | A_{fo}G_{yo}U_{yo}U_{yo}U_{yo}C_{ys}U_{ys}G_{y} | |
| 1601963 | U_{yz}A_{fs}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{ys}U_{ys}G_{y} | |
| 1601964 | U_{ys}A_{fz}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{ys}U_{ys}G_{y} | |
| 1601965 | U_{ys}A_{fs}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{yz}U_{ys}G_{y} | |
| 1601966 | U_{ys}A_{fs}A_{yo}A_{yo}G_{yo}C_{fo}A_{yo}C_{yo} | 24 |
| | U_{yo}U_{yo}U_{yo}A_{yo}U_{yo}U_{fo}G_{yo}A_{fo} | |
| | G_{yo}U_{yo}U_{yo}U_{yo}C_{ys}U_{yz}G_{y} | |
| 1599527 | Z.U_{yz}A_{fs}A_{yo}A_{yo}G_{yo}C_{fo}A_{yo} | 24 |
| | C_{yo}U_{yo}U_{yo}U_{yo}A_{yo}U_{yo}U_{fo}G_{yo} | |
| | A_{fo}G_{yo}U_{yo}U_{yo}U_{yo}C_{ys}U_{ys}G_{y} | |

In the table above, a “z,” represents a 5′-mesyl phosphoramidate terminal group, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, a subscript “z” represents a mesyl phosphoramidate internucleoside linkage, and a subscript “o” represents a phosphodiester internucleoside linkage.

The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Each sense oligomeric compound further contains a GalNAc moiety conjugated to the 3′-oxygen as shown below:

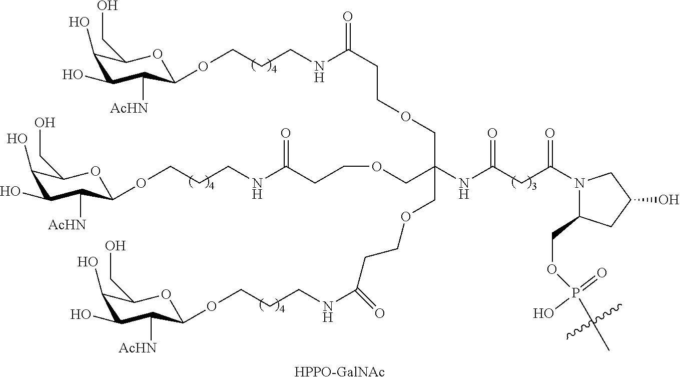

TABLE 38
Design of sense RNAi oligomeric compounds
targeted to mouse FXII containing mesyl
phosphoramidate internucleoside linkages

| | | SEQ |
|---|---|---|
| Compound | Chemistry Notation | ID |
| No. | (5′ to 3′) | NO. |
| 1523578 | G_{ys}A_{ys}A_{yo}A_{yo}C_{yo}U_{yo}C_{fo}A_{yo} | 26 |
| | A_{fo}U_{fo}A_{fo}A_{yo}A_{yo}G_{yo}U_{yo}G_{yo} | |
| | C_{yo}U_{yo}U_{yo}U_{yo}A_{y}-HPPO-GalNAc | |
| 1526458 | G_{yz}A_{yz}A_{yo}A_{yo}C_{yo}U_{yo}C_{fo}A_{yo} | 26 |
| | A_{fo}U_{fo}A_{fo}A_{yo}A_{yo}G_{yo}U_{yo}G_{yo} | |
| | C_{yo}U_{yo}U_{yo}U_{yo}A_{y}-HPPO-GalNAc | |
| 1599528 | G_{yz}A_{ys}A_{yo}A_{yo}C_{yo}U_{yo}C_{fo}A_{yo} | 26 |
| | A_{fo}U_{fo}A_{fo}A_{yo}A_{yo}G_{yo}U_{yo}G_{yo} | |
| | C_{yo}U_{yo}U_{ys}U_{yz}A_{y}-HPPO-GalNAc | |

In the table above, a subscript “f” represents a 2′-fluoro-β-D-ribosyl sugar moiety, a subscript “y” represents a 2′-OMe-β-D-ribosyl sugar moiety, a subscript “s” represents a phosphorothioate internucleoside linkage, a subscript “z” represents a mesyl phosphoramidate internucleoside linkage, and a subscript “o” represents a phosphodiester internucleoside linkage.

TABLE 39
Design of siRNA targeted to mouse FXII containing
mesyl phosphoramidate internucleoside linkages

| Duplex | Antisense Strand | Sense Strand |
|---|---|---|
| Compound No. | Compound No. | Compound No. |
| 1523582 | 1523579 | 1523578 |
| 1610011 | 1601822 | 1523578 |
| 1610012 | 1601823 | 1523578 |
| 1610013 | 1601824 | 1523578 |
| 1610014 | 1601962 | 1523578 |
| 1529980 | 1528440 | 1523578 |
| 1610015 | 1601963 | 1523578 |
| 1610016 | 1601964 | 1523578 |
| 1610019 | 1601965 | 1523578 |
| 1610020 | 1601966 | 1523578 |
| 1610021 | 1599527 | 1523578 |
| 1529979 | 1523579 | 1526458 |
| 1610022 | 1523579 | 1599528 |

### Example 18: In Vivo Activity of siRNA with Mesyl Phosphoramidate Internucleoside Linkages in Wild-Type Mice

### In Vivo Study Design

In vivo studies were carried out to evaluate whether mesyl phosphoramidate internucleoside linkages improved potency of RNAi agents. RNAi agents described above were tested in C57Bl6/J male mice. The mice were divided into groups of 4 mice each. Each mouse received a single subcutaneous injection of RNAi agent at a dose of 1 mg/kg and sacrificed one week later. A group of 4 mice received PBS as a negative control.

### RNA Analysis

After one week, mice were sacrificed, and RNA was extracted from liver for quantitative RTPCR analysis of measurement of RNA expression of FXII using primer-probe set RTS2959 (forward sequence CAAAGGAGGGACATGTATCAACAC, designated herein as SEQ ID NO: 27; reverse sequence CTGGCAATGTTTCCCAGTGA, designated as herein SEQ ID NO: 28; probe sequence CCCAATGGGCCACACTGTCTCTGC, designated herein as SEQ ID NO: 29). Results are presented as percent mouse FXII RNA relative to the amount in PBS treated mice (% control), normalized to mouse cyclophilin A, measured by primer-probe set m_cyclo24 (described herein above).

TABLE 40
Reduction of mouse FXII RNA by siRNA with mesyl
phosphoramidate internucleoside linkages

| | FXII RNA |
|---|---|
| Compound No. | (% control) |
| PBS | 100 |
| 1523582 | 19 |
| 1610011 | 57 |
| 1610012 | 87 |
| 1610013 | 13 |
| 1610014 | 23 |
| 1529980 | 16 |
| 1610015 | 50 |
| 1610016 | 16 |
| 1610019 | 15 |
| 1610020 | 21 |
| 1610021 | 34 |
| 1529979 | 17 |
| 1610022 | 22 |

### Example 19: In Vivo Duration of Action of siRNA with Mesyl Phosphoramidate Internucleoside Linkages in Wild-Type Mice

### In Vivo Study Design

In vivo studies were carried out to evaluate whether mesyl phosphoramidate internucleoside linkages affected duration of action of RNAi agents. The RNAi agents described above were tested in C57Bl6/J male mice. The mice were divided into groups of 4 mice each. Each mouse received a single subcutaneous injection of RNAi agent at a dose of 1 mg/kg. A group of 4 mice received PBS as a negative control. Prior to the first dose, a tail bleed was performed to determine plasma FXII protein levels at baseline (BL). Tail bleeds were also performed at 1, 2, 4, 6, and 8 weeks following the dose.

### Protein Analysis

Mouse FXII protein levels in plasma were determined using a FXII ELISA kit (Molecular Innovations catalog number: MFXIIKT-TOT). Results are presented in Table 39 as percent change from baseline within each treatment group (% baseline).

TABLE 41
Reduction of mouse FXII RNA by siRNA with mesyl phosphoramidate internucleoside
linkages at various time points

| | FXII protein (% baseline) in plasma at indicated time after injection | | | | | |
|---|---|---|---|---|---|---|
| Compound | Day 0 | | | | | |
| No. | (baseline) | 1 week | 2 weeks | 4 weeks | 6 weeks | 8 weeks |
| PBS | 100 | 73 | 83 | 95 | 103 | 109 |
| 1523582 | 100 | 8 | 12 | 27 | 67 | 98 |
| 1610011 | 100 | 26 | 28 | 29 | 79 | 93 |
| 1610012 | 100 | 79 | 76 | 95 | 117 | 114 |
| 1610013 | 100 | 5 | 6 | 11 | 35 | 63 |
| 1610014 | 100 | 8 | 13 | 26 | 62 | 89 |
| 1529980 | 100 | 8 | 8 | 22 | 50 | 80 |
| 1610015 | 100 | 41 | 33 | 42 | 71 | 99 |
| 1610016 | 100 | 5 | 9 | 16 | 52 | 80 |
| 1610019 | 100 | 11 | 7 | 25 | 50 | 81 |
| 1610020 | 100 | 27 | 12 | 20 | 44 | 75 |
| 1610021 | 100 | 19 | 25 | 32 | 64 | 82 |
| 1529979 | 100 | 10 | 15 | 47 | 89 | 108 |
| 1610022 | 100 | 20 | 16 | 31 | 63 | 87 |

### Example 20: Design of siRNA Targeted to HPRT1 Containing 2′-O-Methyl Nucleosides in the Sense RNAi Oligonucleotide

Modified oligonucleotides in the table below having either stereo-standard nucleosides or stereo-non-standard nucleosides in the antisense RNAi oligonucleotides and/or the sense RNAi oligonucleotide were synthesized using standard techniques. Each antisense RNAi oligonucleotide described in the table below has the sequence UUAAAAUCUACAGUCAUAGGAAU (SEQ ID NO: 30) and is complementary to human HPRT GenBank Accession No. NM_000194.2 (SEQ ID NO: 1, described herein above) from nucleosides 444 to 465, with a single mismatch at position 1 on the 5′ end of the antisense RNAi oligonucleotide. Each antisense RNAi oligonucleotide has a 5′-phosphate.

TABLE 42
Design of antisense RNAi oligonucleotides
targeted to HPRT1

| | | SEQ |
|---|---|---|
| Compound | Chemistry Notation | ID |
| No. | (5′ to 3′) | NO. |
| 1601968 | p.U_{ys}U_{fs}A_{yo}A_{yo}A_{yo}A_{fo}U_{yo}C_{yo} | 30 |
| | U_{yo}A_{yo}C_{yo}A_{yo}G_{yo}U_{fo}C_{yo}A_{fo}U_{yo} | |
| | A_{yo}G_{yo}G_{yo}A_{ys}A_{ys}U_{y} | |
| 1616886 | p.U_{ys}U_{fs}A_{yo}A_{fo}A_{yo}A_{fo}U_{yo}C_{fo} | 30 |
| | U_{yo}A_{fo}C_{yo}A_{fo}G_{yo}U_{fo}C_{yo}A_{fo}U_{yo} | |
| | A_{fo}G_{yo}G_{fo}A_{ys}A_{fs}U_{y} | |

In the table above, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage.

The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Compound No. 1586323 is described herein above.

TABLE 43
Design of sense RNAi oligonucleotides

| | | SEQ |
|---|---|---|
| Compound | Chemistry Notation | ID |
| No. | (5′ to 3′) | NO. |
| 1616428 | U_{ys}C_{ys}C_{yo}U_{yo}A_{yo}U_{yo}G_{yo}A_{yo}C_{yo} | 10 |
| | U_{yo}G_{yo}U_{yo}A_{yo}G_{yo}A_{yo}U_{yo}U_{yo}U_{yo} | |
| | U_{ys}A_{ys}A_{y} | |
| 1616429 | U_{ys}C_{ys}C_{yo}U_{yo}A_{yo}U_{yo}G_{yo}A_{yo}C_{fo} | 10 |
| | U_{fo}G_{fo}U_{yo}A_{yo}G_{yo}A_{yo}U_{yo}U_{yo}U_{yo} | |
| | U_{ys}A_{ys}A_{y} | |
| 1616430 | U_{ys}C_{ys}C_{yo}U_{yo}A_{yo}U_{yo}G_{fo}A_{yo}C_{yo} | 10 |
| | U_{fo}G_{fo}U_{yo}A_{yo}G_{yo}A_{yo}U_{yo}U_{yo}U_{yo} | |
| | U_{ys}A_{ys}A_{y} | |
| 1616431 | U_{ys}C_{ys}C_{yo}U_{yo}A_{yo}U_{yo}G_{fo}A_{yo}C_{fo} | 10 |
| | U_{yo}G_{fo}U_{yo}A_{yo}G_{yo}A_{yo}U_{yo}U_{yo}U_{yo} | |
| | U_{ys}A_{ys}A_{y} | |
| 1616432 | U_{ys}C_{ys}C_{yo}U_{yo}A_{yo}U_{yo}G_{fo}A_{yo}C_{fo} | 10 |
| | U_{fo}G_{yo}U_{yo}A_{yo}G_{yo}A_{yo}U_{yo}U_{yo}U_{yo} | |
| | U_{ys}A_{ys}A_{y} | |
| 1616443 | U_{ys}C_{ys}C_{yo}U_{yo}A_{yo}U_{yo}G_{yo}A_{yo}C_{yo} | 10 |
| | U_{fo}G_{fo}U_{yo}A_{yo}G_{yo}A_{yo}U_{yo}U_{yo}U_{yo} | |
| | U_{ys}A_{ys}A_{y} | |
| 1616444 | U_{ys}C_{ys}C_{yo}U_{yo}A_{yo}U_{yo}G_{fo}A_{yo}C_{yo} | 10 |
| | U_{yo}G_{fo}U_{yo}A_{yo}G_{yo}A_{yo}U_{yo}U_{yo}U_{yo} | |
| | U_{ys}A_{ys}A_{y} | |
| 1616445 | U_{ys}C_{ys}C_{yo}U_{yo}A_{yo}U_{yo}G_{fo}A_{yo}C_{fo} | 10 |
| | U_{yo}G_{yo}U_{yo}A_{yo}G_{yo}A_{yo}U_{yo}U_{yo}U_{yo} | |
| | U_{ys}A_{ys}A_{y} | |
| 1616446 | U_{ys}C_{ys}C_{yo}U_{yo}A_{yo}U_{yo}G_{yo}A_{yo}C_{fo} | 10 |
| | U_{yo}G_{fo}U_{yo}A_{yo}G_{yo}A_{yo}U_{yo}U_{yo}U_{yo} | |
| | U_{ys}A_{ys}A_{y} | |
| 1616447 | U_{ys}C_{ys}C_{yo}U_{yo}A_{yo}U_{yo}G_{yo}A_{yo}C_{fo} | 10 |
| | U_{fo}G_{yo}U_{yo}A_{yo}G_{yo}A_{yo}U_{yo}U_{yo}U_{yo} | |
| | U_{ys}A_{ys}A_{y} | |
| 1616480 | U_{ys}C_{ys}C_{yo}U_{yo}A_{yo}U_{yo}G_{fo}A_{yo}C_{yo} | 10 |
| | U_{fo}G_{yo}U_{yo}A_{yo}G_{yo}A_{yo}U_{yo}U_{yo}U_{yo} | |
| | U_{ys}A_{ys}A_{y} | |
| 1616887 | U_{fs}C_{ys}C_{fo}U_{yo}A_{fo}U_{yo}G_{fo}A_{yo}C_{fo} | 10 |
| | U_{yo}G_{fo}U_{yo}A_{fo}G_{yo}A_{fo}U_{yo}U_{fo}U_{yo} | |
| | U_{fs}A_{ys}A_{f} | |

In the table above, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage.

TABLE 44
Design of siRNA targeted to HPRT1
containing 2′-O-methyl nucleosides

| Duplex | Antisense Strand | Sense Strand |
|---|---|---|
| Compound No. | Compound No. | Compound No. |
| 1640504 | 1601968 | 1586323 |
| 1640505 | 1601968 | 1616428 |
| 1640506 | 1601968 | 1616429 |
| 1640507 | 1601968 | 1616430 |
| 1640508 | 1601968 | 1616431 |
| 1640509 | 1601968 | 1616432 |
| 1640510 | 1601968 | 1616443 |
| 1640511 | 1601968 | 1616444 |
| 1640512 | 1601968 | 1616445 |
| 1640513 | 1601968 | 1616446 |
| 1640514 | 1601968 | 1616447 |
| 1640515 | 1601968 | 1616480 |
| 1647742 | 1616886 | 1616887 |

### Example 21: Dose-Dependent Inhibition of Human HPRT1 in A431 Cells by siRNA Containing 2′-O-Methyl Nucleosides in the Sense RNAi Oligonucleotide

The RNAi agents described above were tested at various doses in A431 cells. Cultured A431 cells at a density of 10,000 cells per well were treated by RNAiMAX with siRNA at concentrations indicated in the table below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment. N.C. refers to data points that were not calculated.

TABLE 45
Dose-dependent reduction of human HPRT1 RNA in A431 cells by siRNA containing
2′-O-methyl nucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 3 | 6 | 33 | 77 | 84 | 105 | 3.89 |
| 1647742 | 4 | 3 | 3 | 8 | 41 | 105 | 125 | 121 | 9.38 |
| 1640505 | N.C. | 19 | 13 | 46 | 87 | 113 | 119 | 120 | 95.11 |
| 1640506 | 3 | 2 | 3 | 6 | 27 | 72 | 106 | 101 | 3.33 |
| 1640507 | 4 | 3 | 3 | 7 | 42 | 94 | 106 | 107 | 8.05 |
| 1640508 | 3 | 3 | 4 | 17 | 83 | 99 | 100 | 97 | 31.50 |
| 1640509 | 3 | 2 | 3 | 8 | 39 | 87 | 107 | 110 | 6.70 |
| 1640510 | 6 | 3 | 3 | 7 | 41 | 84 | 99 | 122 | 6.60 |
| 1640511 | 33 | 6 | 7 | 31 | 78 | 91 | 90 | 101 | 39.84 |

TABLE 46
Dose-dependent reduction of human HPRT1 RNA in A431 cells by siRNA containing
2′-O-methyl nucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 2 | 4 | 29 | 77 | 95 | 101 | 3.67 |
| 1640512 | 10 | 4 | 5 | 19 | 78 | 116 | 130 | 117 | 30.27 |
| 1640513 | 3 | 4 | 3 | 12 | 75 | 100 | 119 | 109 | 22.49 |
| 1640514 | 4 | 2 | 3 | 7 | 50 | 96 | 114 | 118 | 10.21 |
| 1640515 | N.C. | 7 | 6 | 24 | 92 | 94 | 114 | 122 | 4.62 |

### Example 22: Design of siRNA Targeted to HPRT1 Containing 2′-MOE Nucleosides in the Sense RNAi Oligonucleotide

Modified oligonucleotides in the table below having either stereo-standard nucleosides or stereo-non-standard nucleosides in the antisense RNAi oligonucleotides and/or the sense RNAi oligonucleotide were synthesized using standard techniques. antisense RNAi oligonucleotide Compound No. 1601968 is described herein above

The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

TABLE 47
Design of sense RNAi oligonucleotides
containing 2′-MOE nucleosides

| | | SEQ |
|---|---|---|
| Compound | Chemistry Notation | ID |
| No. | (5′ to 3′) | NO. |
| 1616481 | U_{ys}C_{ys}C_{yo}U_{yo}A_{yo}U_{yo}GₑₒA_{yo} | 10 |
| | CₑₒUₑₒGₑₒU_{yo}A_{yo}G_{yo}A_{yo}U_{yo} | |
| | U_{yo}U_{yo}U_{ys}A_{ys}A_{y} | |
| 1616482 | U_{ys}C_{ys}C_{yo}U_{yo}A_{yo}U_{yo}GₑₒA_{yo} | 10 |
| | C_{fo}U_{fo}G_{fo}U_{yo}A_{yo}G_{yo}A_{yo}U_{yo} | |
| | U_{yo}U_{yo}U_{ys}A_{ys}A_{y} | |
| 1616483 | U_{ys}C_{ys}C_{yo}U_{yo}A_{yo}U_{yo}G_{fo}A_{yo} | 10 |
| | CₑₒU_{fo}G_{fo}U_{yo}A_{yo}G_{yo}A_{yo}U_{yo} | |
| | U_{yo}U_{yo}U_{ys}A_{ys}A_{y} | |
| 1616484 | U_{ys}C_{ys}C_{yo}U_{yo}A_{yo}U_{yo}G_{fo}A_{yo} | 10 |
| | C_{fo}UₑₒG_{fo}U_{yo}A_{yo}G_{yo}A_{yo}U_{yo} | |
| | U_{yo}U_{yo}U_{ys}A_{ys}A_{y} | |
| 1616485 | U_{ys}C_{ys}C_{yo}U_{yo}A_{yo}U_{yo}G_{fo}A_{yo} | 10 |
| | C_{fo}U_{fo}GₑₒU_{yo}A_{yo}G_{yo}A_{yo}U_{yo} | |
| | U_{yo}U_{yo}U_{ys}A_{ys}A_{y} | |
| 1616486 | U_{ys}C_{ys}C_{yo}U_{yo}A_{yo}U_{yo}GₑₒA_{yo} | 10 |
| | CₑₒU_{fo}G_{fo}U_{yo}A_{yo}G_{yo}A_{yo}U_{yo} | |
| | U_{yo}U_{yo}U_{ys}A_{ys}A_{y} | |
| 1616487 | U_{ys}C_{ys}C_{yo}U_{yo}A_{yo}U_{yo}G_{fo}A_{yo} | 10 |
| | CₑₒUₑₒG_{fo}U_{yo}A_{yo}G_{yo}A_{yo}U_{yo} | |
| | U_{yo}U_{yo}U_{ys}A_{ys}A_{y} | |
| 1616566 | U_{ys}C_{ys}C_{yo}U_{yo}A_{yo}U_{yo}G_{fo}A_{yo} | 10 |
| | C_{fo}UₑₒGₑₒU_{yo}A_{yo}G_{yo}A_{yo}U_{yo} | |
| | U_{yo}U_{yo}U_{ys}A_{ys}A_{y} | |
| 1616567 | U_{ys}C_{ys}C_{yo}U_{yo}A_{yo}U_{yo}GₑₒA_{yo} | 10 |
| | C_{fo}UₑₒG_{fo}U_{yo}A_{yo}G_{yo}A_{yo}U_{yo} | |
| | U_{yo}U_{yo}U_{ys}A_{ys}A_{y} | |
| 1616568 | U_{ys}C_{ys}C_{yo}U_{yo}A_{yo}U_{yo}GₑₒA_{yo} | 10 |
| | C_{fo}U_{fo}GₑₒU_{yo}A_{yo}G_{yo}A_{yo}U_{yo} | |
| | U_{yo}U_{yo}U_{ys}A_{ys}A_{y} | |
| 1616569 | U_{ys}C_{ys}C_{yo}U_{yo}A_{yo}U_{yo}G_{fo}A_{yo} | 10 |
| | CₑₒU_{fo}GₑₒU_{yo}A_{yo}G_{yo}A_{yo}U_{yo} | |
| | U_{yo}U_{yo}U_{ys}A_{ys}A_{y} | |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “s” represents a phosphorothioate internucleoside linkage, and a subscript “o” represents a phosphodiester internucleoside linkage.

TABLE 48
Design of siRNA targeted to HPRT1
containing 2′- MOE nucleosides

| Duplex | Antisense Strand | Sense Strand |
|---|---|---|
| Compound No. | Compound No. | Compound No. |
| 1642105 | 1601968 | 1616481 |
| 1642106 | 1601968 | 1616482 |
| 1642108 | 1601968 | 1616483 |
| 1642109 | 1601968 | 1616484 |
| 1642110 | 1601968 | 1616485 |
| 1642111 | 1601968 | 1616486 |
| 1642112 | 1601968 | 1616487 |
| 1642113 | 1601968 | 1616566 |
| 1642114 | 1601968 | 1616567 |
| 1642115 | 1601968 | 1616568 |
| 1642116 | 1601968 | 1616569 |

### Example 23: Dose-Dependent Inhibition of Human HPRT1 in A431 Cells by siRNA Containing 2′-MOE Nucleosides in the Sense RNAi Oligonucleotide

The RNAi agents described above were tested at various doses in A431 cells. Cultured A431 cells at a density of 10,000 cells per well were treated by RNAiMAX with siRNA at concentrations indicated in the table below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment. N.C. refers to data points that were not calculated.

49: Dose-Dependent Reduction of Human HPRT1 RNA in A431 Cells by siRNA Containing 2′-MOE Nucleosides in the Sense RNAi Oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 2 | 4 | 29 | 77 | 95 | 101 | 3.67 |
| 1642105 | N.C. | 17 | 9 | 32 | 78 | 101 | 101 | 110 | 45.50 |
| 1642106 | 2 | 2 | 2 | 5 | 43 | 78 | 106 | 101 | 6.19 |
| 1642108 | 3 | 2 | 4 | 9 | 55 | 84 | 114 | 110 | 11.13 |

TABLE 50
Dose-dependent reduction of human HPRT1 RNA in A431 cells by siRNA containing
2′-MOE nucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 3 | 4 | 23 | 64 | 94 | 92 | 2.13 |
| 1642109 | 7 | 3 | 6 | 18 | 71 | 106 | 103 | 115 | 24.81 |
| 1642110 | 3 | 3 | 3 | 7 | 45 | 91 | 110 | 110 | 8.48 |
| 1642111 | 4 | 3 | 4 | 9 | 47 | 96 | 97 | 93 | 9.31 |
| 1642112 | N.C. | 7 | 7 | 24 | 84 | 101 | 87 | 105 | 39.43 |
| 1642113 | 15 | 4 | 5 | 26 | 84 | 94 | 96 | 94 | 40.17 |
| 1642114 | 9 | 4 | 5 | 13 | 80 | 89 | 105 | 86 | 26.34 |
| 1642115 | 9 | 4 | 4 | 15 | 55 | 86 | 105 | 91 | 12.41 |
| 1642116 | N.C. | 12 | 6 | 18 | 68 | 98 | 93 | 91 | 22.55 |

### Example 24: Design of siRNA Targeted to HPRT1 Containing 2′-Deoxyribonucleosides in the Antisense RNAi Oligonucleotide

Modified oligonucleotides in the table below having either stereo-standard nucleosides or stereo-non-standard nucleosides in the antisense RNAi oligonucleotides and/or the sense RNAi oligonucleotide were synthesized using standard techniques. Each antisense RNAi oligonucleotide described in the table below has the sequence UUAAAAUCUACAGUCAUAGGAAU (SEQ ID NO: 30) and is complementary to human HPRT GenBank Accession No. NM_000194.2 (SEQ ID NO: 1, described herein above) from nucleosides 444 to 465, with a single mismatch at position 1 on the 5′ end of the antisense RNAi oligonucleotide. Each antisense RNAi oligonucleotide has a 5′-phosphate.

TABLE 51
Design of antisense RNAi oligonucleotides
targeted to HPRT1 containing deoxyribosyl
nucleosides

| | | SEQ |
|---|---|---|
| Compound | Chemistry Notation | ID |
| No. | (5′ to 3′) | NO. |
| 1616680 | p.U_{ys}U_{fs}A_{yo}A_{yo}A_{yo}A_{do}U_{yo} | 30 |
| | C_{yo}U_{yo}A_{yo}C_{yo}A_{yo}G_{yo}U_{fo}C_{yo} | |
| | A_{fo}U_{yo}A_{yo}G_{yo}G_{yo}A_{ys}A_{ys}U_{y} | |
| 1616681 | p.U_{ys}U_{fs}A_{yo}A_{yo}A_{yo}A_{fo}U_{yo} | 30 |
| | C_{yo}U_{yo}A_{yo}C_{yo}A_{yo}G_{yo}U_{do}C_{yo} | |
| | A_{fo}U_{yo}A_{yo}G_{yo}G_{yo}A_{ys}A_{ys}U_{y} | |
| 1616682 | p.U_{ys}U_{fs}A_{yo}A_{yo}A_{yo}A_{fo}U_{yo} | 30 |
| | C_{yo}U_{yo}A_{yo}C_{yo}A_{yo}G_{yo}U_{fo}C_{yo} | |
| | A_{do}U_{yo}A_{yo}G_{yo}G_{yo}A_{ys}A_{ys}U_{y} | |
| 1616683 | p.U_{ys}U_{fs}A_{yo}A_{yo}A_{yo}A_{do}U_{yo} | 30 |
| | C_{yo}U_{yo}A_{yo}C_{yo}A_{yo}G_{yo}U_{do}C_{yo} | |
| | A_{do}U_{yo}A_{yo}G_{yo}G_{yo}A_{ys}A_{ys}U_{y} | |

In the table above, a subscript “f” represents a 2′-fluoro-β-D-ribosyl sugar moiety, a subscript “y” represents a 2′-O-methyl-β-D-ribosyl sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

The sense RNAi oligonucleotide Compound No. 1586323, described herein above, is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

TABLE 52
Design of siRNA targeted to HPRT1
containing 2′-deoxyribonucleosides

| Duplex | Antisense Strand | Sense Strand |
|---|---|---|
| Compound No. | Compound No. | Compound No. |
| 1642119 | 1616680 | 1586323 |
| 1642120 | 1616681 | 1586323 |
| 1642303 | 1616682 | 1586323 |
| 1642304 | 1616683 | 1586323 |

### Example 25: Dose-Dependent Inhibition of Human HPRT1 in A431 Cells by siRNA Containing 2′-Deoxyribonucleosides in the Antisense RNAi Oligonucleotide

The RNAi agents described above were tested at various doses in A431 cells. Cultured A431 cells at a density of 10,000 cells per well were treated by RNAiMAX with siRNA at concentrations indicated in the table below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100.

TABLE 53
Dose-dependent reduction of human HPRT1 RNA in A431 cells by siRNA containing
2′-deoxyribonucleosides in the antisense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 3 | 4 | 34 | 70 | 107 | 99 | 3.87 |
| 1642119 | 2 | 2 | 3 | 7 | 39 | 90 | 104 | 111 | 6.86 |
| 1642120 | 2 | 3 | 3 | 8 | 37 | 90 | 108 | 109 | 6.67 |
| 1642303 | 2 | 2 | 3 | 7 | 30 | 82 | 103 | 109 | 4.49 |
| 1642304 | 3 | 3 | 3 | 8 | 42 | 84 | 100 | 105 | 6.93 |

### Example 26: Design of siRNA Targeted to HPRT1 Containing 2′-β-D-Deoxyribonucleosides in the Sense RNAi Oligonucleotide

Modified oligonucleotides in the table below having either stereo-standard nucleosides or stereo-non-standard nucleosides in the antisense RNAi oligonucleotides and/or the sense RNAi oligonucleotides were synthesized using standard techniques. Antisense RNAi oligonucleotide Compound No. 1601968 is described here above.

The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Compound No. 1586323 is described herein above.

TABLE 54
Design of sense RNAi oligonucleotides
containing 2′-β-D-deoxyribonucleosides

| Compound | | SEQ ID |
|---|---|---|
| No. | Chemistry Notation (5′ to 3′) | NO. |
| 1616684 | U_{ys}C_{ys}C_{yo}U_{yo}A_{yo}U_{yo}G_{do}A_{yo}C_{fo}U_{fo}G_{fo}U_{yo}A_{yo}G_{yo}A_{yo}U_{yo}U_{yo}U_{yo}U_{ys}A_{ys}A_{y} | 10 |
| 1616685 | U_{ys}C_{ys}C_{yo}U_{yo}A_{yo}U_{yo}G_{fo}A_{yo}C_{do}U_{fo}G_{fo}U_{yo}A_{yo}G_{yo}A_{yo}U_{yo}U_{yo}U_{yo}U_{ys}A_{ys}A_{y} | 10 |
| 1616686 | U_{ys}C_{ys}C_{yo}U_{yo}A_{yo}U_{yo}G_{fo}A_{yo}C_{fo}U_{do}G_{fo}U_{yo}A_{yo}G_{yo}A_{yo}U_{yo}U_{yo}U_{yo}U_{ys}A_{ys}A_{y} | 10 |
| 1616687 | U_{ys}C_{ys}C_{yo}U_{yo}A_{yo}U_{yo}G_{fo}A_{yo}C_{fo}U_{fo}G_{do}U_{yo}A_{yo}G_{yo}A_{yo}U_{yo}U_{yo}U_{yo}U_{ys}A_{ys}A_{y} | 10 |
| 1616688 | U_{ys}C_{ys}C_{yo}U_{yo}A_{yo}U_{yo}G_{do}A_{yo}C_{do}U_{fo}G_{fo}U_{yo}A_{yo}G_{yo}A_{yo}U_{yo}U_{yo}U_{yo}U_{ys}A_{ys}A_{y} | 10 |
| 1616689 | U_{ys}C_{ys}C_{yo}U_{yo}A_{yo}U_{yo}G_{do}A_{yo}C_{fo}U_{do}G_{fo}U_{yo}A_{yo}G_{yo}A_{yo}U_{yo}U_{yo}U_{yo}U_{ys}A_{ys}A_{y} | 10 |
| 1616690 | U_{ys}C_{ys}C_{yo}U_{yo}A_{yo}U_{yo}G_{do}A_{yo}C_{fo}U_{fo}G_{do}U_{yo}A_{yo}G_{yo}A_{yo}U_{yo}U_{yo}U_{yo}U_{ys}A_{ys}A_{y} | 10 |
| 1616691 | U_{ys}C_{ys}C_{yo}U_{yo}A_{yo}U_{yo}G_{do}A_{yo}C_{do}U_{fo}G_{do}U_{yo}A_{yo}G_{yo}A_{yo}U_{yo}U_{yo}U_{yo}U_{ys}A_{ys}A_{y} | 10 |
| 1616692 | U_{ys}C_{ys}C_{yo}U_{yo}A_{yo}U_{yo}G_{fo}A_{yo}CaₒU_{do}G_{do}U_{yo}A_{yo}G_{yo}A_{yo}U_{yo}U_{yo}U_{yo}U_{ys}A_{ys}A_{y} | 10 |
| 1616693 | U_{ys}C_{ys}C_{yo}U_{yo}A_{yo}U_{yo}G_{do}A_{yo}C_{do}U_{do}G_{fo}U_{yo}A_{yo}G_{yo}A_{yo}U_{yo}U_{yo}U_{yo}U_{ys}A_{ys}A_{y} | 10 |
| 1616694 | U_{ys}C_{ys}C_{yo}U_{yo}A_{yo}U_{yo}G_{do}A_{yo}C_{do}U_{do}G_{do}U_{yo}A_{yo}G_{yo}A_{yo}U_{yo}U_{yo}U_{yo}U_{ys}A_{ys}A_{y} | 10 |

In the table above, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, a subscript “d” represents a 2′-β-D-deoxyribosyl sugar moiety, and a subscript “o” represents a phosphodiester internucleoside linkage.

TABLE 55
Design of siRNA targeted to HPRT1
containing 2′-deoxyribonucleosides

| Duplex | Antisense Strand | Sense Strand |
|---|---|---|
| Compound No. | Compound No. | Compound No. |
| 1642305 | 1601968 | 1616684 |
| 1642306 | 1601968 | 1616685 |
| 1642307 | 1601968 | 1616686 |
| 1642308 | 1601968 | 1616687 |
| 1642309 | 1601968 | 1616688 |
| 1642310 | 1601968 | 1616689 |
| 1642311 | 1601968 | 1616690 |
| 1642312 | 1601968 | 1616691 |
| 1642313 | 1601968 | 1616692 |
| 1642314 | 1601968 | 1616693 |
| 1642315 | 1601968 | 1616694 |
| 1642769 | 1616680 | 1616684 |
| 1642770 | 1616680 | 1616685 |
| 1642771 | 1616680 | 1616686 |
| 1642772 | 1616680 | 1616687 |
| 1642773 | 1616680 | 1616688 |
| 1642777 | 1616680 | 1616689 |
| 1642778 | 1616680 | 1616690 |
| 1642779 | 1616680 | 1616691 |
| 1642780 | 1616680 | 1616692 |
| 1642781 | 1616680 | 1616693 |
| 1642784 | 1616680 | 1616694 |
| 1642789 | 1616681 | 1616684 |
| 1642790 | 1616681 | 1616685 |
| 1642796 | 1616681 | 1616686 |
| 1642797 | 1616681 | 1616687 |
| 1642798 | 1616681 | 1616688 |
| 1642802 | 1616681 | 1616689 |
| 1642803 | 1616681 | 1616690 |
| 1642804 | 1616681 | 1616691 |
| 1642805 | 1616681 | 1616692 |
| 1642806 | 1616681 | 1616693 |
| 1642807 | 1616681 | 1616694 |
| 1642838 | 1616682 | 1616684 |
| 1642839 | 1616682 | 1616685 |
| 1642840 | 1616682 | 1616686 |
| 1642841 | 1616682 | 1616687 |
| 1642842 | 1616682 | 1616688 |
| 1642855 | 1616682 | 1616689 |
| 1642856 | 1616682 | 1616690 |
| 1642857 | 1616682 | 1616691 |
| 1642858 | 1616682 | 1616692 |
| 1642859 | 1616682 | 1616693 |
| 1642860 | 1616682 | 1616694 |
| 1645223 | 1616683 | 1616684 |
| 1645224 | 1616683 | 1616685 |
| 1645225 | 1616683 | 1616686 |
| 1645271 | 1616683 | 1616687 |
| 1645341 | 1616683 | 1616688 |
| 1645342 | 1616683 | 1616689 |
| 1645343 | 1616683 | 1616690 |
| 1645344 | 1616683 | 1616691 |
| 1645345 | 1616683 | 1616692 |
| 1645346 | 1616683 | 1616693 |
| 1645347 | 1616683 | 1616694 |

### Example 27: Dose-Dependent Inhibition of Human HPRT1 in A431 Cells by siRNA Containing 2′-Deoxyribonucleosides in the Sense RNAi Oligonucleotide

The RNAi agents described above were tested at various doses in A431 cells. Cultured A431 cells at a density of 10,000 cells per well were treated by RNAiMAX with siRNA at concentrations indicated in the tables below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment. Compound 1640504 was included as a control.

TABLE 56
Dose-dependent reduction of human HPRT1 RNA in A431 cells by siRNA containing
2′-deoxyribonucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 3 | 4 | 34 | 70 | 107 | 99 | 3.87 |
| 1642305 | 3 | 2 | 3 | 5 | 24 | 86 | 100 | 106 | 4.17 |
| 1642306 | 2 | 2 | 3 | 5 | 25 | 80 | 111 | 105 | 3.76 |
| 1642307 | 3 | 2 | 2 | 6 | 32 | 64 | 104 | 108 | 3.17 |
| 1642308 | 3 | 2 | 2 | 4 | 32 | 72 | 105 | 102 | 3.70 |

TABLE 57
Dose-dependent reduction of human HPRT1 RNA in
A431 cells by siRNA containing 2′-
deoxyribonucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 3 | 4 | 32 | 92 | 98 | 92 | 5.80 |
| 1642309 | 2 | 3 | 3 | 6 | 30 | 78 | 89 | 96 | 3.82 |
| 1642310 | 2 | 3 | 3 | 5 | 18 | 68 | 84 | 84 | 1.86 |
| 1642311 | 2 | 2 | 2 | 6 | 30 | 57 | 88 | 82 | 1.75 |
| 1642312 | 2 | 2 | 2 | 8 | 33 | 66 | 85 | 92 | 2.64 |
| 1642313 | 2 | 2 | 2 | 5 | 30 | 62 | 88 | 96 | 2.25 |
| 1642314 | 3 | 2 | 3 | 5 | 33 | 80 | 94 | 96 | 4.55 |
| 1642315 | 2 | 2 | 2 | 7 | 26 | 77 | 89 | 90 | 3.30 |

TABLE 58
Dose-dependent reduction of human HPRT1
RNA in A431 cells by siRNA containing 2′-
deoxyribonucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 3 | 11 | 40 | 76 | 96 | 119 | 5.67 |
| 1642769 | 2 | 2 | 3 | 6 | 31 | 70 | 97 | 91 | 3.30 |
| 1642770 | 1 | 3 | 2 | 8 | 37 | 104 | 113 | 106 | 8.98 |
| 1642771 | 2 | 3 | 4 | 9 | 42 | 101 | 119 | 111 | 8.40 |
| 1642772 | 2 | 3 | 3 | 8 | 37 | 101 | 122 | 116 | 7.75 |
| 1642773 | 2 | 3 | 3 | 8 | 33 | 102 | 133 | 126 | 8.29 |
| 1642777 | 2 | 2 | 3 | 5 | 32 | 102 | 108 | 103 | 8.45 |
| 1642778 | 2 | 2 | 2 | 6 | 23 | 81 | 109 | 102 | 3.64 |
| 1642779 | 3 | 2 | 2 | 5 | 19 | 62 | 119 | 119 | 2.02 |
| 1642780 | 3 | 2 | 2 | 6 | 37 | 59 | 103 | 92 | 3.06 |

TABLE 59
Dose-dependent reduction of human HPRT1
RNA in A431 cells by siRNA containing 2′-
deoxyribonucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 1 | 2 | 2 | 9 | 48 | 69 | 93 | 82 | 5.20 |
| 1642781 | 2 | 2 | 3 | 6 | 35 | 84 | 93 | 93 | 5.28 |
| 1642784 | 2 | 3 | 2 | 8 | 35 | 83 | 89 | 101 | 5.19 |
| 1642789 | 2 | 2 | 2 | 7 | 40 | 67 | 88 | 103 | 3.83 |
| 1642790 | 1 | 3 | 3 | 11 | 31 | 81 | 87 | 89 | 4.28 |
| 1642796 | 2 | 2 | 2 | 7 | 36 | 73 | 89 | 93 | 3.92 |
| 1642797 | 2 | 2 | 4 | 6 | 46 | 71 | 103 | 98 | 5.85 |
| 1642798 | 1 | 2 | 2 | 7 | 52 | 86 | 89 | 114 | 9.67 |
| 1642802 | 1 | 2 | 3 | 8 | 50 | 91 | 103 | 105 | 10.00 |

TABLE 60
Dose-dependent reduction of human HPRT1
RNA in A431 cells by siRNA containing 2′-
deoxyribonucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 1 | 2 | 4 | 10 | 57 | 71 | 101 | 106 | 9.12 |
| 1642803 | 3 | 3 | 3 | 11 | 51 | 91 | 97 | 94 | 10.77 |
| 1642804 | 3 | 2 | 6 | 13 | 60 | 77 | 91 | 93 | 10.63 |
| 1642805 | 2 | 3 | 5 | 12 | 55 | 88 | 96 | 98 | 11.61 |
| 1642806 | 2 | 2 | 4 | 12 | 62 | 65 | 98 | 90 | 8.82 |
| 1642807 | 2 | 3 | 4 | 16 | 47 | 79 | 88 | 100 | 7.47 |
| 1642838 | 2 | 2 | 4 | 9 | 36 | 78 | 94 | 100 | 4.81 |
| 1642839 | 1 | 3 | 3 | 9 | 46 | 85 | 91 | 103 | 7.72 |
| 1642840 | 2 | 2 | 3 | 10 | 46 | 76 | 91 | 99 | 6.39 |

TABLE 61
Dose-dependent reduction of human HPRT1 RNA
in A431 cells by siRNA containing 2′-
deoxyribonucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 1 | 2 | 4 | 6 | 51 | 100 | 103 | 117 | 10.56 |
| 1642841 | 2 | 3 | 3 | 6 | 26 | 86 | 96 | 111 | 4.34 |
| 1642842 | 2 | 2 | 2 | 6 | 34 | 78 | 102 | 96 | 4.72 |
| 1642855 | 2 | 2 | 3 | 5 | 26 | 71 | 96 | 92 | 2.95 |
| 1642856 | 2 | 2 | 3 | 6 | 25 | 66 | 87 | 94 | 2.27 |
| 1642857 | 2 | 2 | 2 | 4 | 26 | 71 | 77 | 91 | 2.15 |
| 1642858 | 2 | 2 | 2 | 5 | 24 | 65 | 88 | 85 | 2.02 |
| 1642859 | 2 | 2 | 2 | 5 | 24 | 74 | 94 | 102 | 2.98 |
| 1642860 | 2 | 2 | 3 | 6 | 41 | 80 | 99 | 98 | 5.83 |

TABLE 62
Dose-dependent reduction of human HPRT1 RNA
in A431 cells by siRNA containing 2′-
deoxyribonucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 3 | 9 | 28 | 78 | 112 | 119 | 4.13 |
| 1645223 | 2 | 3 | 5 | 10 | 49 | 97 | 112 | 132 | 10.29 |
| 1645224 | 2 | 3 | 6 | 13 | 55 | 91 | 120 | 109 | 12.53 |
| 1645225 | 2 | 2 | 3 | 11 | 46 | 97 | 92 | 93 | 9.42 |
| 1645271 | 2 | 4 | 3 | 18 | 56 | 78 | 92 | 104 | 10.95 |
| 1645341 | 2 | 3 | 4 | 11 | 44 | 72 | 96 | 108 | 5.68 |
| 1645342 | 2 | 2 | 5 | 13 | 55 | 77 | 100 | 97 | 9.95 |
| 1645343 | 3 | 3 | 3 | 16 | 61 | 77 | 86 | 120 | 12.25 |
| 1645344 | 4 | 4 | 2 | 7 | 64 | 80 | 95 | 95 | 12.95 |

TABLE 63
Dose-dependent reduction of human HPRT1
RNA in A431 cells by siRNA containing 2′-
deoxyribonucleosides in the sense RNAi oligonucleotide

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 2 | 4 | 20 | 70 | 88 | 100 | 2.26 |
| 1645345 | 2 | 3 | 4 | 9 | 48 | 94 | 91 | 123 | 9.54 |
| 1645346 | 2 | 2 | 4 | 10 | 45 | 95 | 101 | 113 | 8.86 |
| 1645347 | 2 | 2 | 3 | 9 | 42 | 93 | 95 | 86 | 7.92 |

### Example 28: Design of siRNA Targeted to HPRT1 Containing 2′-β-D-Deoxyribonucleosides in the Sense RNAi Oligonucleotide

Modified oligonucleotides in the table below having either stereo-standard nucleosides or stereo-non-standard nucleosides in the antisense RNAi oligonucleotides and/or the sense RNAi oligonucleotides were synthesized using standard techniques. Antisense RNAi oligonucleotide Compound No. 1601968 is described herein above.

The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Compound No. 1586323 is described herein above.

TABLE 64
Design of sense RNAi oligonucleotide modified oligonucleotides
containing 2′-β-D-deoxyribonucleosides

| Compound | | SEQ ID |
|---|---|---|
| No. | Chemistry Notation (5′ to 3′) | NO. |
| 1616648 | U_{ys}C_{ys}C_{yo}U_{yo}A_{yo}U_{do}G_{do}A_{do}C_{do}U_{do}G_{do}U_{do}A_{do}G_{do}A_{do}U_{do}U_{yo}U_{yo}U_{ys}A_{ys}A_{y} | 10 |
| 1616649 | U_{ys}C_{ys}C_{yo}U_{yo}A_{yo}U_{yo}G_{do}A_{do}C_{do}U_{do}G_{do}U_{do}A_{do}G_{do}A_{do}U_{yo}U_{yo}U_{yo}U_{ys}A_{ys}A_{y} | 10 |
| 1616650 | U_{ys}C_{ys}C_{yo}U_{yo}A_{yo}U_{yo}G_{yo}A_{do}C_{do}U_{do}G_{do}U_{do}A_{do}G_{do}A_{yo}U_{yo}U_{yo}U_{yo}U_{ys}A_{ys}A_{y} | 10 |
| 1616661 | U_{ys}C_{ys}C_{yo}U_{yo}A_{yo}U_{yo}G_{yo}A_{yo}C_{do}U_{do}G_{do}U_{do}A_{do}G_{yo}A_{yo}U_{yo}U_{yo}U_{yo}U_{ys}A_{ys}A_{y} | 10 |
| 1616668 | UₑₛCₑₛCₑₒUₑₒAₑₒU_{do}G_{do}A_{do}C_{do}U_{do}G_{do}U_{do}A_{do}G_{do}A_{do}U_{do}UₑₒUₑₒUₑₛAₑₛAₑ | 10 |
| 1616669 | UₑₛCₑₛCₑₒUₑₒAₑₒUₑₒG_{do}A_{do}C_{do}U_{do}G_{do}U_{do}A_{do}G_{do}A_{do}UₑₒUₑₒUₑₒUₑₛAₑₛAₑ | 10 |
| 1616670 | UₑₛCₑₛCₑₒUₑₒAₑₒUₑₒGₑₒA_{do}C_{do}U_{do}G_{do}U_{do}A_{do}G_{do}AₑₒUₑₒUₑₒUₑₒUₑₛAₑₛAₑ | 10 |
| 1616672 | UₑₛCₑₛCₑₒUₑₒAₑₒUₑₒGₑₒAₑₒC_{do}U_{do}G_{do}U_{do}A_{do}GₑₒAₑₒUₑₒUₑₒUₑₒUₑₛAₑₛAₑ | 10 |

In the table above, a subscript “y” represents a 2′-O-methyl-β-D-ribosyl sugar moiety, a subscript “e” represents a 2′-MOE sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

TABLE 65
Design of siRNA targeted to HPRT1 containing
2′-β-D-deoxyribonucleosides

| Duplex | Antisense Strand | Sense Strand |
|---|---|---|
| Compound No. | Compound No. | Compound No. |
| 1645372 | 1601968 | 1616648 |
| 1645384 | 1601968 | 1616649 |
| 1645377 | 1601968 | 1616650 |
| 1645532 | 1601968 | 1616661 |
| 1645855 | 1601968 | 1616668 |
| 1645858 | 1601968 | 1616669 |
| 1646795 | 1601968 | 1616670 |
| 1646796 | 1601968 | 1616672 |

### Example 29: Dose-Dependent Inhibition of Human HPRT1 in A431 Cells by siRNA Containing 2′-β-D-Deoxyribonucleosides

The RNAi agents described above were tested at various doses in A431 cells. Cultured A431 cells at a density of 10,000 cells per well were treated by RNAiMAX with siRNA at concentrations indicated in the tables below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment. Compound 1640504 was included as a control.

TABLE 66
Dose-dependent reduction of human HPRT1 RNA
in A431 cells by siRNA containing 2′-O-methyl
nucleosides and 2′-deoxyribonucleosides

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 2 | 4 | 20 | 70 | 88 | 100 | 2.26 |
| 1645372 | 2 | 2 | 2 | 6 | 24 | 82 | 101 | 112 | 3.82 |
| 1645384 | 2 | 2 | 2 | 4 | 22 | 72 | 98 | 116 | 2.72 |
| 1645377 | 2 | 2 | 2 | 5 | 19 | 57 | 89 | 103 | 1.51 |
| 1645532 | 2 | 2 | 3 | 5 | 25 | 83 | 93 | 119 | 3.85 |

TABLE 67
Dose-dependent reduction of human HPRT1 RNA in
A431 cells by siRNA containing 2′-MOE and 2′-
deoxyribonucleosides

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 3 | 3 | 6 | 26 | 75 | 103 | 113 | 3.39 |
| 1645855 | 3 | 5 | 5 | 10 | 34 | 85 | 106 | 114 | 5.61 |
| 1645858 | 2 | 5 | 6 | 11 | 40 | 96 | 112 | 112 | 7.80 |
| 1646795 | 3 | 4 | 5 | 11 | 51 | 94 | 112 | 98 | 11.08 |
| 1646796 | 2 | 4 | 4 | 10 | 47 | 98 | 109 | 110 | 9.84 |

### Example 30: Design of siRNA Targeted to HPRT1 Containing 2′-β-D-Deoxyribonucleosides in the Sense RNAi Oligonucleotide

Modified oligonucleotides in the table below having either stereo-standard nucleosides or stereo-non-standard nucleosides in the antisense RNAi oligonucleotides and/or the sense RNAi oligonucleotides were synthesized using standard techniques. Antisense RNAi oligonucleotide Compound No. 1601968 is described herein above. The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). The sense RNAi oligonucleotide Compound No. 1586323 is described herein above.

TABLE 68
Design of sense RNAi oligonucleotides
containing 2′-β-D-deoxyribonucleosides

| Compound | | SEQ ID |
|---|---|---|
| No. | Chemistry Notation (5′ to 3′) | NO. |
| 1616665 | U_{ys}C_{ys}C_{yo}U_{yo}A_{yo}U_{yo}G_{do}A_{yo}C_{do}U_{yo}G_{do}U_{yo}A_{do}G_{yo}A_{do}U_{do}U_{yo}U_{yo}U_{ys}A_{ys}A_{y} | 10 |
| 1616675 | UₑₛCₑₛCₑₒUₑₒAₑₒUₑₒG_{do}AₑₒC_{do}UₑₒG_{do}UₑₒA_{do}GₑₒA_{do}U_{do}UₑₒUₑₒUₑₛAₑₛAₑ | 10 |
| 1616697 | U_{ds}C_{ys}C_{do}U_{yo}A_{do}U_{yo}G_{do}A_{yo}C_{do}U_{yo}G_{do}U_{yo}A_{do}G_{yo}A_{do}U_{yo}U_{do}U_{yo}U_{ds}A_{ys}A_{d} | 10 |
| 1616698 | U_{ds}C_{ys}C_{do}U_{yo}A_{do}U_{yo}G_{fo}A_{yo}C_{fo}U_{fo}G_{fo}U_{yo}A_{do}G_{yo}A_{do}U_{yo}U_{do}U_{yo}U_{ds}A_{ys}A_{d} | 10 |
| 1616699 | U_{ds}CₑₛC_{do}UₑₒA_{do}UₑₒG_{do}AₑₒC_{do}UₑₒG_{do}UₑₒA_{do}GₑₒA_{do}UₑₒU_{do}UₑₒU_{ds}AₑₛA_{d} | 10 |
| 1616778 | U_{ds}CₑₛC_{do}UₑₒA_{do}UₑₒG_{fo}AₑₒC_{fo}U_{fo}G_{fo}UₑₒA_{do}GₑₒA_{do}UₑₒU_{do}UₑₒU_{ds}AₑₛA_{d} | 10 |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “r” represents a β-D-ribosyl sugar moiety, a subscript “s” represents a phosphorothioate internucleoside linkage, a subscript “d” represents a 2′-β-D-deoxyribosyl sugar moiety, and a subscript “o” represents a phosphodiester internucleoside linkage.

TABLE 69
Design of siRNA targeted to HPRT1
containing 2′-deoxyribonucleosides

| Duplex | Antisense Strand | Sense Strand |
|---|---|---|
| Compound No. | Compound No. | Compound No. |
| 1645755 | 1601968 | 1616665 |
| 1646797 | 1601968 | 1616675 |
| 1647734 | 1601968 | 1616697 |
| 1647735 | 1601968 | 1616698 |
| 1647736 | 1601968 | 1616699 |
| 1647737 | 1601968 | 1616778 |

### Example 31: Dose-Dependent Inhibition of Human HPRT1 in A431 Cells by siRNA Containing 2′-Deoxyribonucleosides

The RNAi agents described above were tested at various doses in A431 cells. Cultured A431 cells at a density of 10,000 cells per well were treated by RNAiMAX with siRNA at concentrations indicated in the tables below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment. Compound 1640504 was included as a control.

TABLE 70
Dose-dependent reduction of human HPRT1 RNA
in A431 cells by siRNA containing 2′-O-methyl
nucleosides and 2′-deoxyribonucleosides

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 2 | 2 | 4 | 20 | 70 | 88 | 100 | 2.26 |
| 1645755 | 2 | 2 | 3 | 6 | 36 | 98 | 103 | 98 | 7.10 |

TABLE 71
Dose-dependent reduction of human HPRT1 RNA in
A431 cells by siRNA containing 2′-O-methyl
nucleosides and 2′-deoxyribonucleosides

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 3 | 3 | 6 | 26 | 75 | 103 | 113 | 3.39 |
| 1647734 | 2 | 2 | 3 | 9 | 38 | 84 | 131 | 120 | 6.56 |
| 1647735 | 2 | 2 | 3 | 9 | 34 | 81 | 113 | 114 | 5.30 |
| 1647736 | 2 | 3 | 4 | 10 | 41 | 93 | 108 | 108 | 7.68 |
| 1646797 | 2 | 3 | 5 | 9 | 74 | 108 | — | 104 | 20.70 |

TABLE 72
Dose-dependent reduction of human HPRT1 RNA
in A431 cells by siRNA containing 2′-O-methyl
nucleosides and an alternating 2′-deoxyribonucleoside motif

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 3 | 3 | 3 | 6 | 23 | 77 | 96 | 107 | 3.17 |
| 1647737 | 3 | 3 | 5 | 8 | 40 | 81 | 98 | 102 | 5.97 |

### Example 32: Design of siRNA Targeted to HPRT1 Containing Ribonucleosides

Modified oligonucleotides in the table below having either stereo-standard nucleosides or stereo-non-standard nucleosides in the antisense RNAi oligonucleotide and/or the sense RNAi oligonucleotides were synthesized using standard techniques. Antisense RNAi oligonucleotide Compound No. 1601968 is described herein above.

The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Compound No. 1586323 is described herein above.

TABLE 73
Design of sense RNAi oligonucleotides
containing ribonucleosides

| Compound | | SEQ ID |
|---|---|---|
| No. | Chemistry Notation (5′ to 3′) | NO. |
| 1616779 | UᵣₛC_{ys}CᵣₒU_{yo}AᵣₒU_{yo}GᵣₒA_{yo}CᵣₒU_{yo}Gᵣₒ | 10 |
| | U_{yo}AᵣₒG_{yo}AᵣₒU_{yo}UᵣₒU_{yo}UᵣₛA_{ys}Aᵣ | |
| 1616780 | UᵣₛC_{ys}CᵣₒU_{yo}AᵣₒU_{yo}G_{fo}A_{yo}C_{fo}U_{fo}G_{fo} | 10 |
| | U_{yo}AᵣₒG_{yo}AᵣₒU_{yo}UᵣₒU_{yo}UᵣₛA_{ys}Aᵣ | |
| 1616781 | UᵣₛCₑₛCᵣₒUₑₒAᵣₒUₑₒGᵣₒAₑₒCᵣₒUₑₒGᵣₒ | 10 |
| | UₑₒAᵣₒGₑₒAᵣUₑₒUᵣₒUₑₒUᵣₛAₑₛAᵣ | |
| 1616782 | UᵣₛCₑₛCᵣₒUₑₒAᵣₒUₑₒG_{fo}AₑₒC_{fo}U_{fo}G_{fo} | 10 |
| | UₑₒAᵣₒGₑₒAᵣₒUₑₒUᵣₒUₑₒUᵣₛAₑₛAᵣ | |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “r” represents a β-D-ribosyl sugar moiety, a subscript “s” represents a phosphorothioate internucleoside linkage, a subscript “d” represents a 2′-β-D-deoxyribosyl sugar moiety, and a subscript “o” represents a phosphodiester internucleoside linkage.

TABLE 74
Design of siRNA targeted to HPRT1 containing ribonucleosides

| Duplex | Antisense Strand | Sense Strand |
|---|---|---|
| Compound No. | Compound No. | Compound No. |
| 1647738 | 1601968 | 1616779 |
| 1647739 | 1601968 | 1616780 |
| 1647740 | 1601968 | 1616781 |
| 1647741 | 1601968 | 1616782 |

### Example 33: Dose-Dependent Inhibition of Human HPRT1 in A431 Cells by siRNA Containing Ribonucleosides

The RNAi agents described above were tested at various doses in A431 cells. Cultured A431 cells at a density of 10,000 cells per well were treated by RNAiMAX with siRNA at concentrations indicated in the table below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment.

TABLE 75
Dose-dependent reduction of human HPRT1 RNA in
A431 cells by siRNA containing 2′-O-methyl
nucleosides and ribonucleoside motifs

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 3 | 3 | 3 | 6 | 23 | 77 | 96 | 107 | 3.17 |
| 1647738 | 3 | 4 | 5 | 12 | 41 | 81 | 99 | 117 | 6.75 |
| 1647739 | 3 | 3 | 5 | 10 | 31 | 83 | 106 | 107 | 4.86 |
| 1647740 | 3 | 5 | 6 | 15 | 54 | 89 | 94 | 97 | 12.42 |
| 1647741 | 3 | 4 | 5 | 12 | 51 | 88 | 96 | 104 | 10.34 |

### Example 34: Design of RNAi Agents Containing 3′-Lipid Conjugates Targeted to Both Human and Mouse HPRT1

Modified oligonucleotides in the table below having either stereo-standard nucleosides or stereo-non-standard nucleosides in the antisense RNAi oligonucleotide and/or the sense RNAi oligonucleotides were synthesized using standard techniques. The antisense RNAi oligonucleotide described in the table below has the sequence TUAAAAUCUACAGUCAUAGGAAU (SEQ ID NO: 9) and is complementary to human HPRT1 GenBank Accession No. NM_000194.2 (SEQ ID NO: 1, described herein above) from nucleosides 444 to 465 with a single mismatch at position 1 on the 5′ end of the antisense RNAi oligonucleotide. The sequence TUAAAAUCUACAGUCAUAGGAAU (SEQ ID NO: 9) is also complementary to mouse HPRT1 ENSEMBL ID ENSMUST00000026723.9, from ENSEMBL Release 104 (May 2021)(SEQ ID NO: 36) from nucleoside 364 to 385 with a single mismatch at position 1 on the 5′ end of the antisense RNAi oligonucleotide. Compound No. 1586322 is described herein above.

The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Compound No. 1591095 is described herein above. The sense RNAi oligomeric compounds comprise an alkyl conjugate group, as indicated in the table below.

TABLE 76
Design of sense RNAi oligomeric compounds
containing 3′-lipid conjugates

| Compound | | SEQ ID |
|---|---|---|
| No. | Chemistry Notation (5′ to 3′) | NO. |
| 1653520 | U_{ys}C_{ys}C_{yo}U_{yo}A_{yo}U_{yo}G_{fo}A_{yo}C_{fo}U_{fo}G_{fo}U_{yo}A_{yo} | 10 |
| | G_{yo}A_{yo}U_{yo}U_{yo}U_{yo}U_{ys}A_{ys}A_{yo}[3nC7-C10] | |
| 1653521 | U_{ys}C_{ys}C_{yo}U_{yo}A_{yo}U_{yo}G_{fo}A_{yo}C_{fo}U_{fo}G_{fo}U_{yo}A_{yo} | 10 |
| | G_{yo}A_{yo}U_{yo}U_{yo}U_{yo}U_{ys}A_{ys}A_{yo}[3nC7-C8] | |
| 1653533 | U_{ys}C_{ys}C_{yo}U_{yo}A_{yo}U_{yo}G_{fo}A_{yo}C_{fo}U_{fo}G_{fo}U_{yo}A_{yo} | 10 |
| | G_{yo}A_{yo}U_{yo}U_{yo}U_{yo}U_{ys}A_{ys}A_{yo}[3nC7-C18] | |

In the table above, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage.

“[3nC7-C8]” represents a caprylate moiety linked to a 3′-C7 amino modifier, as shown below, which is attached to the 3′-nucleoside via a phosphodiester linkage.

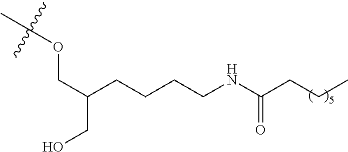

“[3nC7-C10]” represents a caprate moiety linked to a 3′-C7 amino modifier, as shown below, which is attached to the 3′-nucleoside via a phosphodiester linkage.

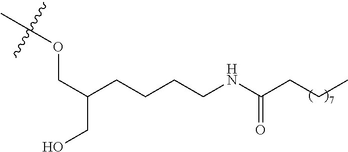

“[3nC7-C18]” represents an oleate moiety linked to a 3′-C7 amino modifier, as shown below, which is attached to the 3′-nucleoside via a phosphodiester linkage.

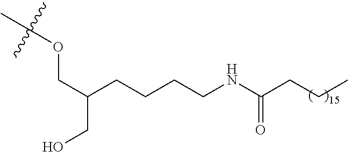

TABLE 77
Design of siRNA targeted to HPRT1

| Duplex | Antisense Strand | Sense Strand |
|---|---|---|
| Compound No. | Compound No. | Compound No. |
| 1599476 | 1586322 | 1591095 |
| 1653542 | 1586322 | 1653520 |
| 1653543 | 1586322 | 1653521 |
| 1653544 | 1586322 | 1653533 |

### Example 35: Dose-Dependent Inhibition of Human HPRT in A431 Cells by siRNA Containing 3′-Lipid Conjugates

The RNAi agents described above were tested at various doses in A431 cells. Cultured A431 cells at a density of 10,000 cells per well were treated by RNAiMAX with siRNA at concentrations indicated in the table below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100.

TABLE 78
Dose-dependent inhibition of human HPRT1 in A431
cells by siRNA containing 3′-lipid conjugates

| | HPRT1 RNA (% UTC) | | | | | | | | |
|---|---|---|---|---|---|---|---|---|---|
| Compound | 100 | 10 | 1 | 0.1 | 0.01 | 0.001 | 0.0001 | 0.00001 | IC₅₀ |
| No. | nM | nM | nM | nM | nM | nM | nM | nM | (pM) |
| 1640504 | 2 | 3 | 3 | 5 | 25 | 71 | 91 | 104 | 2.76 |
| 1599476 | 4 | 4 | 4 | 7 | 30 | 78 | 108 | 98 | 4.15 |
| 1653542 | 3 | 3 | 6 | 8 | 36 | 80 | 100 | 109 | 5.12 |
| 1653543 | 2 | 4 | 4 | 8 | 32 | 82 | 103 | 101 | 4.89 |
| 1653544 | 2 | 2 | 3 | 6 | 30 | 86 | 90 | 98 | 4.73 |

### Example 36: Activity of siRNAs Containing 3′-Lipid Conjugates Targeted to Mouse HPRT, In Vivo

The activity of RNAi agents containing lipids was tested in wild type C57BL/6 mice (Taconic Biosciences).

Mice were divided into groups of 2 mice each. Each mouse received a single ICV bolus of 1 μg, 10 μg, 100 μg, or 500 μg of RNAi agent. A group of 4 mice received PBS as a control. Two weeks post treatment, mice were sacrificed, and RNA was extracted from cortical brain tissue, spinal cord, and liver tissue for quantitative real-time RTPCR analysis of RNA expression of HPRT using primer probe set RTS43125 (described herein above). Results are presented as percent change of RNA, relative to PBS control, normalized to mouse cyclophilin A (% control). Mouse cyclophilin A was amplified using primer probe set m_cyclo24 (designated herein above). As shown in the table below, treatment with modified oligonucleotides resulted in reduction of HPRT RNA in comparison to the PBS control.

The half maximal dose (EC₅₀) of each RNAi agent was calculated with GraphPad Prism software (v8.2.0, San Diego, CA) using the log (agonist) vs. response function: Y=Bottom+(Top−Bottom)/(1+10\hat{ }((Log EC50−X)*HillSlope)), with the following constraints: Bottom=0, Top=100.

TABLE 79
Reduction of mouse HPRT RNA in wild type C57BL/6 mice

| | | Spinal Cord | | Cortex | | Liver | |
|---|---|---|---|---|---|---|---|
| | | HPRT | | HPRT | | HPRT | |
| | | RNA | | RNA | | RNA | |
| Compound | Dose | (% | ED₅₀ | (% | ED₅₀ | (% | ED₅₀ |
| No. | (μg) | control) | (μg) | control) | (μg) | control) | (μg) |
| 1599476 | 1 | 96 | 42 | 96 | 64 | 104 | 99 |
| | 10 | 91 | | 79 | | 96 | |
| | 100 | 19 | | 39 | | 49 | |
| | 500 | 8 | | 23 | | 12 | |
| 1653542 | 1 | 91 | 63 | 92 | 255 | 105 | 332 |
| | 10 | 98 | | 99 | | 104 | |
| | 100 | 31 | | 70 | | 70 | |
| | 500 | 16 | | 35 | | 43 | |
| 1653543 | 1 | 99 | 77 | 98 | 77 | 104 | 445 |
| | 10 | 82 | | 95 | | 104 | |
| | 100 | 45 | | 36 | | 82 | |
| | 500 | 19 | | 24 | | 47 | |
| 1653544 | 1 | 100 | 60 | 100 | 87 | 101 | 164 |
| | 10 | 90 | | 94 | | 96 | |
| | 100 | 34 | | 42 | | 68 | |
| | 500 | 14 | | 21 | | 15 | |

### Example 37: Design of RNAi Agents Containing Mesyl Phosphoramidate Linkages Targeted to Both Human and Mouse HPRT1

Modified oligonucleotides in the table below having either stereo-standard nucleosides or stereo-non-standard nucleosides in the antisense RNAi oligonucleotide and/or the sense RNAi oligonucleotides were synthesized using standard techniques. The antisense RNAi oligonucleotide described in the table below has the sequence TUAAAAUCUACAGUCAUAGGAAU (SEQ ID NO: 9), as described herein above. Antisense RNAi oligonucleotide Compounds no. 1586322, 1595969, 1595970, 1595971 are described herein above.

TABLE 80
Design of antisense strand modified
oligonuclₑₒtides containing mesyl
phosphoramidate linkages

| Compound | Chemistry Notation | SEQ |
|---|---|---|
| No. | (5′ to 3′) | ID NO. |
| 1625828 | VP-Tes [symbol] A_{yo}A_{yo}A_{yo}A_{fo} | 9 |
| | U_{yo}C_{yo}U_{yo}A_{yo}C_{yo}A_{yo}G_{yo} | |
| | U_{fo}C_{yo}A_{fo}U_{yo}A_{yo}G_{yo}G_{yo} [symbol] | |
| | A_{ys}Uy | |

In the table above, a “vP-” represents a vinyl phosphonate (vP) moiety on the 5′-end, a subscript “e” represents a 2′-MOE sugar moiety, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage, and a subscript “z” represents a mesyl phosphoramidate internucleoside linkage (Formula II).

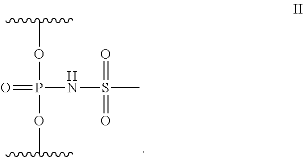 [SMILES: *OP(=O)(NS(C)(=O)=O)O*]

The sense RNAi oligonucleotide in the table below is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). The sense RNAi oligomeric compound further comprises a C16 conjugate group, as indicated in the table below. Compound No. 1586324 is described herein above.

TABLE 81
Design of sense RNAi oligomeric compounds
containing mesyl phosphoramidate linkages

| | | SEQ |
|---|---|---|
| Compound | | ID |
| No. | Chemistry Notation (5′ to 3′) | NO. |
| 1633343 | [symbol] C_{ys}C_{yo}U_{yo}A_{yo}U_{[16C2r]o}G_{fo}A_{yo}C_{fo}U_{fo} | 10 |
| | G_{fo}U_{yo}A_{yo}G_{yo}A_{yo}U_{yo}U_{yo}U_{yo}U_{ys} [symbol] A_{y} | |

In the table above, a subscript “f” represents a 2′-fluoro-β-D-ribosyl sugar moiety, a subscript “y” represents a 2′-O-methyl-β-D-ribosyl sugar moiety, a subscript “[16C2r]” represents a 2′-O-hexadecylribosyl sugar moiety, a subscript “s” represents a phosphorothioate internucleoside linkage, a subscript “o” represents a phosphodiester internucleoside linkage, and a subscript “z” represents a mesyl phosphoramidate internucleoside linkage (Formula II).

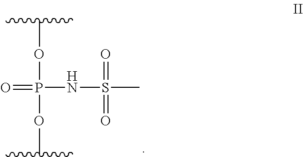 [SMILES: *OP(=O)(NS(C)(=O)=O)O*]

TABLE 82
Design of siRNA targeted to HPRT1

| Duplex | Antisense Strand | Sense Strand |
|---|---|---|
| Compound No. | Compound No. | Compound No. |
| 1652937 | 1595969 | 1586324 |
| 1652944 | 1595970 | 1586324 |
| 1652945 | 1595971 | 1586324 |
| 1652946 | 1595971 | 1633343 |
| 1652947 | 1625828 | 1586324 |
| 1652951 | 1625828 | 1633343 |
| 1653518 | 1595969 | 1633343 |
| 1653519 | 1595970 | 1633343 |

### Example 38: Dose-Dependent Inhibition of Human HPRT in A431 Cells by siRNA Containing Mesyl Phosphoramidate Linkages

The RNAi agents described above were tested at various doses in A431 cells. Cultured A431 cells at a density of 10,000 cells per well were treated by RNAiMAX with siRNA at concentrations indicated in the table below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC). Parent Compound No. 1588822, described herein above, was included as a control.

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment.

TABLE 83
Dose-dependent inhibition of human HPRT1 in HeLa cells by
siRNA containing mesyl phosphoramidate nucleosides

| | HPRT1 RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1588822 | 3 | 3 | 3 | 5 | 30 | 67 | 89 | 89 | 2.63 |
| 1652937 | 4 | 3 | 4 | 8 | 48 | 86 | 96 | 97 | 8.67 |
| 1652944 | 1 | 3 | 4 | 8 | 37 | 84 | 98 | 93 | 5.82 |
| 1652945 | 3 | 3 | 3 | 6 | 40 | 84 | 88 | 92 | 5.86 |
| 1652946 | 6 | 3 | 4 | 18 | 47 | 83 | 87 | 86 | 8.29 |
| 1652947 | 15 | 4 | 3 | 6 | 30 | 88 | 92 | 99 | 4.87 |
| 1652951 | 7 | 3 | 5 | 22 | 70 | 98 | 98 | 92 | 25.74 |
| 1653518 | 10 | 4 | 4 | 14 | 57 | 80 | 95 | 94 | 11.42 |
| 1653519 | 5 | 3 | 3 | 10 | 51 | 81 | 99 | 96 | 9.16 |

### Example 39: Activity of siRNAs Containing Mesyl Phosphoramidate Linkages Targeted to Mouse HPRT, In Vivo

The activity of RNAi agents having lipid conjugates was tested in wild type C57BL/6 mice (Taconic Biosciences).

Mice were divided into groups of 2 mice each. Each mouse received a single ICV bolus of 1 μg, 10 μg, 100 μg, or 500 μg of RNAi agent. A group of 4 mice received PBS as a control. Two weeks post treatment, mice were sacrificed, and RNA was extracted from cortical brain tissue, spinal cord, and liver tissue for quantitative real-time RTPCR analysis of RNA expression of HPRT using primer probe set RTS43125 (described herein above). Results are presented as percent change of RNA, relative to PBS control, normalized to mouse cyclophilin A (% control). Mouse cyclophilin A was amplified using primer probe set m_cyclo24 (designated herein above). As shown in the table below, treatment with modified oligonucleotides resulted in reduction of HPRT RNA in comparison to the PBS control.

The half maximal dose (EC₅₀) of each RNAi agent was calculated with GraphPad Prism software (v8.2.0, San Diego, CA) using the log (agonist) vs. response function: Y=Bottom+(Top−Bottom)/(1+10\hat{ }((Log EC50−X)*HillSlope)), with the following constraints: Bottom=0, Top=100. N.C. refers to data points that were not calculated.

TABLE 84
Reduction of mouse HPRT RNA in wild type C57BL/6 mice

| | | Spinal Cord | | Cortex | | Liver | |
|---|---|---|---|---|---|---|---|
| | | HPRT | | HPRT | | HPRT | |
| Compound | Dose | RNA | ED₅₀ | RNA | ED₅₀ | RNA | ED₅₀ |
| No. | (μg) | (% control) | (μg) | (% control) | (μg) | (% control) | (μg) |
| 1588822 | 1 | 101 | 24 | 102 | 56 | 112 | 42 |
| | 10 | 62 | | 79 | | 84 | |
| | 100 | 24 | | 35 | | 25 | |
| | 500 | 15 | | 21 | | 10 | |
| 1652937 | 1 | 88 | 25 | 90 | 31 | 104 | 65 |
| | 10 | 59 | | 57 | | 91 | |
| | 100 | 33 | | 37 | | 34 | |
| | 500 | 19 | | 24 | | 16 | |
| 1652944 | 1 | 95 | 15 | 83 | 55 | 102 | 62 |
| | 10 | 52 | | 77 | | 85 | |
| | 100 | 21 | | 46 | | 38 | |
| | 500 | 13 | | 14 | | 12 | |
| 1652945 | 1 | 89 | 15 | 90 | 73 | 102 | 52 |
| | 10 | 51 | | 83 | | 83 | |
| | 100 | 26 | | 43 | | 33 | |
| | 500 | 12 | | 22 | | 12 | |
| 1652946 | 1 | 95 | 78 | 93 | 196 | 110 | N.C. |
| | 10 | 93 | | 91 | | 102 | |
| | 100 | 42 | | 61 | | 88 | |
| 1652947 | 1 | 88 | 14 | 85 | 31 | 96 | 39 |
| | 10 | 51 | | 66 | | 87 | |
| | 100 | 26 | | 34 | | 21 | |
| 1652951 | 1 | 92 | 57 | 104 | 125 | 106 | >500 |
| | 10 | 74 | | 102 | | 111 | |
| | 100 | 41 | | 53 | | 84 | |
| | 500 | 22 | | 20 | | 51 | |
| 1653518 | 1 | 93 | 83 | 101 | 185 | 106 | 379 |
| | 10 | 79 | | 91 | | 101 | |
| | 100 | 48 | | 76 | | 82 | |
| | 500 | 24 | | 14 | | 42 | |
| 1653519 | 1 | 94 | 74 | 93 | 102 | 102 | 310 |
| | 10 | 79 | | 85 | | 106 | |
| | 100 | 44 | | 55 | | 76 | |
| | 500 | 23 | | 13 | | 38 | |

### Example 40: Design of RNAi Agents Containing 2′-Fluoro Xylosyl Nucleosides Targeted to HPRT

Modified oligonucleotides in the table below having either stereo-standard nucleosides or stereo-non-standard nucleosides in the antisense RNAi oligonucleotide and/or the sense RNAi oligonucleotides were synthesized using standard techniques. The antisense RNAi oligonucleotide has the sequence TUAAAAUCUACAGUCAUAGGAAU (SEQ ID NO: 9), as described herein above. Compound Nos. 1586322, 1591247, 1591249, and 1591250 are described herein above.

The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Compound No. 1586324 is described herein above.

TABLE 85
Design of sense RNAi oligonucleotides

| | | SEQ |
|---|---|---|
| Compound | Chemistry Notation | ID |
| No. | (5′ to 3′) | NO. |
| 1600510 | U_{ys}C_{ys}C_{yo}U_{yo}A_{yo}U_{[16C2r]o}G_{fo}A_{yo} | 10 |
| | C_{fo}U_{fo}G_{[f2bDx]o}U_{yo}A_{yo}G_{yo}A_{yo}U_{yo} | |
| | U_{yo}U_{yo}U_{ys}A_{ys}A_{y} | |
| 1600516 | U_{ys}C_{ys}C_{yo}U_{yo}A_{yo}U_{[16C2r]o}G_{[f2bDx]o} | 10 |
| | A_{yo}C_{fo}U_{fo}G_{fo}U_{yo}A_{yo}G_{yo}A_{yo}U_{yo}U_{yo} | |
| | U_{yo}U_{ys}A_{ys}A_{y} | |
| 1600517 | U_{ys}C_{ys}C_{yo}U_{yo}A_{yo}U_{[16C2r]o}G_{[f2bDx]o} | 10 |
| | A_{yo}C_{[f2bDx]o}U_{[f2bDx]o}G_{[f2bDx]o}U_{yo} | |
| | A_{yo}G_{yo}A_{yo}U_{yo}U_{yo}U_{yo}U_{ys}A_{ys}A_{y} | |
| 1601241 | U_{ys}C_{ys}C_{yo}U_{yo}A_{yo}U_{[16C2r]o}G_{[f2bDx]o} | 10 |
| | A_{yo}C_{fo}U_{fo}G_{[f2bDx]o}U_{yo}A_{yo}G_{yo}A_{yo}U_{yo} | |
| | U_{yo}U_{yo}U_{ys}A_{ys}A_{y} | |

In the table above, a subscript “[f2bDx]” represents a 2′-fluoro-β-D-xylosyl sugar moiety, a subscript “f” represents a 2′-fluoro-β-D-ribosyl sugar moiety, a subscript “y” represents a 2′-O-methyl-β-D-ribosyl sugar moiety, a subscript “[16C2r]” represents a 2′-O-hexadecylribosyl sugar moiety, a subscript “s” represents a phosphorothioate internucleoside linkage, and a subscript “o” represents a phosphodiester internucleoside linkage.

TABLE 86
Design of siRNA containing 2′-fluoro
xylosyl nucleosides targeted to HPRT1

| Duplex | Antisense Strand | Sense Strand |
|---|---|---|
| Compound No. | Compound No. | Compound No. |
| 1638654 | 1591247 | 1586324 |
| 1638665 | 1591249 | 1586324 |
| 1638672 | 1591250 | 1586324 |
| 1638673 | 1586322 | 1600516 |
| 1638674 | 1586322 | 1600510 |
| 1638675 | 1586322 | 1601241 |
| 1638676 | 1586322 | 1600517 |
| 1638677 | 1591247 | 1600516 |
| 1638678 | 1591247 | 1600510 |
| 1638679 | 1591247 | 1601241 |
| 1638680 | 1591247 | 1600517 |
| 1638697 | 1591249 | 1600516 |
| 1638699 | 1591249 | 1600510 |
| 1638704 | 1591249 | 1601241 |
| 1638779 | 1591249 | 1600517 |

### Example 41: Dose-Dependent Inhibition of Human HPRT in HeLa Cells by Cross-Reactive siRNA Containing 2′-Fluoro Xylosyl Nucleosides

The RNAi agents described above were tested at various doses in HeLa cells. Cultured HeLa cells at a density of 7,000 cells per well were treated by RNAiMAX with siRNA at concentrations indicated in the table below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC). Parent Compound No. 1588822, described herein above was included as a control.

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment.

TABLE 87
Dose-dependent inhibition of human HPRT1 in HeLa cells by
siRNA containing 2′-fluoro xylosyl nucleosides

| | HPRT1 RNA (% UTC) | | | | | | | | |
|---|---|---|---|---|---|---|---|---|---|
| Compound | 3 | 1 | 0.33 | 0.11 | 0.037 | 0.012 | 0.0041 | 0.00014 | IC₅₀ |
| No. | nM | nM | nM | nM | nM | nM | nM | nM | (pM) |
| 1588822 | 2 | 3 | 4 | 6 | 12 | 32 | 36 | 47 | 16 |
| 1638654 | 3 | 6 | 12 | 21 | 31 | 51 | 60 | 73 | 527 |
| 1638665 | 8 | 6 | 8 | 16 | 30 | 37 | 56 | 62 | 273 |
| 1638672 | 85 | 108 | 109 | 105 | 106 | 107 | 102 | 106 | 3310 |
| 1638673 | 1 | 2 | 5 | 9 | 17 | 26 | 37 | 47 | 120 |
| 1638674 | 2 | 2 | 4 | 8 | 20 | 32 | 38 | 36 | 64 |
| 1638675 | 2 | 3 | 5 | 9 | 16 | 25 | 59 | 40 | 126 |
| 1638676 | 1 | 3 | 3 | 5 | 11 | 16 | 24 | 43 | 41 |

TABLE 88
Dose-dependent inhibition of human HPRT1 in HeLa cells by
siRNA containing 2′-fluoro xylosyl nucleosides

| | HPRT1 RNA (% UTC) | | | | | | | | |
|---|---|---|---|---|---|---|---|---|---|
| Compound | 30 | 10 | 3.33 | 1.11 | 0.37 | 0.12 | 0.041 | 0.0014 | IC₅₀ |
| No. | nM | nM | nM | nM | nM | nM | nM | nM | (pM) |
| 1588822 | 4 | 5 | 9 | 17 | 28 | 27 | 36 | 42 | 7 |
| 1638677 | 7 | 16 | 28 | 42 | 63 | 86 | 82 | 86 | 782 |
| 1638678 | 8 | 19 | 29 | 60 | 61 | 86 | 95 | 73 | 1127 |
| 1638679 | 7 | 16 | 25 | 46 | 65 | 82 | 86 | 90 | 826 |
| 1638680 | 15 | 16 | 29 | 52 | 84 | 91 | 95 | 81 | 145 |

TABLE 89
Dose-dependent inhibition of human HPRT1 in HeLa cells by
siRNA containing 2′-fluoro xylosyl nucleosides

| | HPRT1 RNA (% UTC) | | | | | | | | |
|---|---|---|---|---|---|---|---|---|---|
| Compound | 3 | 1 | 0.33 | 0.11 | 0.037 | 0.012 | 0.0041 | 0.00014 | IC₅₀ |
| No. | nM | nM | nM | nM | nM | nM | nM | nM | (pM) |
| 1588822 | 6 | 9 | 19 | 34 | 54 | 66 | 80 | 80 | 4 |
| 1638697 | 6 | 10 | 16 | 26 | 44 | 51 | 64 | 69 | 13 |
| 1638699 | 11 | 16 | 24 | 46 | 57 | 66 | 75 | 60 | 37 |
| 1638704 | 7 | 19 | 25 | 38 | 56 | 70 | 73 | 73 | 41 |
| 1638779 | 10 | 13 | 24 | 32 | 53 | 73 | 73 | 79 | 30 |

### Example 42: Activity of siRNAs Containing 2′-Fluoro Xylosyl Nucleosides that Target Mouse HPRT, In Vivo

The activity of RNAi agents containing lipids was tested in wild type C57BL/6 mice (Taconic Biosciences).

Mice were divided into groups of 2 mice each. Each mouse received a single ICV bolus of 1 μg, 10 μg, 100 μg, and/or 500 μg of RNAi agent. A group of 4 mice received PBS as a control. Two weeks post treatment, mice were sacrificed, and RNA was extracted from cortical brain tissue, spinal cord, and liver tissue for quantitative real-time RTPCR analysis of RNA expression of HPRT using primer probe set RTS43125 (described herein above). Results are presented as percent change of RNA, relative to PBS control, normalized to mouse cyclophilin A (% control). Mouse cyclophilin A was amplified using primer probe set m_cyclo24 (designated herein above). As shown in the table below, treatment with modified oligonucleotides resulted in reduction of HPRT RNA in comparison to the PBS control.

The half maximal dose (EC₅₀) of each RNAi agent was calculated with GraphPad Prism software (v8.2.0, San Diego, CA) using the log (agonist) vs. response function: Y=Bottom+(Top−Bottom)/(1+10\hat{ }((Log EC50−X)*HillSlope)), with the following constraints: Bottom=0, Top=100. N.C. refers to data points that were not calculated.

TABLE 90
Reduction of mouse HPRT RNA in wild type C57BL/6 mice

| | | Cortex | | Spinal Cord | | Liver | |
|---|---|---|---|---|---|---|---|
| Com- | | HPRT | | HPRT | | HPRT | |
| pound | Dose | RNA | ED₅₀ | RNA | ED₅₀ | RNA | ED₅₀ |
| No. | (μg) | (% control) | (μg) | (% control) | (μg) | (% control) | (μg) |
| 1588822 | 1 | 95 | 18 | 91 | 16 | 101 | 228 |
| | 10 | 62 | | 61 | | 93 | |
| | 100 | 17 | | 15 | | 69 | |
| | 500 | 8 | | 8 | | 32 | |
| 1638654 | 1 | 94 | 174 | 94 | 138 | 101 | 51 |
| | 10 | 87 | | 94 | | 84 | |
| | 100 | 68 | | 46 | | 32 | |
| | 500 | 23 | | 36 | | 13 | |
| 1638665 | 1 | 91 | 78 | 97 | 62 | 77‡ | 149 |
| | 10 | 85 | | 85 | | 83‡ | |
| | 100 | 46 | | 35 | | 62 | |
| | 500 | 17 | | 22 | | 29 | |
| 1638673 | 1 | 87 | 18 | 93 | 19 | 107 | 8 |
| | 10 | 65 | | 64 | | 32 | |
| | 100 | 16 | | 19 | | 13 | |
| 1638674 | 1 | 85 | 14 | 97 | 20 | 91 | 34 |
| | 10 | 58 | | 64 | | 80 | |
| | 100 | 18 | | 19 | | 23 | |
| | 500 | 8 | | 9 | | 10 | |
| 1638675 | 1 | 88 | 14 | 93 | 10 | — | 57 |
| | 10 | 56 | | 45 | | 93 | |
| | 100 | 20 | | 16 | | 30 | |
| | 500 | 7 | | 8 | | 13 | |
| 1638676 | 1 | 83 | 14 | 94 | 26 | 100 | 71 |
| | 10 | 59 | | 72 | | 89 | |
| | 100 | 19 | | 20 | | 40 | |
| | 500 | 10‡ | | 13‡ | | 15‡ | |

‡indicates fewer than two subjects

### Example 43: Activity of siRNAs Containing 2′-Fluoro Xylosyl Nucleosides that Target Mouse HPRT, In Vivo

The activity of RNAi agents containing lipids was tested in wild type C57BL/6 mice (Taconic Biosciences).

Mice were divided into groups of 2 mice each. Each mouse received a single ICV bolus of 1 μg, 10 μg, 100 μg, and/or 500 μg of RNAi agent. A group of 4 mice received PBS as a control. Two weeks post treatment, mice were sacrificed, and RNA was extracted from cortical brain tissue, spinal cord, and liver tissue for quantitative real-time RTPCR analysis of RNA expression of HPRT using primer probe set RTS43125 (described herein above). Results are presented as percent change of RNA, relative to PBS control, normalized to mouse cyclophilin A (% control). Mouse cyclophilin A was amplified using primer probe set m_cyclo24 (designated herein above). As shown in the table below, treatment with modified oligonucleotides resulted in reduction of HPRT RNA in comparison to the PBS control.

The half maximal dose (EC₅₀) of each RNAi agent was calculated with GraphPad Prism software (v8.2.0, San Diego, CA) using the log (agonist) vs. response function: Y=Bottom+(Top−Bottom)/(1+10\hat{ }((Log EC50−X)*HillSlope)), with the following constraints: Bottom=0, Top=100.

TABLE 91
Reduction of mouse HPRT RNA in wild type C57BL/6 mice

| | | Spinal Cord | | Cortex | | Liver | |
|---|---|---|---|---|---|---|---|
| Compound | | HPRT RNA | ED₅₀ | HPRT RNA | ED₅₀ | HPRT RNA | ED₅₀ |
| No. | Dose (μg) | (% control) | (μg) | (% control) | (μg) | (% control) | (μg) |
| 1588822 | 1 | 911 | 8 | 95 | 11 | 102 | 23 |
| | 10 | 43 | | 49 | | 66 | |
| | 100 | 15 | | 14 | | 20 | |
| | 500 | 11 | | 10 | | 10 | |
| 1638677 | 1 | 88 | 109 | 98 | >100 | 97 | >100 |
| | 10 | 84 | | 94 | | 99 | |
| | 100 | 50 | | 62 | | 69 | |
| 1638678 | 1 | 91 | 113 | 101 | 174 | 103 | 163 |
| | 10 | 77 | | 97 | | 95 | |
| | 100 | 50 | | 60 | | 61 | |
| | 500 | 30 | | 29 | | 21 | |
| 1638679 | 1 | 92 | 123 | 104 | 263 | 91 | 139 |
| | 10 | 88 | | 99 | | 92 | |
| | 100 | 45 | | 64 | | 55 | |
| | 500 | 37‡ | | 42‡ | | 23‡ | |
| 1638680 | 1 | 86 | 299 | 99 | 232 | 91 | 230 |
| | 10 | 83 | | 93 | | 64 | |
| | 100 | 50 | | 66 | | 64 | |
| | 500 | 47 | | 35 | | 38 | |
| 1638699 | 1 | 89 | 52 | 101 | 70 | 91 | 55 |
| | 10 | 67 | | 88 | | 88 | |
| | 100 | 41 | | 39 | | 31 | |
| | 500 | 25 | | 17 | | 18 | |
| 1638779 | 1 | 90 | 83 | 105 | 135 | 86 | 116 |
| | 10 | 80 | | 92 | | 103 | |
| | 100 | 45 | | 53 | | 50 | |
| | 500 | 24 | | 28 | | 17 | |

‡indicates fewer than two subjects

### Example 44: Design of RNAi Agents Targeted to FXII Containing 5′-Vinylphosphonate Moieties or 5′-Mesyl Phosphoramidate Moieties

Modified oligonucleotides in the table below having either stereo-standard nucleosides or stereo-non-standard nucleosides in the antisense RNAi oligonucleotide and/or the sense RNAi oligonucleotides were synthesized using standard techniques. Aside from a single mismatch at position 1 on the 5′-end, each antisense strand consists of the sequence TAAAGCACUUUAUUGAGUUUCUG (SEQ ID NO: 31) and is complementary to the complement of GenBank Accession No. NC_000079.6, truncated from nucleosides 55415001 to 55430000, (SEQ ID NO. 25, described herein above) from nucleosides 12005 to 12026 (SEQ ID NO: 39).

TABLE 92
Design of antisense strand modified oligonucleotides targeted
to FXII containing 5′-vinylphosphonate moieties or
5′-mesyl phosphoramidate moieties

| Compound | | SEQ ID |
|---|---|---|
| No. | Chemistry Notation (5′ to 3′) | NO. |
| 1526195 | vP-TₑₛA_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 31 |
| 1599518 | vP- [symbol] A_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo} [symbol] U_{ys}G_{y} | 31 |
| 1625847 | z. [symbol] A_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo} [symbol] U_{ys}G_{y} | 24 |
| 1599520 | vP-Tₑₛ [symbol] A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo} [symbol] U_{ys}G_{y} | 31 |
| 1625848 | z.U_{ys} [symbol] A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo} [symbol] U_{ys}G_{y} | 24 |
| 1599524 | vP-Tₑₛ [symbol] A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 31 |
| 1625849 | z.U_{ys} [symbol] A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |

In the table above, a “vP-” represents a vinyl phosphonate (vP) moiety on the 5′-end, a “z.” represents a 5′-mesyl phosphoramidate terminal group having Formula XIII, a subscript “e” represents a 2′-MOE sugar moiety, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, a subscript “o” represents a phosphodiester internucleoside linkage, and a subscript “z” represents a mesyl phosphoramidate internucleoside linkage (Formula II).

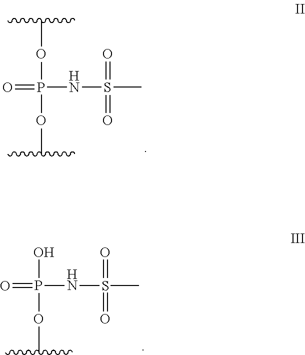

Compound No. 1523578 is described herein above and was previously disclosed in WO 2021/030778. Compound No. 1523578 is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

TABLE 93
Design of siRNA targeted to FXII containing 5′-vinylphosphonate
moieties or 5′-mesyl phosphoramidate moieties

| Duplex | Antisense Strand | Sense Strand |
|---|---|---|
| Compound No. | Compound No. | Compound No. |
| 1526196 | 1526195 | 1523578 |
| 1645351 | 1599518 | 1523578 |
| 1645354 | 1625847 | 1523578 |
| 1645352 | 1599520 | 1523578 |
| 1645355 | 1625848 | 1523578 |
| 1645353 | 1599524 | 1523578 |
| 1645356 | 1625849 | 1523578 |

### Example 45: Design of RNAi Agents Targeted to FXII with Ribonucleoside Moieties

Modified oligonucleotides in the table below having either stereo-standard nucleosides or stereo-non-standard nucleosides in the antisense RNAi oligonucleotide and/or the sense RNAi oligonucleotides were synthesized using standard techniques. Each antisense strand described in the table below has the sequence UAAAGCACUUUAUUGAGUUUCUG (SEQ ID NO: 24). Aside from a single mismatch at position 1 on the 5′-end, each antisense strand is complementary to the complement of GenBank Accession No. NC_000079.6, truncated from nucleosides 55415001 to 55430000, (SEQ ID NO: 25, described herein above) from nucleosides 12005 to 12026 (SEQ ID NO: 39). Each antisense strand has a 5′-phosphate.

TABLE 94
Design of antisense strand modified oligonucleotides targeted to FXII
with ribonucleoside moieties

| Compound | | SEQ ID |
|---|---|---|
| No. | Chemistry Notation (5′ to 3′) | NO. |
| 1626280 | p.U_{ys}A_{fs}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1626281 | p.U_{ys}AᵣₛA_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1626282 | p.U_{ys}A_{fs}A_{yo}A_{yo}G_{yo}CᵣₒA_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1626283 | p.U_{ys}A_{fs}A_{yo}A_{yo}G_{yo}C_{fo}A_{yo}C_{yo}U_{yo}U_{yo}U_{yo}A_{yo}U_{yo}UᵣₒG_{yo}A_{fo}G_{yo}U_{yo}U_{yo}U_{yo}C_{ys}U_{ys}G_{y} | 24 |
| 1626284 | p.U_{ys}A_{fs}A_{yo}A_{yo}G_{yo}C_{fo}A_{yo}C_{yo}U_{yo}U_{yo}U_{yo}A_{yo}U_{yo}U_{fo}G_{yo}AᵣₒG_{yo}U_{yo}U_{yo}U_{yo}C_{ys}U_{ys}G_{y} | 24 |
| 1626285 | p.U_{ys}AᵣₛA_{yo}A_{yo}G_{yo}CᵣₒA_{yo}C_{yo}U_{yo}U_{yo}U_{yo}A_{yo}U_{yo}UᵣₒG_{yo}AᵣₒG_{yo}U_{yo}U_{yo}U_{yo}C_{ys}U_{ys}G_{y} | 24 |

In the table above, a subscript “f” represents a 2′-fluoro-β-D-ribosyl sugar moiety, a subscript “y” represents a 2′-O-methyl-β-D-ribosyl sugar moiety, a subscript “r” represents a β-D-ribosyl sugar moiety, a subscript “s” represents a phosphorothioate internucleoside linkage, and a subscript “o” represents a phosphodiester internucleoside linkage.

The sense RNAi oligonucleotide is complementary to the first of the 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Each sense RNAi oligomeric compound further contains a GalNAc moiety conjugated to the 3′-oxygen as shown below:

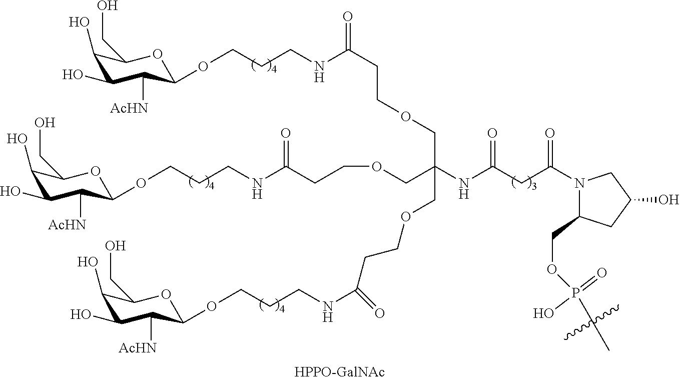

Compound No. 1523578 is described herein above and was previously disclosed in WO 2021/030778.

TABLE 95
Design of GaINAc-conjugated sense RNAi oligomeric compounds
with ribonucleoside moieties

| Compound | | SEQ ID |
|---|---|---|
| No. | Chemistry Notation (5′ to 3′) | NO. |
| 1626286 | G_{ys}A_{ys}A_{yo}A_{yo}C_{yo}U_{yo}CᵣₒA_{yo}A_{fo}U_{fo}A_{fo}A_{yo}A_{yo}G_{yo}U_{yo}G_{yo}C_{yo}U_{yo}U_{yo}U_{yo}A_{y}-HPPO-GalNAc | 26 |
| 1626287 | G_{ys}A_{ys}A_{yo}A_{yo}C_{yo}U_{yo}C_{fo}A_{yo}AᵣₒU_{fo}A_{fo}A_{yo}A_{yo}G_{yo}U_{yo}G_{yo}C_{yo}U_{yo}U_{yo}U_{yo}A_{y}-HPPO-GalNAc | 26 |
| 1626288 | G_{ys}A_{ys}A_{yo}A_{yo}C_{yo}U_{yo}C_{fo}A_{yo}A_{fo}UᵣₒA_{fo}A_{yo}A_{yo}G_{yo}U_{yo}G_{yo}C_{yo}U_{yo}U_{yo}U_{yo}A_{y}-HPPO-GalNAc | 26 |
| 1626289 | G_{ys}A_{ys}A_{yo}A_{yo}C_{yo}U_{yo}C_{fo}A_{yo}A_{fo}U_{fo}AᵣₒA_{yo}A_{yo}G_{yo}U_{yo}G_{yo}C_{yo}U_{yo}U_{yo}U_{yo}A_{y}-HPPO-GalNAc | 26 |
| 1626290 | G_{ys}A_{ys}A_{yo}A_{yo}C_{yo}U_{yo}CᵣₒA_{yo}AᵣₒUmAᵣₒA_{yo}A_{yo}G_{yo}U_{yo}G_{yo}C_{yo}U_{yo}U_{yo}U_{yo}A_{y}-HPPO-GalNAc | 26 |

In the table above, a subscript “f” represents a 2′-fluoro-β-D-ribosyl sugar moiety, a subscript “y” represents a 2′-O-methyl-β-D-ribosyl sugar moiety, a subscript “r” represents a β-D-ribosyl sugar moiety, a subscript “s” represents a phosphorothioate internucleoside linkage, and a subscript “o” represents a phosphodiester internucleoside linkage.

TABLE 96
Design of siRNA targeted to FXII with ribonucleoside moieties

| Duplex | Antisense Strand | Sense Strand |
|---|---|---|
| Compound No. | Compound No. | Compound No. |
| 1632812 | 1626280 | 1523578 |
| 1645198 | 1626281 | 1523578 |
| 1645199 | 1626282 | 1523578 |
| 1645200 | 1626283 | 1523578 |
| 1645201 | 1626284 | 1523578 |
| 1645202 | 1626285 | 1523578 |
| 1645203 | 1626280 | 1626286 |
| 1645204 | 1626280 | 1626287 |
| 1645205 | 1626280 | 1626288 |
| 1645206 | 1626280 | 1626289 |
| 1645207 | 1626280 | 1626290 |
| 1645208 | 1626281 | 1626290 |

### Example 46: Design of siRNA Targeted to FXII Containing F-HNA and 2′-β-D-Deoxyxylosyl Nucleosides

Modified oligonucleotides in the table below having either stereo-standard nucleosides or stereo-non-standard nucleosides in the antisense RNAi oligonucleotide and/or the sense RNAi oligonucleotides were synthesized using standard techniques.

Each antisense strand described in the table below has the sequence UAAAGCACUUUAUUGAGUUUCUG (SEQ ID NO: 24), described herein above. Aside from a single mismatch at position 1 on the 5′-end, each antisense strand is complementary to the complement of GenBank Accession No. NC_000079.6, truncated from nucleosides 55415001 to 55430000, (SEQ ID NO: 25, described herein above) from nucleosides 12005 to 12026 (SEQ ID NO: 39). Compound No. 1523579 is described herein above and was previously disclosed in WO 2021/030778.

TABLE 97
Design of antisense strand modified oligonucleotides targeted to FXII
containing F-HNA and 2′-β-D-deoxyxylosyl nucleosides

| | | SEQ |
|---|---|---|
| Compound | | ID |
| No. | Chemistry Notation (5′ to 3′) | NO. |
| 1620649 | U_{ys}A_{[bDdx]s}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1620650 | U_{ys}A_{fs}A_{yo}A_{yo}G_{yo}^{m}C_{[bDdx]o}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1620651 | U_{ys}A_{fs}A_{yo}A_{yo}G_{yo}C_{fo}A_{yo}C_{yo}U_{yo}U_{yo}U_{yo}A_{yo}U_{yo}T_{[bDdx]o}G_{yo}A_{fo}G_{yo}U_{yo}U_{yo}U_{yo}C_{ys}U_{ys}G_{y} | 32 |
| 1620652 | U_{ys}A_{fs}A_{yo}A_{yo}G_{yo}C_{fo}A_{yo}C_{yo}U_{yo}U_{yo}U_{yo}A_{yo}U_{yo}U_{fo}G_{yo}A_{[bDdx]o}G_{yo}U_{yo}U_{yo}U_{yo}C_{ys}U_{ys}G_{y} | 24 |
| 1620653 | U_{ys}A_{[bDdx]s}A_{yo}A_{yo}G_{yo}^{m}C_{[bDdx]o}A_{yo}C_{yo}U_{yo}U_{yo}U_{yo}A_{yo}U_{yo}T_{[bDdx]o}G_{yo}A_{[bDdx]o}G_{yo}U_{yo} | 32 |
| | U_{yo}U_{yo}C_{ys}U_{ys}G_{y} | |
| 1620654 | U_{ys}A_{[F-HNA]s}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1620655 | U_{ys}A_{fs}A_{yo}A_{yo}G_{yo}^{m}C_{[F-HNA]o}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1620656 | U_{ys}A_{fs}A_{yo}A_{yo}G_{yo}C_{fo}A_{yo}C_{yo}U_{yo}U_{yo}U_{yo}A_{yo}U_{yo}T_{[F-HNA]o}G_{yo}A_{fo}G_{yo}U_{yo}U_{yo}U_{yo}C_{ys}U_{ys}G_{y} | 32 |
| 1620657 | U_{ys}A_{fs}A_{yo}A_{yo}G_{yo}C_{fo}A_{yo}C_{yo}U_{yo}U_{yo}U_{yo}A_{yo}U_{yo}U_{fo}G_{yo}A_{[F-HNA]o}G_{yo}U_{yo}U_{yo}U_{yo}C_{ys}U_{ys}G_{y} | 24 |
| 1620658 | U_{ys}A_{[F-HNA]s}A_{yo}A_{yo}G_{yo}^{m}C_{[F-HNA]o}A_{yo}C_{yo}U_{yo}U_{yo}U_{yo}A_{yo}U_{yo}T_{[F-HNA]O}G_{yo}A_{[F-HNA]o} | 32 |
| | G_{yo}U_{yo}U_{yo}U_{yo}C_{ys}U_{ys}G_{y} | |
| 1633631 | p.U_{ys}A_{[bDdx]s}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1633632 | p.U_{ys}A_{[bDdx]s}A_{yo}A_{yo}G_{yo}^{m}_{[bDdx]o}A_{yo}C_{yo}U_{yo}U_{yo}U_{yo}A_{yo}U_{yo}T_{[bDdx]o}G_{yo}A_{[bDdx]o} | 32 |
| | G_{yo}U_{yo}U_{yo}U_{yo}C_{ys}U_{ys}G_{y} | |
| 1633633 | p.U_{ys}A_{[F-HNA]s}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 24 |
| 1633634 | p.U_{ys}A_{[F-HNA]s}A_{yo}A_{yo}G_{yo}^{m}C_{[F-HNA]o}A_{yo}C_{yo}U_{yo}U_{yo}U_{yo}A_{yo}U_{yo}T_{[F-HNA]O}G_{yo}A_{[F-HNA]o} | 32 |
| | G_{yo}U_{yo}U_{yo}U_{yo}C_{ys}U_{ys}G_{y} | |

In the table above, a “p.” represents a 5′ terminal phosphate group, a subscript “f” represents a 2′-fluoro-β-D-ribosyl sugar moiety, a subscript “y” represents a 2′-O-methyl-β-D-ribosyl sugar moiety, a subscript “r” represents a β-D-ribosyl sugar moiety, a subscript “[F-HNA]” represents a 3′-fluoro-hexitol sugar surrogate, a subscript “[bDdx]” represents a 2′β-D-deoxyxylosyl sugar moiety, a subscript “s” represents a phosphorothioate internucleoside linkage, and a subscript “o” represents a phosphodiester internucleoside linkage. “^{m}C” in the table above represents a 5-methylcytosine.

The sense RNAi oligonucleotides in the table below are complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Each sense RNAi oligomeric compound further contains a GalNAc moiety conjugated to the 3′-oxygen as shown below:

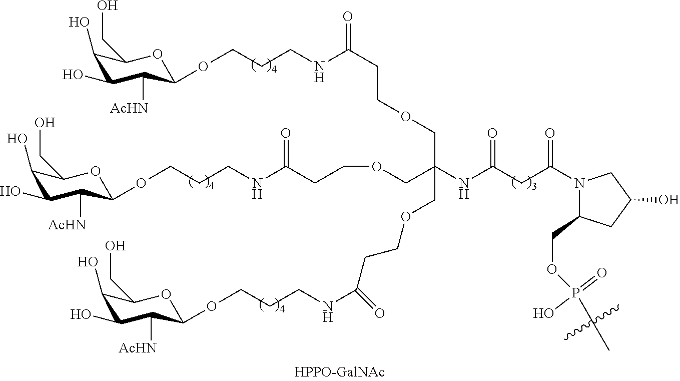

Compound No. 1523578 is described herein above and was previously disclosed in WO 2021/030778.

TABLE 98
Design of sense RNAi oligonucleotides
containing F-HNA and 2′-β-D-deoxyxylosyl nucleosides

| Compound | | SEQ ID |
|---|---|---|
| No. | Chemistry Notation (5′ to 3′) | NO. |
| 1625998 | G_{ys}A_{ys}A_{yo}A_{yo}C_{yo}U_{yo}C_{fo}A_{yo}A_{fo}U_{fo}A_{[bDdx]o}A_{yo}A_{yo}G_{yo}U_{yo}G_{yo}C_{yo}U_{yo}U_{yo}U_{yo}A_{y}-HPPO- | 26 |
| | GalNAc | |
| 1625999 | G_{ys}A_{ys}A_{yo}A_{yo}C_{yo}U_{yo}C_{fo}A_{yo}A_{fo}T_{[bDdx]o}A_{fo}A_{yo}A_{yo}G_{yo}U_{yo}G_{yo}C_{yo}U_{yo}U_{yo}U_{yo}A_{y}-HPPO- | 33 |
| | GalNAc | |
| 1626000 | G_{ys}A_{ys}A_{yo}A_{yo}C_{yo}U_{yo}C_{fo}A_{yo}A_{[bDdx]o}U_{fo}A_{fo}A_{yo}A_{yo}G_{yo}U_{yo}G_{yo}C_{yo}U_{yo}U_{yo}U_{yo}A_{y}-HPPO- | 26 |
| | GalNAc | |
| 1626001 | G_{ys}A_{ys}A_{yo}A_{yo}C_{yo}U_{yo}^{m}C_{[bDdx]o}A_{yo}A_{fo}U_{fo}A_{fo}A_{yo}A_{yo}G_{yo}U_{yo}G_{yo}C_{yo}U_{yo}U_{yo}U_{yo}A_{y}-HPPO- | 26 |
| | GalNAc | |
| 1626002 | G_{ys}A_{ys}A_{yo}A_{yo}C_{yo}U_{yo}^{m}C_{[bDdx]o}A_{yo}A_{[bDdx]o}T_{[bDdx]o}A_{[bDdx]o}A_{yo}A_{yo}G_{yo}U_{yo}G_{yo}C_{yo}U_{yo} | 33 |
| | U_{yo}U_{yo}A_{y}-HPPO-GalNAc | |
| 1626003 | G_{ys}A_{ys}A_{yo}A_{yo}C_{yo}U_{yo}C_{fo}A_{yo}A_{fo}U_{fo}A_{[F-HNA]o}A_{yo}A_{yo}G_{yo}U_{yo}G_{yo}C_{yo}U_{yo}U_{yo}U_{yo}A_{y}-HPPO- | 26 |
| | GalNAc | |
| 1626004 | G_{ys}A_{ys}A_{yo}A_{yo}C_{yo}U_{yo}C_{fo}A_{yo}A_{fo}T_{[F-HNA]o}A_{fo}A_{yo}A_{yo}G_{yo}U_{yo}G_{yo}C_{yo}U_{yo}U_{yo}U_{yo}A_{y}-HPPO- | 33 |
| | GalNAc | |
| 1626005 | G_{ys}A_{ys}A_{yo}A_{yo}C_{yo}U_{yo}C_{fo}A_{yo}A_{[F-HNA]o}U_{fo}A_{fo}A_{yo}A_{yo}G_{yo}U_{yo}G_{yo}C_{yo}U_{yo}U_{yo}U_{yo}A_{y}-HPPO- | 26 |
| | GalNAc | |
| 1626006 | G_{ys}A_{ys}A_{yo}A_{yo}C_{yo}U_{yo}^{m}C_{[F-HNA]o}A_{yo}A_{fo}U_{fo}A_{fo}A_{yo}A_{yo}G_{yo}U_{yo}G_{yo}C_{yo}U_{yo}U_{yo}U_{yo}A_{y}-HPPO- | 26 |
| | GalNAc | |
| 1626007 | G_{ys}A_{ys}A_{yo}A_{yo}C_{yo}U_{yo}^{m}C_{[F-HNA]o}A_{yo}A_{[F-HNA]o}T_{[F-HNA]o}A_{[F-HNA]o}A_{yo}A_{yo}G_{yo}U_{yo}G_{yo}C_{yo}U_{yo} | 33 |
| | U_{yo}U_{yo}A_{y}-HPPO-GalNAc | |

In the table above, a subscript “f” represents a 2′-fluoro-β-D-ribosyl sugar moiety, a subscript “y” represents a 2′-O-methyl-β-D-ribosyl sugar moiety, a subscript “r” represents a β-D-ribosyl sugar moiety, a subscript “[F-HNA]” represents a 3′-fluoro-hexitol sugar surrogate, a subscript “[bDdx]” represents a 2′-β-D-deoxyxylosyl sugar moiety, a subscript “s” represents a phosphorothioate internucleoside linkage, and a subscript “o” represents a phosphodiester internucleoside linkage. “mC” in the table above represents a 5-methylcytosine.

TABLE 99
Design of siRNA targeted to FXII containing F-HNA
and 2′-β-D-deoxyxylosyl nucleosides

| Duplex | Antisense Strand | Sense Strand |
|---|---|---|
| Compound No. | Compound No. | Compound No. |
| 1633605 | 1620649 | 1523578 |
| 1633606 | 1620650 | 1523578 |
| 1633607 | 1620651 | 1523578 |
| 1633608 | 1620652 | 1523578 |
| 1633609 | 1620653 | 1523578 |
| 1633610 | 1620654 | 1523578 |
| 1633611 | 1620655 | 1523578 |
| 1633612 | 1620656 | 1523578 |
| 1633613 | 1620657 | 1523578 |
| 1633614 | 1620658 | 1523578 |
| 1633615 | 1523579 | 1625998 |
| 1633616 | 1523579 | 1625999 |
| 1633617 | 1523579 | 1626000 |
| 1633618 | 1523579 | 1626001 |
| 1633619 | 1523579 | 1626002 |
| 1633620 | 1523579 | 1626003 |
| 1633621 | 1523579 | 1626004 |
| 1633622 | 1523579 | 1626005 |
| 1633623 | 1523579 | 1626006 |
| 1633624 | 1523579 | 1626007 |
| 1633635 | 1633631 | 1523578 |
| 1633636 | 1633632 | 1523578 |
| 1633637 | 1633633 | 1523578 |
| 1633638 | 1633634 | 1523578 |

### Example 47: Design of siRNA Targeted to FXII Containing 2′-O-Methyl-β-D-Xylosyl Nucleosides

Modified oligonucleotides in the table below having either stereo-standard nucleosides or stereo-non-standard nucleosides in the antisense RNAi oligonucleotide and/or the sense RNAi oligonucleotides were synthesized using standard techniques.

Each antisense RNAi oligonucleotide described in the table below has the sequence UAAAGCACUUUAUUGAGUUUCUG (SEQ ID NO: 24). Aside from a single mismatch at position 1 on the 5′-end, each antisense RNAi oligonucleotide is complementary to the complement of GenBank Accession No. NC_000079.6, truncated from nucleosides 55415001 to 55430000, (SEQ ID NO: 25, described herein above) from nucleosides 12005 to 12026 (SEQ ID NO: 39). Compound No. 1523579 is described herein above and was previously disclosed in WO 2021/030778.

TABLE 100
Design of antisense strand modified
oligonucleotides targeted to FXII
containing 2′-O-methyl-β-D-xylosyl
nucleosides

| Compound | | SEQ ID |
|---|---|---|
| No. | Chemistry Notation (5′ to 3′) | NO. |
| 1632760 | U_{ys}A_{fs}A_{yo}A_{yo}G_{yo}C_{fo}A_{yo}C_{yo}U_{yo}U_{yo}U_{yo}A_{yo}U_{yo} | 32 |
| | T_{[m2bDx]o}G_{yo}A_{fo}G_{yo}U_{yo}U_{yo}U_{yo}C_{ys}U_{ys}G_{y} | |
| 1631342 | p.U_{ys}A_{fs}A_{yo}A_{yo}G_{yo}C_{fo}A_{yo}C_{yo}U_{yo}U_{yo}U_{yo}A_{yo} | 32 |
| | U_{yo}T_{[m2bDx]o}G_{yo}A_{fo}G_{yo}U_{yo}U_{yo}U_{yo}C_{ys}U_{ys}G_{y} | |

In the table above, a “p.” represents a 5′ terminal phosphate group, a subscript “f” represents a 2′-fluoro-β-D-ribosyl sugar moiety, a subscript “y” represents a 2′-O-methyl-β-D-ribosyl sugar moiety, a subscript “[m2bDx]” represents a 2′-O-methyl-β-D-xylosyl sugar moiety, a subscript “s” represents a phosphorothioate internucleoside linkage, a subscript “o” represents a phosphodiester internucleoside linkage.

The sense RNAi oligonucleotide described in the table below is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Each sense RNAi oligomeric compound further contains a GalNAc moiety conjugated to the 3′-oxygen as shown below:

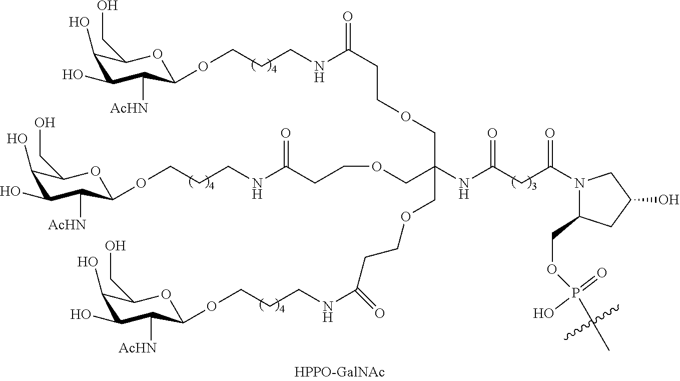

TABLE 101
Design of sense RNAi oligomeric compounds
containing 2′-O-methyl-β-D-xylosyl
nucleosides and a GalNAc conjugate

| Compound | | SEQ ID |
|---|---|---|
| No. | Chemistry Notation (5′ to 3′) | NO. |
| 1631343 | G_{ys}A_{ys}A_{yo}A_{yo}C_{yo}U_{yo}C_{fo}A_{yo}A_{fo}T[_{m2bDx]o}A_{fo} | 33 |
| | A_{yo}A_{yo}G_{yo}U_{yo}G_{yo}C_{yo}U_{yo}U_{yo}U_{yo}A_{y}-HPPO- | |
| | GalNAc | |

In the table above, a subscript “f” represents a 2′-fluoro-β-D-ribosyl sugar moiety, a subscript “y” represents a 2′-O-methyl-β-D-ribosyl sugar moiety, a subscript “r” represents a β-D-ribosyl sugar moiety, a subscript “[m2bDx]” represents a 2′-O-methyl-β-D-xylosyl sugar, a subscript “s” represents a phosphorothioate internucleoside linkage, and subscript “o” represents a phosphodiester internucleoside linkage.

Compound No. 1523578 is described herein above and was previously disclosed in WO 2021/030778.

TABLE 102
Design of siRNA targeted to FXII containing
2′-O-methyl-β-D-xylosyl nucleosides

| Duplex | Antisense Strand | Sense Strand |
|---|---|---|
| Compound No. | Compound No. | Compound No. |
| 1650412 | 1632760 | 1523578 |
| 1650450 | 1523579 | 1631343 |
| 1650487 | 1632760 | 1631343 |
| 1632813 | 1631342 | 1523578 |
| 1632814 | 1626280 | 1631343 |
| 1632815 | 1631342 | 1631343 |

### Example 48: Design of siRNA Targeted to FXII Containing β-D-Arabinosyl Nucleosides

Modified oligonucleotides in the table below having either stereo-standard nucleosides or stereo-non-standard nucleosides in the antisense RNAi oligonucleotide and/or the sense RNAi oligonucleotide were synthesized using standard techniques. Each antisense RNAi oligonucleotide described in the table below has the sequence UAAAGCACUUUAUUGAGUUUCUG (SEQ ID NO: 24). Aside from a single mismatch at position 1 on the 5′-end, each antisense RNAi oligonucleotide is complementary to the complement of GenBank Accession No. NC_000079.6, truncated from nucleosides 55415001 to 55430000, (SEQ ID NO: 25) from nucleosides 12005 to 12026 (SEQ ID NO: 39). Each antisense RNAi oligonucleotide has a 5′-phosphate.

TABLE 103
Design of antisense RNAi oligonucleotides
targeted to FXII containing
β-D-arabinosyl nucleosides

| Compound | | SEQ ID |
|---|---|---|
| No. | Chemistry Notation (5′ to 3′) | NO. |
| 1652693 | p.U_{ys}A_{[bDa]s}A_{yo}A_{yo}G_{yo}C_{fo}A_{yo}C_{yo}U_{yo} | 24 |
| | U_{yo}U_{yo}A_{yo}U_{yo}U_{fo}G_{yo}A_{fo}G_{yo}U_{yo}U_{yo}U_{yo} | |
| | C_{ys}U_{ys}G_{y} | |
| 1652694 | p.U_{ys}A_{fs}A_{yo}A_{yo}G_{yo}C_{[bDa]o}A_{yo}C_{yo}U_{yo} | 24 |
| | U_{yo}U_{yo}A_{yo}U_{yo}U_{fo}G_{yo}A_{fo}G_{yo}U_{yo}U_{yo}U_{yo} | |
| | C_{ys}U_{ys}G_{y} | |
| 1652695 | p.U_{ys}A_{fs}A_{yo}A_{yo}G_{yo}C_{fo}A_{yo}C_{yo}U_{yo}U_{yo} | 24 |
| | U_{yo}A_{yo}U_{yo}U_{[bDa]o}G_{yo}A_{fo}G_{yo}U_{yo}U_{yo}U_{yo} | |
| | C_{ys}U_{ys}G_{y} | |
| 1652697 | p.U_{ys}A_{fs}A_{yo}A_{yo}G_{yo}C_{fo}A_{yo}C_{yo}U_{yo}U_{yo} | 24 |
| | U_{yo}A_{yo}U_{yo}U_{fo}G_{yo}A_{[bDa]o}G_{yo}U_{yo}U_{yo}U_{yo} | |
| | C_{ys}U_{ys}G_{y} | |
| 1652698 | p.U_{ys}A_{[bDa]s}A_{yo}A_{yo}G_{yo}C_{[bDa]o}A_{yo}C_{yo} | 24 |
| | U_{yo}U_{yo}U_{yo}A_{yo}U_{yo}U_{[bDa]o}G_{yo}A_{[bDa]o}G_{yo} | |
| | U_{yo}U_{yo}U_{yo}C_{ys}U_{ys}G_{y} | |

In the table above, a subscript “f” represents a 2′-fluoro-β-D-ribosyl sugar moiety, a subscript “y” represents a 2′-O-methyl-β-D-ribosyl sugar moiety, a subscript “[bDa]” represents a β-D-arabinosyl sugar moiety, a subscript “s” represents a phosphorothioate internucleoside linkage, and a subscript “o” represents a phosphodiester internucleoside linkage.

Compound No. 1523578 is described herein above and was previously disclosed in WO 2021/030778. Compound No. 1523578 is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides).

TABLE 104
Design of siRNA targeted to FXII containing
β-D-arabinosyl nucleosides

| Duplex | Antisense Strand | Sense Strand |
|---|---|---|
| Compound No. | Compound No. | Compound No. |
| 1652862 | 1652693 | 1523578 |
| 1652868 | 1652694 | 1523578 |
| 1652869 | 1652695 | 1523578 |
| 1652865 | 1652697 | 1523578 |
| 1652870 | 1652698 | 1523578 |

### Example 49: Design of siRNA Targeted to FXII Containing β-D-Xylosyl Nucleosides

Modified oligonucleotides in the table below having either stereo-standard nucleosides or stereo-non-standard nucleosides in the antisense RNAi oligonucleotides and/or the sense RNAi oligonucleotides were synthesized using standard techniques. Each antisense RNAi oligonucleotide described in the table below has the sequence UAAAGCACUUUAUUGAGUUUCUG (SEQ ID NO: 24). Aside from a single mismatch at position 1 on the 5′-end, each antisense RNAi oligonucleotide is complementary to the complement of GenBank Accession No. NC_000079.6, truncated from nucleosides 55415001 to 55430000 (SEQ ID NO: 25) from nucleosides 12005 to 12026 (SEQ ID NO: 39). Each antisense RNAi oligonucleotide has a 5′-phosphate. Antisense RNAi oligonucleotide Compound No. 1626280 is described herein above.

TABLE 105
Design of antisense RNAi oligonucleotides
targeted to FXII containing
β-D-xylosyl nucleosides

| Compound | | SEQ ID |
|---|---|---|
| No. | Chemistry Notation (5′ to 3′) | NO. |
| 1659242 | p.U_{ys}A_{fs}A_{yo}A_{yo}G_{yo}C_{fo}A_{yo}C_{yo}U_{yo}U_{yo} | 24 |
| | U_{yo}A_{yo}U_{yo}U_{[bDx]o}G_{yo}A_{fo}G_{yo}U_{yo}U_{yo}U_{yo} | |
| | C_{ys}U_{ys}G_{y} | |

In the table above, a subscript “f” represents a 2′-fluoro-β-D-ribosyl sugar moiety, a subscript “y” represents a 2′-O-methyl-β-D-ribosyl sugar moiety, a subscript “[bDx]” represents a β-D-xylosyl sugar moiety, a subscript “s” represents a phosphorothioate internucleoside linkage, and a subscript “o” represents a phosphodiester internucleoside linkage.

The sense RNAi oligonucleotide described in the table below is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). The sense RNAi oligomeric compound further contains a GalNAc moiety conjugated to the 3′-oxygen as shown below:

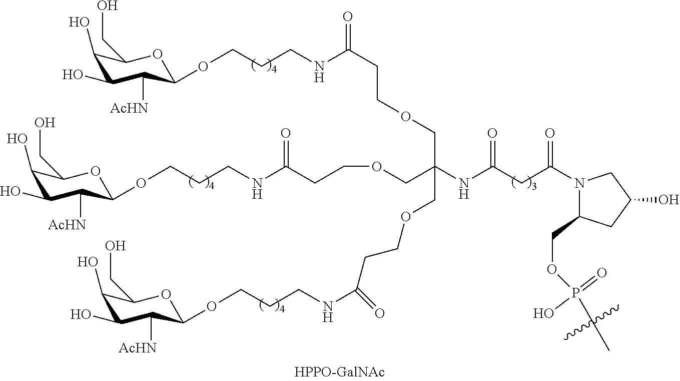

Compound No. 1523578 is described herein above and was previously disclosed in WO 2021/030778.

TABLE 106
Design of sense strand modified
oligonucleotides containing
β-D-xylosyl nucleosides

| Compound | | SEQ ID |
|---|---|---|
| No. | Chemistry Notation (5′ to 3′) | NO. |
| 1659243 | G_{ys}A_{ys}A_{yo}A_{yo}C_{yo}U_{yo}C_{fo}A_{yo}A_{fo}U_{[bDx]o} | 26 |
| | A_{fo}A_{yo}A_{yo}G_{yo}U_{yo}G_{yo}C_{yo}U_{yo}U_{yo}U_{yo}A_{y}- | |
| | HPPO-GalNAc | |

In the table above, a subscript “f” represents a 2′-fluoro-β-D-ribosyl sugar moiety, a subscript “y” represents a 2′-O-methyl-β-D-ribosyl sugar moiety, a subscript “[bDx]” represents a β-D-xylosyl sugar moiety, a subscript “s” represents a phosphorothioate internucleoside linkage, and a subscript “o” represents a phosphodiester internucleoside linkage.

TABLE 107
Design of siRNA targeted to FXII
containing β-D-xylosyl nucleosides

| Duplex | Antisense Strand | Sense Strand |
|---|---|---|
| Compound No. | Compound No. | Compound No. |
| 1659244 | 1659242 | 1523578 |
| 1659245 | 1626280 | 1659243 |
| 1659246 | 1659242 | 1659243 |

### Example 50: Design of RNAi Agents Containing 2′-Deoxyribonucleosides or 2′-Deoxyxylonucleosides Targeted to HPRT1

Modified oligonucleotides in the table below having either stereo-standard nucleosides or stereo-non-standard nucleosides in the antisense RNAi oligonucleotides and/or the sense RNAi oligonucleotide were synthesized using standard techniques. Compound No. 1455005 has the sequence AUAAAAUCUACAGUCAUAGGATT (SEQ ID NO: 34) and is complementary to human HPRT GenBank Accession No. NM_000194.2 (SEQ ID NO: 1) from nucleosides 444 to 466, with a single mismatch at position 22 (from 5′ to 3′) of the antisense RNAi oligonucleotide. Other antisense RNAi oligonucleotides described in the table below have the sequence TUAAAAUCUACAGUCAUAGGATT (SEQ ID NO: 35) and are complementary to human HPRT GenBank Accession No. NM_000194.2 (SEQ ID NO: 1, described herein above) from nucleosides 444 to 465, with a single mismatch at position 1 (from 5′ to 3′) of the antisense RNAi oligonucleotide, and a single mismatch at position 22 (from 5′ to 3′) of the antisense RNAi oligonucleotide. Each antisense RNAi oligonucleotide has a 5′-phosphate.

Compound No. 1505889, described herein above, is 100% complementary to the first 21 nucleosides of the Compound No 1455005 (from 5′ to 3′), leaving two overhanging 3′ nucleosides on the antisense RNAi oligonucleotide that are not paired with the sense RNAi oligonucleotide.

The sense RNAi oligonucleotide Compound No. 1505889, described herein above, is complementary to nucleosides 2 to 21 (from 5′ to 3′) of the remaining antisense compounds described in table 106, leaving two overhanging 3′ nucleosides on the antisense RNAi oligonucleotides that are not paired with the sense RNAi oligonucleotide.

TABLE 108
Design of antisense RNAi oligonucleotides

| Compound | | SEQ |
|---|---|---|
| No. | Chemistry Notation (5′ to 3′) | ID NO. |
| 1455005 | p.A_{ys}U_{fs}A_{yo}A_{yo}A_{yo}A_{fo}U_{yo}C_{fo}U_{fo}A_{yo} | 34 |
| | C_{yo}A_{yo}G_{yo}U_{fo}C_{yo}A_{fo}U_{yo}A_{yo}G_{yo}G_{yo}A_{ys} | |
| | T_{ds}T_{d} | |
| 1512935 | p.T_{ys}U_{fs}A_{yo}A_{yo}A_{yo}A_{fo}U_{yo}C_{fo}U_{fo}A_{yo} | 35 |
| | C_{yo}A_{yo}G_{yo}U_{fo}C_{yo}A_{fo}U_{yo}A_{yo}G_{yo}G_{yo}A_{ys} | |
| | T_{ds}T_{d} | |
| 1512936 | p.T_{ys}U_{fs}A_{yo}A_{yo}A_{yo}A_{fo}U_{yo}C_{fo}U_{fo}A_{yo} | 35 |
| | C_{yo}A_{yo}G_{yo}U_{fo}C_{yo}A_{fo}U_{yo}A_{yo}G_{yo}G_{yo}A_{ys} | |
| | T_{[bLdr]s}T_{[bLdr]} | |
| 1512937 | p.T_{ys}U_{fs}A_{yo}A_{yo}A_{yo}A_{fo}U_{yo}C_{fo}U_{fo}A_{yo} | 35 |
| | C_{yo}A_{yo}G_{yo}U_{fo}C_{yo}A_{fo}U_{yo}A_{yo}G_{yo}G_{yo}A_{ys} | |
| | T_{[aDdr]s}T_{[aDdr]} | |
| 1512938 | p.T_{ys}U_{fs}A_{yo}A_{yo}A_{yo}A_{fo}U_{yo}C_{fo}U_{fo}A_{yo} | 35 |
| | C_{yo}A_{yo}G_{yo}U_{fo}C_{yo}A_{fo}U_{yo}A_{yo}G_{yo}G_{yo}A_{ys} | |
| | T_{[aLdr]s}T_{[aLdr]} | |
| 1512939 | p.T_{ys}U_{fs}A_{yo}A_{yo}A_{yo}A_{fo}U_{yo}C_{fo}U_{fo}A_{yo} | 35 |
| | C_{yo}A_{yo}G_{yo}U_{fo}C_{yo}A_{fo}U_{yo}A_{yo}G_{yo}G_{yo}A_{ys} | |
| | T_{[bDdx]s}T_{[bDdx]} | |
| 1512940 | p.T_{ys}U_{fs}A_{yo}A_{yo}A_{yo}A_{fo}U_{yo}C_{fo}U_{fo}A_{yo} | 35 |
| | C_{yo}A_{yo}G_{yo}U_{fo}C_{yo}A_{fo}U_{yo}A_{yo}G_{yo}G_{yo}A_{ys} | |
| | T_{[bLdx]s}T_{[bLdx]} | |
| 1512941 | p.T_{ys}U_{fs}A_{yo}A_{yo}A_{yo}A_{fo}U_{yo}C_{fo}U_{fo}A_{yo} | 35 |
| | C_{yo}A_{yo}G_{yo}U_{fo}C_{yo}A_{fo}U_{yo}A_{yo}G_{yo}G_{yo}A_{ys} | |
| | T_{[aDdx]s}T_{[aDdx]} | |
| 1512942 | p.T_{ys}U_{fs}A_{yo}A_{yo}A_{yo}A_{fo}U_{yo}C_{fo}U_{fo}A_{yo} | 35 |
| | C_{yo}A_{yo}G_{yo}U_{fo}C_{yo}A_{fo}U_{yo}A_{yo}G_{yo}G_{yo}A_{ys} | |
| | T_{[aLdx]s}T_{[aLdx]} | |

In the table above, a “p.” represents a 5′ terminal phosphate group, a subscript “d” represents a 2′-β-D-deoxyribosyl sugar moiety, a subscript “[bLdr]” represents a 2′-β-L-deoxyribosyl sugar moiety, a subscript “[aDdr]” represents a 2′-α-D-deoxyribosyl sugar moiety, a subscript “[aLdr]” represents a 2′-α-L-deoxyribosyl sugar moiety, a subscript “[bDdx]” represents a 2′-β-D-deoxyxylosyl sugar moiety, a subscript “[bLdx]” represents a 2′-β-L-deoxyxylosyl sugar moiety, a subscript “[aDdx]” represents an 2′-α-D-deoxyxylosyl sugar moiety, a subscript “[aLdx]” represents an 2′-α-L-deoxyxylosyl sugar moiety, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage.

TABLE 109
Design of siRNA containing 2′-deoxyribonucleosides
or 2′-deoxyxylonucleosides targeted to HPRT1

| Duplex | Antisense Strand | Sense Strand |
|---|---|---|
| Compound No. | Compound No. | Compound No. |
| 1519796 | 1455005 | 1505889 |
| 1519797 | 1512935 | 1505889 |
| 1519798 | 1512936 | 1505889 |
| 1519799 | 1512937 | 1505889 |
| 1519800 | 1512938 | 1505889 |
| 1519801 | 1512939 | 1505889 |
| 1519802 | 1512940 | 1505889 |
| 1519803 | 1512941 | 1505889 |
| 1519804 | 1512942 | 1505889 |

### Example 51: Dose-Dependent Inhibition of Human HPRT in Hela Cells by siRNA Containing 2′-Deoxyribonucleosides or 2′-Deoxyxylonucleosides Targeted to HPRT1

The RNAi agents described above were tested at various doses in HeLa cells. Cultured HeLa cells at a density of 7,000 cells per well were treated by RNAiMAX with siRNA at concentrations indicated in the table below. After a treatment period of approximately 6 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount in untreated control cells (% UTC).

The half maximal inhibitory concentration (IC₅₀) of each RNAi agent was calculated with GraphPad Prism software (v8.2.0, San Diego, CA) using the log (inhibitor) vs. response—Variable slope (four parameters) function: Y=Bottom+(Top−Bottom)/(1+10\hat{ }((Log IC50−X)*HillSlope)), with the following constraints: Bottom=0, Top=100. Each table represents a separate experiment.

TABLE 110
Dose-dependent inhibition of human HPRT1 in HeLa cells by siRNA
containing 2′-deoxyribonucleosides or 2′-deoxyxylonucleosides

| Com- | HPRT1 RNA (% UTC) | | | | | | | | |
|---|---|---|---|---|---|---|---|---|---|
| pound | 10 | 2 | 0.4 | 0.08 | 0.016 | 0.0032 | 0.00064 | 0.000128 | IC₅₀ |
| No. | nM | nM | nM | nM | nM | nM | nM | nM | (pM) |
| 1519796 | 4 | 6 | 16 | 36 | 71 | 92 | 97 | 106 | 0.05 |
| 1519797 | 4 | 7 | 18 | 39 | 83 | 90 | 91 | 100 | 0.06 |
| 1519798 | 4 | 6 | 16 | 45 | 78 | 91 | 111 | 94 | 0.07 |
| 1519799 | 4 | 7 | 17 | 47 | 81 | 97 | 94 | 96 | 0.07 |
| 1519800 | 5 | 7 | 18 | 50 | 88 | 96 | 91 | 96 | 0.09 |
| 1519801 | 6 | 13 | 42 | 85 | 106 | 110 | 99 | 98 | 0.33 |
| 1519802 | 5 | 9 | 30 | 66 | 101 | 104 | 104 | 108 | 0.18 |
| 1519803 | 4 | 5 | 13 | 41 | 85 | 98 | 101 | 106 | 0.06 |
| 1519804 | 3 | 4 | 9 | 29 | 67 | 96 | 98 | 103 | 0.03 |

### Example 52: In Vivo Activity of siRNA in Wild-Type Mice

### In Vivo Study Design

In vivo studies were carried out to evaluate whether mesyl phosphoramidate internucleoside linkages improved potency of RNAi agents. RNAi agents described above were tested in C57Bl6/J male mice. The mice were divided into groups of 4 mice each. Each mouse received a single subcutaneous injection of RNAi agent at a dose of 1 mg/kg and sacrificed one week later. A group of 4 mice received PBS as a negative control.

### RNA Analysis

After one week, mice were sacrificed, and RNA was extracted from liver for quantitative RTPCR analysis of measurement of RNA expression of FXII using primer-probe set RTS2959 (forward sequence CAAAGGAGGGACATGTATCAACAC, designated herein as SEQ ID NO: 27; reverse sequence CTGGCAATGTTTCCCAGTGA, designated herein as SEQ ID NO: 28; probe sequence CCCAATGGGCCACACTGTCTCTGC, designated herein as SEQ ID NO: 29). Results are presented as percent mouse FXII RNA relative to the amount in PBS treated mice (% control), normalized to mouse cyclophilin A, measured by primer-probe set m_cyclo24 (described herein above).

TABLE 111
Reduction of mouse FXII RNA by siRNA with mesyl
phosphoramidate internucleoside linkages

| | FXII RNA |
|---|---|
| Compound No. | (% control) |
| PBS | 100 |
| 1632812 | 16 |
| 1526196 | 12.1 |
| 1645351 | 13 |
| 1645352 | 11.5 |
| 1645355 | 15.3 |
| 1645198 | 17 |
| 1645201 | 20.8 |
| 1652868 | 24 |
| 1633612 | 18.4 |

### Example 53: Design of siRNA Targeted to Mouse FXII

Modified oligonucleotides in the table below having either stereo-standard nucleosides or stereo-non-standard nucleosides in the antisense RNAi oligonucleotide and/or the sense RNAi oligonucleotide were synthesized using standard techniques.

Each antisense strand described in the table below has the sequence UAAAGCACUUUAUUGAGUUUCUG (SEQ ID NO: 24), described herein above, or the sequence TAAAGCACUUUAUUGAGUUUCUG (SEQ ID NO: 31), described herein above. Aside from a single mismatch at position 1 on the 5′-end, each antisense strand is complementary to the complement of GenBank Accession No. NC_000079.6, truncated from nucleosides 55415001 to 55430000, (SEQ ID NO. 25) from nucleosides 12005 to 12026 (SEQ ID NO: 39). Antisense RNAi oligonucleotide Compound No. 1526195 is described herein above.

TABLE 112
Design of antisense RNAi oligonucleotides
targeted to FXII

| Compound | | SEQ ID |
|---|---|---|
| No. | Chemistry Notation (5′ to 3′) | NO. |
| 1666847 | VP-TₑₛA_{fz}A_{yo}A_{yo}G_{yo}C_{fz}A_{yo}C_{yo}U_{yo} | 31 |
| | U_{yo}U_{yo}A_{yo}U_{yo}U_{fz}G_{yo}A_{fz}G_{yo}U_{yo}U_{yo} | |
| | U_{yo}C_{ys}U_{ys}G_{y} | |
| 1666848 | VP-TₑₛA_{fs}A_{yo}A_{yo}G_{yo}C_{fz}A_{yo}C_{yo}U_{yo} | 31 |
| | U_{yo}U_{yo}A_{yo}U_{yo}U_{fz}G_{yo}A_{fz}G_{yo}U_{yo}U_{yo} | |
| | U_{yo}C_{ys}U_{ys}G_{y} | |
| 1653512 | VP-TₑₛA[B2_{bDx]s}A_{yo}A_{yo}G_{yo}C_{fo}A_{yo} | 31 |
| | C_{yo}U_{yo}U_{yo}U_{yo}A_{yo}U_{yo}U_{fo}G_{yo}A_{fo}G_{yo} | |
| | U_{yo}U_{yo}U_{yo}C_{ys}U_{ys}G_{y} | |
| 1653515 | p.U_{ys}A_{fs}A_{yo}A_{yo}G_{yo}C_{fo}A_{yo}C_{yo}U_{yo} | 24 |
| | U_{yo}U_{yo}A_{yo}U_{yo}U_{[f2bDx]o}G_{yo}A_{fo}G_{yo} | |
| | U_{yo}U_{yo}U_{yo}C_{ys}U_{ys}G_{y} | |
| 1653516 | p.U_{ys}A_{fs}A_{yo}A_{yo}G_{yo}C_{fo}A_{yo}C_{yo}U_{yo} | 24 |
| | U_{yo}U_{yo}A_{yo}U_{yo}U_{fo}G_{yo}A_{[f2bDx]o}G_{yo} | |
| | U_{yo}U_{yo}U_{yo}C_{ys}U_{ys}G_{y} | |

In the table above, a “vP-” represents a vinyl phosphonate (vP) moiety on the 5′-end, a “p.” represents a 5′ terminal phosphate group, a subscript “f” represents a 2′-fluoro-β-D-ribosyl sugar moiety, a subscript “y” represents a 2′-O-methyl-β-D-ribosyl sugar moiety, a subscript “e” represents a MOE ribosyl sugar moiety, a subscript “[f2bDx]” represents a 2′-fluoro-β-D-xylosyl sugar moiety, a subscript “s” represents a phosphorothioate internucleoside linkage, a subscript “z” represents a mesyl phosphoramidate internucleoside linkage, and a subscript “o” represents a phosphodiester internucleoside linkage.

Each sense strand described in the table below is complementary to the first 21 nucleosides of the antisense RNAi oligonucleotide (from 5′ to 3′) wherein the last two 3′-nucleosides of the antisense RNAi oligonucleotides are not paired with the sense RNAi oligonucleotide (are overhanging nucleosides). Each sense RNAi oligomeric compound further contains a GalNAc moiety conjugated to the 3′-oxygen as shown below:

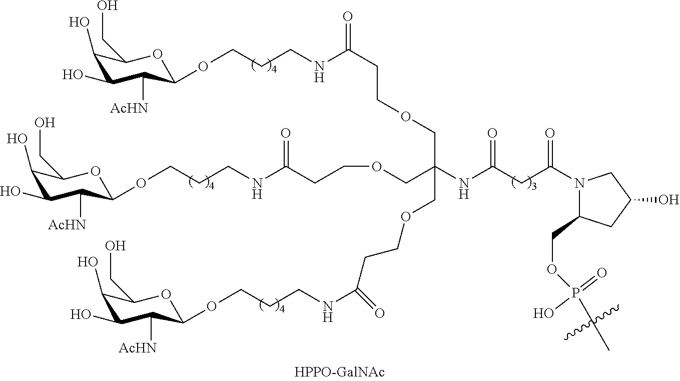

Compound No. 1523578 is described herein above and was previously disclosed in WO 2021/030778.

TABLE 113
Design of sense RNAi oligonucleotides

| Compound | | SEQ ID |
|---|---|---|
| No. | Chemistry Notation (5′ to 3′) | NO. |
| 1657707 | G_{ys}A_{ys}A_{yo}A_{yo}C_{yo}U_{yo}C_{yo}A_{yo}A_{fo} | 26 |
| | U_{fo}A_{fo}A_{yo}A_{yo}G_{yo}U_{yo}G_{yo}C_{yo}U_{yo} | |
| | U_{yo}U_{yo}A_{yo}-HPPO-GalNAc | |
| 1657708 | G_{ys}A_{ys}A_{yo}A_{yo}C_{yo}U_{yo}C_{yo}A_{yo}A_{yo} | 26 |
| | U_{fo}A_{fo}A_{yo}A_{yo}G_{yo}U_{yo}G_{yo}C_{yo}U_{yo} | |
| | U_{yo}U_{yo}A_{yo}-HPPO-GalNAc | |
| 1657712 | G_{ys}A_{ys}A_{yo}A_{yo}C_{yo}U_{yo}C_{yo}A_{yo}A_{yo} | 26 |
| | U_{fo}A_{yo}A_{yo}A_{yo}G_{yo}U_{yo}G_{yo}C_{yo}U_{yo} | |
| | U_{yo}U_{yo}A_{yo}-HPPO-GalNAc | |

In the table above, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage.

TABLE 114
Design of siRNA targeted to FXII c

| Duplex | Antisense Strand | Sense Strand |
|---|---|---|
| Compound No. | Compound No. | Compound No. |
| 1669050 | 1666847 | 1523578 |
| 1669051 | 1666848 | 1523578 |
| 1669054 | 1653512 | 1523578 |
| 1669154 | 1653515 | 1523578 |
| 1669155 | 1653516 | 1523578 |
| 1669149 | 1526195 | 1657707 |
| 1669151 | 1526195 | 1657708 |
| 1669152 | 1526195 | 1657712 |

### Example 54: In Vivo Activity of siRNAs Targeted to Mouse FXII in Wild-Type Mice

### In Vivo Study Design

In vivo studies were carried out to evaluate whether mesyl phosphoramidate internucleoside linkages improved potency of RNAi agents. RNAi agents described above were tested in C57Bl6/J male mice (The Jackson Laboratory). The mice were divided into groups of 4 mice each. Each mouse received a single subcutaneous injection of RNAi agent at a dose of 1 mg/kg and sacrificed one week later. A group of 4 mice received PBS as a negative control.

### RNA Analysis

After one week, mice were sacrificed, and RNA was extracted from liver for quantitative RTPCR analysis of measurement of RNA expression of FXII using primer-probe set RTS2959 (described herein above). Results are presented as percent mouse FXII RNA relative to the amount in PBS treated mice (% control), normalized to mouse cyclophilin A, measured by primer-probe set m_cyclo24 (described herein above).

TABLE 115
Reduction of mouse FXII RNA by siRNA with mesyl
phosphoramidate internucleoside linkages

| | FXII RNA |
|---|---|
| Compound No. | (% control) |
| 1669050 | 17.8 |
| 1669051 | 15.7 |
| 1645198 | 17.0 |
| 1645201 | 20.8 |
| 1652868 | 24.0 |
| 1669054 | 12.5 |
| 1669154 | 23.9 |
| 1669155 | 17.2 |
| 1633612 | 18.4 |
| 1669149 | 10.1 |
| 1669151 | 12.6 |
| 1669152 | 15.2 |

### Example 55: Design of RNAi Compounds Targeted to HPRT1 Containing Altritol Nucleic Acids (ANA)

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotides described in the table below have the sequence UUAAAAUCUACAGUCAUAGGAAU (SEQ ID NO: 30) and are complementary to human HPRT GenBank Accession No. NM_000194.2 (SEQ ID NO: 1, described herein above) from nucleoside start site 444 to 465 (SEQ ID NO: 37), with a single mismatch at position 1 on the 5′ end. Each antisense RNAi oligonucleotide has a phosphate moiety (p.) at the 5′ end.

Antisense RNAi oligonucleotide Compound No. 1601968 is described herein above.

TABLE 116
Design of antisense strand modified
oligonucleotides targeted to HPRT1

| Compound | | SEQ |
|---|---|---|
| No. | Chemistry Notation (5′ to 3′) | ID NO. |
| 1657738 | p.U_{[ANA]s}U_{fs}A_{yo}A_{yo}A_{yo}A_{fo}U_{yo}C_{yo}U_{yo} | 30 |
| | A_{yo}C_{yo}A_{yo}G_{yo}U_{fo}C_{yo}A_{fo}U_{yo}A_{yo}G_{yo}G_{yo} | |
| | A_{ys}A_{ys}U_{y} | |
| 1659599 | p.U_{ys}U_{[ANA]s}A_{yo}A_{yo}A_{yo}A_{fo}U_{yo}C_{yo}U_{yo} | 30 |
| | A_{yo}C_{yo}A_{yo}G_{yo}U_{fo}C_{yo}A_{fo}U_{yo}A_{yo}G_{yo}G_{yo} | |
| | A_{ys}A_{ys}U_{y} | |
| 1659600 | p.U_{ys}U_{fs}A_{yo}A_{yo}A_{yo}A_{fo}U_{[ANA]o}C_{yo}U_{yo} | 30 |
| | A_{yo}C_{yo}A_{yo}G_{yo}U_{fo}C_{yo}A_{fo}U_{yo}A_{yo}G_{yo}G_{yo} | |
| | A_{ys}A_{ys}U_{y} | |
| 1659601 | p.U_{ys}U_{fs}A_{yo}A_{yo}A_{yo}A_{fo}U_{yo}C_{yo}U_{[ANA]o} | 30 |
| | A_{yo}C_{yo}A_{yo}G_{yo}U_{fo}C_{yo}A_{fo}U_{yo}A_{yo}G_{yo}G_{yo} | |
| | A_{ys}A_{ys}U_{y} | |
| 1659602 | p.U_{ys}U_{fs}A_{yo}A_{yo}A_{yo}A_{fo}U_{yo}C_{yo}U_{yo}A_{yo} | 30 |
| | C_{yo}A_{yo}G_{yo}U_{[ANA]o}C_{yo}A_{fo}U_{yo}A_{yo}G_{yo}G_{yo} | |
| | A_{ys}A_{ys}U_{y} | |
| 1659603 | p.U_{ys}U_{fs}A_{yo}A_{yo}A_{yo}A_{fo}U_{yo}C_{yo}U_{yo}A_{yo} | 30 |
| | C_{yo}A_{yo}G_{yo}U_{fo}C_{yo}A_{fo}U_{[ANA]o}A_{yo}G_{yo}G_{yo} | |
| | A_{ys}A_{ys}U_{y} | |
| 1659605 | p.U_{ys}U_{fs}A_{yo}A_{yo}A_{yo}A_{fo}U_{yo}C_{yo}U_{[ANA]o} | 30 |
| | A_{yo}C_{yo}A_{yo}G_{yo}U_{[ANA]o}C_{yo}A_{fo}U_{[ANA]o}A_{yo} | |
| | G_{yo}G_{yo}A_{ys}A_{ys}U_{[ANA]} | |
| 1677653 | p.U_{ys}U_{fs}A_{yo}A_{yo}A_{yo}A_{fo}U_{yo}C_{yo}U_{yo}A_{yo} | 30 |
| | C_{yo}A_{yo}G_{yo}U_{[ANA]o}C_{yo}A_{f}OU_{[ANA]o}A_{yo} | |
| | G_{yo}G_{yo}A_{ys}A_{ys}U_{y} | |

In the table above, a subscript “f” represents a 2′-fluoro-β-D-ribosyl sugar moiety, a subscript “y” represents a 2′-O-methyl ribosyl sugar moiety, a subscript “[ANA]” represents an ANA sugar surrogate, a subscript “s” represents a phosphorothioate internucleoside linkage, and a subscript “o” represents a phosphodiester internucleoside linkage.

The antisense RNAi oligonucleotides described in the table below has the sequence UUAAAAUCUACAGUCAUAGGAUU (SEQ ID NO: 40) and is complementary to human HPRT GenBank Accession No. NM_000194.2 (SEQ ID NO: 1, described herein above) from nucleoside start site 444 to 465 (SEQ ID NO: 37), with a mismatch at position 1 on the 5′ end and a mismatch at position 22 at the 3′ end. The antisense RNAi oligonucleotide has a phosphate moiety (p.) at the 5′ end.

Antisense RNAi oligonucleotide Compound No. 1601968 is described herein above.

TABLE 117
Design of antisense strand modified
oligonucleotides targeted to HPRT1

| Compound | | SEQ |
|---|---|---|
| No. | Chemistry Notation (5′ to 3′) | ID NO. |
| 1677654 | p.U_{ys}U_{fs}A_{yo}A_{yo}A_{yo}A_{fo}U_{yo}C_{yo}U_{yo}A_{yo} | 40 |
| | C_{yo}A_{yo}G_{yo}U_{fo}C_{yo}A_{fo}U_{yo}A_{yo}G_{yo}G_{yo}A_{ys} | |
| | U_{[ANA]s}U_{y} | |

In the table above, a subscript “f” represents a 2′-fluoro-β-D-ribosyl sugar moiety, a subscript “y” represents a 2′-O-methyl ribosyl sugar moiety, a subscript “[ANA]” represents an ANA sugar surrogate, a subscript “s” represents a phosphorothioate internucleoside linkage, and a subscript “o” represents a phosphodiester internucleoside linkage.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Sense RNAi oligonucleotides Compound No. 1586323 and Compound No. 1586324 are described herein above.

TABLE 118
Design of sense strand modified
oligonucleotides targeted to HPRT1

| Compound | | SEQ ID |
|---|---|---|
| No. | Chemistry Notation (5′ to 3′) | NO. |
| 1659614 | U_{ys}C_{ys}C_{yo}U_{yo}A_{yo}U_{yo}G_{fo}A_{yo}C_{fo}U_{[ANA]o} | 10 |
| | G_{fo}U_{yo}A_{yo}G_{yo}A_{yo}U_{yo}U_{yo}U_{yo}U_{ys}A_{ys}A_{y} | |
| 1659615 | U_{[ANA]s}C_{ys}C_{yo}U_{yo}A_{yo}U_{yo}G_{fo}A_{yo}C_{fo} | 10 |
| | U_{[ANA]o}G_{fo}U_{yo}A_{yo}G_{yo}A_{yo}U_{yo}U_{yo}U_{[ANA]o} | |
| | U_{ys}A_{ys}A_{y} | |
| 1659616 | U_{[ANA]s}C_{ys}C_{yo}U_{[ANA]o}A_{yo}U_{yo}G_{fo}A_{yo}C_{fo} | 10 |
| | U_{[ANA]o}G_{fo}U_{yo}A_{yo}G_{yo}A_{yo}U_{yo}U_{yo}U_{yo}U_{ys} | |
| | A_{ys}A_{y} | |
| 1659617 | U_{ys}C_{ys}C_{yo}U_{[ANA]}OA_{yo}U_{[ANA]o}G_{fo}A_{yo}C_{fo} | 10 |
| | U_{[ANA]o}G_{fo}U_{yo}A_{yo}G_{yo}A_{yo}U_{yo}U_{yo}U_{yo}U_{ys} | |
| | A_{ys}A_{y} | |
| 1659618 | U_{ys}C_{ys}C_{yo}U_{yo}A_{yo}U_{yo}G_{fo}A_{yo}C_{fo}U_{[ANA]o} | 10 |
| | G_{fo}U_{[ANA]o}A_{yo}G_{yo}A_{yo}U_{yo}U_{yo}U_{[ANA]o}U_{ys} | |
| | A_{ys}A_{y} | |
| 1659619 | U_{[ANA]s}C_{ys}C_{yo}U_{yo}A_{yo}U_{[ANA]o}G_{fo}A_{yo}C_{fo} | 10 |
| | U_{[ANA]o}G_{fo}U_{[ANA]o}A_{yo}G_{yo}A_{yo}U_{yo}U_{yo}U_{yo} | |
| | U_{ys}A_{ys}A_{y} | |
| 1659620 | U_{[ANA]s}C_{ys}C_{yo}U_{yo}A_{yo}U_{[ANA]o}G_{fo}A_{yo}C_{fo} | 10 |
| | U_{[ANA]o}G_{fo}U_{yo}A_{yo}G_{yo}A_{yo}U_{[ANA]o}U_{yo}U_{yo} | |
| | U_{ys}A_{ys}A_{y} | |
| 1670674 | U_{[ANA]s}C_{ys}C_{yo}U_{yo}A_{yo}U_{[ANA]o}G_{fo}A_{yo}C_{fo} | 10 |
| | U_{[ANA]o}G_{fo}U_{[ANA]o}A_{yo}G_{yo}A_{yo}U_{[ANA]o}U_{yo} | |
| | U_{yo}U_{ys}A_{ys}A_{y} | |

In the table above, a subscript “f” represents a 2′-fluoro-β-D-ribosyl sugar moiety, a subscript “y” represents a 2′-O-methyl ribosyl sugar moiety, a subscript “[ANA]” represents an ANA sugar surrogate, a subscript “s” represents a phosphorothioate internucleoside linkage, and a subscript “o” represents a phosphodiester internucleoside linkage.

TABLE 119
Design of RNAi compounds containing altritol
nucleic acids targeted to HPRT1

| Duplex | Antisense Strand | Sense Strand |
|---|---|---|
| Compound No. | Compound No. | Compound No. |
| 1678709 | 1601968 | 1586323 |
| 1679604 | 1657738 | 1586323 |
| 1679607 | 1659599 | 1586323 |
| 1679622 | 1659600 | 1586323 |
| 1679625 | 1659601 | 1586323 |
| 1679628 | 1659602 | 1586323 |
| 1679634 | 1659603 | 1586323 |
| 1679640 | 1659605 | 1586323 |
| 1679643 | 1677653 | 1586323 |
| 1679646 | 1677654 | 1586323 |
| 1679649 | 1601968 | 1659614 |
| 1679655 | 1601968 | 1659615 |
| 1679658 | 1601968 | 1659616 |
| 1679661 | 1601968 | 1659617 |
| 1679664 | 1601968 | 1659618 |
| 1679667 | 1601968 | 1659620 |
| 1679670 | 1601968 | 1659619 |
| 1679673 | 1601968 | 1670674 |
| 1679676 | 1657738 | 1659614 |
| 1679679 | 1659599 | 1659614 |
| 1679682 | 1659600 | 1659614 |
| 1679685 | 1659601 | 1659614 |
| 1679688 | 1659602 | 1659614 |
| 1679691 | 1659603 | 1659614 |
| 1679694 | 1659605 | 1659614 |
| 1679697 | 1677653 | 1659614 |
| 1679700 | 1677654 | 1659614 |
| 1679703 | 1657738 | 1659615 |
| 1679706 | 1659599 | 1659615 |
| 1679709 | 1659600 | 1659615 |
| 1679712 | 1659601 | 1659615 |
| 1679715 | 1659602 | 1659615 |
| 1679718 | 1659603 | 1659615 |
| 1679721 | 1659605 | 1659615 |
| 1679724 | 1677653 | 1659615 |
| 1679727 | 1677654 | 1659615 |
| 1679739 | 1657738 | 1659616 |
| 1679742 | 1659599 | 1659616 |
| 1679745 | 1659600 | 1659616 |
| 1679748 | 1659601 | 1659616 |
| 1679751 | 1659602 | 1659616 |
| 1679754 | 1659603 | 1659616 |
| 1679757 | 1659605 | 1659616 |
| 1679760 | 1677653 | 1659616 |
| 1679763 | 1677654 | 1659616 |
| 1679766 | 1657738 | 1659617 |
| 1679769 | 1659599 | 1659617 |
| 1679772 | 1659600 | 1659617 |
| 1679775 | 1659601 | 1659617 |
| 1679778 | 1659602 | 1659617 |
| 1679781 | 1659603 | 1659617 |
| 1679784 | 1659605 | 1659617 |
| 1679787 | 1677653 | 1659617 |
| 1679790 | 1677654 | 1659617 |
| 1679793 | 1657738 | 1659618 |
| 1679796 | 1659599 | 1659618 |
| 1679799 | 1659600 | 1659618 |
| 1679802 | 1659601 | 1659618 |
| 1679805 | 1659602 | 1659618 |
| 1679808 | 1659603 | 1659618 |
| 1679811 | 1659605 | 1659618 |
| 1679814 | 1677653 | 1659618 |
| 1679817 | 1677654 | 1659618 |
| 1679820 | 1657738 | 1659619 |
| 1679823 | 1659599 | 1659619 |
| 1679826 | 1659600 | 1659619 |
| 1679832 | 1659601 | 1659619 |
| 1679835 | 1659602 | 1659619 |
| 1679838 | 1659603 | 1659619 |
| 1679841 | 1659605 | 1659619 |
| 1679844 | 1677653 | 1659619 |
| 1679847 | 1677654 | 1659619 |
| 1679850 | 1657738 | 1670674 |
| 1679853 | 1659599 | 1670674 |
| 1679856 | 1659600 | 1670674 |
| 1679859 | 1659601 | 1670674 |
| 1679862 | 1659602 | 1670674 |
| 1679865 | 1659603 | 1670674 |
| 1679868 | 1659605 | 1670674 |
| 1679871 | 1677653 | 1670674 |
| 1679874 | 1677654 | 1670674 |

### Example 56: Dose-Dependent Inhibition of Human HPRT1 in A431 Cells by RNAi Compounds Containing ANA Modifications

The RNAi agents described above were tested at various doses in A431 cells. Cultured A431 cells at a density of 20,000 cells per well were treated by RNAiMAX with RNAi compounds at concentrations indicated in the tables below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount of HPRT1 RNA in untreated control cells (% UTC). “N.D.” in the table below refers to data that was not determined.

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Compound No. 1640504 (described herein above) and Compound No. 1678709 (described herein above) was included as a benchmark.

TABLE 120
Dose-dependent reduction of human HPRT1 RNA in A431 cells by
RNAi compounds containing ANA modifications

| | HPRT1 RNA (% UTC) | | | | | | | | | | | |
|---|---|---|---|---|---|---|---|---|---|---|---|---|
| Compound | 100 | 10 | | 0.1 | 0.01 | 0.001 | 0.0001 | 0.00001 | | 0.25 | 0.0125 | IC₅₀ |
| No. | nM | nM | 1 nM | nM | nM | nM | nM | nM | 5 nM | nM | nM | (pM) |
| 1679604 | 3 | 3 | 5 | 10 | 42 | 73 | 96 | 104 | N.D. | N.D. | N.D. | 5.5 |
| 1679607 | 3 | 4 | 7 | 9 | 53 | 78 | 95 | 102 | N.D. | N.D. | N.D. | 8.7 |
| 1679622 | 3 | 4 | 5 | 7 | 36 | 71 | 97 | 95 | N.D. | N.D. | N.D. | 4.0 |
| 1679625 | 3 | 3 | 5 | 6 | 29 | 59 | 102 | 97 | N.D. | N.D. | N.D. | 2.4 |
| 1679628 | 4 | 4 | 6 | 11 | 52 | 86 | 91 | 99 | N.D. | N.D. | N.D. | 10.0 |
| 1679634 | 3 | 4 | 5 | 6 | 30 | 61 | 91 | 105 | N.D. | N.D. | N.D. | 2.4 |
| 1679640 | 4 | 4 | 5 | 7 | 33 | 67 | 93 | 98 | N.D. | N.D. | N.D. | 3.1 |
| 1679643 | 3 | 4 | 7 | 9 | 41 | 83 | 96 | 99 | N.D. | N.D. | N.D. | 6.5 |
| 1679646 | 3 | 4 | 5 | 6 | 28 | 61 | 90 | 98 | N.D. | N.D. | N.D. | 2.2 |
| 1678709 | 3 | 4 | 5 | 7 | 28 | 48 | 89 | 98 | N.D. | N.D. | N.D. | 1.4 |
| 1640504 | 3 | N.D. | N.D. | N.D. | N.D. | N.D. | N.D. | N.D. | 3 | 7 | 60 | 18.6 |

TABLE 121
Dose-dependent reduction of human HPRT1 RNA in A431 cells by
RNAi compounds containing ANA modifications

| | HPRT1 RNA (% UTC) | | | | | | | | | | | |
|---|---|---|---|---|---|---|---|---|---|---|---|---|
| Compound | 100 | 10 | | 0.1 | 0.01 | 0.001 | 0.0001 | 0.00001 | | 0.25 | 0.0125 | IC₅₀ |
| No. | nM | nM | 1 nM | nM | nM | nM | nM | nM | 5 nM | nM | nM | (pM) |
| 1679649 | 5 | 5 | 7 | 10 | 43 | 68 | 106 | 111 | N.D. | N.D. | N.D. | 5.4 |
| 1679655 | 5 | 6 | 10 | 14 | 61 | 90 | 99 | 108 | N.D. | N.D. | N.D. | 16.2 |
| 1679658 | 4 | 5 | 10 | 13 | 49 | 80 | 99 | 104 | N.D. | N.D. | N.D. | 8.9 |
| 1679661 | 6 | 5 | 7 | 10 | 38 | 77 | 93 | 106 | N.D. | N.D. | N.D. | 5.1 |
| 1679664 | 5 | 5 | 9 | 18 | 65 | 89 | 102 | 103 | N.D. | N.D. | N.D. | 19.8 |
| 1679667 | 4 | 5 | 9 | 15 | 56 | 91 | 92 | 107 | N.D. | N.D. | N.D. | 13.6 |
| 1679670 | 4 | 5 | 9 | 16 | 59 | 84 | 108 | 111 | N.D. | N.D. | N.D. | 14.7 |
| 1679673 | 4 | 6 | 11 | 18 | 68 | 94 | 98 | 104 | N.D. | N.D. | N.D. | 22.5 |
| 1678709 | 4 | 4 | 7 | 9 | 29 | 65 | 96 | 109 | N.D. | N.D. | N.D. | 2.8 |
| 1640504 | 4 | N.D. | N.D. | N.D. | N.D. | N.D. | N.D. | N.D. | 4 | 8 | 66 | 23.7 |

TABLE 122
Dose-dependent reduction of human HPRT1 RNA in A431 cells by
RNAi compounds containing ANA modifications

| | HPRT1 RNA (% UTC) | | | | | | | | | | | |
|---|---|---|---|---|---|---|---|---|---|---|---|---|
| Compound | 100 | 10 | | 0.1 | 0.01 | 0.001 | 0.0001 | 0.00001 | | 0.25 | 0.0125 | IC₅₀ |
| No. | nM | nM | 1 nM | nM | nM | nM | nM | nM | 5 nM | nM | nM | (pM) |
| 1679676 | 4 | 5 | 9 | 13 | 61 | 79 | 93 | 100 | N.D. | N.D. | N.D. | 12.5 |
| 1679679 | 4 | 5 | 8 | 12 | 55 | 79 | 92 | 98 | N.D. | N.D. | N.D. | 9.8 |
| 1679682 | 4 | 5 | 6 | 9 | 37 | 61 | 91 | 97 | N.D. | N.D. | N.D. | 3.1 |
| 1679685 | 5 | 5 | 6 | 7 | 32 | 58 | 91 | 93 | N.D. | N.D. | N.D. | 2.3 |
| 1679688 | 20 | 7 | 11 | 18 | 70 | 81 | 91 | 95 | N.D. | N.D. | N.D. | 2.0 |
| 1679691 | 4 | 5 | 7 | 8 | 34 | 57 | 93 | 101 | N.D. | N.D. | N.D. | 2.5 |
| 1679694 | 8 | 5 | 7 | 10 | 39 | 71 | 95 | 98 | N.D. | N.D. | N.D. | 4.6 |
| 1679697 | 14 | 7 | 9 | 17 | 65 | 82 | 95 | 95 | N.D. | N.D. | N.D. | 17.0 |
| 1679700 | 5 | 6 | 7 | 8 | 32 | 68 | 97 | 96 | N.D. | N.D. | N.D. | 3.4 |
| 1678709 | 4 | 4 | 6 | 8 | 33 | 62 | 98 | 104 | N.D. | N.D. | N.D. | 3.0 |
| 1640504 | 4 | N.D. | N.D. | N.D. | N.D. | N.D. | N.D. | N.D. | 4 | 9 | 66 | 24.7 |

TABLE 123
Dose-dependent reduction of human HPRT1 RNA in A431 cells by
RNAi compounds containing ANA modifications

| | HPRT1 RNA (% UTC) | | | | | | | | | | | |
|---|---|---|---|---|---|---|---|---|---|---|---|---|
| Compound | 100 | 10 | | 0.1 | 0.01 | 0.001 | 0.0001 | 0.00001 | | 0.25 | 0.0125 | IC₅₀ |
| No. | nM | nM | 1 nM | nM | nM | nM | nM | nM | 5 nM | nM | nM | (pM) |
| 1679703 | 4 | 5 | 10 | 14 | 64 | 83 | 100 | 109 | N.D. | N.D. | N.D. | 16.4 |
| 1679706 | 3 | 5 | 11 | 16 | 70 | 85 | 89 | 101 | N.D. | N.D. | N.D. | 20.4 |
| 1679709 | 3 | 4 | 7 | 11 | 50 | 79 | 94 | 104 | N.D. | N.D. | N.D. | 8.6 |
| 1679712 | 4 | 4 | 6 | 9 | 39 | 67 | 94 | 107 | N.D. | N.D. | N.D. | 4.0 |
| 1679715 | 7 | 7 | 14 | 26 | 73 | 84 | 98 | 107 | N.D. | N.D. | N.D. | 3.1 |
| 1679718 | 3 | 4 | 7 | 11 | 52 | 77 | 99 | 109 | N.D. | N.D. | N.D. | 8.9 |
| 1679721 | 5 | 4 | 8 | 13 | 53 | 82 | 92 | 110 | N.D. | N.D. | N.D. | 10.2 |
| 1679724 | 4 | 5 | 12 | 20 | 66 | 88 | 99 | 105 | N.D. | N.D. | N.D. | 21.3 |
| 1679727 | 3 | 5 | 8 | 12 | 41 | 80 | 103 | 95 | N.D. | N.D. | N.D. | 6.7 |
| 1678709 | 3 | 3 | 5 | 6 | 31 | 66 | 99 | 106 | N.D. | N.D. | N.D. | 3.2 |
| 1640504 | 3 | N.D. | N.D. | N.D. | N.D. | N.D. | N.D. | N.D. | 3 | 8 | 64 | 22.4 |

TABLE 124
Dose-dependent reduction of human HPRT1 RNA in A431 cells by RNAi
compounds containing ANA modifications

| | HPRT1 RNA (% UTC) | | | | | | | | | | | |
|---|---|---|---|---|---|---|---|---|---|---|---|---|
| Compound | 100 | 10 | | 0.1 | 0.01 | 0.001 | 0.0001 | 0.00001 | | 0.25 | 0.0125 | IC₅₀ |
| No. | nM | nM | 1 nM | nM | nM | nM | nM | nM | 5 nM | nM | nM | (pM) |
| 1679739 | 3 | 4 | 8 | 10 | 43 | 73 | 96 | 102 | N.D. | N.D. | N.D. | 5.6 |
| 1679742 | 3 | 4 | 7 | 10 | 50 | 73 | 92 | 92 | N.D. | N.D. | N.D. | 6.7 |
| 1679745 | 3 | 5 | 8 | 9 | 42 | 77 | 92 | 101 | N.D. | N.D. | N.D. | 5.8 |
| 1679748 | 3 | 5 | 7 | 8 | 32 | 67 | 94 | 99 | N.D. | N.D. | N.D. | 3.2 |
| 1679751 | 6 | 6 | 12 | 22 | 71 | 93 | 95 | 103 | N.D. | N.D. | N.D. | 2.7 |
| 1679754 | 3 | 4 | 8 | 9 | 40 | 71 | 98 | 104 | N.D. | N.D. | N.D. | 4.9 |
| 1679757 | 4 | 5 | 8 | 12 | 50 | 77 | 100 | 107 | N.D. | N.D. | N.D. | 8.6 |
| 1679760 | 4 | 7 | 11 | 18 | 69 | 96 | 98 | 104 | N.D. | N.D. | N.D. | 23.4 |
| 1679763 | 2 | 5 | 7 | 9 | 40 | 68 | 96 | 99 | N.D. | N.D. | N.D. | 4.4 |
| 1678709 | 3 | 5 | 6 | 8 | 36 | 64 | 91 | 95 | N.D. | N.D. | N.D. | 3.1 |
| 1640504 | 3 | N.D. | N.D. | N.D. | N.D. | N.D. | N.D. | N.D. | 4 | 9 | 61 | 20.3 |

TABLE 125
Dose-dependent reduction of human HPRT1 RNA in A431 cells by RNAi
compounds containing ANA modifications

| | HPRT1 RNA (% UTC) | | | | | | | | | | | |
|---|---|---|---|---|---|---|---|---|---|---|---|---|
| Compound | 100 | 10 | | 0.1 | 0.01 | 0.001 | 0.0001 | 0.00001 | | 0.25 | 0.0125 | IC₅₀ |
| No. | nM | nM | 1 nM | nM | nM | nM | nM | nM | 5 nM | nM | nM | (pM) |
| 1679766 | 3 | 3 | 5 | 11 | 60 | 78 | 104 | 104 | N.D. | N.D. | N.D. | 12.1 |
| 1679769 | 4 | 3 | 5 | 10 | 56 | 79 | 93 | 100 | N.D. | N.D. | N.D. | 9.8 |
| 1679772 | 4 | 3 | 4 | 9 | 50 | 75 | 97 | 103 | N.D. | N.D. | N.D. | 7.3 |
| 1679775 | 4 | 4 | 3 | 6 | 40 | 71 | 101 | 101 | N.D. | N.D. | N.D. | 4.7 |
| 1679778 | 14 | 5 | 11 | 29 | 87 | 94 | 101 | 100 | N.D. | N.D. | N.D. | 48.6 |
| 1679781 | 4 | 3 | 5 | 8 | 47 | 75 | 105 | 107 | N.D. | N.D. | N.D. | 7.1 |
| 1679784 | 11 | 3 | 5 | 12 | 62 | 81 | 106 | 109 | N.D. | N.D. | N.D. | 14.1 |
| 1679787 | 13 | 5 | 9 | 22 | 87 | 90 | 101 | 109 | N.D. | N.D. | N.D. | 39.5 |
| 1679790 | 4 | 3 | 4 | 6 | 29 | 63 | 101 | 103 | N.D. | N.D. | N.D. | 2.7 |
| 1678709 | 3 | 3 | 3 | 6 | 40 | 71 | 97 | 105 | N.D. | N.D. | N.D. | 4.7 |
| 1640504 | 3 | N.D. | N.D. | N.D. | N.D. | N.D. | N.D. | N.D. | 3 | 7 | 68 | 24.3 |

TABLE 126
Dose-dependent reduction of human HPRT1 RNA in A431 cells by
RNAi compounds containing ANA modifications

| | HPRT1 RNA (% UTC) | | | | | | | | | | | |
|---|---|---|---|---|---|---|---|---|---|---|---|---|
| Compound | 100 | 10 | | 0.1 | 0.01 | 0.001 | 0.0001 | 0.00001 | | 0.25 | 0.0125 | IC₅₀ |
| No. | nM | nM | 1 nM | nM | nM | nM | nM | nM | 5 nM | nM | nM | (pM) |
| 1679793 | 4 | 3 | 9 | 23 | 92 | 89 | 96 | 103 | N.D. | N.D. | N.D. | 45.6 |
| 1679796 | 4 | 3 | 8 | 22 | 93 | 95 | 99 | 98 | N.D. | N.D. | N.D. | 46.4 |
| 1679799 | 3 | 3 | 6 | 12 | 74 | 101 | 94 | 92 | N.D. | N.D. | N.D. | 22.2 |
| 1679802 | 4 | 3 | 5 | 12 | 70 | 88 | 100 | 112 | N.D. | N.D. | N.D. | 19.8 |
| 1679805 | 6 | 4 | 10 | 28 | 95 | 93 | 99 | 110 | N.D. | N.D. | N.D. | 54.8 |
| 1679808 | 4 | 3 | 6 | 15 | 71 | 97 | 108 | 106 | N.D. | N.D. | N.D. | 22.6 |
| 1679811 | 4 | 3 | 6 | 16 | 82 | 87 | 90 | 107 | N.D. | N.D. | N.D. | 29.6 |
| 1679814 | 5 | 4 | 9 | 22 | 86 | 100 | 99 | 97 | N.D. | N.D. | N.D. | 39.7 |
| 1679817 | 6 | 4 | 8 | 19 | 77 | 89 | 98 | 96 | N.D. | N.D. | N.D. | 28.2 |
| 1678709 | 4 | 3 | 4 | 6 | 37 | 70 | 96 | 102 | N.D. | N.D. | N.D. | 4.1 |
| 1640504 | 3 | N.D. | N.D. | N.D. | N.D. | N.D. | N.D. | N.D. | 3 | 6 | 65 | 22.1 |

TABLE 127
Dose-dependent reduction of human HPRT1 RNA in A431 cells by RNAi compounds
containing ANA modifications

| | HPRT1 RNA (% UTC) | | | | | | | | | | | |
|---|---|---|---|---|---|---|---|---|---|---|---|---|
| Compound | 100 | 10 | | 0.1 | 0.01 | 0.001 | 0.0001 | 0.00001 | | 0.25 | 0.0125 | IC₅₀ |
| No. | nM | nM | 1 nM | nM | nM | nM | nM | nM | 5 nM | nM | nM | (pM) |
| 1679820 | 4 | 3 | 6 | 13 | 66 | 84 | 101 | 93 | N.D. | N.D. | N.D. | 16.7 |
| 1679823 | 3 | 3 | 6 | 15 | 71 | 84 | 105 | 98 | N.D. | N.D. | N.D. | 21.3 |
| 1679826 | 3 | 3 | 4 | 10 | 52 | 79 | 98 | 97 | N.D. | N.D. | N.D. | 8.8 |
| 1679832 | 3 | 3 | 4 | 7 | 42 | 66 | 95 | 102 | N.D. | N.D. | N.D. | 4.2 |
| 1679835 | 5 | 4 | 9 | 31 | 93 | 88 | 95 | 102 | N.D. | N.D. | N.D. | 5.6 |
| 1679838 | 3 | 3 | 5 | 12 | 56 | 90 | 99 | 104 | N.D. | N.D. | N.D. | 12.6 |
| 1679841 | 5 | 3 | 7 | 19 | 86 | 89 | 93 | 100 | N.D. | N.D. | N.D. | 35.2 |
| 1679844 | 7 | 5 | 10 | 36 | 100 | 90 | 94 | 93 | N.D. | N.D. | N.D. | 71.7 |
| 1679847 | 4 | 3 | 5 | 9 | 54 | 77 | 89 | 99 | N.D. | N.D. | N.D. | 83.8 |
| 1678709 | 4 | 3 | 4 | 6 | 48 | 72 | 102 | 94 | N.D. | N.D. | N.D. | 6.3 |
| 1640504 | 4 | N.D. | N.D. | N.D. | N.D. | N.D. | N.D. | N.D. | 3 | 6 | 70 | 24.9 |

TABLE 128
Dose-dependent reduction of human HPRT1 RNA in A431 cells by RNAi compounds
containing ANA modifications

| | HPRT1 RNA (% UTC) | | | | | | | | | | | |
|---|---|---|---|---|---|---|---|---|---|---|---|---|
| Compound | 100 | IC₅₀ | | 0.1 | 0.01 | 0.001 | 0.0001 | 0.00001 | | 0.25 | 0.0125 | IC₅₀ |
| No. | nM | (pM) | 1 nM | nM | nM | nM | nM | nM | 5 nM | nM | nM | (pM) |
| 1679850 | 4 | 4 | 20 | 20 | 81 | 88 | 101 | 110 | N.D. | N.D. | N.D. | 34.6 |
| 1679853 | 4 | 3 | 8 | 19 | 79 | 96 | 101 | 105 | N.D. | N.D. | N.D. | 30.6 |
| 1679856 | 4 | 3 | 6 | 14 | 71 | 89 | 94 | 101 | N.D. | N.D. | N.D. | 20.9 |
| 1679859 | 4 | 3 | 5 | 12 | 64 | 86 | 100 | 107 | N.D. | N.D. | N.D. | 16.3 |
| 1679862 | 6 | 4 | 10 | 26 | 90 | 101 | 102 | 109 | N.D. | N.D. | N.D. | 48.0 |
| 1679865 | 4 | 3 | 6 | 20 | 80 | 86 | 98 | 106 | N.D. | N.D. | N.D. | 31.2 |
| 1679868 | 4 | 3 | 7 | 21 | 84 | 91 | 109 | 105 | N.D. | N.D. | N.D. | 35.2 |
| 1679871 | 6 | 5 | 11 | 35 | 81 | 92 | 95 | 101 | N.D. | N.D. | N.D. | 52.5 |
| 1679874 | 5 | 4 | 6 | 17 | 71 | 85 | 103 | 104 | N.D. | N.D. | N.D. | 21.7 |
| 1678709 | 3 | 2 | 3 | 6 | 36 | 69 | 96 | 96 | N.D. | N.D. | N.D. | 3.8 |
| 1640504 | 3 | N.D. | N.D. | N.D. | N.D. | N.D. | N.D. | N.D. | 3 | 6 | 66 | 22.0 |

### Example 57: Design of RNAi Compounds Targeted to HPRT1 with Modifications in the 3′ Overhang of the Antisense Strand

Modified oligonucleotides in the tables below having either stereo-standard nucleosides or stereo-non-standard nucleosides in the antisense RNAi oligonucleotides and/or the sense RNAi oligonucleotides were synthesized using standard techniques. The antisense modified oligonucleotides described in the table below have the sequence UUAAAAUCUACAGUCAUAGGAAA (SEQ ID NO: 41) and are complementary to human HPRT GenBank Accession No. NM_000194.2 (SEQ ID NO: 1, described herein above) from nucleoside start site 444 to 464 (SEQ ID NO: 37), with one mismatch at position 1 on the 5′ end, and one mismatch at position 23 on the 3′ end. Each antisense oligonucleotide has a terminal phosphate group (p.) on the 5′ end.

TABLE 129
Design of antisense strand modified
oligonucleotides targeted to HPRT1

| Compound | | SEQ |
|---|---|---|
| No. | Chemistry Notation (5′ to 3′) | ID NO. |
| 1653448 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | A_{ys}A_{y} | |
| 1653449 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | AₑₛAₑ | |
| 1653450 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | AₖₛAₖ | |
| 1653451 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{yo} | |
| | AₖₛAₖ | |
| 1653452 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{yo} | |
| | AₖₒAₖ | |
| 1653457 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | A_{[LNA]s}A_{[LNA]} | |
| 1653461 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | A_{[F-HNA]s}A_{[F-HNA]} | |
| 1653462 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{yo} | |
| | A_{[F-HNA]o}A_{[F-HNA]} | |
| 1660172 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | A_{yo}Aₖ | |
| 1660173 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{yo} | |
| | AₖₛAₑ | |
| 1660174 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{yo} | |
| | AₖₛA_{y} | |
| 1677745 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | A_{[F-HNA]o}A_{[F-HNA]} | |
| 1677786 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | A_{[f2bDa]s}A_{[f2bDa]} | |
| 1677792 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | AₙₛAₙ | |
| 1678717 | p.U_{ys}U_{fs}A_{yo}A_{yo}A_{yo}A_{fo}U_{yo}C_{yo}U_{yo}A_{yo} | 41 |
| | C_{yo}A_{yo}G_{yo}U_{fo}C_{yo}A_{fo}U_{yo}A_{yo}G_{yo}G_{yo}A_{ys} | |
| | A_{[DMAOE]s}A_{[DMAOE]} | |

In the table above, a subscript “f” represents a 2′-fluoro-β-D-ribosyl sugar moiety, a subscript “y” represents a 2′-O-methyl-β-D-ribosyl sugar moiety, a subscript “[F-HNA]” represents a 3′-fluoro-hexitol sugar surrogate, a subscript “[LNA]” represents a β-D-LNA sugar moiety, a subscript “[f2bDa]” represents a 2′-fluoro-β-D-arabinosyl sugar moiety, a subscript “n” represents a 2′-O—(N-methylacetamide) ribosyl sugar moiety, a subscript “[DMAOE]” represents a 2′-O-dimethylaminooxyethyl ribosyl sugar moiety, a subscript “s” represents a phosphorothioate internucleoside linkage, and a subscript “o” represents a phosphodiester internucleoside linkage.

The antisense modified oligonucleotides described in the table below have the sequence UUAAAAUCUACAGUCAUAGGAUU (SEQ ID NO: 41) or UUAAAAUCUACAGUCAUAGGATT (SEQ ID NO: 42) and are complementary to human HPRT human HPRT GenBank Accession No. NM_000194.2 (SEQ ID NO: 1, described herein above) from nucleoside start site 444 to 465 (SEQ ID NO: 37), with one mismatch at position 1 on the 5′ end, and one mismatch at position 22 on the 3′ end. Each antisense oligonucleotide has a terminal phosphate group (p.) on the 5′ end

TABLE 130
Design of antisense strand modified
oligonucleotides targeted to HPRT1

| Compound | | SEQ ID |
|---|---|---|
| No. | Chemistry Notation (5′ to 3′) | NO. |
| 1677863 | p.U_{ys}U_{fs}A_{yo}A_{yo}A_{yo}A_{fo}U_{yo}C_{yo}U_{yo}A_{yo}C_{yo} | 40 |
| | A_{yo}G_{yo}U_{fo}C_{yo}A_{fo}U_{yo}A_{yo}G_{yo}G_{yo}A_{ys}U_{[HNA]s} | |
| | U_{[HNA]} | |
| 1677864 | p.U_{ys}U_{fs}A_{yo}A_{yo}A_{yo}A_{fo}U_{yo}C_{yo}U_{yo}A_{yo}C_{yo} | 42 |
| | A_{yo}G_{yo}U_{fo}C_{yo}A_{fo}U_{yo}A_{yo}G_{yo}G_{yo}A_{ys} | |
| | T_{[SM5LNA]s}T_{[SM5LNA]} | |
| 1677793 | p.U_{ys}U_{fs}A_{yo}A_{yo}A_{yo}A_{fo}U_{yo}C_{yo}U_{yo}A_{yo}C_{yo} | 42 |
| | A_{yo}G_{yo}U_{fo}C_{yo}A_{fo}U_{yo}A_{yo}G_{yo}G_{yo}A_{ys} | |
| | T_{[DMAEOE]s}T_{[DMAEOE]} | |
| 1680219 | p.U_{ys}U_{fs}A_{yo}A_{yo}A_{yo}A_{fo}U_{yo}C_{yo}U_{yo}A_{yo}C_{yo} | 40 |
| | A_{yo}G_{yo}U_{fo}C_{yo}A_{fo}U_{yo}A_{yo}G_{yo}G_{yo}A_{ys}U_{ys}U_{y} | |
| 1680220 | p.U_{ys}U_{fs}A_{yo}A_{yo}A_{yo}A_{fo}U_{yo}C_{yo}U_{yo}A_{yo}C_{yo} | 42 |
| | A_{yo}G_{yo}U_{fo}C_{yo}A_{fo}U_{yo}A_{yo}G_{yo}G_{yo}A_{ys}T_{ds}T_{d} | |
| 1681002 | p.U_{ys}U_{fs}A_{yo}A_{yo}A_{yo}A_{fo}U_{yo}C_{yo}U_{yo}A_{yo}C_{yo} | |
| | A_{yo}G_{yo}U_{fo}C_{yo}A_{fo}U_{yo}A_{yo}G_{yo}G_{yo}A_{ys} | 42 |
| | T_{[aLdr]s}T_{[aLdr]} | |

In the table above, a subscript “f” represents a 2′-fluoro-β-D-ribosyl sugar moiety, a subscript “y” represents a 2′-O-methyl-β-D-ribosyl sugar moiety, a subscript “d” represents a 2′-β-D-deoxyribosyl sugar moiety, a subscript “[HNA]” represents an HNA sugar surrogate, a subscript “[SM5LNA]” represents a (5'S)-5′methyl-LNA sugar moiety, a subscript “[f2bDa]” represents a 2′-fluoro-β-D-arabinosyl sugar moiety, a subscript “[DMAEOE]” represents a 2′-O-(2-(2-(N,N-dimethyl)aminoethoxy)ethyl)(DMAEOE) ribosyl sugar moiety, a subscript “[aLdr]” represents a 2′-α-L-deoxyribosyl sugar moiety, a subscript “s” represents a phosphorothioate internucleoside linkage, and a subscript “o” represents a phosphodiester internucleoside linkage.

The sense RNAi oligonucleotide is complementary to the first 21 nucleosides of the antisense oligonucleotide (from 5′ to 3′) wherein the last two 3′-nucleosides of the antisense oligonucleotides are not paired with the sense oligonucleotide (are overhanging nucleosides). Sense oligonucleotide Compound No. 1586323 is described herein above.

TABLE 131
Design of RNAi compounds targeted to HPRT1 with modifications
in the 3′ overhang of the antisense strand

| Duplex | Antisense Strand | Sense Strand |
|---|---|---|
| Compound No. | Compound No. | Compound No. |
| 1680240 | 1653448 | 1586323 |
| 1680245 | 1653449 | 1586323 |
| 1680249 | 1653450 | 1586323 |
| 1680255 | 1653451 | 1586323 |
| 1680258 | 1653452 | 1586323 |
| 1680261 | 1653457 | 1586323 |
| 1680267 | 1653461 | 1586323 |
| 1680270 | 1677745 | 1586323 |
| 1680273 | 1653462 | 1586323 |
| 1680279 | 1677786 | 1586323 |
| 1680282 | 1677792 | 1586323 |
| 1680288 | 1678717 | 1586323 |
| 1680309 | 1660172 | 1586323 |
| 1680312 | 1660173 | 1586323 |
| 1680315 | 1660174 | 1586323 |
| 1680318 | 1680219 | 1586323 |
| 1680300 | 1677863 | 1586323 |
| 1680321 | 1680220 | 1586323 |
| 1681092 | 1677864 | 1586323 |
| 1681045 | 1681002 | 1586323 |
| 1680294 | 1677793 | 1586323 |

### Example 58: Dose-Dependent Inhibition of Human HPRT1 in A431 Cells by RNAi Compounds with Modifications in the 3′ Overhang of the Antisense Strand

The RNAi agents described above were tested at various doses in A431 cells. Cultured A431 cells at a density of 20,000 cells per well were treated by RNAiMAX with RNAi compound at concentrations indicated in the tables below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount of HPRT1 RNA in untreated control cells (% UTC).

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Compound No. 1640504 (described herein above) and Compound No. 1678709 (described herein above) were included as a benchmarks.

TABLE 132
Dose-dependent reduction of human HPRT1 RNA in A431 cells by RNAi compounds

| | HPRT1 RNA (% UTC) | | | | | | | | |
|---|---|---|---|---|---|---|---|---|---|
| Compound | | | 0.2 | 0.04 | 0.008 | 0.0016 | 0.00032 | 0.000064 | IC₅₀ |
| No. | 5 nM | 1 nM | nM | nM | nM | nM | nM | nM | (pM) |
| 1640504 | 3 | 4 | 7 | 14 | 42 | 74 | 97 | 108 | 5.72 |
| 1678709 | 2 | 3 | 5 | 9 | 26 | 52 | 91 | 101 | 2.26 |
| 1680240 | 3 | 4 | 5 | 8 | 21 | 51 | 81 | 112 | 1.81 |
| 1680245 | 3 | 4 | 5 | 7 | 20 | 50 | 81 | 103 | 1.69 |
| 1680249 | 3 | 3 | 5 | 9 | 22 | 50 | 87 | 105 | 1.95 |
| 1680255 | 3 | 3 | 5 | 9 | 24 | 60 | 86 | 102 | 2.46 |
| 1680258 | 3 | 4 | 5 | 11 | 30 | 58 | 88 | 99 | 2.72 |
| 1680261 | 3 | 3 | 4 | 7 | 19 | 43 | 70 | 106 | 1.23 |
| 1680267 | 3 | 3 | 4 | 9 | 25 | 45 | 78 | 111 | 1.65 |
| 1680270 | 2 | 3 | 5 | 8 | 21 | 42 | 85 | 102 | 1.49 |
| 1680273 | 2 | 2 | 5 | 6 | 20 | 41 | 98 | 108 | 1.54 |
| 1680279 | 2 | 5 | 3 | 6 | 21 | 44 | 93 | 113 | 1.68 |

TABLE 133
Dose-dependent reduction of human HPRT1 RNA in A431 cells by RNAi compounds

| | HPRT1 RNA (% UTC) | | | | | | | | |
|---|---|---|---|---|---|---|---|---|---|
| Compound | | | 0.2 | 0.04 | 0.008 | 0.0016 | 0.00032 | 0.000064 | IC₅₀ |
| No. | 5 nM | 1 nM | nM | nM | nM | nM | nM | nM | (pM) |
| 1640504 | 3 | 4 | 9 | 14 | 67 | 72 | 115 | 108 | 11.14 |
| 1678709 | 2 | 3 | 4 | 6 | 18 | 47 | 108 | 121 | 1.66 |
| 1680282 | 2 | 3 | 3 | 7 | 19 | 47 | 104 | 101 | 1.79 |
| 1680288 | 2 | 2 | 11 | 6 | 16 | 44 | 79 | 110 | 1.36 |
| 1680294 | 3 | 4 | 9 | 8 | 20 | 47 | 81 | 88 | 1.50 |
| 1680300 | 3 | 2 | 3 | 7 | 20 | 43 | 81 | 85 | 1.29 |
| 1680309 | 2 | 2 | 6 | 8 | 22 | 48 | 90 | 116 | 1.87 |
| 1680312 | 2 | 5 | 3 | 7 | 34 | 43 | 85 | 103 | 2.00 |
| 1680315 | 3 | 6 | 8 | 6 | 23 | 49 | 93 | 110 | 2.00 |
| 1680318 | 2 | 4 | 5 | 5 | 17 | 36 | 81 | 115 | 1.15 |
| 1680321 | 2 | 3 | 3 | 11 | 21 | 40 | 73 | 89 | 1.14 |
| 1681045 | 3 | 5 | 4 | 8 | 17 | 45 | 84 | 90 | 1.43 |
| 1681092 | 2 | 3 | 4 | 6 | 16 | 47 | 77 | 92 | 1.34 |

### Example 59: Dose-Dependent Inhibition of FXII in Mouse Hepatocyte Cells by RNAi Compounds

The RNAi agents described above were tested at various doses in mouse hepatocyte cells. Mouse hepatocyte cells at a density of 20,000 cells per well were treated by RNAiMAX with RNAi compound at concentrations indicated in the tables below. After a treatment period of approximately 24 hours, total RNA was isolated from the cells and FXII RNA levels were measured by quantitative real-time RTPCR. Mouse FXII primer-probe set RTS2959 (described herein above) was used to measure RNA levels. FXII RNA levels were normalized to total RNA content, as measured by RIBOGREEN®. Reduction of FXII RNA is presented in the tables below as percent FXII RNA, relative to the amount of HPRT1 RNA in untreated control cells (% UTC). “N.D.” indicates data that was not determined.

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Each table represents a separate experiment. Parent Compound No. 1632812 (described herein above) or parent Compound No. 1523582 (described herein above) was included as a benchmark.

TABLE 134
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1526196 | 9 | 5 | 6 | 11 | 16 | 64 | 91 | 97 | 1.87 |
| 1645351 | 9 | 8 | 7 | 13 | 19 | 46 | 89 | 89 | 1.07 |
| 1645352 | 7 | 6 | 5 | 9 | 16 | 50 | 94 | 87 | 1.18 |
| 1645353 | 9 | 7 | 4 | 8 | 19 | 54 | 93 | 96 | 1.46 |
| 1645354 | 8 | 5 | 7 | 13 | 28 | 77 | 109 | 103 | 4.05 |
| 1645355 | 9 | 5 | 5 | 8 | 22 | 69 | 99 | 90 | 2.62 |
| 1645356 | 6 | 8 | 6 | 14 | 19 | 78 | 105 | 95 | 3.03 |

TABLE 135
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1645198 | 11 | 7 | 7 | 17 | 30 | 84 | 95 | 99 | 5.10 |
| 1645199 | 10 | 8 | 6 | 13 | 22 | 64 | 94 | 99 | 2.32 |
| 1645200 | 12 | 8 | 7 | 12 | 18 | 59 | 100 | 99 | 1.79 |
| 1645201 | 7 | 6 | 5 | 9 | 20 | 54 | 96 | 88 | 1.49 |
| 1645202 | 10 | 9 | 9 | 15 | 24 | 69 | 109 | 92 | 3.10 |
| 1632812 | 6 | 6 | 5 | 10 | 16 | 39 | 81 | 105 | 0.71 |

TABLE 136
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1645203 | 6 | 5 | 7 | 8 | 17 | 76 | 99 | 99 | 2.65 |
| 1645204 | 7 | 6 | 7 | 9 | 21 | 66 | 95 | 93 | 2.26 |
| 1645205 | 7 | 7 | 7 | 10 | 24 | 82 | 96 | 102 | 3.80 |
| 1645206 | 8 | 6 | 8 | 9 | 21 | 72 | 79 | 99 | 2.31 |
| 1645207 | 8 | 6 | 7 | 8 | 29 | 90 | 86 | 97 | 4.98 |
| 1645208 | 11 | 10 | 10 | 12 | 60 | 98 | 80 | 110 | 15.27 |
| 1632812 | 10 | 8 | 8 | 9 | 19 | 72 | 52 | 106 | 2.13 |

TABLE 137
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1633605 | 13 | 16 | 30 | 61 | 111 | 110 | 102 | 112 | 333 |
| 1633606 | 17 | 12 | 12 | 15 | 67 | 108 | 97 | 109 | 21 |
| 1633607 | 25 | 13 | 14 | 22 | 84 | 116 | 105 | 118 | 39 |
| 1633608 | 17 | 11 | 11 | 13 | 72 | 106 | 98 | 114 | 22 |
| 1633609 | N.D. | 30 | 57 | 83 | 121 | 111 | 102 | 113 | 2033 |
| 1650412 | 14 | 9 | 15 | 28 | 104 | 100 | 91 | 113 | 83 |
| 1650450 | 17 | 10 | 10 | 15 | 76 | 94 | 90 | 115 | 25 |
| 1650487 | 50 | 25 | 23 | 47 | 96 | 96 | 86 | 116 | 462 |
| 1523582 | 13 | 9 | 8 | 9 | 42 | 87 | 80 | 119 | 7 |

TABLE 138
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1633610 | 5 | 13 | 20 | 75 | 66 | 98 | 103 | 146 | 199 |
| 1633611 | 14 | 14 | 14 | 18 | 68 | 102 | 104 | 105 | 24 |
| 1633612 | 13 | 11 | 11 | 13 | 47 | 101 | 106 | 110 | 10 |
| 1633613 | 14 | 9 | 10 | 12 | 49 | 98 | 92 | 101 | 11 |
| 1633614 | 24 | 15 | 34 | 67 | 104 | 109 | 104 | 103 | 474 |
| 1523582 | 13 | 10 | 9 | 9 | 46 | 88 | 73 | 110 | 8 |

TABLE 139
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1633615 | 9 | 9 | 8 | 19 | 83 | 113 | 106 | 126 | 35 |
| 1633616 | 13 | 9 | 10 | 15 | 67 | 115 | 110 | 126 | 21 |
| 1633617 | 6 | 6 | 8 | 11 | 58 | 104 | 101 | 118 | 14 |
| 1633618 | 9 | 7 | 8 | 9 | 44 | 83 | 89 | 117 | 7 |
| 1633619 | 8 | 6 | 9 | 15 | 63 | 104 | 84 | 115 | 18 |
| 1523582 | 9 | 5 | 8 | 8 | 36 | 72 | 78 | 113 | 4 |

TABLE 140
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1633620 | 12 | 8 | 10 | 17 | 56 | 100 | 106 | 122 | 16 |
| 1633621 | 18 | 11 | 13 | 25 | 78 | 114 | 109 | 118 | 39 |
| 1633622 | 14 | 10 | 11 | 16 | 49 | 96 | 96 | 94 | 12 |
| 1633623 | 8 | 9 | 9 | 14 | 56 | 117 | 107 | 106 | 13 |
| 1633624 | 24 | 14 | 14 | 31 | 86 | 117 | 108 | 112 | 54 |
| 1523582 | 8 | 8 | 8 | 13 | 47 | 81 | 61 | 98 | 5 |

TABLE 141
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1633635 | 7 | 12 | 12 | 17 | 65 | 108 | 116 | 138 | 20 |
| 1633636 | 28 | 14 | 21 | 60 | 138 | 117 | 125 | 135 | 205 |
| 1633637 | 9 | 9 | 9 | 13 | 32 | 104 | 116 | 141 | 9 |
| 1633638 | 18 | 12 | 13 | 18 | 61 | 119 | 124 | 137 | 19 |
| 1632813 | 21 | 15 | 20 | 37 | 91 | 116 | 121 | 134 | 80 |
| 1632814 | 14 | 10 | 13 | 14 | 53 | 105 | 130 | 152 | 13 |
| 1632815 | 37 | 33 | 31 | 41 | 103 | 88 | 97 | 146 | 537 |
| 1632812 | 7 | 6 | 8 | 10 | 17 | 48 | 61 | 142 | 1 |

TABLE 142
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1652862 | 12 | 12 | 14 | 14 | 53 | 93 | 113 | 110 | 13 |
| 1652868 | 11 | 11 | 9 | 11 | 25 | 91 | 120 | 121 | 5 |
| 1652869 | 25 | 20 | 12 | 14 | 52 | 111 | 107 | 117 | 12 |
| 1652865 | 10 | 14 | 9 | 9 | 20 | 79 | 101 | 109 | 3 |
| 1652870 | N.D. | N.D. | 44 | 60 | 95 | 97 | 100 | 111 | 445 |
| 1632812 | 6 | 5 | 5 | 8 | 19 | 67 | 74 | 104 | 2 |

TABLE 143
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | 100 | 10 | | 0.1 | 0.01 | 0.001 | 0.0001 | 0.00001 | IC₅₀ |
| No. | nM | nM | 1 nM | nM | nM | nM | nM | nM | (pM) |
| 1659244 | 15 | 13 | 15 | 15 | 43 | 90 | 119 | 99 | 9 |
| 1659245 | 10 | 7 | 10 | 16 | 35 | 76 | 102 | 104 | 5 |
| 1659246 | 15 | 16 | 16 | 22 | 44 | 86 | 103 | 100 | 12 |
| 1632812 | 6 | 6 | 5 | 10 | 16 | 39 | 81 | 105 | 1 |

### Example 60: Design of RNAi Compounds Targeted to HPRT1 Having Lipid Modified Nucleosides in the Sense Strand

Modified oligonucleotides in the tables below having either stereo-standard nucleosides or stereo-non-standard nucleosides in the antisense RNAi oligonucleotides and/or the sense RNAi oligonucleotides were synthesized using standard techniques. Antisense oligonucleotides Compound No. 1586322 and Compound No. 1601968 are described herein above.

The sense RNAi oligonucleotide is complementary to the first 21 nucleosides of the antisense oligonucleotide (from 5′ to 3′) wherein the last two 3′-nucleosides of the antisense oligonucleotides are not paired with the sense oligonucleotide (are overhanging nucleosides). Sense oligonucleotide Compound No. 1591095 is described herein above.

TABLE 144
Design of sense RNAi oligomeric compounds
targeted to human/mouse HPRT1
containing lipid-modified nucleosides

| Compound | | SEQ |
|---|---|---|
| No. | Chemistry Notation (5′ to 3′) | ID NO. |
| 1687209 | U_{ys}C_{ys}C_{yo}U_{yo}A_{yo}U_{yo}G_{fo}A_{yo}C | 10 |
| | _{fo}U_{fo}G_{fo}U_{yo}A_{yo}G_{yo}A_{yo}U_{yo}U_{y} | |
| | ₒU_{yo}U_{ys}A_{ys}A_{yo}[3nC7-G.G.G.-C8] | |
| 1687210 | U_{ys}C_{ys}C_{yo}U_{yo}A_{yo}U_{yo}G_{fo}A_{yo}C | 10 |
| | _{fo}U_{fo}G_{fo}U_{yo}A_{yo}G_{yo}A_{yo}U_{yo}U_{y} | |
| | ₒU_{yo}U_{ys}A_{ys}A_{yo}[3nC7-G.G.G.-C16] | |
| 1687232 | U_{ys}C_{ys}C_{yo}U_{yo}A_{yo}U_{yo}G_{fo}A_{yo}C | 10 |
| | _{fo}U_{fo}G_{fo}U_{yo}A_{yo}G_{yo}A_{yo}U_{yo}U_{y} | |
| | ₒU_{yo}U_{ys}A_{ys}A_{yo}[3nC7-5OC5-C16] | |
| 1687246 | U_{ys}C_{ys}C_{yo}U_{yo}A_{yo}U_{yo}G_{do}A_{yo}C | 10 |
| | _{do}U_{yo}G_{do}U_{yo}A_{do}G_{yo}A_{do}U_{yo}U_{y} | |
| | ₒU_{yo}U_{ys}A_{ys}A_{yo}[3nC7-C16] | |
| 1687247 | U_{ys}C_{ys}C_{yo}U_{yo}A_{yo}U_{yo}G_{do}A_{yo}C | 10 |
| | _{do}U_{yo}G_{do}U_{yo}A_{do}G_{yo}A_{do}U_{do}U_{y} | |
| | ₒU_{yo}U_{ys}A_{ys}A_{yo}[3nC7-C16] | |
| 1687248 | U_{ys}C_{ys}C_{yo}U_{yo}A_{yo}U_{yo}G_{yo}A_{yo}C | 10 |
| | _{do}U_{do}G_{do}U_{do}A_{do}G_{yo}A_{yo}U_{yo}U_{y} | |
| | ₒU_{yo}U_{ys}A_{ys}A_{yo}[3nC7-C16] | |

In the table above, a subscript “f” represents a 2′-fluoro-β-D-ribosyl sugar moiety, a subscript “y” represents a 2′-O-methyl-β-D-ribosyl sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

“[3nC7-C16]” represents a palmitate moiety linked to a 3′-C7 amino modifier, as shown below, which is attached to the 3′-nucleoside via a phosphodiester linkage.

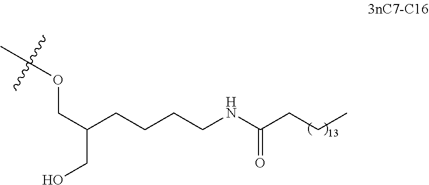

“[3nC7-G.G.G.-C8]” represents an octanoate moiety linked by a glycine tripeptide to a 3′-C7 amino modifier, as shown below, which is attached to the 3′-nucleoside via a phosphodiester linkage.

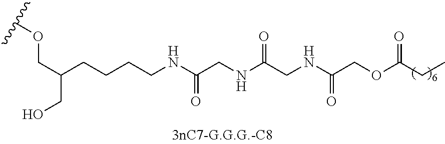

“[3nC7-G.G.G.-C16]” represents a palmitate moiety linked by a glycine tripeptide to a 3′-C7 amino modifier, as shown below, which is attached to the 3′-nucleoside via a phosphodiester linkage.

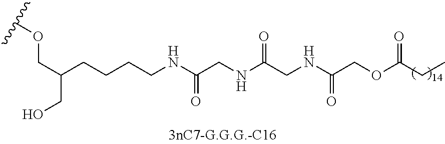

“[3nC7-50C5-C16]” represents a palmitate moiety linked by a pentanoic acid linker to a 3′-C7 amino modifier, as shown below, which is attached to the 3′-nucleoside via a phosphodiester linkage.

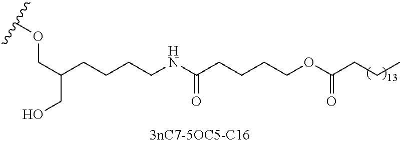

TABLE 145
Design of RNAi compounds targeted to human/mouse HPRT1 containing
lipid modified nucleosides in the sense strand

| Duplex | Antisense Strand | Sense Strand |
|---|---|---|
| Compound No. | Compound No. | Compound No. |
| 1687253 | 1586322 | 1687210 |
| 1687254 | 1586322 | 1687232 |
| 1687255 | 1586322 | 1687209 |
| 1687261 | 1601968 | 1591095 |
| 1687264 | 1586322 | 1687246 |
| 1687267 | 1586322 | 1687247 |
| 1687270 | 1586322 | 1687248 |

### Example 61: Activity of siRNAs Containing C16-Modified Nucleosides in the Sense Strand that Target Mouse HPRT, In Vivo

The activity of RNAi agents containing lipid conjugates was tested in wild type C57BL/6 mice (Taconic Biosciences).

Mice were divided into groups of 2 mice each. Each mouse received a single ICV bolus of 1 μg, 10 μg, 100 μg, or 500 μg of RNAi agent. A group of 4 mice received PBS as a control. Two weeks post treatment, mice were sacrificed, and RNA was extracted from cortical brain tissue, thoracic cord, and liver tissue for quantitative real-time RTPCR analysis of RNA expression of mouse HPRT using primer probe set RTS43125 (described herein above). Results are presented as percent change of mouse HPRT RNA, relative to the amount of HPRT RNA in PBS control treated mice, normalized to mouse cyclophilin A (% control). Mouse cyclophilin A was amplified using primer probe set m_cyclo24 (designated herein above). As shown in the table below, treatment with modified oligonucleotides resulted in reduction of mouse HPRT RNA in comparison to the PBS control.

The half maximal dose (ED₅₀) of each RNAi agent was calculated with GraphPad Prism software (v8.2.0, San Diego, CA) using the log (agonist) vs. response function: Y=Bottom+(Top−Bottom)/(1+10\hat{ }((Log EC50−X)*HillSlope)), with the following constraints: Bottom=0, Top=100.

TABLE 146
Reduction of mouse HPRT RNA in wild type C57BL/6 mice

| | | Cortex | | Thoracic Cord | | Liver | |
|---|---|---|---|---|---|---|---|
| Compound | Dose | HPRT RNA | ED₅₀ | HPRT RNA | ED₅₀ | HPRT RNA | ED₅₀ |
| Number | (μg) | (% control) | (μg) | (% control) | (μg) | (% control) | (μg) |
| 1599476 | 1 | 96 | 34 | 97 | 15 | 87 | 16 |
| | 10 | 67 | | 54 | | 57 | |
| | 100 | 35 | | 19 | | 22 | |
| | 500 | 13 | | 11 | | 10 | |
| 1687253 | 1 | 101 | 144 | 99 | 33 | 90 | 56 |
| | 10 | 90 | | 75 | | 87 | |
| | 100 | 64 | | 26 | | 35 | |
| | 500 | 13‡ | | 16‡ | | 16‡ | |
| 1687254 | 1 | 94 | 172 | 99 | 48 | 99 | 264 |
| | 10 | 92 | | 93 | | 111 | |
| | 100 | 71 | | 23 | | 71 | |
| | 500 | 15 | | 16 | | 35 | |
| 1653543 | 1 | 95 | 130 | 101 | 67 | 88 | >500 |
| | 10 | 84 | | 86 | | 91 | |
| | 100 | 57 | | 38 | | 87 | |
| | 500 | 27 | | 18 | | 53 | |
| 1687255 | 1 | 94 | 110 | 99 | 85 | 93 | >500 |
| | 10 | 84 | | 93 | | 94 | |
| | 100 | 59 | | 45 | | 76 | |
| | 500 | 61 | | 15‡ | | 671 | |
| 1640504 | 1 | 99 | >500 | 97 | 389 | 86 | >500 |
| | 10 | 103 | | 98 | | 87 | |
| | 100 | 90 | | 88 | | 86 | |
| | 500 | 67 | | 41 | | 67 | |
| 1588821 | 1 | 96 | 39 | 97 | 47 | 91 | >500 |
| | 10 | 78 | | 86 | | 94 | |
| | 100 | 30 | | 29 | | 79 | |
| | 500 | 10 | | 10 | | 49 | |
| 1687261 | 1 | 102 | 138 | 89 | 107 | 96 | 63 |
| | 10 | 94 | | 62 | | 89 | |
| | 100 | 50 | | 66 | | 35 | |
| | 500 | 32 | | 26 | | 16 | |
| 1687264 | 1 | 98 | 112 | 95 | 38 | 93 | 126 |
| | 10 | 76 | | 83 | | 95 | |
| | 100 | 65 | | 23 | | 57 | |
| | 500 | 11 | | 13 | | 17 | |
| 1687267 | 1 | 97 | 87 | 98 | 35 | 87 | >500 |
| | 10 | 73 | | 73 | | 84 | |
| | 100 | 56 | | 30 | | 81 | |
| | 500 | 19 | | 11 | | 49 | |
| 1687270 | 1 | 107 | >500 | 94 | 193 | 102 | >500 |
| | 10 | 74 | | 76 | | 89 | |
| | 100 | 80 | | 59 | | 96 | |
| | 500 | 59 | | 39 | | 85 | |

‡indicates fewer than two subjects

### Example 62: Design of RNAi Compounds Targeted to HPRT1 Containing Hexitol Nucleic Acids (HNA)

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotides described in the table below have the sequence UUAAAAUCUACAGUCAUAGGAAU (SEQ ID NO: 30) or TUAAAAUCUACAGUCAUAGGAAU (SEQ ID NO: 9) and are complementary to human HPRT GenBank Accession No. NM_000194.2 (SEQ ID NO: 1) from nucleoside start site 444 to 465, with a single mismatch at position 1 on the 5′ end which has been bolded and underlined. Antisense RNAi oligonucleotides Compound No. 1601968 and Compound No. 1677863 are described herein above.

TABLE 147
Design of antisense strand modified
oligonucleotides targeted to HPRT1
containing HNA

| Compound | | SEQ |
|---|---|---|
| No. | Chemistry Notation (5′ to 3′) | ID NO. |
| 1681054 | p.U_{[HNA]s}U_{fs}A_{yo}A_{yo}A_{yo}A_{fo}U_{yo} | 30 |
| | C_{yo}U_{yo}A_{yo}C_{yo}A_{yo}G_{yo}U_{fo}C_{yo}A_{fo} | |
| | U_{yo}A_{yo}G_{yo}G_{yo}A_{ys}A_{ys}U_{y} | |
| 1687227 | p.U_{ys}U_{[HNA]s}A_{yo}A_{yo}A_{yo}A_{fo}U_{yo} | 30 |
| | C_{yo}U_{yo}A_{yo}C_{yo}A_{yo}G_{yo}U_{fo}C_{yo}A_{fo} | |
| | U_{yo}A_{yo}G_{yo}G_{yo}A_{ys}A_{ys}U_{y} | |
| 1687228 | VP-TₑₛU_{[HNA]s}A_{yo}A_{yo}A_{yo}A_{fo} | 9 |
| | U_{yo}C_{yo}U_{yo}A_{yo}C_{yo}A_{yo}G_{yo}U_{fo} | |
| | C_{yo}A_{fo}U_{yo}A_{yo}G_{yo}G_{yo}A_{ys}A_{ys}U_{y} | |
| 1687229 | p.U_{ys}U_{fs}A_{yo}A_{yo}A_{yo}A_{fo}U_{[HNA]o} | 30 |
| | C_{yo}U_{yo}A_{yo}C_{yo}A_{yo}G_{yo}U_{fo}C_{yo} | |
| | A_{fo}U_{yo}A_{yo}G_{yo}G_{yo}A_{ys}A_{ys}U_{y} | |
| 1687230 | p.U_{ys}U_{fs}A_{yo}A_{yo}A_{yo}A_{fo}U_{yo}C_{yo} | 30 |
| | U_{[HNA]}OA_{yo}C_{yo}A_{yo}G_{yo}U_{fo}C_{yo} | |
| | A_{fo}U_{yo}A_{yo}G_{yo}G_{yo}A_{ys}A_{ys}U_{y} | |
| 1687231 | p.U_{ys}U_{fs}A_{yo}A_{yo}A_{yo}A_{fo}U_{yo}C_{yo} | 30 |
| | U_{yo}A_{yo}C_{yo}A_{yo}G_{yo}U_{[HNA]o}C_{yo} | |
| | A_{fo}U_{yo}A_{yo}G_{yo}G_{yo}A_{ys}A_{ys}U_{y} | |
| 1687233 | p.U_{ys}U_{fs}A_{yo}A_{yo}A_{yo}A_{fo}U_{yo}C_{y} | 30 |
| | ₒU_{yo}A_{yo}C_{yo}A_{yo}G_{yo}U_{fo}C_{yo}A_{fo} | |
| | U_{[HNA]o}A_{yo}G_{yo}G_{yo}A_{ys}A_{ys}U_{y} | |
| 1687234 | p.U_{ys}U_{fs}A_{yo}A_{yo}A_{yo}A_{fo}U_{yo}C_{y} | 30 |
| | ₒU_{[HNA]o}A_{yo}C_{yo}A_{yo}G_{yo}U_{[HNA]o} | |
| | C_{yo}A_{fo}U_{[HNA]o}A_{yo}G_{yo}G_{yo}A_{ys}A_{ys} | |
| | U_{[HNA]} | |
| 1687235 | p.U_{ys}U_{fs}A_{yo}A_{yo}A_{yo}A_{fo}U_{yo}C_{y} | 30 |
| | ₒU_{yo}A_{yo}C_{yo}A_{yo}G_{yo}U_{[HNA]o}C_{yo}A_{fo} | |
| | U_{[HNA]o}A_{yo}G_{yo}G_{yo}A_{ys}A_{ys}U_{y} | |

In the table above, a “p.” represents a 5′ terminal phosphate group, a “vP-” represents a vinyl phosphonate (vP) moiety on the 5′-end, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “[HNA]” represents a an HNA sugar surrogate, a subscript “s” represents a phosphorothioate internucleoside linkage, and a subscript “o” represents a phosphodiester internucleoside linkage.

The antisense RNAi oligonucleotides described in the table below has the sequence UUAAAAUCUACAGUCAUAGGAUU (SEQ ID NO: 40) and are complementary to human HPRT GenBank Accession No. NM_000194.2 (SEQ ID NO: 1, described herein above) from nucleoside start site 444 to 465 (SEQ ID NO: 37), with a mismatch at position 1 on the 5′ end and a mismatch at position 22 at the 3′ end. The antisense RNAi oligonucleotide has a phosphate moiety (p.) at the 5′ end.

TABLE 148
Design of antisense strand modified
oligonucleotides containing HNA
targeted to HPRT1

| Compound | | SEQ |
|---|---|---|
| No. | Chemistry Notation (5′ to 3′) | ID NO. |
| 1687249 | p.U_{ys}U_{fs}A_{yo}A_{yo}A_{yo}A_{fo}U_{yo}C_{yo} | 40 |
| | U_{yo}A_{yo}C_{yo}A_{yo}G_{yo}U_{fo}C_{yo}A_{fo}U_{yo} | |
| | A_{yo}G_{yo}G_{yo}A_{ys}U_{[HNA]s}U_{y} | |

In the table above, a “p.” represents a 5′-phosphate, a “vP-” represents a vinyl phosphonate (vP) moiety on the 5′-end, a subscript “f” represents a 2′-fluoro-β-D-ribosyl sugar moiety, a subscript “y” represents a 2′-O-methyl-β-D-ribosyl sugar moiety, a subscript “[HNA]” represents an HNA sugar surrogate, a subscript “s” represents a phosphorothioate internucleoside linkage, and a subscript “o” represents a phosphodiester internucleoside linkage.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Sense RNAi oligonucleotide Compound No. 1586323 is described herein above.

TABLE 149
Design of sense strand modified oligonucleotides
containing HNA targeted to HPRT1

| Compound | | SEQ ID |
|---|---|---|
| No. | Chemistry Notation (5′ to 3′) | NO. |
| 1687238 | U_{ys}C_{ys}C_{yo}U_{yo}A_{yo}U_{yo}G_{fo}A_{yo}C_{fo} | 10 |
| | U_{[HNA]o}G_{fo}U_{yo}A_{yo}G_{yo}A_{yo}U_{yo}U_{yo} | |
| | U_{yo}U_{ys}A_{ys}A_{y} | |
| 1687239 | U_{[HNA]s}C_{ys}C_{yo}U_{yo}A_{yo}U_{yo}G_{fo}A_{yo}C_{fo} | 10 |
| | U_{[HNA]o}G_{fo}U_{yo}A_{yo}G_{yo}A_{yo}U_{yo}U_{yo} | |
| | U_{[HNA]o}U_{ys}A_{ys}A_{y} | |
| 1687240 | U_{[HNA]s}C_{ys}C_{yo}U_{[HNA]o}A_{yo}U_{yo}G_{fo} | 10 |
| | A_{yo}C_{fo}U_{[HNA]o}G_{fo}U_{yo}A_{yo}G_{yo}A_{yo} | |
| | U_{yo}U_{yo}U_{yo}U_{ys}A_{ys}A_{y} | |
| 1687241 | U_{ys}C_{ys}C_{yo}U_{[HNA]o}A_{yo}U_{[HNA]o}G_{fo} | 10 |
| | A_{yo}C_{fo}U_{[HNA]o}G_{fo}U_{yo}A_{yo}G_{yo}A_{yo} | |
| | U_{yo}U_{yo}U_{yo}U_{ys}A_{ys}A_{y} | |
| 1687242 | U_{ys}C_{ys}C_{yo}U_{yo}A_{yo}U_{yo}G_{fo}A_{yo}C_{fo} | 10 |
| | U_{[HNA]o}G_{fo}U_{[HNA]o}A_{yo}G_{yo}A_{yo} | |
| | U_{yo}U_{yo}U_{[HNA]o}U_{ys}A_{ys}A_{y} | |
| 1687243 | U_{[HNA]s}C_{ys}C_{yo}U_{yo}A_{yo}U_{[HNA]o}G_{fo} | 10 |
| | A_{yo}C_{fo}U_{[HNA]o}G_{fo}U_{yo}A_{yo}G_{yo}A_{yo} | |
| | U_{[HNA]o}U_{yo}U_{yo}U_{ys}A_{ys}A_{y} | |
| 1687244 | U_{[HNA]s}C_{ys}C_{yo}U_{yo}A_{yo}U_{[HNA]o}G_{fo} | 10 |
| | A_{yo}C_{fo}U_{[HNA]o}G_{fo}U_{[HNA]o}A_{yo}G_{yo} | |
| | A_{yo}U_{yo}U_{yo}U_{yo}U_{ys}A_{ys}A_{y} | |
| 1687245 | U_{[HNA]s}C_{ys}C_{yo}U_{yo}A_{yo}U_{[HNA]o}G_{fo} | 10 |
| | A_{yo}C_{fo}U_{[HNA]o}G_{fo}U_{[HNA]o}A_{yo}G_{yo} | |
| | A_{yo}U_{[HNA]o}U_{yo}U_{yo}U_{ys}A_{ys}A_{y} | |

In the table above, a subscript “f” represents a 2′-fluoro-β-D-ribosyl sugar moiety, a subscript “y” represents a 2′-O-methyl-β-D-ribosyl sugar moiety, a subscript “[HNA]” represents an HNA sugar surrogate, a subscript “s” represents a phosphorothioate internucleoside linkage, and a subscript “o” represents a phosphodiester internucleoside linkage.

TABLE 150
Design of RNAi compounds containing hexitol
nucleic acids targeted to HPRT1

| Duplex | Antisense Strand | Sense Strand |
|---|---|---|
| Compound No. | Compound No. | Compound No. |
| 1678709 | 1601968 | 1586323 |
| 1692202 | 1681054 | 1586323 |
| 1692208 | 1687227 | 1586323 |
| 1692211 | 1687228 | 1586323 |
| 1692214 | 1687229 | 1586323 |
| 1692217 | 1687230 | 1586323 |
| 1692220 | 1687231 | 1586323 |
| 1692223 | 1687233 | 1586323 |
| 1692226 | 1687234 | 1586323 |
| 1692229 | 1687235 | 1586323 |
| 1692232 | 1687249 | 1586323 |
| 1680300 | 1677863 | 1586323 |
| 1692550 | 1601968 | 1687238 |
| 1692553 | 1601968 | 1687239 |
| 1692556 | 1601968 | 1687240 |
| 1692559 | 1601968 | 1687241 |
| 1692562 | 1601968 | 1687242 |
| 1692565 | 1601968 | 1687243 |
| 1692568 | 1601968 | 1687244 |
| 1692571 | 1601968 | 1687245 |
| 1692582 | 1681054 | 1687238 |
| 1692619 | 1687227 | 1687238 |
| 1692622 | 1687228 | 1687238 |
| 1692625 | 1687229 | 1687238 |
| 1692628 | 1687230 | 1687238 |
| 1692631 | 1687231 | 1687238 |
| 1692634 | 1687233 | 1687238 |
| 1692637 | 1687234 | 1687238 |
| 1692640 | 1687235 | 1687238 |
| 1692643 | 1687249 | 1687238 |
| 1692646 | 1677863 | 1687238 |
| 1692649 | 1681054 | 1687239 |
| 1692652 | 1687227 | 1687239 |
| 1692655 | 1687228 | 1687239 |
| 1692658 | 1687229 | 1687239 |
| 1692661 | 1687230 | 1687239 |
| 1692664 | 1687231 | 1687239 |
| 1692667 | 1687233 | 1687239 |
| 1692670 | 1687234 | 1687239 |
| 1692673 | 1687235 | 1687239 |
| 1692676 | 1687249 | 1687239 |
| 1692679 | 1677863 | 1687239 |
| 1692715 | 1681054 | 1687241 |
| 1692718 | 1687227 | 1687241 |
| 1692721 | 1687228 | 1687241 |
| 1692724 | 1687229 | 1687241 |
| 1692727 | 1687230 | 1687241 |
| 1692730 | 1687231 | 1687241 |
| 1692733 | 1687233 | 1687241 |
| 1692736 | 1687234 | 1687241 |
| 1692739 | 1687235 | 1687241 |
| 1692742 | 1687249 | 1687241 |
| 1692745 | 1677863 | 1687241 |
| 1692748 | 1681054 | 1687242 |
| 1692751 | 1687227 | 1687242 |
| 1692754 | 1687228 | 1687242 |
| 1692757 | 1687229 | 1687242 |
| 1692760 | 1687230 | 1687242 |
| 1692763 | 1687231 | 1687242 |
| 1692766 | 1687233 | 1687242 |
| 1692769 | 1687234 | 1687242 |
| 1692772 | 1687235 | 1687242 |
| 1692775 | 1687249 | 1687242 |
| 1692778 | 1677863 | 1687242 |
| 1692781 | 1681054 | 1687243 |
| 1692784 | 1687227 | 1687243 |
| 1692787 | 1687228 | 1687243 |
| 1692790 | 1687229 | 1687243 |
| 1692793 | 1687230 | 1687243 |
| 1692796 | 1687231 | 1687243 |
| 1692799 | 1687233 | 1687243 |
| 1692802 | 1687234 | 1687243 |
| 1692805 | 1687235 | 1687243 |
| 1692808 | 1687249 | 1687243 |
| 1692811 | 1677863 | 1687243 |
| 1692847 | 1681054 | 1687245 |
| 1692850 | 1687227 | 1687245 |
| 1692853 | 1687228 | 1687245 |
| 1692856 | 1687229 | 1687245 |
| 1692859 | 1687230 | 1687245 |
| 1692862 | 1687231 | 1687245 |
| 1692865 | 1687233 | 1687245 |
| 1692868 | 1687234 | 1687245 |
| 1692871 | 1687235 | 1687245 |
| 1692874 | 1687249 | 1687245 |
| 1692877 | 1677863 | 1687245 |
| 1692814 | 1681054 | 1687244 |
| 1692817 | 1687227 | 1687244 |
| 1692820 | 1687228 | 1687244 |
| 1692823 | 1687229 | 1687244 |
| 1692826 | 1687230 | 1687244 |
| 1692829 | 1687231 | 1687244 |
| 1692832 | 1687233 | 1687244 |
| 1692835 | 1687234 | 1687244 |
| 1692838 | 1687235 | 1687244 |
| 1692841 | 1687249 | 1687244 |
| 1692844 | 1677863 | 1687244 |

### Example 63: Dose-Dependent Inhibition of Human HPRT1 in A431 Cells by RNAi Compounds Containing Hexitol Nucleic Acids (HNA)

The RNAi agents described above were tested at various doses in A431 cells. Cultured A431 cells at a density of 20,000 cells per well were treated by RNAiMAX with RNAi compounds at concentrations indicated in the tables below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount of HPRT1 RNA in untreated control cells (% UTC).

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Compound No. 1640504 (described herein above) was included as a benchmark.

TABLE 151
Dose-dependent reduction of human HPRT1 RNA in A431 cells
by RNAi compounds containing hexitol nucleic acids (HNA)

| | HPRT1 RNA (% UTC) | | | | | | | | |
|---|---|---|---|---|---|---|---|---|---|
| Compound | | | | | | 0.001 | 0.0001 | 0.00001 | IC₅₀ |
| Number | 100 nM | 10 nM | 1 nM | 0.1 nM | 0.01 nM | nM | nM | nM | (pM) |
| 1678709 | 3 | 2 | 2 | 3 | 8 | 45 | 86 | 99 | 0.79 |
| 1692202 | 3 | 3 | 2 | 3 | 6 | 33 | 92 | 89 | 0.58 |
| 1692208 | 3 | 2 | 3 | 4 | 11 | 45 | 84 | 101 | 0.79 |
| 1692211 | 3 | 2 | 3 | 4 | 13 | 51 | 88 | 104 | 1.05 |
| 1692214 | 3 | 2 | 3 | 3 | 6 | 29 | 81 | 97 | 0.41 |
| 1692217 | 3 | 2 | 2 | 3 | 5 | 30 | 81 | 102 | 0.43 |
| 1692220 | 4 | 2 | 2 | 4 | 12 | 59 | 90 | 97 | 1.38 |
| 1692223 | 3 | 2 | 2 | 3 | 6 | 29 | 72 | 91 | 0.32 |
| 1692226 | 3 | 2 | 2 | 3 | 10 | 49 | 84 | 95 | 0.87 |
| 1692229 | 4 | 2 | 2 | 3 | 14 | 53 | 81 | 95 | 1.01 |

TABLE 152
Dose-dependent reduction of human HPRT1 RNA in A431 cells by RNAi compounds containing
hexitol nucleic acids (HNA)

| | HPRT1 RNA (% UTC) | | | | | | | | |
|---|---|---|---|---|---|---|---|---|---|
| Compound | | | | | | 0.001 | 0.0001 | 0.00001 | IC₅₀ |
| Number | 100 nM | 10 nM | 1 nM | 0.1 nM | 0.01 nM | nM | nM | nM | (pM) |
| 1678709 | 2 | 2 | 2 | 3 | 7 | 33 | 88 | 102 | 0.55 |
| 1680300 | 4 | 2 | 2 | 4 | 8 | 33 | 83 | 100 | 0.51 |
| 1692232 | 4 | 2 | 2 | 3 | 6 | 30 | 84 | 99 | 0.47 |
| 1692550 | 4 | 2 | 3 | 4 | 7 | 35 | 91 | 98 | 0.61 |
| 1692553 | 3 | 2 | 3 | 4 | 19 | 48 | 91 | 102 | 1.11 |
| 1692556 | 3 | 2 | 2 | 3 | 20 | 39 | 96 | 108 | 0.83 |
| 1692559 | 3 | 2 | 2 | 3 | 9 | 32 | 89 | 106 | 0.56 |
| 1692562 | 4 | 2 | 3 | 4 | 24 | 58 | 83 | 102 | 1.58 |
| 1692565 | 3 | 2 | 2 | 4 | 11 | 52 | 93 | 100 | 1.11 |
| 1692568 | 9 | 3 | 2 | 5 | 38 | 71 | 99 | 102 | 4.30 |

### Example 64: Design of RNAi Compounds Targeted to HPRT1 Containing 5′-Vinylphosphonate Moieties and Mesyl Phosphoramidate Internucleoside Linkages

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotides described in the table below have the sequence TUAAAAUCUACAGUCAUAGGAAU (SEQ ID NO: 9) and are complementary to human HPRT GenBank Accession No. NM_000194.2 (SEQ ID NO: 1) from nucleoside start site 444 to 465, with a single mismatch at position 1 on the 5′ end. Antisense RNAi oligonucleotide Compound No. 1586322 is described herein above.

TABLE 153
Design of antisense strand modified
oligonucleotides targeted to HPRT1
containing 5′-vinylphosphonate
moieties and mesyl phosphoramidate
internucleoside linkages

| Compound | | SEQ |
|---|---|---|
| No. | Chemistry Notation (5′ to 3′) | ID NO. |
| 1690119 | vP-T_{ds}U_{fs}A_{yo}A_{yo}A_{yo}A_{fo}U_{yo}C_{yo} | 9 |
| | U_{yo}A_{yo}C_{yo}A_{yo}G_{yo}U_{fo}C_{yo}A_{fo}U_{yo} | |
| | A_{yo}G_{yo}G_{yo}A_{yz}A_{ys}U_{y} | |
| 1685495 | vP-T_{dz}U_{fs}A_{yo}A_{yo}A_{yo}A_{fo}U_{yo}C_{yo} | 9 |
| | U_{yo}A_{yo}C_{yo}A_{yo}G_{yo}U_{fo}C_{yo}A_{fo}U_{yo} | |
| | A_{yo}G_{yo}G_{yo}A_{yz}A_{ys}U_{y} | |
| 1685497 | vP-T_{dz}U_{fz}A_{yo}A_{yo}A_{yo}A_{fo}U_{yo}C_{yo} | 9 |
| | U_{yo}A_{yo}C_{yo}A_{yo}G_{yo}U_{fo}C_{yo}A_{fo}U_{yo} | |
| | A_{yo}G_{yo}G_{yo}A_{yz}A_{ys}U_{y} | |

In the table above, a “vP-” represents a vinyl phosphonate (vP) moiety on the 5′-end, a subscript “f” represents a 2′-fluoro-β-D-ribosyl sugar moiety, a subscript “y” represents a 2′-O-methyl-β-D-ribosyl sugar moiety, a subscript “d” represents a 2′-β-D-deoxyribosyl sugar moiety, a subscript “z” represents a mesyl phosphoramidate internucleoside linkage, a subscript “s” represents a phosphorothioate internucleoside linkage, and a subscript “o” represents a phosphodiester internucleoside linkage.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Sense RNAi oligonucleotide Compound No. 1591095 is described herein above.

TABLE 154
Design of RNAi compounds targeted to HPRT1 containing
5′-vinylphosphonate moieties and mesyl phosphoramidate
internucleoside linkages

| Duplex | Antisense Strand | Sense Strand |
|---|---|---|
| Compound No. | Compound No. | Compound No. |
| 1599476 | 1586322 | 1591095 |
| 1692880 | 1690119 | 1591095 |
| 1692883 | 1685495 | 1591095 |
| 1692886 | 1685497 | 1591095 |

### Example 65: Dose-Dependent Inhibition of Human HPRT1 in A431 Cells by RNAi Compounds Containing 5′-Vinylphosphonate Moieties and Mesyl Phosphoramidate Internucleoside Linkages

The RNAi agents described above were tested at various doses in A431 cells. Cultured A431 cells at a density of 20,000 cells per well were treated by RNAiMAX with RNAi compounds at concentrations indicated in the tables below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount of HPRT1 RNA in untreated control cells (% UTC).

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment.

TABLE 155
Dose-dependent reduction of human HPRT1 RNA in A431 cells
by RNAi compounds containing 5′-vinylphosphonate moieties
and mesyl phosphoramidate internucleoside linkages

| | HPRT1 RNA (% UTC) | | | | | | | | |
|---|---|---|---|---|---|---|---|---|---|
| Compound | | | | | | 0.001 | 0.0001 | 0.00001 | IC₅₀ |
| Number | 100 nM | 10 nM | 1 nM | 0.1 nM | 0.01 nM | nM | nM | nM | (pM) |
| 1692880 | 7 | 2 | 2 | 3 | 8 | 34 | 87 | 111 | 0.57 |
| 1692883 | 5 | 3 | 2 | 3 | 17 | 28 | 81 | 100 | 0.46 |
| 1692886 | 5 | 3 | 3 | 3 | 8 | 36 | 85 | 102 | 0.57 |
| 1599476 | 4 | 2 | 2 | 2 | 5 | 26 | 78 | 105 | 0.36 |

### Example 66: Design of RNAi Compounds Targeted to HPRT1

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotides described in the table below have the sequence TUAAAAUCUACAGUCAUAGGAAU (SEQ ID NO: 9) and are complementary to human HPRT GenBank Accession No. NM_000194.2 (SEQ ID NO: 1) from nucleoside start site 444 to 465, with a single mismatch at position 1 on the 5′ end. Antisense RNAi oligonucleotide Compound No. 1586322 is described herein above.

TABLE 156
Design of antisense strand modified
oligonucleotides targeted to HPRT1

| Compound | Chemistry Notation | SEQ |
|---|---|---|
| No. | (5′ to 3′) | ID NO. |
| 1685106 | vP-TₑₛU_{fs}A_{yo}A_{yo}A_{yo}A_{do}U_{yo}C_{yo} | 9 |
| | U_{yo}A_{yo}C_{yo}A_{yo}G_{yo}U_{do}C_{yo}A_{do}U_{yo} | |
| | A_{yo}G_{yo}G_{yo}A_{ys}A_{ys}U_{y} | |
| 1691628 | vP-TₑₛU_{fz}A_{yo}A_{yo}A_{yo}A_{do}U_{yo}C_{yo} | 9 |
| | U_{yo}A_{yo}C_{yo}A_{yo}G_{yo}U_{do}C_{yo}A_{do}U_{yo} | |
| | A_{yo}G_{yo}G_{yo}A_{ys}A_{ys}U_{y} | |
| 1691629 | vP-TₑₛU_{[F-HNA]z}A_{yo}A_{yo}A_{yo}A_{do} | 9 |
| | U_{yo}C_{yo}U_{yo}A_{yo}C_{yo}A_{yo}G_{yo}U_{do}C_{yo} | |
| | A_{do}U_{yo}A_{yo}G_{yo}G_{yo}A_{ys}A_{ys}U_{y} | |
| 1691630 | vP-TₑₛU_{[F-HNA]s}A_{yo}A_{yo}A_{yo}A_{do} | 9 |
| | U_{yo}C_{yo}U_{yo}A_{yo}C_{yo}A_{yo}G_{yo}U_{do}C_{yo} | |
| | A_{do}U_{yo}A_{yo}G_{yo}G_{yo}A_{ys}A_{ys}U_{y} | |
| 1691631 | vP-TₑₛU_{[LNA]s}A_{yo}A_{yo}A_{yo}A_{do} | 9 |
| | U_{yo}C_{yo}U_{yo}A_{yo}C_{yo}A_{yo}G_{yo}U_{do}C_{yo} | |
| | A_{do}U_{yo}A_{yo}G_{yo}G_{yo}A_{ys}A_{ys}U_{y} | |
| 1691632 | vP-TₑₛU_{[f2bDx]s}A_{yo}A_{yo}A_{yo}A_{do} | 9 |
| | U_{yo}C_{yo}U_{yo}A_{yo}C_{yo}A_{yo}G_{yo}U_{do}C_{yo} | |
| | A_{do}U_{yo}A_{yo}G_{yo}G_{yo}A_{ys}A_{ys}U_{y} | |
| 1691633 | vP-TₑₛU_{dz}A_{yo}A_{yo}A_{yo}A_{do}U_{yo}C_{yo} | 9 |
| | U_{yo}A_{yo}C_{yo}A_{yo}G_{yo}U_{do}C_{yo}A_{do}U_{yo} | |
| | A_{yo}G_{yo}G_{yo}A_{ys}A_{ys}U_{y} | |

In the table above, a ““vP-” represents a vinyl phosphonate (vP) moiety on the 5′-end, a subscript “[f2bDx]” represents a 2′-fluoro-β-D-xylosyl sugar moiety, a subscript “[F-HNA]” represents a 3′-fluoro-hexitol sugar surrogate, a subscript “[LNA]” represents a β-D-LNA sugar moiety, a subscript “f” represents a 2′-fluoro-β-D-ribosyl sugar moiety, a subscript “y” represents a 2′-O-methyl-β-D-ribosyl sugar moiety, a subscript “d” represents a 2′-β-D-deoxyribosyl sugar moiety, a subscript “e” represents a 2′-MOE sugar moiety, a subscript “z” represents a mesyl phosphoramidate internucleoside linkage, a subscript “s” represents a phosphorothioate internucleoside linkage, and a subscript “o” represents a phosphodiester internucleoside linkage.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Sense RNAi oligonucleotides Compound No. 1591095 and Compound No. 1687246 are described herein above.

TABLE 157
Design of RNAi compounds targeted to HPRT1

| Duplex | Antisense Strand | Sense Strand |
|---|---|---|
| Compound No. | Compound No. | Compound No. |
| 1599476 | 1586322 | 1591095 |
| 1692889 | 1685106 | 1591095 |
| 1692892 | 1691628 | 1591095 |
| 1692895 | 1691630 | 1591095 |
| 1692898 | 1691629 | 1591095 |
| 1692901 | 1691631 | 1591095 |
| 1692904 | 1691632 | 1591095 |
| 1692907 | 1691633 | 1591095 |
| 1687264 | 1586322 | 1687246 |
| 1692917 | 1685106 | 1687246 |
| 1692920 | 1691628 | 1687246 |
| 1692923 | 1691630 | 1687246 |
| 1692926 | 1691629 | 1687246 |
| 1692929 | 1691631 | 1687246 |
| 1692932 | 1691632 | 1687246 |
| 1692935 | 1691633 | 1687246 |

### Example 67: Dose-Dependent Inhibition of Human HPRT1 in A431 Cells by RNAi Compounds

The RNAi agents described above were tested at various doses in A431 cells. Cultured A431 cells at a density of 20,000 cells per well were treated by RNAiMAX with RNAi compounds at concentrations indicated in the tables below. After a treatment period of approximately 24 hours, total RNA was isolated from the cells and HPRT1 RNA levels were measured by quantitative real-time RTPCR. Human HPRT1 primer-probe set RTS35336 (described herein above) was used to measure RNA levels. HPRT1 RNA levels were normalized to total RNA content, as measured by RIBOGREEN®. Reduction of HPRT1 RNA is presented in the tables below as percent HPRT1 RNA, relative to the amount of HPRT1 RNA in untreated control cells (% UTC). Compound No. 1599476 (described herein above) was included as a benchmark.

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment.

TABLE 158
Dose-dependent reduction of human HPRT1
RNA in A431 cells by RNAi compounds

| | HPRT1 RNA (% UTC) | | | | | | | |
|---|---|---|---|---|---|---|---|---|
| Compound | | | | | 0.001 | 0.0001 | 0.00001 | IC₅₀ |
| Number | 10 nM | 1 nM | 0.1 nM | 0.01 nM | nM | nM | nM | (pM) |
| 1692889 | 3 | 3 | 4 | 9 | 55 | 101 | 101 | 1.23 |
| 1692892 | 4 | 3 | 4 | 12 | 61 | 94 | 102 | 1.56 |
| 1692895 | 3 | 3 | 3 | 9 | 41 | 89 | 105 | 0.74 |
| 1692898 | 3 | 3 | 4 | 15 | 60 | 93 | 99 | 1.56 |
| 1692901 | 3 | 3 | 4 | 8 | 55 | 92 | 100 | 1.21 |
| 1692904 | 3 | 3 | 4 | 12 | 60 | 94 | 97 | 1.46 |
| 1692907 | 4 | 3 | 4 | 17 | 75 | 93 | 100 | 2.66 |
| 1687264 | 3 | 3 | 4 | 12 | 53 | 91 | 98 | 1.16 |
| 1692917 | 4 | 3 | 5 | 23 | 79 | 90 | 94 | 3.22 |
| 1599476 | 3 | 2 | 3 | 6 | 30 | 85 | 111 | 0.51 |

TABLE 159
Dose-dependent reduction of human HPRT1 RNA in A431 cells by RNAi compounds

| | HPRT1 RNA (% UTC) | | | | | | | | |
|---|---|---|---|---|---|---|---|---|---|
| Compound | | | | | | 0.001 | 0.0001 | 0.00001 | IC₅₀ |
| Number | 100 nM | 10 nM | 1 nM | 0.1 nM | 0.01 nM | nM | nM | nM | (pM) |
| 1692920 | 4 | 3 | 3 | 5 | 24 | 80 | 105 | 109 | 3.60 |
| 1692923 | 3 | 3 | 2 | 4 | 17 | 70 | 99 | 99 | 2.25 |
| 1692926 | 4 | 3 | 3 | 5 | 26 | 80 | 100 | 103 | 3.79 |
| 1692929 | 3 | 3 | 3 | 6 | 26 | 79 | 102 | 104 | 3.75 |
| 1692932 | 3 | 3 | 4 | 8 | 46 | 93 | 100 | 104 | 8.91 |
| 1692935 | 4 | 3 | 4 | 7 | 53 | 89 | 97 | 102 | 10.65 |
| 1599476 | 4 | 2 | 2 | 2 | 5 | 26 | 78 | 105 | 0.36 |

### Example 68: Design of siRNA Targeted to FXII Containing 2′-Fluoro Nucleosides

Modified oligonucleotides in the table below having either stereo-standard nucleosides or stereo-non-standard nucleosides in the antisense RNAi oligonucleotide and/or the sense RNAi oligonucleotide were synthesized using standard techniques. The antisense RNAi oligonucleotide described in the table below has the sequence TAAAGCACUUUAUUGAGUUUCUG (SEQ ID NO: 31) and is complementary to the complement of GenBank Accession No. NC_000079.6, truncated from nucleosides 55415001 to 55430000, (SEQ ID NO. 25) from nucleosides 12005 to 12026 (SEQ ID NO: 39). Antisense RNAi oligonucleotide Compound No. 1526195 is described herein above.

TABLE 160
Design of antisense RNAi oligonucleotides
targeted to FXII containing
2′-fluoro nucleosides

| Compound | Chemistry Notation | SEQ ID |
|---|---|---|
| No. | (5′ to 3′) | NO. |
| 1670875 | vP-TₑₛA_{fs}A_{yo}A_{yo}G_{yo}C_{do}A_{yo}C_{yo} | 31 |
| | U_{yo}U_{yo}U_{yo}A_{yo}U_{yo}U_{do}G_{yo}A_{do}G_{yo} | |
| | U_{yo}U_{yo}U_{yo}C_{ys}U_{ys}G_{y} | |

In the table above, a “vP-” represents a vinyl phosphonate (vP) moiety on the 5′-end, a subscript “f” represents a 2′-fluoro-β-D-ribosyl sugar moiety, a subscript “y” represents a 2′-O-methyl-β-D-ribosyl sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Each sense oligomeric compound further contains a GalNAc moiety conjugated to the 3′-oxygen as shown below:

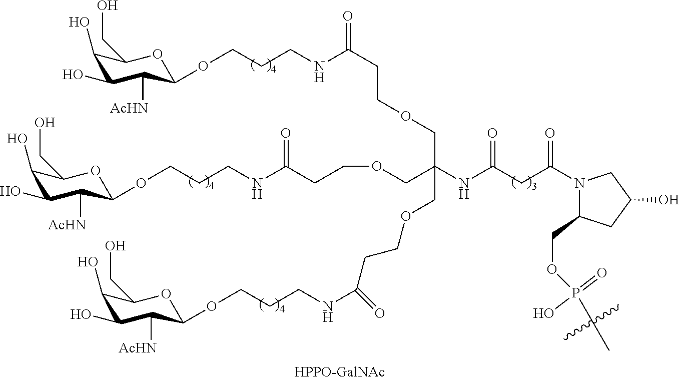

Sense RNAi oligonucleotide Compound No. 1523578 is described herein above and was previously disclosed in WO 2021/030778.

TABLE 161
Design of sense RNAi oligonucleotides
targeted to FXII containing
2′-fluoro nucleosides

| Compound | Chemistry Notation | SEQ ID |
|---|---|---|
| No. | (5′ to 3′) | NO. |
| 1670885 | G_{ys}A_{ys}A_{yo}A_{yo}C_{yo}U_{yo}C_{yo}A_{do}A_{do} | 26 |
| | U_{do}A_{do}A_{do}A_{do}G_{do}U_{yo}G_{yo}C_{yo}U_{yo} | |
| | U_{yo}U_{yo}A_{yo}-HPPO-GalNAc | |
| 1670886 | G_{ys}A_{ys}A_{yo}A_{yo}C_{yo}U_{yo}C_{do}A_{yo}A_{do} | 26 |
| | U_{do}A_{do}A_{yo}A_{yo}G_{yo}U_{yo}G_{yo}C_{yo}U_{yo} | |
| | U_{yo}U_{yo}A_{yo}-HPPO-GalNAc | |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

TABLE 162
Design of siRNA targeted to FXII containing 2′-fluoro nucleosides

| Duplex | Antisense Strand | Sense Strand |
|---|---|---|
| Compound No. | Compound No. | Compound No. |
| 1675710 | 1670875 | 1523578 |
| 1675711 | 1526195 | 1670885 |
| 1675712 | 1526195 | 1670886 |

### Example 69: Duration of Effect of siRNA Targets to FXII in Wild-Type C57BL/6 Mice

The duration of effect of RNAi agents on reduction of FXII plasma protein was tested in wild type C57BL/6 mice (Taconic Biosciences).

Mice were divided into groups of 4 male each. Each mouse received a single subcutaneous injection of RNAi agent at a dose of 1 mg/kg. A group of 8 mice received PBS as a control. Prior to the first dose, a cheek bleed was performed to determine plasma FXII protein levels at baseline. Cheek bleeds were also performed at 1 week, 2 weeks, 4 weeks, 6 weeks, 8 weeks, and 10 weeks following the dose. A cardiac puncture was performed at 12 weeks.

### Plasma Protein Analysis

Mouse FXII protein levels in plasma were determined using a Molecular Innovations FXII ELISA kit (catalog number: MFXIIKT-TOT). The data is presented as concentration of mouse FXII protein, in μg/mL.

TABLE 163
Reduction of mouse FXII protein by siRNA in wild type C57BL/6 mice over 12 weeks

| Compound | Plasma FXII Protein (μg/mL) | | | | | | | |
|---|---|---|---|---|---|---|---|---|
| Number | Baseline | Week 1 | Week 2 | Week 4 | Week 6 | Week 8 | Week 10 | Week 12 |
| PBS | 26 | 25 | 20 | 21 | 23 | 24 | 23‡ | 27‡ |
| 1526196 | 28 | 1 | 1 | 1 | 2 | 5 | 11 | 19 |
| 1645351 | 24 | 2 | 1 | 2 | 5 | 7 | 18 | 22 |
| 1645352 | 28 | 2 | 1 | 2 | 4 | 8 | 15 | 21 |
| 1645355 | 22 | 2 | 2 | 2 | 7 | 10 | 22 | 26 |
| 1632812 | 26 | 2 | 2 | 3 | 12 | 13 | 28 | 26 |
| 1645198 | 27 | 3 | 4 | 7 | 17 | 22‡ | 31‡ | 29‡ |
| 1633612 | 27 | 3 | 4 | 5 | 13 | 16 | 29 | 27 |
| 1669050 | 24 | 2 | 2 | 2 | 8 | 9 | 24 | 23 |
| 1669051 | 29 | 2 | 2 | 2 | 4 | 9 | 20 | 23 |
| 1669054 | 26 | 2 | 2 | 2 | 4 | 6 | 15 | 17 |
| 1645201 | 25 | 3 | 5 | 9 | 25 | 19 | 32 | 25 |
| 1669154 | 25 | 5 | 6 | 8 | 12 | 18 | 27 | 24 |
| 1669155 | 25 | 3 | 5 | 6 | 15 | 16 | 29 | 21 |
| 1652868 | 26 | 3 | 4 | 5 | 16 | 14 | 28 | 18 |
| 1669149 | 26 | 1 | 1 | 1 | 2 | 5 | 11 | 15 |
| 1669151 | 25 | 2 | 1 | 1 | 4‡ | 61 | 16‡ | 12‡ |
| 1669152 | 23 | 2 | 2 | 2 | 4 | 6 | 16 | 10 |
| 1675710 | 24 | 4 | 3 | 5 | 8 | 13 | 18 | 21 |
| 1675711 | 23 | 22 | 21 | 20 | 19 | 19 | 25 | 20 |
| 1675712 | 21 | 14 | 15 | 17 | 22 | 15 | 28 | 17 |

‡indicates that fewer than 8 samples were available for PBS treated mice, or fewer than 4 samples were available for RNAi agent treated mice

### RNA Analysis

Twelve weeks post treatment, mice were sacrificed. RNA was extracted from liver tissue for quantitative real-time RTPCR analysis of RNA expression of mouse FXII using primer probe set RTS2959 (described herein above). Results are presented as percent change of mouse HPRT RNA, relative to the amount of HPRT RNA in PBS control treated mice, normalized to RIBOGREEN (% control).

TABLE 164
Reduction of mouse FXII RNA by siRNA in
wild type C57BL/6 mice after 12 weeks

| Compound Number | FXII RNA (% control) |
|---|---|
| 1645351 | 68 |
| 1645352 | 74 |
| 1645355 | 84 |
| 1632812 | 84 |
| 1645198 | 92‡ |
| 1633612 | 92 |
| 1669050 | 86 |
| 1669051 | 80 |
| 1669054 | 65 |
| 1645201 | 84 |
| 1669154 | 85 |
| 1669155 | 96 |
| 1652868 | 91‡ |
| 1669149 | 65 |
| 1669151 | 73‡ |
| 1669152 | 56 |
| 1675710 | 87 |
| 1675711 | 96 |
| 1675712 | 96 |

‡indicates that fewer than 4 samples were available for RNAi agent treated mice

### Example 70: Design of siRNA Targeted to FXII Containing 2′-Fluoro Xylonucleosides

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotides described in the table below have the sequence UAAAGCACUUUAUUGAGUUUCUG (SEQ ID NO: 24). Aside from a single mismatch at position 1 on the 5′-end, each antisense RNAi oligonucleotide is 100% complementary to the complement of GenBank Accession No. NC_000079.6, truncated from nucleosides 55415001 to 55430000, (SEQ ID NO: 25) from nucleosides 12005 to 12026 (SEQ ID NO: 39). Each antisense RNAi oligonucleotide has a 5′-phosphate. Antisense RNAi oligonucleotide Compound No. 1653515 is described herein above.

TABLE 165
Design of antisense strand modified
oligonucleotides targeted to FXII
containing 2′-fluoroxylonucleosides

| Compound | Chemistry Notation | SEQ |
|---|---|---|
| No. | (5′ to 3′) | ID NO. |
| 1653513 | p.U_{ys}A_{[f2bDx]s}A_{yo}A_{yo}G_{yo}C_{fo} | 24 |
| | A_{yo}C_{yo}U_{yo}U_{yo}U_{yo}A_{yo}U_{yo}U_{fo}G_{yo} | |
| | A_{fo}G_{yo}U_{yo}U_{yo}U_{yo}C_{ys}U_{ys}G_{y} | |
| 1653514 | p.U_{ys}A_{fs}A_{yo}A_{yo}G_{yo}C_{[f2bDx]o} | 24 |
| | A_{yo}C_{yo}U_{yo}U_{yo}U_{yo}A_{yo}U_{yo}U_{fo}G_{yo} | |
| | A_{fo}G_{yo}U_{yo}U_{yo}U_{yo}C_{ys}U_{ys}G_{y} | |
| 1653517 | p.U_{ys}A_{[f2bDx]s}A_{yo}A_{yo}G_{yo}C_{[f2bDx]o} | |
| | A_{yo}C_{yo}U_{yo}U_{yo}U_{yo}A_{yo}U_{yo}U[_{f2bDx}]ₒ | 24 |
| | G_{yo}A_{[f2bDx]o}G_{yo}U_{yo}U_{yo}U_{yo}C_{ys}U_{ys}G_{y} | |

In the table above, a “p.” represents a 5′ terminal phosphate group, a subscript “f” represents a 2′-fluoro-β-D-ribosyl sugar moiety, a subscript “y” represents a 2′-O-methyl-β-D-ribosyl sugar moiety, a subscript “[f2bDx]” represents a 2′-fluoro-β-D-xylosyl sugar moiety, a subscript “s” represents a phosphorothioate internucleoside linkage, and a subscript “o” represents a phosphodiester internucleoside linkage.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Sense RNAi oligonucleotide Compound No. 1523578 is described herein above.

TABLE 166
Design of RNAi compounds targeted
to FXII 2′-fluoroxylonucleosides

| Duplex | Antisense Strand | Sense Strand |
|---|---|---|
| Compound No. | Compound No. | Compound No. |
| 1669055 | 1653513 | 1523578 |
| 1669154 | 1653515 | 1523578 |
| 1679985 | 1653514 | 1523578 |
| 1679986 | 1653517 | 1523578 |

### Example 71: Dose-Dependent Inhibition of FXII in Mouse Hepatocyte Cells by RNAi Compounds

The RNAi agents described above were tested at various doses in mouse hepatocyte cells. Mouse hepatocyte cells at a density of 20,000 cells per well by free uptake with RNAi compound at concentrations indicated in the tables below. After a treatment period of approximately 24 hours, total RNA was isolated from the cells and FXII RNA levels were measured by quantitative real-time RTPCR. Mouse FXII primer-probe set RTS2959 (described herein above) was used to measure RNA levels. FXII RNA levels were normalized to total RNA content, as measured by RIBOGREEN®. Reduction of FXII RNA is presented in the tables below as percent FXII RNA, relative to the amount of FXII RNA in untreated control cells (% UTC). “N.D.” indicates data that was not determined.

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Parent Compound No. 1632812 (described herein above) and parent Compound No. 1523582 (described herein above) were included as a benchmarks.

TABLE 167
Dose-dependent reduction of mouse FXII RNA in mouse hepatocytes by RNAi compounds

| | FXII RNA (% UTC) | | | | | | | | |
|---|---|---|---|---|---|---|---|---|---|
| Compound | | | | 0.01 | 0,001 | 0.0001 | 0.00001 | 0.000001 | IC₅₀ |
| No. | 10 nM | 1 nM | 0.1 nM | nM | nM | nM | nM | nM | (pM) |
| 1632812 | 14 | 19 | 62 | 102 | 112 | 103 | 99 | 101 | 206 |
| 1526196 | 13 | 18 | 50 | 89 | 110 | 105 | 102 | 94 | 121 |
| 1669054 | 15 | 21 | 55 | 105 | 99 | 98 | 103 | 90 | 181 |
| 1669055 | 20 | 39 | 72 | 99 | 110 | 106 | 96 | 97 | 605 |
| 1679985 | 16 | 27 | 70 | 113 | 108 | 96 | 96 | 99 | 347 |
| 1669154 | 17 | 34 | 71 | 95 | 99 | 102 | 92 | 105 | 433 |
| 1669155 | 15 | 18 | 63 | 97 | 103 | 94 | 94 | 96 | 205 |
| 1679986 | 65 | 79 | 101 | 98 | 90 | 89 | 91 | 101 | >10,000 |

### Example 72: Design of siRNA Targeted to FXII Containing 2′-Fluoro Ribonucleosides

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotides described in the table below have the sequence TAAAGCACUUUAUUGAGUUUCUG (SEQ ID NO: 31). Aside from a single mismatch at position 1 on the 5′-end, each antisense RNAi oligonucleotide is 100% complementary to the complement of GenBank Accession No. NC_000079.6, truncated from nucleosides 55415001 to 55430000, (SEQ ID NO: 25) from nucleosides 12005 to 12026 (SEQ ID NO: 39). Each antisense RNAi oligonucleotide has a 5′-phosphate. Antisense RNAi oligonucleotide Compound No. 1653515 is described herein above.

TABLE 168
Design of antisense strand modified
oligonucleotides targeted to FXII
containing 2′-fluoroxylonucleosides

| Compound | Chemistry Notation | SEQ |
|---|---|---|
| No. | (5′ to 3′) | ID NO. |
| 1679412 | vP-TₑₛA_{ys}A_{yo}A_{yo}G_{yo}C_{fo}A_{yo}C_{yo} | 31 |
| | U_{yo}U_{yo}U_{yo}A_{yo}U_{yo}U_{fo}G_{yo}A_{fo}G_{yo} | |
| | U_{yo}U_{yo}U_{yo}C_{ys}U_{ys}G_{y} | |
| 1676703 | vP-TₑₛA_{fo}A_{yo}A_{yo}G_{yo}C_{fo}A_{yo}C_{yo} | 31 |
| | U_{yo}U_{yo}U_{yo}A_{yo}U_{yo}U_{fo}G_{yo}A_{fo}G_{yo} | |
| | U_{yo}U_{yo}U_{yo}C_{ys}U_{ys}G_{y} | |

In the table above, a “vP-” represents a 5′ terminal vinyl phosphate group, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “s” represents a phosphorothioate internucleoside linkage, and a subscript “o” represents a phosphodiester internucleoside linkage.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Each sense RNAi oligomeric compound further contains a GalNAc moiety conjugated to the 3′-oxygen as shown below:

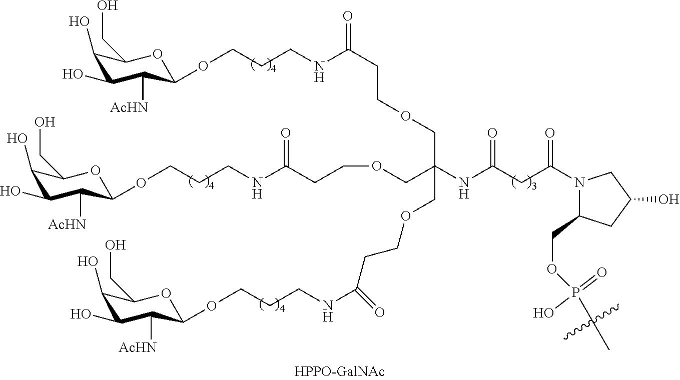

Sense RNAi oligonucleotide Compound No. 1523578 is described herein above.

TABLE 169
Design of sense strand modified oligonucleotides
targeted to FXII containing
2′-fluororibonucleosides

| Compound | Chemistry Notation | SEQ |
|---|---|---|
| No. | (5′ to 3′) | ID NO. |
| 1668945 | G_{ys}A_{ys}A_{yo}A_{yo}C_{yo}U_{yo}C_{fo}A_{yo}A_{fo} | 26 |
| | U_{yo}A_{fo}A_{yo}A_{yo}G_{yo}U_{yo}G_{yo}C_{yo}U_{yo} | |
| | U_{yo}U_{yo}A_{y}-HPPO-GalNAc | |
| 1657709 | G_{ys}A_{ys}A_{yo}A_{yo}C_{yo}U_{yo}C_{yo}A_{yo}A_{fo} | 26 |
| | U_{yo}A_{fo}A_{yo}A_{yo}G_{yo}U_{yo}G_{yo}C_{yo}U_{yo} | |
| | U_{yo}U_{yo}A_{y}-HPPO-GalNAc | |
| 1657710 | G_{ys}A_{ys}A_{yo}A_{yo}C_{yo}U_{yo}C_{yo}A_{yo}A_{fo} | 26 |
| | U_{fo}A_{yo}A_{yo}A_{yo}G_{yo}U_{yo}G_{yo}C_{yo}U_{yo} | |
| | U_{yo}U_{yo}A_{y}-HPPO-GalNAc | |
| 1657711 | G_{ys}A_{ys}A_{yo}A_{yo}C_{yo}U_{yo}C_{yo}A_{yo}A_{yo} | 26 |
| | U_{yo}A_{fo}A_{yo}A_{yo}G_{yo}U_{yo}G_{yo}C_{yo}U_{yo} | |
| | U_{yo}U_{yo}A_{y}-HPPO-GalNAc | |
| 1657713 | G_{ys}A_{ys}A_{yo}A_{yo}C_{yo}U_{yo}C_{yo}A_{yo}A_{fo} | 26 |
| | U_{yo}A_{yo}A_{yo}A_{yo}G_{yo}U_{yo}G_{yo}C_{yo}U_{yo} | |
| | U_{yo}U_{yo}A_{y}-HPPO-GalNAc | |
| 1657714 | G_{ys}A_{ys}A_{yo}A_{yo}C_{yo}U_{yo}C_{fo}A_{yo}A_{yo} | 26 |
| | U_{yo}A_{yo}A_{yo}A_{yo}G_{yo}U_{yo}G_{yo}C_{yo}U_{yo} | |
| | U_{yo}U_{yo}A_{y}-HPPO-GalNAc | |
| 1657715 | G_{ys}A_{ys}A_{yo}A_{yo}C_{yo}U_{yo}C_{yo}A_{yo}A_{yo} | 26 |
| | U_{yo}A_{yo}A_{yo}A_{yo}G_{yo}U_{yo}G_{yo}C_{yo}U_{yo} | |
| | U_{yo}U_{yo}A_{y}-HPPO-GalNAc | |
| 1657716 | G_{ys}A_{ys}A_{yo}A_{yo}C_{yo}U_{yo}C_{yo}A_{yo}A_{yo} | 26 |
| | U_{yo}A_{yo}A_{yo}A_{yo}G_{yo}U_{yo}G_{yo}C_{yo}U_{yo} | |
| | U_{ys}U_{ys}A_{y}-HPPO-GalNAc | |

In the table above, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, and a subscript “o” represents a phosphodiester internucleoside linkage.

TABLE 170
Design of RNAi compounds targeted
to FXII 2′-fluoroxylonucleosides

| Duplex | Antisense Strand | Sense Strand |
|---|---|---|
| Compound No. | Compound No. | Compound No. |
| 1669055 | 1653513 | 1523578 |
| 1679985 | 1653514 | 1523578 |
| 1679986 | 1653517 | 1523578 |
| 1679968 | 1526195 | 1668945 |
| 1679969 | 1526195 | 1657709 |
| 1679970 | 1526195 | 1657710 |
| 1679971 | 1526195 | 1657711 |
| 1679972 | 1526195 | 1657713 |
| 1679973 | 1526195 | 1657714 |
| 1679974 | 1526195 | 1657715 |
| 1679975 | 1526195 | 1657716 |
| 1679413 | 1679412 | 1523578 |
| 1680996 | 1676703 | 1523578 |

### Example 73: Dose-Dependent Inhibition of FXII in Mouse Hepatocyte Cells by RNAi Compounds

The RNAi agents described above were tested at various doses in mouse hepatocyte cells. Mouse hepatocyte cells at a density of 20,000 cells per well by free uptake with RNAi compound at concentrations indicated in the tables below. After a treatment period of approximately 24 hours, total RNA was isolated from the cells and FXII RNA levels were measured by quantitative real-time RTPCR. Mouse FXII primer-probe set RTS2959 (described herein above) was used to measure RNA levels. FXII RNA levels were normalized to total RNA content, as measured by RIBOGREEN®. Reduction of FXII RNA is presented in the tables below as percent FXII RNA, relative to the amount of FXII RNA in untreated control cells (% UTC).

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Parent Compound No. 1632812 (described herein above) and parent Compound No. 1523582 (described herein above) were included as a benchmarks.

TABLE 171
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0.001 | 0.0001 | 0.00001 | 0.000001 | IC₅₀ |
| No. | 10 nM | 1 nM | 0.1 nM | nM | nM | nM | nM | nM | (pM) |
| 1526196 | 14 | 19 | 36 | 94 | 113 | 119 | 110 | 102 | 76 |
| 1669149 | 16 | 19 | 36 | 99 | 122 | 112 | 108 | 98 | 73 |
| 1679968 | 19 | 34 | 70 | 113 | 112 | 117 | 103 | 94 | 476 |
| 1669151 | 18 | 26 | 70 | 96 | 106 | 108 | 96 | 101 | 325 |
| 1679969 | 20 | 26 | 65 | 111 | 105 | 106 | 85 | 87 | 319 |
| 1679970 | 18 | 28 | 71 | 94 | 100 | 109 | 98 | 102 | 349 |
| 1679971 | 17 | 34 | 78 | 93 | 100 | 103 | 90 | 106 | 513 |
| 1669152 | 18 | 32 | 74 | 101 | 103 | 77 | 92 | 97 | 458 |
| 1679972 | 18 | 26 | 69 | 92 | 97 | 98 | 88 | 99 | 301 |
| 1679973 | 20 | 38 | 71 | 93 | 97 | 104 | 92 | 97 | 527 |

TABLE 172
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0.001 | 0.0001 | 0.00001 | 0.000001 | IC₅₀ |
| No. | 10 nM | 1 nM | 0.1 nM | nM | nM | nM | nM | nM | (pM) |
| 1632812 | 14 | 20 | 47 | 98 | 100 | 99 | 101 | 83 | 127 |
| 1526196 | 15 | 19 | 33 | 95 | 115 | 108 | 102 | 92 | 66 |
| 1679974 | 19 | 39 | 70 | 106 | 109 | 109 | 106 | 96 | 576 |
| 1679975 | 20 | 25 | 54 | 94 | 102 | 107 | 98 | 91 | 204 |

### Example 74: Design of siRNA Targeted to FXII Containing 2′-Deoxyribonucleosides

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotides described in the table below have the sequence TAAAGCACUUUAUUGAGUUUCUG (SEQ ID NO: 31) or UAAAGCACUUUAUUGAGUUUCUG (SEQ ID NO: 24). Aside from a single mismatch at position 1 on the 5′-end, each antisense RNAi oligonucleotide in the table below is complementary to the complement of GenBank Accession No. NC_000079.6, truncated from nucleosides 55415001 to 55430000, (SEQ ID NO. 25) from nucleosides 12005 to 12026 (SEQ ID NO: 39). Antisense RNAi oligonucleotide Compound No. 1526195 is described herein above.

TABLE 173
Design of antisense strand modified
oligonucleotides targeted to FXII
containing 2′-deoxyribonucleosides

| Compound | Chemistry Notation | SEQ ID |
|---|---|---|
| No. | (5′ to 3′) | NO. |
| 1670875 | vP-TₑₛA_{fs}A_{yo}A_{yo}G_{yo}C_{do}A_{yo}C_{yo} | 31 |
| | U_{yo}U_{yo}U_{yo}A_{yo}U_{yo}U_{do}G_{yo}A_{do}G_{yo} | |
| | U_{yo}U_{yo}U_{yo}C_{ys}U_{ys}G_{y} | |
| 1670873 | vP-TₑₛA_{ds}A_{yo}A_{yo}G_{yo}C_{fo}A_{yo}C_{yo} | 31 |
| | U_{yo}U_{yo}U_{yo}A_{yo}U_{yo}U_{fo}G_{yo}A_{fo}G_{yo} | |
| | U_{yo}U_{yo}U_{yo}C_{ys}U_{ys}G_{y} | |
| 1670874 | vP-TₑₛA_{ds}A_{yo}A_{yo}G_{yo}C_{do}A_{yo}C_{yo} | 31 |
| | U_{yo}U_{yo}U_{yo}A_{yo}U_{yo}U_{do}G_{yo}A_{do}G_{yo} | |
| | U_{yo}U_{yo}U_{yo}C_{ys}U_{ys}G_{y} | |
| 1670883 | vP-TₑₛA_{ds}A_{yo}A_{yo}G_{do}C_{yo}A_{do}C_{yo} | 31 |
| | U_{yo}U_{yo}U_{yo}A_{do}U_{yo}U_{fo}G_{yo}A_{yo}G_{yo} | |
| | U_{yo}U_{yo}U_{yo}C_{ys}U_{ys}G_{y} | |
| 1670882 | vP-TₑₛA_{fs}A_{yo}A_{yo}G_{fo}C_{yo}A_{fo}C_{yo} | 31 |
| | U_{yo}U_{yo}U_{yo}A_{fo}U_{yo}U_{fo}G_{yo}A_{yo}G_{yo} | |
| | U_{yo}U_{yo}U_{yo}C_{ys}U_{ys}G_{y} | |
| 1670876 | p.U_{ys}A_{ds}A_{yo}A_{yo}G_{yo}C_{fo}A_{yo}C_{yo} | 25 |
| | U_{yo}U_{yo}U_{yo}A_{yo}U_{yo}U_{fo}G_{yo}A_{fo}G_{yo} | |
| | U_{yo}U_{yo}U_{yo}C_{ys}U_{ys}G_{y} | |
| 1670877 | p.U_{ys}A_{fs}A_{yo}A_{yo}G_{yo}C_{do}A_{yo}C_{yo} | 25 |
| | U_{yo}U_{yo}U_{yo}A_{yo}U_{yo}U_{fo}G_{yo}A_{fo}G_{yo} | |
| | U_{yo}U_{yo}U_{yo}C_{ys}U_{ys}G_{y} | |
| 1670879 | p.U_{ys}A_{fs}A_{yo}A_{yo}G_{yo}C_{fo}A_{yo}C_{yo} | 25 |
| | U_{yo}U_{yo}U_{yo}A_{yo}U_{yo}U_{do}G_{yo}A_{fo}G_{yo} | |
| | U_{yo}U_{yo}U_{yo}C_{ys}U_{ys}G_{y} | |
| 1670880 | p.U_{ys}A_{fs}A_{yo}A_{yo}G_{yo}C_{fo}A_{yo}C_{yo} | 25 |
| | U_{yo}U_{yo}U_{yo}A_{yo}U_{yo}U_{fo}G_{yo}A_{do}G_{yo} | |
| | U_{yo}U_{yo}U_{yo}C_{ys}U_{ys}G_{y} | |
| 1670881 | p.U_{ys}A_{ds}A_{yo}A_{yo}G_{yo}C_{do}A_{yo}C_{yo} | 25 |
| | U_{yo}U_{yo}U_{yo}A_{yo}U_{yo}U_{do}G_{yo}A_{do}G_{yo} | |
| | U_{yo}U_{yo}U_{yo}C_{ys}U_{ys}G_{y} | |

In the table above, a “p.” represents a 5′ terminal phosphate group, a “vP-” represents a vinyl phosphonate (vP) moiety on the 5′-end, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

The antisense RNAi oligonucleotide described in the table below has the sequence UAAAGCACUUUAUTGAGUUUCUG (SEQ ID NO: 32). Aside from a single mismatch at position 1 on the 5′-end, each antisense RNAi oligonucleotide in the table below is complementary to the complement of GenBank Accession No. NC_000079.6, truncated from nucleosides 55415001 to 55430000, (SEQ ID NO. 25) from nucleosides 12005 to 12026 (SEQ ID NO: 39). The antisense RNAi oligonucleotide in the table below has a 5′-phosphate (p.).

TABLE 174
Design of antisense strand modified
oligonucleotides targeted to FXII
containing 2′-deoxyribonucleosides

| Compound | Chemistry Notation | SEQ ID |
|---|---|---|
| No. | (5′ to 3′) | NO. |
| 1670878 | p.U_{ys}A_{fs}A_{yo}A_{yo}G_{yo}C_{fo}A_{yo}C_{yo}U_{yo}U_{yo}U_{yo} | 32 |
| | A_{yo}U_{yo}T_{do}G_{yo}A_{fo}G_{yo}U_{yo}U_{yo}U_{yo}C_{ys}U_{ys}G_{y} | |

In the table above, a subscript “f” represents a 2′-fluoro-β-D-ribosyl sugar moiety, a subscript “y” represents a 2′-O-methyl-β-D-ribosyl sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Each sense RNAi oligomeric compound further contains a GalNAc moiety conjugated to the 3′-oxygen as shown below:

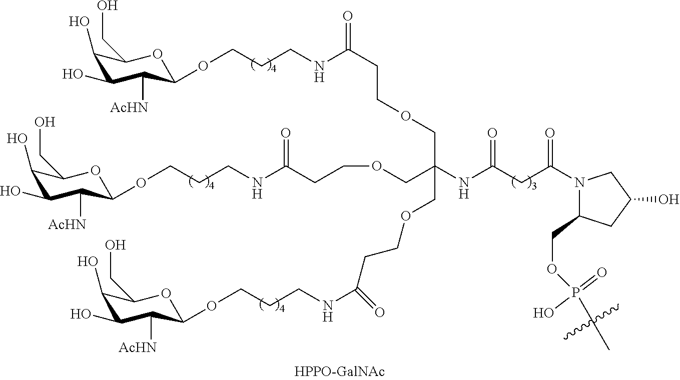

Sense RNAi oligonucleotide Compound No. 1523578 is described herein above.

TABLE 175
Design of sense strand modified
oligonucleotides targeted to FXII
containing 2′-deoxyribonucleosides

| Compound | Chemistry Notation | SEQ ID |
|---|---|---|
| No. | (5′ to 3′) | NO. |
| 1670884 | G_{ys}A_{ys}A_{yo}A_{yo}C_{yo}U_{yo}C_{yo}A_{yo}A_{do}U_{do}A_{do}A_{yo} | 26 |
| | A_{yo}G_{yo}U_{yo}G_{yo}C_{yo}U_{yo}U_{yo}U_{yo}A_{y}-HPPO- | |
| | GalNAc | |
| 1670885 | G_{ys}A_{ys}A_{yo}A_{yo}C_{yo}U_{yo}C_{yo}A_{do}A_{do}U_{do}A_{do}A_{do} | 26 |
| | A_{do}G_{do}U_{yo}G_{yo}C_{yo}U_{yo}U_{yo}U_{yo}A_{y}-HPPO- | |
| | GalNAc | |
| 1670886 | G_{ys}A_{ys}A_{yo}A_{yo}C_{yo}U_{yo}C_{do}A_{yo}A_{do}U_{do}A_{do}A_{yo} | 26 |
| | A_{yo}G_{yo}U_{yo}G_{yo}C_{yo}U_{yo}U_{yo}U_{yo}A_{y}-HPPO- | |
| | GalNAc | |

In the table above, a subscript “f” represents a 2′-fluoro-β-D-ribosyl sugar moiety, a subscript “y” represents a 2′-O-methyl-β-D-ribosyl sugar moiety, a subscript “d” represents a 2′-β-D-deoxyribosyl sugar moiety, a subscript “s” represents a phosphorothioate internucleoside linkage, and a subscript “o” represents a phosphodiester internucleoside linkage.

TABLE 176
Design of RNAi compounds targeted to FXII
containing 2′-deoxyribonucleosides

| Duplex | Antisense Strand | Sense Strand |
|---|---|---|
| Compound No. | Compound No. | Compound No. |
| 1675710 | 1670875 | 1523578 |
| 1675711 | 1526195 | 1670885 |
| 1675712 | 1526195 | 1670886 |
| 1675713 | 1526195 | 1670884 |
| 1679589 | 1670873 | 1523578 |
| 1679590 | 1670874 | 1523578 |
| 1679591 | 1670883 | 1523578 |
| 1679592 | 1670882 | 1523578 |
| 1679593 | 1670876 | 1523578 |
| 1679594 | 1670877 | 1523578 |
| 1679595 | 1670878 | 1523578 |
| 1679596 | 1670879 | 1523578 |
| 1679597 | 1670880 | 1523578 |
| 1679598 | 1670881 | 1523578 |

### Example 75: Dose-Dependent Inhibition of FXII in Mouse Hepatocyte Cells by RNAi Compounds

The RNAi agents described above were tested at various doses in mouse hepatocyte cells. Mouse hepatocyte cells at a density of 20,000 cells per well by free uptake with RNAi compound at concentrations indicated in the tables below. After a treatment period of approximately 24 hours, total RNA was isolated from the cells and FXII RNA levels were measured by quantitative real-time RTPCR. Mouse FXII primer-probe set RTS2959 (described herein above) was used to measure RNA levels. FXII RNA levels were normalized to total RNA content, as measured by RIBOGREEN®. Reduction of FXII RNA is presented in the tables below as percent FXII RNA, relative to the amount of FXII RNA in untreated control cells (% UTC).

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Parent Compound No. 1632812 (described herein above) and parent Compound No. 1523582 (described herein above) were included as a benchmarks.

TABLE 177
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0,001 | 0.0001 | 0.00001 | 0.000001 | IC₅₀ |
| No. | 10 nM | 1 nM | 0.1 nM | nM | nM | nM | nM | nM | (pM) |
| 1632812 | 9 | 9 | 56 | 107 | 131 | 119 | 110 | 109 | 123 |
| 1526196 | 7 | 9 | 24 | 102 | 129 | 114 | 121 | 102 | 81 |
| 1679589 | 7 | 10 | 35 | 102 | 119 | 120 | 104 | 104 | 80 |
| 1679593 | 7 | 7 | 34 | 109 | 122 | 107 | 101 | 95 | 89 |
| 1679594 | 8 | 8 | 34 | 90 | 114 | 102 | 102 | 111 | 62 |
| 1679595 | 8 | 10 | 45 | 103 | 120 | 115 | 98 | 114 | 90 |
| 1679596 | 8 | 13 | 50 | 106 | 118 | 99 | 107 | 115 | 109 |
| 1679597 | 8 | 11 | 50 | 94 | 103 | 97 | 95 | 103 | 106 |
| 1679598 | 10 | 15 | 53 | 94 | 100 | 97 | 98 | 108 | 130 |
| 1679590 | 11 | 13 | 51 | 80 | 96 | 95 | 103 | 111 | 94 |

TABLE 178
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0.001 | 0.0001 | 0.00001 | 0.000001 | IC₅₀ |
| No. | 10 nM | 1 nM | 0.1 nM | nM | nM | nM | nM | nM | (pM) |
| 1632812 | 14 | 20 | 47 | 98 | 100 | 99 | 101 | 83 | 127 |
| 1526196 | 15 | 19 | 33 | 95 | 115 | 108 | 102 | 92 | 66 |
| 1675710 | 19 | 25 | 50 | 87 | 95 | 97 | 101 | 97 | 158 |
| 1675713 | 16 | 21 | 58 | 104 | 97 | 116 | 97 | 89 | 197 |
| 1675711 | 17 | 21 | 47 | 94 | 104 | 104 | 115 | 104 | 131 |
| 1675712 | 17 | 20 | 50 | 97 | 104 | 106 | 86 | 102 | 146 |

TABLE 179
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0.001 | 0.0001 | 0.00001 | 0.000001 | IC₅₀ |
| No. | 10 nM | 1 nM | 0.1 nM | nM | nM | nM | nM | nM | (pM) |
| 1632812 | 14 | 19 | 62 | 102 | 112 | 103 | 99 | 101 | 206 |
| 1526196 | 13 | 18 | 50 | 89 | 110 | 105 | 102 | 94 | 121 |
| 1679591 | 14 | 14 | 33 | 81 | 104 | 95 | 98 | 100 | 55 |
| 1679592 | 13 | 16 | 36 | 102 | 103 | 95 | 93 | 95 | 74 |

### Example 76: Design of siRNA Targeted to FXII Containing Mesyl Phosphoramidate Internucleoside Linkages

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotides described in the table below have the sequence TAAAGCACUUUAUUGAGUUUCUG (SEQ ID NO: 31). Aside from a single mismatch at position 1 on the 5′-end, the sequence is complementary to the complement of GenBank Accession No. NC_000079.6, truncated from nucleosides 55415001 to 55430000, (SEQ ID NO. 25) from nucleosides 12005 to 12026 (SEQ ID NO: 39). Each antisense oligonucleotide in the table below has a 5′ terminal vinyl phosphonate (vP-).

TABLE 180
Design of antisense strand modified
oligonucleotides targeted to FXII
containing mesyl phosphoramidate
internucleoside linkages

| Compound | | SEQ ID |
|---|---|---|
| No. | Chemistry Notation (5′ to 3′) | NO. |
| 1676701 | vP-T_{ez}A_{fz}A_{yo}A_{yo}G_{yo}C_{fo}A_{yo}C_{yo}U_{yo}U_{yo} | 31 |
| | U_{yo}A_{yo}U_{yo}U_{fo}G_{yo}A_{fo}G_{yo}U_{yo}U_{yo}U_{yo}C_{ys} | |
| | U_{ys}G_{y} | |
| 1676702 | vP-T_{ez}A_{fo}A_{yo}A_{yo}G_{yo}C_{fo}A_{yo}C_{yo}U_{yo}U_{yo} | 31 |
| | U_{yo}A_{yo}U_{yo}U_{fo}G_{yo}A_{fo}G_{yo}U_{yo}U_{yo}U_{yo}C_{ys} | |
| | U_{ys}G_{y} | |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “s” represents a phosphorothioate internucleoside linkage, a subscript “o” represents a phosphodiester internucleoside linkage, and a subscript “z” represents a mesyl phosphoramidate internucleoside linkage (Formula II).

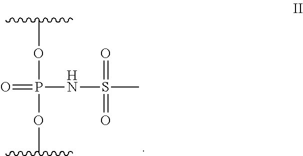 [SMILES: *OP(=O)(NS(C)(=O)=O)O*]

The antisense RNAi oligonucleotides described in the table below have the sequence TAAAGCACUUUAUUGAGUUUCTT (SEQ ID NO: 43). Aside from a mismatch at position 1 on the 5′-end and position 23 on the 3′-end, the sequence is complementary to the complement of GenBank Accession No. NC_000079.6, truncated from nucleosides 55415001 to 55430000, (SEQ ID NO. 25) from nucleosides 12005 to 12026 (SEQ ID NO: 39). Each antisense oligonucleotide in the table below has a 5′ terminal vinyl phosphonate (vP-).

TABLE 181
Design of antisense strand modified
oligonucleotides targeted to FXII
containing mesyl phosphoramidate
internucleoside linkages

| Compound | Chemistry Notation | SEQ ID |
|---|---|---|
| No. | (5′ to 3′) | NO. |
| 1678021 | vP-TₑₛA_{fz}A_{yo}A_{yo}G_{yo}C_{fo}A_{yo}C_{yo} | 43 |
| | U_{yo}U_{yo}U_{yo}A_{yo}U_{yo}U_{fo}G_{yo}A_{fo}G_{yo} | |
| | U_{yo}U_{yo}U_{yo}C_{yz}T_{dz}T_{d} | |
| 1678022 | vP-TₑₛA_{fz}A_{yo}A_{yo}G_{yo}C_{fo}A_{yo}C_{yo} | 43 |
| | U_{yo}U_{yo}U_{yo}A_{yo}U_{yo}U_{fo}G_{yo}A_{fo}G_{yo} | |
| | U_{yo}U_{yo}U_{yo}C_{yz}T_{ds}T_{d} | |
| 1678023 | vP-TₑₛA_{fz}A_{yo}A_{yo}G_{yo}C_{fo}A_{yo}C_{yo} | 43 |
| | U_{yo}U_{yo}U_{yo}A_{yo}U_{yo}U_{fo}G_{yo}A_{fo}G_{yo} | |
| | U_{yo}U_{yo}U_{yo}C_{ys}T_{dz}T_{d} | |
| 1678024 | vP-TₑₛA_{fz}A_{yo}A_{yo}G_{yo}C_{fo}A_{yo}C_{yo} | 43 |
| | U_{yo}U_{yo}U_{yo}A_{yo}U_{yo}U_{fo}G_{yo}A_{fo}G_{yo} | |
| | U_{yo}U_{yo}U_{yo}C_{ys}T_{ds}T_{d} | |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “d” represents a 2′-β-D-deoxyribosyl sugar moiety, a subscript “s” represents a phosphorothioate internucleoside linkage, a subscript “o” represents a phosphodiester internucleoside linkage, and a subscript “z” represents a mesyl phosphoramidate internucleoside linkage (Formula II).

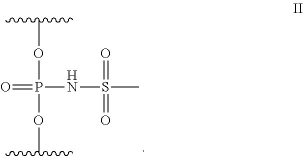 [SMILES: *OP(=O)(NS(C)(=O)=O)O*]

The antisense RNAi oligonucleotides described in the table below have the sequence UAAAGCACUUUAUUGAGUUUCUG (SEQ ID NO: 24). Aside from a single mismatch at position 1 on the 5′-end, each antisense RNAi oligonucleotide is 100% complementary to the complement of GenBank Accession No. NC_000079.6, truncated from nucleosides 55415001 to 55430000, (SEQ ID NO: 25) from nucleosides 12005 to 12026 (SEQ ID NO: 39). Each antisense RNAi oligonucleotide has a 5′-phosphate (p.).

### Table 182: Design of Antisense Strand Modified Oligonucleotides Targeted to FXII Containing Mesyl Phosphoramidate Internucleoside Linkages

TABLE 182
Design of antisense strand modified oligonucleotides targeted to
FXII containing mesyl phosphoramidate internucleoside linkages

| Compound | | SEQ ID |
|---|---|---|
| No. | Chemistry Notation (5′ to 3′) | NO. |
| 1675506 | p.U_{ys}A_{fz}A_{yo}A_{yo}G_{yo}C_{fz}A_{yo}C_{yo}U_{yo}U_{yO}U_{yo}A_{yo}U_{yo}U_{fz}G_{yo}A_{fz}G_{yo}U_{yo}U_{yo}U_{yo}C_{ys}U_{ys}G_{y} | 24 |
| 1675507 | p.U_{ys}A_{fs}A_{yo}A_{yo}G_{yo}C_{fz}A_{yo}C_{yo}U_{yO}U_{yo}U_{yo}A_{yo}U_{yo}U_{fz}G_{yo}A_{fz}G_{yo}U_{yo}U_{yo}U_{yo}C_{ys}U_{ys}G_{y} | 24 |
| 1675508 | p.U_{ys}A_{fs}A_{yo}A_{yo}G_{yo}C_{fo}A_{yo}C_{yo}U_{yo}U_{yo}U_{yo}A_{yo}U_{yo}U_{fz}G_{yo}A_{fz}G_{yo}U_{yo}U_{yo}U_{yo}C_{ys}U_{ys}G_{y} | 24 |
| 1675509 | p.U_{ys}A_{fz}A_{yo}A_{yo}G_{yo}C_{fo}A_{yo}C_{yo}U_{yo}U_{yo}U_{yo}A_{yo}U_{yo}U_{fz}G_{yo}A_{fo}G_{yo}U_{yO}U_{yo}U_{yo}C_{ys}U_{ys}G_{y} | 24 |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE sugar moiety, a subscript “s” represents a phosphorothioate internucleoside linkage, a subscript “o” represents a phosphodiester internucleoside linkage, and a subscript “z” represents a mesyl phosphoramidate internucleoside linkage (Formula II).

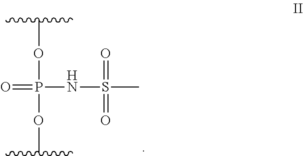 [SMILES: *OP(=O)(NS(C)(=O)=O)O*]

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Each sense RNAi oligomeric compound further contains a GalNAc moiety conjugated to the 5′ oxygen or the 3′-oxygen as shown below:

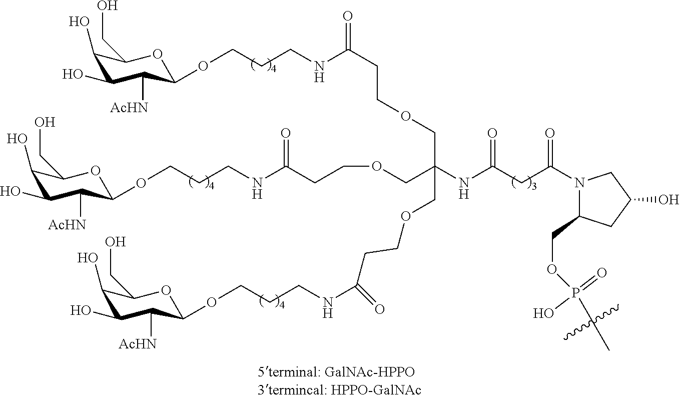

Sense RNAi oligonucleotide Compound No. 1523578 is described herein above.

TABLE 183
Design of sense strand modified oligonucleotides
targeted to FXII containing mesyl
phosphoramidate internucleoside linkages

| Compound | Chemistry Notation | SEQ ID |
|---|---|---|
| No. | (5′ to 3′) | NO. |
| 1671536 | GalNAc-HPPO-G_{yo}A_{yo}A_{yo}A_{yo}C_{yo} | 26 |
| | U_{yo}C_{fo}A_{yo}A_{fo}U_{fo}A_{fo}A_{yo}A_{yo}G_{yo} | |
| | U_{yo}G_{yo}C_{yo}U_{yo}U_{ys}U_{ys}A_{y} | |
| 1675510 | G_{ys}A_{ys}A_{yo}A_{yo}C_{yo}U_{yo}C_{fz}A_{yo}A_{fz} | 26 |
| | U_{fo}A_{fz}A_{yo}A_{yo}G_{yo}U_{yo}G_{yo}C_{yo}U_{yo} | |
| | U_{yo}U_{yo}A_{y}-HPPO-GalNAc | |
| 1675511 | G_{ys}A_{ys}A_{yo}A_{yo}C_{yo}U_{yo}C_{fo}A_{yo}A_{fz} | 26 |
| | U_{fo}A_{fz}A_{yo}A_{yo}G_{yo}U_{yo}G_{yo}C_{yo}U_{yo} | |
| | U_{yo}U_{yo}A_{y}-HPPO-GalNAc | |
| 1675512 | G_{ys}A_{ys}A_{yo}A_{yo}C_{yo}U_{yo}C_{fo}A_{yo}A_{fo} | 26 |
| | U_{fz}A_{fo}A_{yo}A_{yo}G_{yo}U_{yo}G_{yo}C_{yo}U_{yo} | |
| | U_{yo}U_{yo}A_{y}-HPPO-GalNAc | |

In the table above, a subscript “f” represents a 2′-fluoro-β-D-ribosyl sugar moiety, a subscript “y” represents a 2′-O-methyl-β-D-ribosyl sugar moiety, a subscript “s” represents a phosphorothioate internucleoside linkage, a subscript “o” represents a phosphodiester internucleoside linkage, and a subscript “z” represents a mesyl phosphoramidate internucleoside linkage (Formula II).

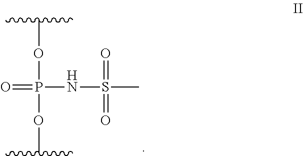 [SMILES: *OP(=O)(NS(C)(=O)=O)O*]

TABLE 184
Design of RNAi compounds targeted to FXII containing
mesyl phosphoramidate internucleoside linkages

| Duplex | Antisense Strand | Sense Strand |
|---|---|---|
| Compound No. | Compound No. | Compound No. |
| 1680994 | 1676701 | 1523578 |
| 1680995 | 1676702 | 1523578 |
| 1680997 | 1678021 | 1523578 |
| 1680998 | 1678022 | 1523578 |
| 1680999 | 1678023 | 1523578 |
| 1681000 | 1678024 | 1523578 |
| 1681001 | 1526195 | 1671536 |
| 1681022 | 1675506 | 1523578 |
| 1681023 | 1675507 | 1523578 |
| 1681026 | 1675508 | 1523578 |
| 1681027 | 1675509 | 1523578 |
| 1681032 | 1626280 | 1675510 |
| 1681034 | 1626280 | 1675511 |
| 1681037 | 1626280 | 1675512 |
| 1681040 | 1675509 | 1675512 |
| 1681041 | 1675506 | 1675510 |

### Example 77: Dose-Dependent Inhibition of FXII in Mouse Hepatocyte Cells by RNAi Compounds

The RNAi agents described above were tested at various doses in mouse hepatocyte cells. Mouse hepatocyte cells at a density of 20,000 cells per well by free uptake with RNAi compound at concentrations indicated in the tables below. After a treatment period of approximately 24 hours, total RNA was isolated from the cells and FXII RNA levels were measured by quantitative real-time RTPCR. Mouse FXII primer-probe set RTS2959 (described herein above) was used to measure RNA levels. FXII RNA levels were normalized to total RNA content, as measured by RIBOGREEN®. Reduction of FXII RNA is presented in the tables below as percent FXII RNA, relative to the amount of FXII RNA in untreated control cells (% UTC). “N.D.” indicates data that was not determined.

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Parent Compound No. 1632812 (described herein above) and parent Compound No. 1523582 (described herein above) were included as a benchmarks.

TABLE 185
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0.001 | 0.0001 | 0.00001 | 0.000001 | IC₅₀ |
| No. | 10 nM | 1 nM | 0.1 nM | nM | nM | nM | nM | nM | (pM) |
| 1526196 | 8 | 8 | 30 | 90 | 103 | 103 | 92 | 97 | 53 |
| 1679413 | 13 | 33 | 75 | 114 | 119 | 106 | 103 | 88 | 465 |
| 1680994 | 8 | 11 | 38 | 102 | 107 | 117 | 107 | 104 | 78 |
| 1680995 | 10 | 10 | 38 | 102 | 121 | 109 | 92 | 98 | 79 |
| 1680996 | 8 | 9 | 21 | 77 | 97 | 110 | 90 | 104 | 31 |
| 1680997 | 10 | 11 | 44 | 95 | 116 | 124 | 110 | 92 | 90 |
| 1680998 | 8 | 10 | 38 | 91 | 96 | 91 | 97 | 98 | 68 |
| 1680999 | 9 | 10 | 38 | 76 | 94 | 90 | 92 | 98 | 49 |
| 1681000 | 9 | 8 | 29 | 78 | 95 | 94 | 85 | 95 | 40 |
| 1681001 | 7 | 7 | 15 | 65 | 82 | 92 | 96 | 91 | 16 |

TABLE 186
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0.001 | 0.0001 | 0.00001 | 0.000001 | IC₅₀ |
| No. | 10 nM | 1 nM | 0.1 nM | nM | nM | nM | nM | nM | (pM) |
| 1632812 | 7 | 13 | N.D. | 111 | 122 | 121 | 95 | 106 | 424 |
| 1681022 | 9 | 29 | 106 | 127 | 126 | 132 | 120 | 99 | 85 |
| 1681023 | 9 | 15 | 85 | 96 | 126 | 111 | 100 | 108 | 317 |
| 1681026 | 9 | 10 | 101 | 122 | 121 | 100 | 98 | 104 | 647 |
| 1681027 | 8 | 13 | 97 | 92 | 123 | 95 | 96 | 106 | 435 |
| 1681032 | 13 | 22 | 97 | 95 | 110 | 105 | 102 | 110 | 524 |
| 1681034 | 14 | 30 | 103 | 89 | 125 | 98 | 97 | 121 | 834 |
| 1681037 | 10 | 29 | 97 | 94 | 85 | 98 | 96 | 104 | 596 |
| 1681040 | 11 | 34 | 92 | 92 | 97 | 89 | 91 | 109 | 598 |
| 1681041 | 24 | 47 | 90 | 87 | 94 | 88 | 92 | 108 | 1177 |

TABLE 187
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0.001 | 0.0001 | 0.00001 | 0.000001 | IC₅₀ |
| No. | 10 nM | 1 nM | 0.1 nM | nM | nM | nM | nM | nM | (pM) |
| 1632812 | 7 | 9 | 59 | 116 | 99 | 110 | 108 | 122 | 117 |
| 1669050 | 7 | 9 | 68 | 98 | 110 | 105 | 109 | 117 | 177 |
| 1669051 | 7 | 9 | 53 | 125 | 133 | 115 | 121 | 95 | 101 |

### Example 78: Design of siRNA Targeted to FXII Containing β-D-Arabinosyl Nucleosides in the Sense Strand

Modified oligonucleotides in the table below having either stereo-standard nucleosides or stereo-non-standard nucleosides in the antisense RNAi oligonucleotides and/or the sense RNAi oligonucleotides were synthesized using standard techniques. Antisense RNAi oligonucleotide Compound No. 1626280 is described herein above.

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Each sense RNAi oligomeric compound further contains a GalNAc moiety conjugated to the 5′ oxygen or the 3′-oxygen as shown below:

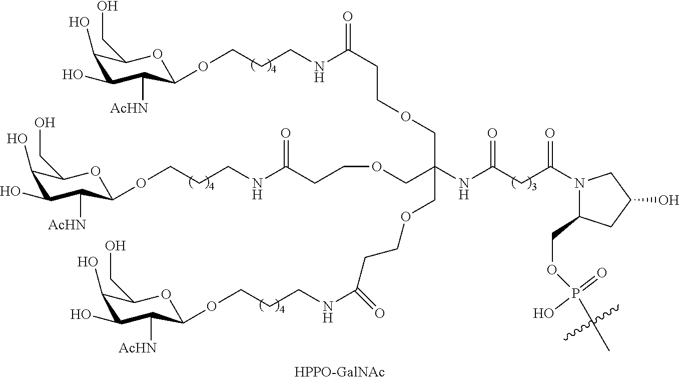

TABLE 188
Design of sense strand modified oligonucleotides
targeted to FXII containing β-D-arabinosyl
nucleosides

| Compound | | SEQ ID |
|---|---|---|
| No. | Chemistry Notation (5′ to 3′) | NO. |
| 1652699 | G_{ys}A_{ys}A_{yo}A_{yo}C_{yo}U_{yo}C_{[bDa]o}A_{yo}A_{fo} | 26 |
| | U_{fo}A_{fo}A_{yo}A_{yo}G_{yo}U_{yo}G_{yo}C_{yo}U_{yo}U_{yo} | |
| | U_{yo}A_{y}-HPPO-GalNAc | |
| 1652700 | G_{ys}A_{ys}A_{yo}A_{yo}C_{yo}U_{yo}C_{fo}A_{yo}A_{[bDa]o} | 26 |
| | U_{fo}A_{fo}A_{yo}A_{yo}G_{yo}U_{yo}G_{yo}C_{yo}U_{yo}U_{yo} | |
| | U_{yo}A_{y}-HPPO-GalNAc | |
| 1652701 | G_{ys}A_{ys}A_{yo}A_{yo}C_{yo}U_{yo}C_{fo}A_{yo}A_{fo}U_{[bDa]o} | 26 |
| | A_{fo}A_{yo}A_{yo}G_{yo}U_{yo}G_{yo}C_{yo}U_{yo}U_{yo}U_{yo} | |
| | A_{y}-HPPO-GalNAc | |
| 1652702 | G_{ys}A_{ys}A_{yo}A_{yo}C_{yo}U_{yo}C_{fo}A_{yo}A_{fo}U_{fo} | 26 |
| | A_{[bDa]o}A_{yo}A_{yo}G_{yo}U_{yo}G_{yo}C_{yo}U_{yo}U_{yo} | |
| | U_{yo}A_{y}-HPPO-GalNAc | 26 |
| 1652703 | G_{ys}A_{ys}A_{yo}A_{yo}C_{yo}U_{yo}C_{[bDa]o}A_{yo}A_{[bDa]o} | |
| | U_{[bDa]o}A_{[bDa]o}A_{yo}A_{yo}G_{yo}U_{yo}G_{yo}C_{yo}U_{yo} | |
| | U_{yo}U_{yo}A_{y}-HPPO-GalNAc | |

In the table above, a subscript “f” represents a 2′-fluoro-β-D-ribosyl sugar moiety, a subscript “y” represents a 2′-O-methyl-β-D-ribosyl sugar moiety, a subscript “[bDa]” represents a β-D-arabinosyl sugar moiety, a subscript “s” represents a phosphorothioate internucleoside linkage, and a subscript “o” represents a phosphodiester internucleoside linkage.

TABLE 189
Design of RNAi compounds targeted
to FXII β-D-arabinosyl nucleosides

| Duplex | Antisense Strand | Sense Strand |
|---|---|---|
| Compound No. | Compound No. | Compound No. |
| 1652867 | 1626280 | 1652702 |
| 1652871 | 1626280 | 1652701 |
| 1652872 | 1626280 | 1652700 |
| 1652873 | 1626280 | 1652699 |
| 1652874 | 1626280 | 1652703 |

### Example 79: Dose-Dependent Inhibition of FXII in Mouse Hepatocyte Cells by RNAi Compounds

The RNAi agents described above were tested at various doses in mouse hepatocyte cells. Mouse hepatocyte cells at a density of 20,000 cells per well by free uptake with RNAi compound at concentrations indicated in the tables below. After a treatment period of approximately 24 hours, total RNA was isolated from the cells and FXII RNA levels were measured by quantitative real-time RTPCR. Mouse FXII primer-probe set RTS2959 (described herein above) was used to measure RNA levels. FXII RNA levels were normalized to total RNA content, as measured by RIBOGREEN®. Reduction of FXII RNA is presented in the tables below as percent FXII RNA, relative to the amount of FXII RNA in untreated control cells (% UTC).

The half maximal inhibitory concentration (IC₅₀) of each RNAi agent was calculated with GraphPad Prism software (v8.2.0, San Diego, CA) using the log (inhibitor) vs. normalized response—Variable slope function: Y=100/(1+10\hat{ }((Log IC50−X)*HillSlope)). Each table represents a separate experiment. Parent Compound No. 1632812 (described herein above) and parent Compound No. 1523582 (described herein above) were included as a benchmarks.

TABLE 190
Dose-dependent reduction of mouse FXII RNA
in mouse hepatocytes by RNAi compounds

| | FXII RNA (% UTC) | | | | | | | | |
|---|---|---|---|---|---|---|---|---|---|
| Compound | | | | 0.01 | 0.001 | 0.0001 | 0.00001 | 0.000001 | IC₅₀ |
| No. | 10 nM | 1 nM | 0.1 nM | nM | nM | nM | nM | nM | (pM) |
| 1632812 | 7 | 9 | 59 | 116 | 99 | 110 | 108 | 122 | 117 |
| 1652867 | 12 | 28 | 90 | 107 | 112 | 113 | 115 | 128 | 514 |
| 1652871 | 7 | 10 | 61 | 108 | 123 | 105 | 102 | 123 | 147 |
| 1652872 | 7 | 15 | 69 | 109 | 115 | 106 | 107 | 127 | 214 |
| 1652873 | 7 | 13 | 76 | 109 | 102 | 99 | 94 | 112 | 242 |
| 1652874 | 9 | 15 | 65 | 110 | 116 | 99 | 95 | 112 | 192 |

### Example 80: Design of siRNA Targeted to FXII

Modified oligonucleotides in the table below having either stereo-standard nucleosides or stereo-non-standard nucleosides in the antisense RNAi oligonucleotides and/or the sense RNAi oligonucleotides were synthesized using standard techniques. The antisense RNAi oligonucleotide described in the table below has the sequence TAAAGCACUUUAUUGAGUUUCUG (SEQ ID NO: 31). Aside from a single mismatch at position 1 on the 5′-end, the sequence is complementary to the complement of GenBank Accession No. NC_000079.6, truncated from nucleosides 55415001 to 55430000, (SEQ ID NO. 25) from nucleosides 12005 to 12026 (SEQ ID NO: 39). Each antisense RNAi oligonucleotide has a 5′-vinylphosphonate. Antisense RNAi oligonucleotides Compound No. 1526195, Compound No. 1666847, Compound No. 1599520 and Compound No. 1670874 are described herein above.

### Table 191: Design of Antisense Strand Modified Oligonucleotides Targeted to FXII

TABLE 191
Design of antisense strand modified oligonucleotides
targeted to FXII

| Compound | | SEQ ID |
|---|---|---|
| No. | Chemistry Notation (5′ to 3′) | NO. |
| 1670873 | vP-TₑₛA_{ds}A_{yo}A_{yo}G_{yo}C_{fo}A_{yo}C_{yo}U_{yo}U_{yO}U_{yo}A_{yo}U_{yo}U_{fo}G_{yo}A_{fo}G_{yo}U_{yo}U_{yo}U_{yo}C_{ys}U_{ys}G_{y} | 31 |
| 1681059 | vP-TₑₛAᵣₛA_{yo}A_{yo}G_{yo}CᵣₛA_{yo}C_{yo}U_{yo}U_{yo}U_{yo}A_{yo}U_{yo}UᵣₛG_{yo}AᵣₛG_{yo}U_{yo}U_{yo}U_{yo}C_{ys}U_{ys}G_{y} | 31 |
| 1681893 | vP-TₑₛA_{dz}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yO}U_{yo}U_{yo}C_{ys}U_{ys}G_{y} | 31 |
| 1681894 | vP-TₑₛA_{fs}A_{yo}A_{yo}G_{yo}C_{az}A_{yo}C_{yo}U_{yo}U_{yo}U_{yo}A_{yo}U_{yo}U_{fo}G_{yo}A_{fo}G_{yo}U_{yo}U_{yo}U_{yo}C_{ys}U_{ys}G_{y} | 31 |
| 1681896 | vP-TₑₛA_{fs}A_{yo}A_{yo}G_{yo}C_{fo}A_{yo}C_{yo}U_{yo}U_{yo}U_{yo}A_{yo}U_{yo}U_{fo}G_{yo}A_{dz}G_{yo}U_{yo}U_{yo}U_{yo}C_{ys}U_{ys}G_{y} | 31 |
| 1685420 | vP-TₑₛA_{[F-HNA]S}A_{yo}A_{yo}G_{yo}C_{fo}A_{yo}C_{yo}U_{yo}U_{yo}U_{yo}A_{yo}U_{yo}U_{fo}G_{yo}A_{fo}G_{yo}U_{yO}U_{yo}U_{yo}C_{ys} | 31 |
| | U_{ys}G_{y} | |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE ribosyl sugar moiety, a subscript “d” represents a 2′-β-D-deoxyribosyl sugar moiety, a subscript “r” represents a β-D-ribosyl sugar moiety sugar moiety, a subscript “[F-HNA]” represents a 3′-fluoro-hexitol sugar surrogate, a subscript “s” represents a phosphorothioate internucleoside linkage, a subscript “o” represents a phosphodiester internucleoside linkage, and a subscript “z” represents a mesyl phosphoramidate internucleoside linkage (Formula II).

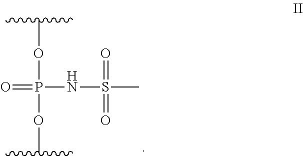 [SMILES: *OP(=O)(NS(C)(=O)=O)O*]

The antisense RNAi oligonucleotides described in the table below have the sequence TAAAGCACUUUAUTGAGUUUCUG (SEQ ID NO: 44). Aside from a single mismatch at position 1 on the 5′-end, each antisense RNAi oligonucleotide is 100% complementary to the complement of GenBank Accession No. NC_000079.6, truncated from nucleosides 55415001 to 55430000, (SEQ ID NO: 25) from nucleosides 12005 to 12026 (SEQ ID NO: 39). Each antisense RNAi oligonucleotide has a 5′-vinylphosphonate.

TABLE 192
Design of antisense strand modified
oligonucleotides targeted to FXII

| Compound | | SEQ ID |
|---|---|---|
| No. | Chemistry Notation (5′ to 3′) | NO. |
| 1681898 | vP-TₑₛA_{dz}A_{yo}A_{yo}G_{yo}C_{dz}A_{yo}C_{yo}U_{yo} | 44 |
| | U_{yo}U_{yo}A_{yo}U_{yo}T_{dz}G_{yo}A_{dz}G_{yo}U_{yo}U_{yo} | |
| | U_{yo}C_{ys}U_{ys}G_{y} | |
| 1681899 | vP-TₑₛA_{fs}A_{yo}A_{yo}G_{yo}C_{fo}A_{yo}C_{yo}U_{yo} | 44 |
| | U_{yo}U_{yo}A_{yo}U_{yo}T_{az}G_{yo}A_{fo}G_{yo}U_{yo}U_{yo} | |
| | U_{yo}C_{ys}U_{ys}G_{y} | |

In the table above, a subscript “f” represents a 2′-fluoro-β-D-ribosyl sugar moiety, a subscript “y” represents a 2′-O-methyl-β-D-ribosyl sugar moiety, a subscript “e” represents a 2′-MOE ribosyl sugar moiety, a subscript “d” represents a 2′-β-D-deoxyribosyl sugar moiety, a subscript “r” represents a β-D-ribosyl sugar moiety sugar moiety, a subscript “[F-HNA]” represents a 3′-fluoro-hexitol sugar surrogate, a subscript “s” represents a phosphorothioate internucleoside linkage, a subscript “o” represents a phosphodiester internucleoside linkage, and a subscript “z” represents a mesyl phosphoramidate internucleoside linkage (Formula II).

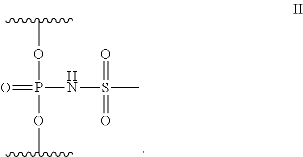 [SMILES: *OP(=O)(NS(C)(=O)=O)O*]

The sense RNAi oligonucleotide is complementary to the first 21 nucleosides of the antisense RNAi oligonucleotide (from 5′ to 3′) wherein the last two 3′-nucleosides of the antisense RNAi oligonucleotides are not paired with the sense oligonucleotide (are overhanging nucleosides). Sense RNAi oligonucleotide Compound No. 1523578 is described herein above.

TABLE 193
Design of RNAi compounds targeted to FXII

| Duplex | Antisense Strand | Sense Strand |
|---|---|---|
| Compound No. | Compound No. | Compound No. |
| 1685421 | 1685420 | 1523578 |
| 1686728 | 1681893 | 1523578 |
| 1686729 | 1681894 | 1523578 |
| 1686730 | 1681899 | 1523578 |
| 1686731 | 1681896 | 1523578 |
| 1686732 | 1681898 | 1523578 |
| 1686737 | 1681059 | 1523578 |
| 1679589 | 1670873 | 1523578 |
| 1685422 | 1526195 | 1675510 |
| 1685425 | 1526195 | 1675511 |
| 1685427 | 1666847 | 1675510 |
| 1687205 | 1599520 | 1670885 |
| 1687206 | 1599520 | 1657708 |
| 1686738 | 1670874 | 1670886 |

### Example 81: Activity of siRNA Targeted to Mouse FXII in Wild-Type Mice, In Vivo Single Dose

RNAi agents described above were tested in wild-type C57BL/6 male mice (Jackson Laboratory). The mice were divided into groups of 3 mice each. Each mouse received a single subcutaneous injection of RNAi agent at a dose of 1 mg/kg. A group of 4 mice received PBS as a negative control.

### RNA Analysis

Two weeks post treatment, mice were sacrificed. RNA was extracted from liver tissue for quantitative real-time RTPCR analysis of RNA expression of mouse FXII using primer probe set RTS2959 (described herein above). Results are presented as percent change of mouse FXII RNA, relative to the amount of FXII RNA in PBS control treated mice, normalized to RIBOGREEN (% control).

TABLE 194
Reduction of mouse FXII RNA by siRNA in
wild type C57BL/6 mice after 2 weeks

| Compound Number | FXII RNA (% control) |
|---|---|
| 1526196 | 11 |
| 1685421 | 10 |
| 1686728 | 16 |
| 1686729 | 13 |
| 1686730 | 16 |
| 1686731 | 12 |
| 1686732 | 83 |
| 1686737 | 42 |
| 1679413 | 18 |
| 1679589 | 14 |
| 1679590 | 49 |
| 1675710 | 22 |
| 1679592 | 10 |
| 1679591 | 13 |
| 1680994 | 13 |
| 1680995 | 14 |
| 1680996 | 15 |
| 1680997 | 18 |
| 1680998 | 13 |
| 1680999 | 22 |
| 1681000 | 12 |
| 1681001 | 41 |
| 1685422 | 16 |
| 1685425 | 15 |
| 1685427 | 27 |
| 1687205 | 101‡ |
| 1687206 | 18 |
| 1686738 | 107 |

‡indicates that fewer than 3 samples were available

### Plasma Protein Analysis

Mouse FXII protein levels in plasma were determined using a Molecular Innovations FXII ELISA kit (catalog number: MFXIIKT-TOT). The data is presented as concentration of mouse FXII protein, in μg/mL.

TABLE 195
Reduction of mouse FXII protein by siRNA
in wild type C57BL/6 mice after 2 weeks

| | Plasma FXII Protein |
|---|---|
| Compound Number | (μg/mL) |
| PBS | 18 |
| 1526196 | 1 |
| 1685421 | 1 |
| 1686728 | 2 |
| 1686729 | 1 |
| 1686730 | 2 |
| 1686731 | 1 |
| 1686732 | 15 |
| 1686737 | 6 |
| 1679413 | 3 |
| 1679589 | 1 |
| 1679590 | 8 |
| 1675710 | 2 |
| 1679592 | 1 |
| 1679591 | 1 |
| 1680994 | 1 |
| 1680995 | 1 |
| 1680996 | 2 |
| 1680997 | 2 |
| 1680998 | 1 |
| 1680999 | 3 |
| 1681000 | 1 |
| 1681001 | 7 |
| 1685422 | 2 |
| 1685425 | 1 |
| 1685427 | 4 |
| 1687205 | 18‡ |
| 1687206 | 2 |
| 1686738 | 15 |

‡indicates that fewer than 3 samples were available

1.-25. (canceled)

26. An RNAi agent, comprising an antisense RNAi oligomeric compound comprising an antisense RNAi oligonucleotide consisting of 21-25 linked nucleosides and a sense RNAi oligomeric compound comprising a sense RNAi oligonucleotide consisting of 19-23 linked nucleosides, wherein the antisense RNAi oligonucleotide comprises a 5′-stabilized phosphate group; and at least one internucleoside linkage of Formula I: 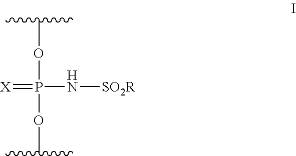
wherein independently for each internucleoside linkage of Formula I:
X is selected from O or S, and
R is selected from aryl, a substituted aryl, a heterocycle, a substituted heterocycle, an aromatic heterocycle, a substituted aromatic heterocycle, a diazole, a substituted diazole, a C₁-C₆ alkoxy, C₁-C₂₀ alkyl, C₂-C₆ alkenyl, C₂-C₆ alkynyl, substituted C₁-C₂₀ alkyl, substituted C₂-C₆ alkenyl substituted C₂-C₆ alkynyl, and a conjugate group; and
wherein each of at least three sugar moieties of the nucleosides of the antisense RNAi oligonucleotide is selected from a 2′-O-methyl-β-D-ribosyl sugar moiety, a 2′-O-methoxyethyl β-D-ribosyl sugar moiety, a sugar surrogate, a stereo-non-standard sugar moiety, a 2′-β-D-deoxyribosyl sugar moiety, a β-D-ribosyl sugar moiety, and 2′-fluoro-β-D-ribosyl sugar moiety, and wherein each of at least three such sugar moieties are different from one another.

27. The RNAi agent of claim 26, wherein for at least one internucleoside linkage of Formula I, X is O and R is methyl.

28. The RNAi agent of any of claim 26, wherein for each internucleoside linkage of Formula I, X is O and R is methyl.

29. The RNAi agent of claim 26, wherein each internucleoside linkage of the antisense RNAi oligonucleotide is selected from an internucleoside linkage of Formula I, a phosphodiester internucleoside linkage, and a phosphorothioate internucleoside linkage.

30. The RNAi agent of claim 26, wherein the antisense RNAi oligonucleotide has an internucleoside linkage motif selected from sz(o)ₙzs, ss(o)ₙzs, and sz(o)ₙss, wherein each “s” is a phosphorothioate internucleoside linkage, each “z” is an internucleoside linkage of Formula I, each “o” is a phosphodiester internucleoside linkage, and n is from 16-20.

31. The RNAi agent of claim 26, wherein the antisense RNAi oligonucleotide consists of 23 linked nucleosides.

32. The RNAi agent of claim 31, wherein the antisense RNAi oligonucleotide has an internucleoside linkage motif selected from zsooooooooooooooooooss, szooooooooooooooooooss, zzooooooooooooooooooss, zzoooooooooooooooooozs, ssoooozoooooooooooooss, ssoooooooooooooooooozz, zsoooooooooooooooooozz, zsooozoooooooooooooozz, ssoooooooooooooooooosz, ssoooooooooooooooooozs, szoooooooooooooooooosz, zsoooooooooooooooooosz, zsoooooooooooooooooozs, szoooooooooooooooooozs, szooozooooooozozooooss, ssooozooooooozozooooss, ssooooooooooozozooooss, szooooooooooozooooooss, zoooooooooooooooooooss, szoooooooooooooooooozz, ssooosooooooososooooss, ssooozooooooooooooooss, ssooooooooooooozooooss, ssooooooooooozooooooss wherein each “s” is a phosphorohioate internucleoside linkage, each “z” is an internucleoside linkage of Formula I or of Formula XIV, and each “o” is a phosphodiester internucleoside linkage.

33. The RNAi agent of claim 32, wherein for each internucleoside linkage of Formula I, X is O and R is methyl.

34. The RNAi agent of claim 26, wherein each stereo-non-standard sugar moiety is independently selected from a 2′-fluoro-β-D-xylosyl sugar moiety, a 2′-fluoro-β-D-arabinosyl sugar moiety, 2′-O-methyl-β-D-xylosyl sugar moiety, and a 2′-β-D-deoxyxylosyl sugar moiety.

35. The RNAi agent of claim 26, wherein each sugar surrogate is independently selected from fluoro hexitol nucleic acid (F-HNA), hexitol nucleic acid (HNA) or altritol nucleic acid (ANA).

36. The RNAi agent of claim 26, wherein the antisense RNAi oligonucleotide comprises no more than three 2′-fluoro-β-D-ribosyl sugar moieties.

37.-45. (canceled)

46. The RNAi agent of claim 31, wherein the antisense RNAi oligonucleotide has a sugar motif selected from: y[f2bDx]yyyfyyyyyyyfyfyyyyyyy, yfyyy[f2bDx]yyyyyyyfyfyyyyyyy, yfyyyfyyyyyyy[f2bDx]yfyyyyyyy, yfyyyfyyyyyyyfy[f2bDx]yyyyyyy, y[f2bDx]yyy[f2bDx]yyyyyyy[f2bDx]y[f2bDx]yyyyyyy, efyyyfy[16C₂r]yyyyyfyfyyyyyyy, e[f2bDx]yyyfyyyyyyyfyfyyyyyyy, efyyy[f2bDx]yyyyyyyfyfyyyyyyy, efyyyfyyyyyyy[f2bDx]yfyyyyyyy, efyyyfyyyyyyyfy[f2bDx]yyyyyyy, e[f2bD) x]yyy[f2bDx]yyyyyyy[f2bDx]y[f2bDx]yyyyyyy, efyyy[F-HNA]yyyyyyyfyfyyyyyyy, efyyyfyyyyyyy[F-HNA]yfyyyyyyy, efyyyfyyyyyyyfy[F-HNA]yyyyyyy, yfyyydyyyyyyyfyfyyyyyyy, yfyyyfyyyyyyydyfyyyyyyy, yfyyyfyyyyyyyfydyyyyyyy, yfyyydyyyyyyydydyyyyyyy, yryyyfyyyyyyyfyfyyyyyyy, yfyyyyyyyyyyfyfyyyyyyy, yfyyyfyyyyyyyryfyyyyyyy, yfyyyfyyyyyyyfyryyyyyyy, yryyyryyyyyyyryryyyyyyy, y[bDdx]yyyfyyyyyyyfyfyyyyyyy, yfyyy[bDdx]yyyyyyyfyfyyyyyyy, yfyyyfyyyyyyy[bDdx]yfyyyyyyy, yfyyyfyyyyyyyfy[bDdx]yyyyyyy, y[bDdx]yyy[bDdx]yyyyyyy[bDdx]y[bDdx]yyyyyyy, y[F-HNA]yyyfyyyyyyyfyfyyyyyyy, yfyyy[F-HNA]yyyyyyyfyfyyyyyyy, yfyyyfyyyyyyy[F-HNA]yfyyyyyyy, yfyyyfyyyyyyyfy[F-HNA]yyyyyyy, y[F-HNA]yyy[F-HNA]yyyyyyy[F-HNA]y[F-HNA]yyyyyyy, yfyyyfyyyyyyy[2bDx]yfyyyyyyy, y[bDa]yyyfyyyyyyyfyfyyyyyyy, yfyyy[bDa]yyyyyyyfyfyyyyyyy, yfyyyfyyyyyyy[bDa]yfyyyyyyy, yfyyyfyyyyyyyfy[bDa]yyyyyyy, y[bDa]yyy[bDa]yyyyyyy[bDa]y[bDa]yyyyyyy, yfyyyfyyyyyyy[bDx]yfyyyyyyy, yfyyyfyffyyyyfyfyyyyydd, yfyyyfyffyyyyfyfyyyyy[bLdr][bLdr], yfyyyfyffyyyyfyfyyyyy[aDdr][aDdr], yfyyyfyffyyyyfyfyyyyy[bDdx][bDdx], yfyyyfyffyyyyfyfyyyyy[bLdx][bLdx], yfyyyfyffyyyyfyfyyyyy[aDdx][aDdx], yfyyyfyffyyyyfyfyyyyy[aLdx][aLdx], yfyyyfyyyyyyyfyfyyyyyee, yfyyyfyyyyyyyfyfyyyyykk, yfyyyfyyyyyyyfyfyyyyy[LNA][LNA], yfyyyfyyyyyyyfyfyyyyy[F-HNA][F-HNA], [ANA]fyyyfyyyyyyyfyfyyyyyyy, y[ANA]yyyfyyyyyyyfyfyyyyyyy, yfyyyf[ANA]yyyyyyfyfyyyyyyy, yfyyyfyy[ANA]yyyyfyfyyyyyyy, yfyyyfyyyyyyy[ANA]yfyyyyyyy, yfyyyfyyyyyyyfyf[ANA]yyyyyy, yfyyyfyy[ANA]yyyy[ANA]yf[ANA]yyyyy[ANA], yfyyyfyyyyyyyfyfyyyyyyk, yfyyyfyyyyyyyfyfyyyyyke, yfyyyfyyyyyyyfyfyyyyyky, efyyydyyyyyyydydyyyyyyy, yfyyyfyyyyyyy[ANA]yf[ANA]yyyyyy, yfyyyfyyyyyyyfyfyyyyy[ANA]y, yfyyyfyyyyyyyfyfyyyyy[f2bDa][f2bDa], yfyyyfyyyyyyyfyfyyyyynn, yfyyyfyyyyyyyfyfyyyyy[DMAEOE][DMAEOE], yfyyyfyyyyyyyfyfyyyyy[HNA][HNA], yfyyyfyyyyyyyfyfyyyyy[SM5LNA][SM5LNA], yfyyyfyyyyyyyfyfyyyyy[DMAOE][DMAOE], yfyyyfyyyyyyyfyfyyyyydd, yfyyyfyyyyyyyfyfyyyyy[aLdr][aLdr], [HNA]fyyyfyyyyyyyfyfyyyyyyy, dfyyyfyyyyyyyfyfyyyyyyy, y[HNA]yyyfyyyyyyyfyfyyyyyyy, e[HNA]yyyfyyyyyyyfyfyyyyyyy, yfyyyf[HNA]yyyyyyfyfyyyyyyy, yfyyyfyy[HNA]yyyyfyfyyyyyyy, yfyyyfyyyyyyy[HNA]yfyyyyyyy, yfyyyfyyyyyyyfyf[HNA]yyyyyy, yfyyyfyy[HNA]yyyy[HNA]yf[HNA]yyyyy[HNA], yfyyyfyyyyyyy[HNA]yf[HNA]yyyyyy, yfyyyfyyyyyyyfyfyyyyy[HNA]y, kfyyyfyyyyyyyfyfyyyyyyy, e[F-HNA]yyyfyyyyyyyfyfyyyyyyy, e[LNA]yyyfyyyyyyyfyfyyyyyyy, edyyyfyyyyyyyfyfyyyyyyy, edyyydyyyyyyydydyyyyyyy, ydyyyfyyyyyyyfyfyyyyyyy, ydyyydyyyyyyydydyyyyyyy, efyyfyfyyyyfyfyyyyyyyyy, edyydydyyyydyfyyyyyyyyy, efyyyfyyyyyyyfyfyyyyydd, [F-HNA]fyyyfyyyyyyyfyfyyyyyyy, eyyyyfyyyyyyyfyfyyyyyyy, eryyyryyyyyyyryryyyyyyy, efyyydyyyyyyyfyfyyyyyyy, efyyyfyyyyyyyfydyyyyyyy, efyyyfyyyyyyydyfyyyyyyy; wherein “f” represents a 2′-fluoro-β-D-ribosyl sugar moiety, “y” represents a 2′-O-methyl-β-D-ribosyl sugar moiety, “e” represents a 2′-O-methyoxyethyl-β-D-ribosyl sugar moiety, “d” represents a 2′-β-D-deoxyribosyl sugar moiety, “r” represents a β-D-ribosyl sugar moiety, “[f2bDx]” represents a 2′-fluoro-β-D-xylosyl sugar moiety, “[16C₂r]” represents a 2′-O-hexadecyl-β-D-ribosyl sugar moiety, “[F-HNA]” represents 3′-fluoro-hexitol sugar surrogate, “[bDdx]” represents a 2′-β-D-deoxyxylosyl sugar moiety, “[bDx]” represents a β-D-xylosyl sugar moiety, “[bDa]” represents a β-D-arabinosyl sugar moiety, “[bLdr]” represents a 2′-β-L-deoxyribosyl sugar moiety, “[aDdr]” represents a 2′-α-D-deoxyribosyl sugar moiety, “[aLdr]” represents a 2′-α-L-deoxyribosyl sugar moiety, “[bLdx]” represents a 2′-β-L-deoxyxylosyl sugar moiety, “[aDdx]” represents an 2′-α-D-deoxyxylosyl sugar moiety, “[aLdx]” represents an 2′-α-L-deoxyxylosyl sugar moiety, “[LNA]” represents a β-D-LNA sugar moiety, “[f2bDa]” represents a 2′-fluoro-β-D-arabionsyl sugar moiety, “n” represents a 2′-O—(N-methylacetamide) ribosyl sugar moiety, “[DMAEOE]” represents a 2′-O-(2-(2-(N,N-dimethyl)aminoethoxy)ethyl)-β-D-ribosyl sugar moiety, “[SM5LNA]” represents a (5'S)-5′methyl-LNA sugar moiety, “[ANA]” represents an ANA sugar surrogate, and “[HNA]” represents an HNA sugar surrogate.

47.-211. (canceled)

212. The RNAi agent of claim 26, wherein each nucleobase of the antisense RNAi oligonucleotide is selected from uracil, thymine, guanine, adenine, cytosine, and 5-methylcytosine.

213. The RNAi agent of claim 26, wherein each nucleobase of the sense RNAi oligonucleotide is selected from uracil, thymine, guanine, adenine, cytosine, and 5-methylcytosine.

214. A chirally enriched population of RNAi agents of claim 26, wherein the population is enriched for antisense RNAi oligonucleotides and/or sense RNAi oligonucleotides comprising at least one particular internucleoside linkage having a particular stereochemical configuration.

215. The chirally enriched population of claim 214, wherein the population is enriched for antisense RNAi oligonucleotides and/or sense RNAi oligonucleotides comprising at least one particular internucleoside of Formula I having the (Sp) or (Rp) configuration.

216.-221. (canceled)

222. A population of RNAi agents of claim 26, wherein each internucleoside linkage of the antisense RNAi oligonucleotide and the sense RNAi oligonucleotide is stereorandom.

223. A method comprising administering at least two doses of an RNAi agent of claim 26 to an animal wherein:
the RNAi agent is administered to the animal at a dose frequency of once per 4 months, 5 months, 6 months, 7 months, 8 months, 9 months, 10 months, 11 months, or a year or more than a year.
